(19) United States
(12) Patent Application Publication
(10) Pub. No.: US 2023/0270859 A1
(43) Pub. Date: Aug. 31, 2023

(54) COMPOSITIONS AND METHODS FOR TREATING LUPUS

(71) Applicant: MONASH UNIVERSITY, Clayton, Victoria (AU)

(72) Inventors: Joshua OOI, Melbourne (AU); Peter EGGENHUIZEN, Melbourne (AU); Eric MORAND, Melbourne (AU)

(21) Appl. No.: 17/906,601

(22) PCT Filed: Mar. 19, 2021
(86) PCT No.: PCT/AU2021/050254

§ 371 Date: Sep. 16, 2022

(30) Foreign Application Priority Data
Mar. 20, 2020 (AU) 2020900864

(51) Int. Cl. A61K 39/00; C07K 14/725; C07K 16/28; A61P 37/06

(52) U.S. Cl. CPC A61K 39/4632; C07K 14/7051; C07K 16/2833; A61K 39/4611; A61P 37/06; C07K 2319/00; A61K 2239/13

(57) ABSTRACT

The present invention relates to compositions and methods for the treatment of lupus, particularly systemic lupus erythematosus. The present invention involves, amongst other things, a binding protein comprising a T cell receptor (TCR) α-chain variable (Vα or Valpha) domain and a TCR β-chain variable (Vβ or Vbeta) domain, wherein the binding protein is capable of binding to a complex of a fragment of a Smith protein and an HLA-DR15 or HLA-DR3 molecule.

### FIELD OF THE INVENTION

The present invention relates to compositions and methods for the treatment of lupus, particularly systemic lupus erythematosus.

### RELATED APPLICATION

This application claims priority from Australian provisional application AU 2020900864, the entire contents of which are hereby incorporated by reference.

### BACKGROUND OF THE INVENTION

Systemic lupus erythematosus (SLE) is a chronic, inflammatory autoimmune disease characterized by the production of autoantibodies having specificity for a wide range of self-antigens. SLE autoantibodies mediate organ damage by directly binding to host tissues and by forming immune complexes that deposit in vascular tissues and activate immune cells. Organs targeted in SLE include the skin, kidneys, vasculature, joints, mucosal and serosal membranes, various blood elements, and the central nervous system (CNS). The severity of disease, the spectrum of clinical involvement, and the response to therapy vary widely among patients. This clinical heterogeneity makes it challenging to diagnose and manage lupus.

Due to the great clinical diversity and idiopathic nature of SLE, management of idiopathic SLE depends on its specific manifestations and severity. Therefore, medications suggested to treat SLE generally are not necessarily effective for the treatment of all manifestations of and complications resulting from SLE, e.g., Lupus nephritis (LN). LN usually arises early in the disease course, within 5 years of diagnosis. The pathogenesis of LN is believed to derive from deposition of immune complexes in the kidney glomeruli that initiates an inflammatory response. An estimated 30-50% of patients with SLE develop nephritis that requires medical evaluation and treatment. LN is a progressive disease, running a course of clinical exacerbations and remissions.

Despite significant research into SLE, effective targeted therapies in SLE are lacking. Present treatments such as corticosteroids, methotrexate, hydroxycholorquine, other immunosuppressants (e.g., ciclosporin, leflunamide, azathioprine, to name a few), and non-steroidal anti-inflammatory drugs, non-specifically inhibit activation of the immune system rather than precisely inhibiting the specific autoimmunity associated with the disorder.

While many patients fail to respond or respond only partially to the standard of care medications listed above, the long-term use of high doses of corticosteroids and cytotoxic therapies may have profound side effects such as bone marrow depression, increased infections with opportunistic organisms, irreversible ovarian failure, alopecia and increased risk of malignancy. Infectious complications coincident with active SLE and its treatment with immunosuppressive medications are among the most common cause of death in patients with SLE.

Therefore, there is a need for new or improved treatments for SLE.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a binding protein comprising a T cell receptor (TCR) α-chain variable (Vα or Valpha) domain and a TCR β-chain variable (Vβ or Vbeta) domain, wherein the binding protein is capable of binding to a complex of a fragment of a Smith protein and an HLA-DR15 or HLA-DR3 molecule. Preferably, the HLA-DR15 molecule is an HLA-DRA*01:01 and HLA-DRB1*15:01 molecule. Preferably, the HLA-DR3 is an HLA-DRA*01:01 and HLA-DRB1*03:01 molecule.

For example, the binding protein of the invention may bind to a peptide consisting of 4, 5, 6, 7, 8, 9, 10 or more contiguous amino acid residues of the sequence as set out in any one of SEQ ID NOs: 1, 2, 3, 4, 258 or 259.

In any aspect, the fragment of the Smith protein that is capable of forming a complex with a HLA-DR15 molecule comprises or consists of an amino acid sequence of, or equivalent to, residues 6 to 14 or 62 to 70 of a SmB/B′ protein, preferably the SmB′ protein comprises the sequence of SEQ ID NO: 5. In one embodiment, the fragment of the SmB/B′ protein comprises or consists of the amino acid sequence of SEQ ID NO: 3 or 4.

In any aspect, the fragment of the Smith protein that is capable of forming a complex with a HLA-DR15 molecule comprises or consists of an amino acid sequence of, or equivalent to, residues 1 to 15 of a SmB/B′ protein, preferably the SmB′ protein comprises the sequence of SEQ ID NO: 5. In one embodiment, the fragment of the SmB/B′ protein comprises or consists of the amino acid sequence of SEQ ID NO: 1.

In any aspect, the fragment of the Smith protein that is capable of forming a complex with a HLA-DR15 molecule comprises or consists of an amino acid sequence of, or equivalent to, residues 58 to 72 of a SmB/B′ protein, preferably the SmB′ protein comprises the sequence of SEQ ID NO: 5. In one embodiment, the SmB/B′ protein comprises the amino acid sequence of SEQ ID NO: 2.

In any aspect, the fragment of the Smith protein that is capable of forming a complex with a HLA-DR3 molecule comprises or consists of an amino acid sequence of, or equivalent to, residues 78 to 92 of a SmD1 protein, preferably the SmD1 protein comprises the sequence of SEQ ID NO: 260. In one embodiment, the fragment of the SmD1 protein comprises or consists of the amino acid sequence of SEQ ID NO: 258.

In any aspect, the fragment of the Smith protein that is capable of forming a complex with a HLA-DR3 molecule comprises or consists of an amino acid sequence of, or equivalent to, residues 7-21 of a SmB/B′ protein, preferably the SmB′ protein comprises the sequence of SEQ ID NO: 5. In one embodiment, the fragment of the SmB/B′ protein comprises or consists of the amino acid sequence of SEQ ID NO: 259.

In any aspect, the fragment of the Smith protein that is capable of forming a complex with a HLA-DR15 molecule comprises or consists of an amino acid sequence of any one or more of SEQ ID Nos: 1 to 4.

In any aspect, the fragment of the Smith protein that is capable of forming a complex with a HLA-DR3 molecule comprises or consists of an amino acid sequence of any one or more of SEQ ID Nos: 258 or 259.

In another aspect, the present invention also provides a binding protein comprising a T cell receptor (TCR) α-chain variable (Vα or Valpha) domain and a TCR β-chain variable (Vβ or Vbeta) domain, wherein the Vα domain comprises an amino acid sequence of any “CDR alpha” or any Vα domain (TRA) as defined in any one of Tables 1 to 4, herein; and/or wherein the Vβ domain comprises an amino acid sequence of any “CDR beta” or any Vβ domain (TRB) as defined in any one of Tables 1 to 4, herein.

In another aspect, the present invention also provides a binding protein comprising a T cell receptor (TCR) α-chain variable (Vα or Valpha) domain and a TCR β-chain variable (Vβ or Vbeta) domain,
wherein the Vα domain comprises a CDR3 comprising an amino acid sequence at least about 80%, at least 85%, at least 90%, at least 92%, at least 95%, at least 97%, or at least 99% identical to a sequence of any one of SEQ ID Nos: 8, 20, 32, 44, 56, 68, 80, 92, 104, 107, 122, 134, 146, 158, 170, 182, 194, 197, 212, 224, 236 and 248; and/or
wherein the Vβ domain comprises a CDR3 comprising an amino acid sequence at least about 80%, at least 85%, at least 90%, at least 92%, at least 95%, at least 97%, or at least 99% identical to a sequence of any one of SEQ ID Nos: 11, 23, 35, 47, 59, 71, 83, 95, 110, 125, 137, 149, 161, 173, 185, 200, 215, 227, 239 and 251;
wherein the binding protein is capable of binding to a complex of a fragment of a Smith protein and a HLA-DR15 molecule.

In another aspect, the present invention also provides a binding protein comprising a T cell receptor (TCR) α-chain variable (Vα or Valpha) domain and a TCR β-chain variable (Vβ or Vbeta) domain,
wherein the Vα domain comprises a CDR3 comprising an amino acid sequence at least about 80%, at least 85%, at least 90%, at least 92%, at least 95%, at least 97%, or at least 99% identical to a sequence of any one of SEQ ID Nos: 263, 275, 287, 299, 311, 323, 335, 347, 359, 371, 383, 395, 407, 419, 431, 443, 455, 467, 479 and 491; and/or
wherein the Vβ domain comprises a CDR3 comprising an amino acid sequence at least about 80%, at least 85%, at least 90%, at least 92%, at least 95%, at least 97%, or at least 99% identical to a sequence of any one of SEQ ID Nos: 266, 278, 290, 302, 314, 326, 338, 350, 362, 374, 386, 398, 410, 422, 434, 446, 458, 470, 482 and 494;
wherein the binding protein is capable of binding to a complex of a fragment of a Smith protein and a HLA-DR3 molecule.

In another aspect, the present invention also provides a binding protein comprising a T cell receptor (TCR) α-chain variable (Vα or Valpha) domain and a TCR β-chain variable (Vβ or Vbeta) domain,
wherein the Vα domain comprises a CDR3 comprising an amino acid sequence of any one of SEQ ID Nos: 8, 20, 32, 44, 56, 68, 80, 92, 104, 107, 122, 134, 146, 158, 170, 182, 194, 197, 212, 224, 236 and 248; and/or
wherein the Vβ domain comprises a CDR3 comprising an amino acid sequence of any one of SEQ ID Nos: 11, 23, 35, 47, 59, 71, 83, 95, 110, 125, 137, 149, 161, 173, 185, 200, 215, 227, 239 and 251,
wherein the binding protein is capable of binding to a complex of a fragment of a Smith protein and a HLA-DR15 molecule.

In particularly preferred embodiments, the Vα domain comprises a CDR3 comprising an amino acid sequence of any one of SEQ ID NOs: 8, 20 or 32 and the Vβ domain comprises a CDR3 comprising an amino acid sequence of any one of SEQ ID NOs: 11, 23, or 35.

In another aspect, the present invention also provides a binding protein comprising a T cell receptor (TCR) α-chain variable (Vα or Valpha) domain and a TCR β-chain variable (Vβ or Vbeta) domain,
wherein the Vα domain comprises a CDR3 comprising an amino acid sequence of any one of SEQ ID Nos: 263, 275, 287, 299, 311, 323, 335, 347, 359, 371, 383, 395, 407, 419, 431, 443, 455, 467, 479 and 491; and/or
wherein the Vβ domain comprises a CDR3 comprising an amino acid sequence of any one of SEQ ID Nos: 266, 278, 290, 302, 314, 326, 338, 350, 362, 374, 386, 398, 410, 422, 434, 446, 458, 470, 482 and 494;
wherein the binding protein is capable of binding to a complex of a fragment of a Smith protein and a HLA-DR3 molecule.

In particularly preferred embodiments, the Vα domain comprises a CDR3 comprising an amino acid sequence of SEQ ID NO: 263 and the Vβ domain comprises a CDR3 comprising an amino acid sequence of SEQ ID NOs: 266.

In another aspect, the present invention also provides a binding protein comprising a T cell receptor (TCR) α-chain variable (Vα or Valpha) domain and a TCR β-chain variable (Vβ or Vbeta) domain,
wherein the T cell receptor (TCR) α-chain variable (Vα or Valpha) domain comprises:
(i) a complementarity determining region (CDR) 1 comprising a sequence at least about 80%, at least 85%, at least 90%, at least 92%, at least 95%, at least 97%, at least 99% identical to a sequence set forth in SEQ ID NO: 6, 18, 30, 42, 54, 66, 78, 90, 102, 105, 120, 132, 144, 156, 168, 180, 192, 195, 210, 222, 234 or 246; a CDR2 comprising a sequence at least about 80%, at least 85%, at least 90%, at least 92%, at least 95%, at least 97%, at least 99% identical to a sequence set in SEQ ID NO: 7, 19, 31, 43, 55, 67, 79, 91, 103, 106, 121, 133, 145, 157, 169, 181, 193, 196, 211, 223, 235 or 247; and a CDR3 comprising a sequence at least about 80%, at least 85%, at least 90%, at least 92%, at least 95%, at least 97%, at least 99% identical to a sequence set forth in SEQ ID NO: 8, 20, 32, 44, 56, 68, 80, 92, 104, 107, 122, 134, 146, 158, 170, 182, 194, 197, 212, 224, 236 or 248; or
(ii) a CDR1 comprising a sequence set forth in SEQ ID NO: 6, 18, 30, 42, 54, 66, 78, 90, 102, 105, 120, 132, 144, 156, 168, 180, 192, 195, 210, 222, 234 or 246, a CDR2 comprising a sequence set forth between in SEQ ID NO: 7, 19, 31, 43, 55, 67, 79, 91, 103, 106, 121, 133, 145, 157, 169, 181, 193, 196, 211, 223, 235 or 247 and a CDR3 comprising a sequence set forth in SEQ ID NO: 8, 20, 32, 44, 56, 68, 80, 92, 104, 107, 122, 134, 146, 158, 170, 182, 194, 197, 212, 224, 236 or 248;
and
wherein the TCR β-chain variable (Vβ or Vbeta) domain comprises:
(i) a CDR1 comprising a sequence at least about 80%, at least 85%, at least 90%, at least 92%, at least 95%, at least 97%, at least 99% identical to a sequence set forth in SEQ ID NO: 9, 21, 33, 45, 57, 69, 81, 93, 108, 123, 135, 147, 159, 171, 183, 198, 213, 225, 237 or 249, a CDR2 comprising a sequence at least about 80%, at least 85%, at least 90%, at least 92%, at least 95%, at least 97%, at least 99% identical to a sequence set forth in SEQ ID NO: 10, 22, 34, 46, 58, 70, 82, 94, 109, 124, 136, 148, 160, 172, 184, 199, 214, 226, 238 or 250 and a CDR3 comprising a sequence at least about 80%, at least 85%, at least 90%, at least 92%, at least 95%, at least 97%, at least 99% identical to a sequence set forth in SEQ ID NOs: 11, 23, 35, 47, 59, 71, 83, 95, 110, 125, 137, 149, 161, 173, 185, 200, 215, 227, 239 or 251; or
(ii) a CDR1 comprising a sequence set forth in SEQ ID NO: 9, 21, 33, 45, 57, 69, 81, 93, 108, 123, 135, 147, 159, 171, 183, 198, 213, 225, 237 or 249, a CDR2 comprising a sequence set forth in SEQ ID NO: 10, 22, 34, 46, 58, 70, 82, 94, 109, 124, 136, 148, 160, 172, 184, 199, 214, 226, 238 or 250 and a CDR3 comprising a sequence set forth in SEQ ID NO: 11, 23, 35, 47, 59, 71, 83, 95, 110, 125, 137, 149, 161, 173, 185, 200, 215, 227, 239 or 251.

In another aspect, the present invention also provides a binding protein comprising a T cell receptor (TCR) α-chain variable (Vα or Valpha) domain and a TCR β-chain variable (Vβ or Vbeta) domain,
wherein the T cell receptor (TCR) α-chain variable (Vα or Valpha) domain comprises:
(i) a complementarity determining region (CDR) 1 comprising a sequence at least about 80%, at least 85%, at least 90%, at least 92%, at least 95%, at least 97%, at least 99% identical to a sequence set forth in SEQ ID NO: 261, 273, 285, 297, 309, 321, 333, 345, 357, 369, 381, 393, 405, 417, 429, 441, 453, 465, 477 or 489; a CDR2 comprising a sequence at least about 80%, at least 85%, at least 90%, at least 92%, at least 95%, at least 97%, at least 99% identical to a sequence set in SEQ ID NO: 262, 274, 286, 298, 310, 322, 334, 346, 358, 370, 382, 394, 406, 418, 430, 442, 454, 466, 478, or 490; and a CDR3 comprising a sequence at least about 80%, at least 85%, at least 90%, at least 92%, at least 95%, at least 97%, at least 99% identical to a sequence set forth in SEQ ID NO: 263, 275, 287, 299, 311, 323, 335, 347, 359, 371, 383, 395, 407, 419, 431, 443, 455, 467, 479 and 491; or
(ii) CDR1 comprising a sequence set forth in SEQ ID NO: 261, 273, 285, 297, 309, 321, 333, 345, 357, 369, 381, 393, 405, 417, 429, 441, 453, 465, 477 or 489; a CDR2 comprising a sequence set forth in SEQ ID NO: 262, 274, 286, 298, 310, 322, 334, 346, 358, 370, 382, 394, 406, 418, 430, 442, 454, 466, 478, or 490; and a CDR3 comprising a sequence set forth in SEQ ID NO: 263, 275, 287, 299, 311, 323, 335, 347, 359, 371, 383, 395, 407, 419, 431, 443, 455, 467, 479 and 491;
and
wherein the TCR β-chain variable (Vβ or Vbeta) domain comprises:
(i) a CDR1 comprising a sequence at least about 80%, at least 85%, at least 90%, at least 92%, at least 95%, at least 97%, at least 99% identical to a sequence set forth in SEQ ID NO: 264, 276, 288, 300, 312, 324, 336, 348, 360, 372, 384, 396, 408, 420, 432, 444, 456, 468, 480 or 492 a CDR2 comprising a sequence at least about 80%, at least 85%, at least 90%, at least 92%, at least 95%, at least 97%, at least 99% identical to a sequence set forth in SEQ ID NO: 265, 277, 289, 301, 313, 325, 337, 349, 361, 373, 385, 397, 409, 421, 433, 445, 457, 469, 481 or 493 and a CDR3 comprising a sequence at least about 80%, at least 85%, at least 90%, at least 92%, at least 95%, at least 97%, at least 99% identical to a sequence set forth in SEQ ID Nos: 266, 278, 290, 302, 314, 326, 338, 350, 362, 374, 386, 398, 410, 422, 434, 446, 458, 470, 482 and 494; or
(ii) a CDR1 comprising a sequence set forth in SEQ ID NO: 264, 276, 288, 300, 312, 324, 336, 348, 360, 372, 384, 396, 408, 420, 432, 444, 456, 468, 480 or 492 a CDR2 comprising a sequence set forth in SEQ ID NO: 265, 277, 289, 301, 313, 325, 337, 349, 361, 373, 385, 397, 409, 421, 433, 445, 457, 469, 481 or 493 and a CDR3 comprising a sequence set forth in SEQ ID Nos: 266, 278, 290, 302, 314, 326, 338, 350, 362, 374, 386, 398, 410, 422, 434, 446, 458, 470, 482 and 494.

In another aspect, the present invention also provides a binding protein comprising a T cell receptor (TCR) α-chain variable (Vα or Valpha) domain and a TCR β-chain variable (Vβ or Vbeta) domain,
wherein the Vα domain comprises a CDR1 comprising an amino acid sequence of any one of SEQ ID NOs: 6, 18, 30, 42, 54, 66, 78, 90, 102, 105, 120, 132, 144, 156, 168, 180, 192, 195, 210, 222, 234 and 246, a CDR2 comprising an amino acid sequence of any one of SEQ ID NOs: 7, 19, 31, 43, 55, 67, 79, 91, 103, 106, 121, 133, 145, 157, 169, 181, 193, 196, 211, 223, 235 and 247, a CDR3 comprising an amino acid sequence of any one of SEQ ID NOs: 8, 20, 32, 44, 56, 68, 80, 92, 104, 107, 122, 134, 146, 158, 170, 182, 194, 197, 212, 224, 236 and 248; and/or
wherein the Vβ domain comprises a CDR1 comprising an amino acid sequence of any one of SEQ ID NOs: 9, 21, 33, 45, 57, 69, 81, 93, 108, 123, 135, 147, 159, 171, 183, 198, 213, 225, 237 and 249, a CDR2 comprising an amino acid sequence of any one of SEQ ID NOs: 10, 22, 34, 46, 58, 70, 82, 94, 109, 124, 136, 148, 160, 172, 184, 199, 214, 226, 238 and 250, a CDR3 comprising an amino acid sequence of any one of SEQ ID NOs: 11, 23, 35, 47, 59, 71, 83, 95, 110, 125, 137, 149, 161, 173, 185, 200, 215, 227, 239 and 251,
wherein the binding protein is capable of binding to a complex of a fragment of a Smith protein and a HLA-DR15 molecule.

In another aspect, the present invention also provides a binding protein comprising a T cell receptor (TCR) α-chain variable (Vα or Valpha) domain and a TCR β-chain variable (Vβ or Vbeta) domain,
wherein the Vα domain comprises a CDR1 comprising an amino acid sequence of any one of SEQ ID NOs: 261, 273, 285, 297, 309, 321, 333, 345, 357, 369, 381, 393, 405, 417, 429, 441, 453, 465, 477 or 489, a CDR2 comprising an amino acid sequence of any one of SEQ ID NOs: 262, 274, 286, 298, 310, 322, 334, 346, 358, 370, 382, 394, 406, 418, 430, 442, 454, 466, 478, or 490, a CDR3 comprising an amino acid sequence of any one of SEQ ID NOs: 263, 275, 287, 299, 311, 323, 335, 347, 359, 371, 383, 395, 407, 419, 431, 443, 455, 467, 479 and 491; and/or
wherein the Vβ domain comprises a CDR1 comprising an amino acid sequence of any one of SEQ ID NOs: 264, 276, 288, 300, 312, 324, 336, 348, 360, 372, 384, 396, 408, 420, 432, 444, 456, 468, 480 or 492, a CDR2 comprising an amino acid sequence of any one of SEQ ID NOs: 265, 277, 289, 301, 313, 325, 337, 349, 361, 373, 385, 397, 409, 421, 433, 445, 457, 469, 481 or 493, a CDR3 comprising an amino acid sequence of any one of SEQ ID Nos: 266, 278, 290, 302, 314, 326, 338, 350, 362, 374, 386, 398, 410, 422, 434, 446, 458, 470, 482 and 494,
wherein the binding protein is capable of binding to a complex of a fragment of a Smith protein and a HLA-DR3 molecule.

In another aspect, the present invention also provides a binding protein comprising a T cell receptor (TCR) α-chain variable (Vα or Valpha) domain and a TCR β-chain variable (Vβ or Vbeta) domain,
wherein the T cell receptor (TCR) α-chain variable (Vα or Valpha) domain comprises a CDR1, 2 and 3 from Table 1 or 2; and/or
wherein the T cell receptor (TCR) 8-chain variable (Vβ or Vbeta) domain comprises a CDR1, 2 and 3 from Table 1 or 2.

Preferably, the binding protein comprises the sequences of TCRs 1, 2 or 3 from Table 1, or the sequence of TCR 1 from Table 2.

In any embodiment, the binding protein has a TCRα chain that comprises or consists of an amino acid sequence as set forth in any one of SEQ ID NOS.: 501, 503, 505, 507, 509, 511, 513, 515, 517, 518, 520, 522, 524, 526, 528, 530, 532, 533, 535, 537, 539, and 541; and/or a TCRβ chain that comprises or consists of an amino acid sequence as set forth in any one of SEQ ID NOs: 502, 504, 506, 508, 510, 512, 514, 516, 519, 521, 523, 525, 527, 529, 531, 534, 536, 538, 540, and 542 or any combination thereof.

In any embodiment, the binding protein has a TCRα chain that comprises or consists of an amino acid sequence as set forth in any one of SEQ ID NOS.: 585, 587. 589, 591, 593, 595, 597, 599, 601, 603, 605, 607, 609, 611, 613, 615, 617, 619, 621, and 623; and/or a TCRβ chain that comprises or consists of an amino acid sequence as set forth in any one of SEQ ID NOs: 586, 588, 590, 592, 594, 596, 598, 600, 602, 604, 606, 608, 610, 612, 614, 616, 618, 620, 622 and 624 or any combination thereof.

In any aspect of the present invention, the binding protein comprises a TCRα chain comprising the Vα domain and a TCRβ chain comprising a Vβ domain. Preferably, the TCRα chain and TCRβ chain are modified to include a cysteine residue that allows formation of an additional interchain disulfide bond. The cysteine introduced into each of the TCRα chain and TCRβ chains allows preferential pairing of the TCRα and TCRβ chain when expressed in a cell that expresses endogenous TCRα and TCRβ chains. Preferably, the residue at, or equivalent to, Thr48 on the TCRα chain and the residue at, or equivalent to, Ser57 on the TCRβ chain are replaced with cysteines to facilitate the creation of an additional disulfide bond between the TCR constant regions.

In another aspect, the present invention provides a peptide comprising, consisting essentially of or consisting of an amino acid sequence of or equivalent to residues 1 to 15 or 58 to 72 of a SmB/B′ protein. In one embodiment, the SmB′ protein comprises the amino acid sequence of SEQ ID NO: 5. In one embodiment, the peptide comprises, consists essentially of or consists of the amino acid sequence set forth in any one of SEQ ID NOs: 1, 2, 3 and 4, preferably as set forth in SEQ ID NO: 3 or 4.

In any aspect, a peptide of the invention is capable of binding to, or forming a complex with, a HLA-DR15 molecule, preferably the HLA-DR15 molecule is HLA-DRA*01:01 and HLA-DRB1*15:01 molecule.

In another aspect, the present invention provides a peptide comprising, consisting essentially of or consisting of an amino acid sequence of or equivalent to residues 7 to 21 of a SmB/B′ protein. In one embodiment, the SmB′ protein comprises the amino acid sequence of SEQ ID NO: 5. In one embodiment, the peptide comprises, consists essentially of or consists of the amino acid sequence set forth in 259.

In another aspect, the present invention provides a peptide comprising, consisting essentially of or consisting of an amino acid sequence of or equivalent to residues 78 to 92 of a SmD1 protein. In one embodiment, the SmB/B′ protein comprises the amino acid sequence of SEQ ID NO: 260. In one embodiment, the peptide comprises, consists essentially of or consists of the amino acid sequence set forth in SEQ ID NOs: 258.

In any aspect, a peptide of the invention is capable of binding to, or forming a complex with, a HLA-DR3 molecule, preferably the HLA-DR3 molecule is HLA-DRA*01:01 and HLA-DRB1*03:01 molecule.

In another aspect, the present invention provides a nucleic acid comprising, consisting essentially of or consisting of a nucleotide sequence encoding a binding protein or peptide of the invention.

In another aspect, the present invention provides a vector comprising a nucleotide sequence encoding a binding protein or peptide of the invention. Typically, the vector allows expression of the nucleotide sequence in a cell resulting in the presentation of the binding protein on the surface of the cell. The vector may be a retroviral vector, preferably a lentiviral vector. Typically, the vector allows expression of the nucleotide sequence in a T cell, preferably a T helper cell, for example a CD4+ T cell. A CD4+ T cell may be a CD4+CD25high T cell.

In one embodiment, the vector comprises a nucleic acid of the invention operably linked to a promoter.

In embodiments of the invention directed to single polypeptide chain binding protein, the expression construct may comprise a promoter linked to a nucleic acid encoding that polypeptide chain.

In embodiments of the invention directed to multiple polypeptide chains that form a binding protein, a vector comprises a nucleic acid encoding a polypeptide comprising, e.g., a Vα operably linked to a promoter and a nucleic acid encoding a polypeptide comprising, e.g., a Vβ operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a Vα and the second polypeptide comprises a Vβ, or vice versa. Preferably, the vector allows translation of the nucleotide sequence encoding Vβ before translation of the nucleotide sequence encoding Va.

In any aspect, a vector of the invention may comprise any one of more, or all, of the following:
(i) an EF1α (alpha) promoter;
(ii) a 2A ribosome skipping sequence;
(iii) a woodchuck hepatitis virus post-transcriptional regulatory element (WPRE);
(iv) arrangement to translate TCR β-chain variable (Vβ or Vbeta) domain prior to the (TCR) α-chain variable (Vα or Valpha) domain; or
(v) arrangement to translate TCR β-chain variable (Vβ or Vbeta) chain prior to the (TCR) α-chain variable (Vα or Valpha) chain.

Preferably, the vector is a lentiviral vector. Even more preferably the lentiviral vector has any one or more, or all, of the features shown in FIG. 4.

In another embodiment, the present invention also contemplates separate vectors one of which encodes a first polypeptide comprising a Vα and another of which encodes a second polypeptide comprising a Vβ. For example, the present invention also provides a composition comprising:
(i) a first expression construct comprising a nucleic acid encoding a polypeptide comprising a Vα operably linked to a promoter; and
(ii) a second expression construct comprising a nucleic acid encoding a polypeptide comprising a Vβ operably linked to a promoter.

In another aspect, the invention provides a cell comprising a vector or nucleic acid described herein. Preferably, the cell is isolated, substantially purified or recombinant. In one example, the cell comprises the vector of the invention or:
(i) a first expression construct comprising a nucleic acid encoding a polypeptide comprising a Vα operably linked to a promoter; and
(ii) a second expression construct comprising a nucleic acid encoding a polypeptide comprising a Vβ operably linked to a promoter,
wherein the first and second polypeptides associate to form a binding protein of the present invention. Preferably, the cell is a T cell, more preferably a T helper cell, for example a CD4+ T cell. A CD4+ T cell may be a CD4+CD25high T cell.

In another aspect, the present invention provides a cell expressing on its surface a binding protein of the invention. Preferably, the cell is a T cell, more preferably a CD4+ T cell. A CD4+ T cell may be a CD4+CD25high T cell.

In another aspect, the present invention provides a method of preparing a population of T regulatory cells for use in the treatment of SLE, the method comprising:
providing a population of T regulatory cells,
introducing a nucleic acid or vector of the invention into the population of T regulatory cells,
providing conditions to allow the expression of the binding protein on the surface of the T regulatory cells,

thereby preparing a population of T regulatory cells for use in the treatment of SLE.

In another aspect, the present invention provides a method for treating SLE in a subject, the method comprising:
administering to a subject an effective amount of T regulatory cells that express on their surface a binding protein comprising a T cell receptor (TCR) α-chain variable (Vα or Valpha) domain and a TCR β-chain variable (Vβ or Vbeta) domain, wherein the binding protein is capable of binding to a complex of a fragment of a Smith protein and an HLA-DR15 or HLA-DR3 molecule,
thereby treating SLE in the subject. Preferably, the binding protein is any binding protein of the invention as described herein

In another aspect, the present invention relates to a method for preparing an ex vivo population of Smith protein specific T cells exhibiting at least one property of a regulatory T cell, the method comprising:
providing a population of T cells exhibiting at least one property of a regulatory T cell,
introducing a nucleic acid or vector of the invention into the population of T cells, wherein the nucleic acid or vector encodes a binding protein of the invention;
providing conditions to allow the expression of the binding protein on the surface of the T cells,

thereby preparing an ex vivo population of Smith protein specific T cells exhibiting at least one property of a regulatory T cell. Preferably T cells exhibiting at least one property of a regulatory T cell are derived from a biological sample from a subject having SLE.

The T cells exhibiting at least one property of a regulatory T cell used in a method or use of the invention may be selected from subject diagnosed with SLE or from healthy subjects. The T cells may be isolated from a histocompatible donor.

In alternative embodiments, the present invention provides a method of preparing an ex vivo population of Smith protein specific T cells exhibiting at least one property of a regulatory T cell, the method comprising:
providing a population of T cells exhibiting at least one property of a conventional T cell, optionally wherein the population of T cells is a mixed population of T cells;
introducing a nucleic acid or vector of the invention into the population of T cells, wherein the nucleic acid or vector encodes a binding protein of the invention;
providing conditions to allow the expression of the binding protein on the surface of the T cells,
providing conditions to allow conversion of the population of T cells into T regulatory cells,

thereby preparing an ex vivo population of Smith protein specific T cells exhibiting at least one property of a regulatory T cell. Preferably the T cells exhibiting at least one property of a conventional T cell or mixed population of T cells are derived from a biological sample from a subject having SLE. Alternatively, the T cell may be derived from a histocompatible donor.

The present invention also relates to a composition of T regulatory cells wherein greater than 20% of the cells express a binding protein of the invention. Preferably, the composition includes greater than 30%, 40%, 50%, 60%, 70%, 80%, 85%, 90%, 91%, 92%, 93%, 94%, 95%, 96%, 97%, 98 or 99% of cells that express a binding protein of the invention.

In another aspect, the present invention provides a method of preparing a population of T regulatory cells for use in the treatment of SLE, the method comprising:
culturing a population of T regulatory cells in the presence of a peptide of the invention under conditions and for a sufficient time to allow expansion of a subpopulation which are activated by the peptide, thereby preparing a population of T regulatory cells for use in the treatment of SLE.

Further, the present invention provides a method of preparing a population of T regulatory cells for use in the treatment of SLE, the method comprising:
culturing a mixed population or T cells, or a population of T cells exhibiting at least one property of a conventional T cell, in the presence of a peptide of the invention under conditions and for a sufficient time to allow expansion of a subpopulation which are activated by the peptide,
culturing the T cells in conditions to allow the conversion of the T cells into T regulatory cells,

thereby preparing a population of T regulatory cells for use in the treatment of SLE.

In any embodiment, the conditions for allowing conversion of a conventional T cell or mixed population of T cells, into a T regulatory cell may comprise contacting the conventional T cells or mixed population of T cells with one or more agents, or increasing the expression of one or more factors suitable for conversion of conventional T cells into regulatory T cells. The one or more agents or factors may comprise: TGF-β, Foxp3 or an agent for increasing expression thereof.

In another aspect, the present invention provides a method of adoptive cellular immunotherapy, the method comprising the steps of:
extracting a mixed population of T cells from a subject diagnosed with a condition associated with an aberrant, unwanted or otherwise inappropriate immune response to a Smith protein,
isolating from the population a subpopulation comprising CD4⁺CD25⁺ T cells (Treg cells) by negative and positive immuno-selection and cell sorting,
expanding the Treg cells of the subpopulation by contacting the subpopulation with effective amounts of a peptide of the invention, and
introducing the ex vivo expanded Treg cells into the subject.

In another aspect, the present invention provides a composition comprising a binding protein, peptide, cell or vector of the invention, and a pharmaceutically acceptable carrier, diluent or excipient.

In another aspect, the present invention provides a method of treating or preventing a condition in a subject, wherein the condition is associated with an aberrant, unwanted or otherwise inappropriate immune response to a Smith protein, the method comprising administering to the subject a binding protein, peptide, cell, nucleic acid or composition of the invention, thereby treating or preventing the condition in the subject.

In another aspect, the present invention provides a method of treating or preventing a condition in a subject, wherein the condition is associated with an aberrant, unwanted or otherwise inappropriate immune response to a Smith protein, the method comprising:
providing a population of T cells exhibiting at least one property of a regulatory T cell,
introducing a nucleic acid or vector of the invention into the population of T cells, wherein the nucleic acid or vector encodes a binding protein of the invention;
providing conditions to allow the expression of the binding protein on the surface of the T cells,
administering the T cells expressing the binding protein on their surface,

thereby treating or preventing the condition in the subject. Preferably T cells exhibiting at least one property of a regulatory T cell are derived from a biological sample from a subject having SLE.

In another aspect, the present invention provides use of a binding protein, peptide, cell, nucleic acid or composition of the invention in the manufacture of a medicament for treating or preventing a condition in a subject, wherein the condition is associated with an aberrant, unwanted or otherwise inappropriate immune response to a Smith protein.

In another aspect, the present invention provides a binding protein, peptide, cell, nucleic acid or composition of the invention for use in treating or preventing a condition in a subject, wherein the condition is associated with an aberrant, unwanted or otherwise inappropriate immune response to a Smith protein.

In any aspect, the condition associated with an aberrant, unwanted or otherwise inappropriate immune response to a Smith protein is systemic lupus erythematosus (SLE). Alternatively, the aberrant, unwanted or otherwise inappropriate immune response to a Smith protein is lupus nephritis (LN). Consequently, a subject in need thereof is a subject that is diagnosed with SLE or LN.

Preferably, the subject with SLE is identified has having HLA-DR15 or HLA-DR3 alleles, more preferably HLA-DRA*01:01 and HLA-DRB1*15:01 or HLA-DRA*01:01 and HLA-DRB1*03:01 molecule.

Preferably the peptide for use in treating or preventing SLE is the SmB/B′:1-15 or peptide SmB/B′:58-72 or a fragment thereof as herein described. Preferably the peptide comprises, consists or consists essentially of a sequence set forth in any one of SEQ ID NOs: 1 to 4.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Identification of Sm derived peptides that bind to HLA-DR15.
A. The MHC Class II Preimmune REVEAL assay was employed to identify Sm derived peptides (15-mers overlapping by 12 amino acids) that bind to HLA-DR15. The results of the assay are presented as percentage of binding relative to a positive control at 0 hours (blue bars) and 24 hours (red bars). Based on these scores a stability index (red bars) for each peptide was derived. The positive control scores are 100% at 0 hours, 6.4% at 24 hours; and had a stability index of 6.0.
B-D. Binding scores and stability indices of SmB/B′, SmD1 and SmD3 derived peptides.

FIG. 2: Human T cell reactivity to the top three HLA-DR15 restricted Sm-peptides.
A. To determine if the HLA-DR15 restricted Sm-peptides could induce T cell reactivity, the three highest binders: SmB/B′:1-15, SmB/B′:58-72, or SmD3:43-57 were cultured respectively, with human CD4+ T cells and monocyte derived dendritic cells from a HLA-DRB1*15:01 homozygous donor. T cell reactivity was determined by cell proliferation using Cell Trace Violet (CTV) assays.
B. Representative FACS plots showing the percentages of CTVIo CD4+ T cells. strong proliferative responses in CD4+ T cells cultured with SmB/B′:1-15 and SmB/B′:58-72 compared to No peptide and SmD3:43-57.

FIG. 3: Human T cell reactivity to HLA-DR3 restricted Sm-peptides.
A. To determine if human T cell reactivity to HLA-DR3 restricted Sm-peptides could be measured, the inventors tested the SmD1:78-92 peptide previously identified by Deshmukh U S et al, 2011, and the top in silico (IEDB) predicted binding peptide of SmB/B′, SmB/B′:7-21. These peptides were cultured individually with CD4+ T cells in co-culture cell proliferation assays and reactivity assessed by Cell Trace Violet (CTV) dilution.
B. Representative FACS plots showing the percentages of CTVIo CD4+ T cells. Strong proliferative responses were observed only in CD4+ T cells cultured with SmD1:78-92

FIG. 4: Map of the modified lentiviral construct used to transduce TCRs onto human regulatory T cells. The relative locations of the alpha and beta chains, P2A, T2A as well as the introduced murinised mutations and cysteination are shown.

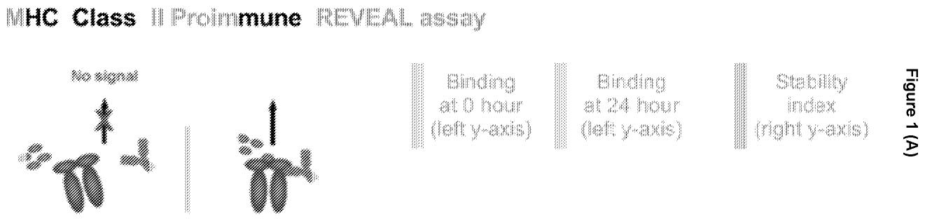

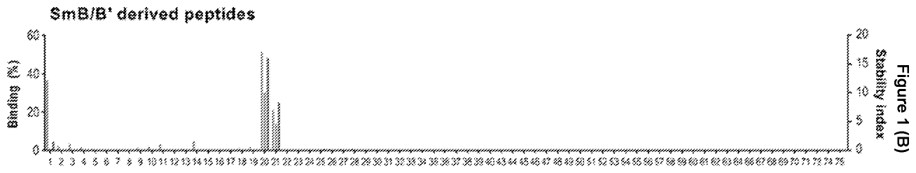

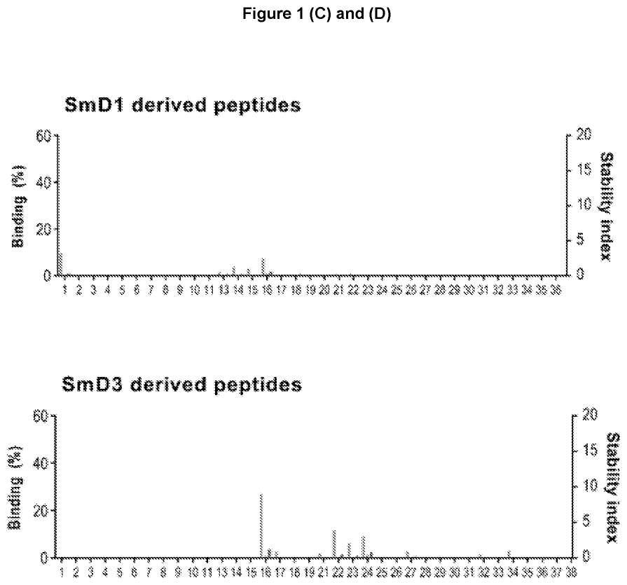

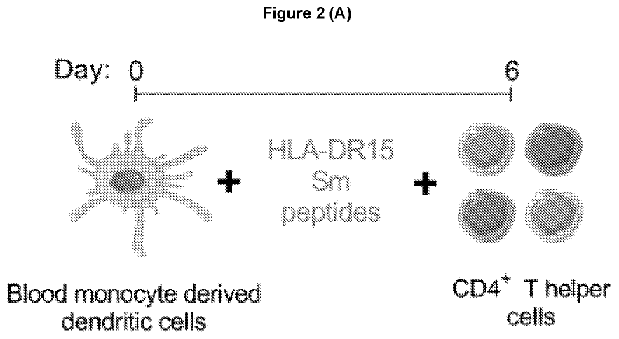

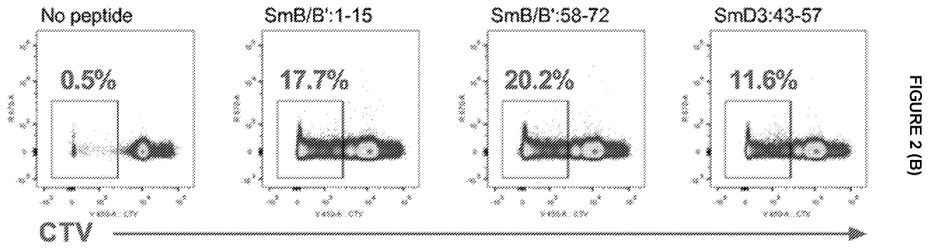

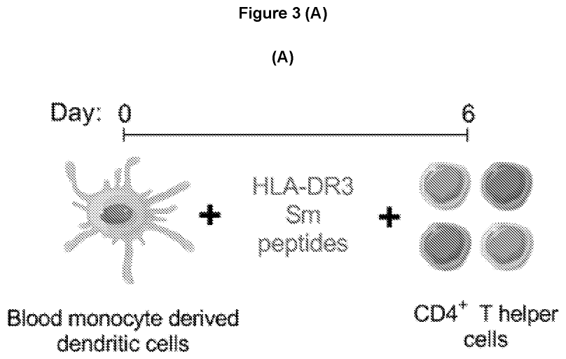

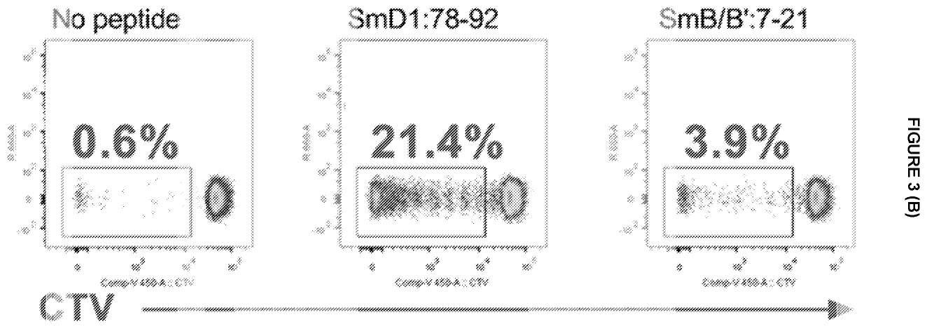

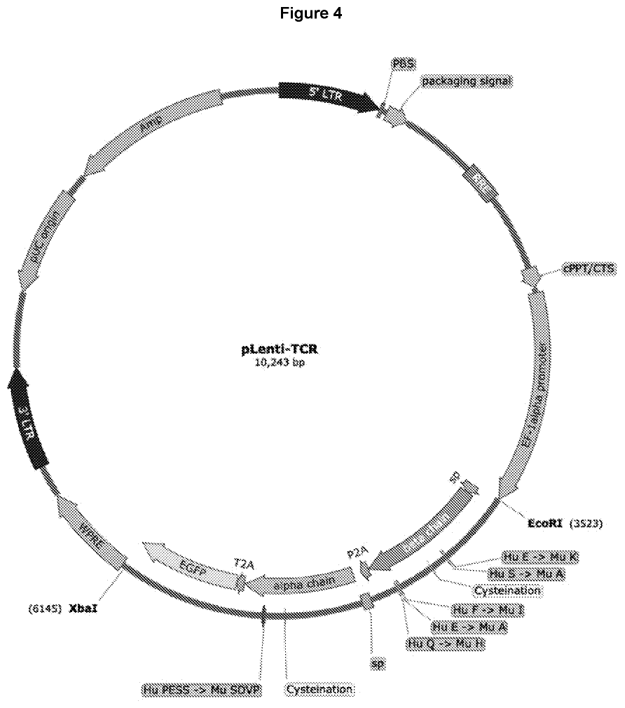

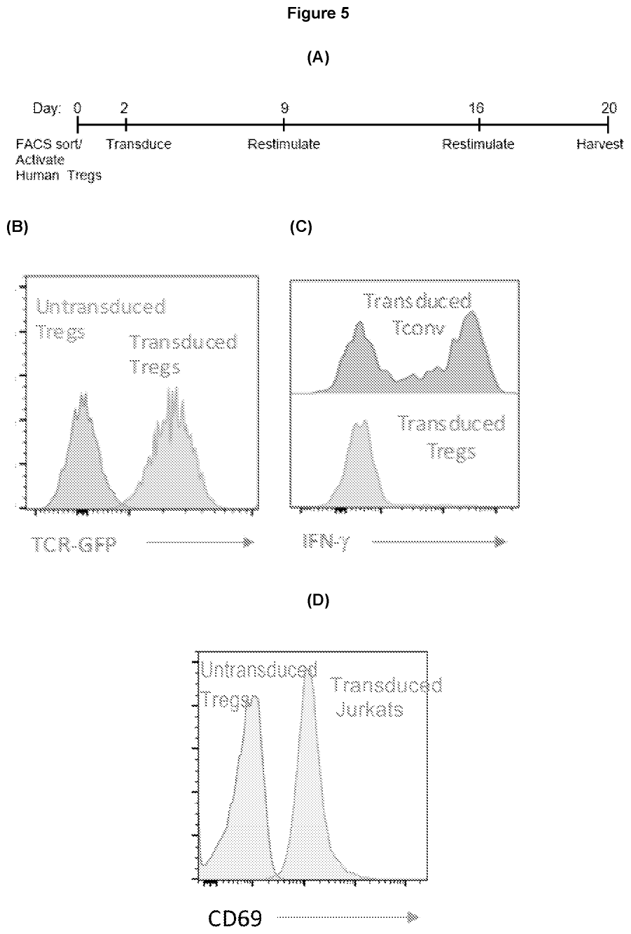

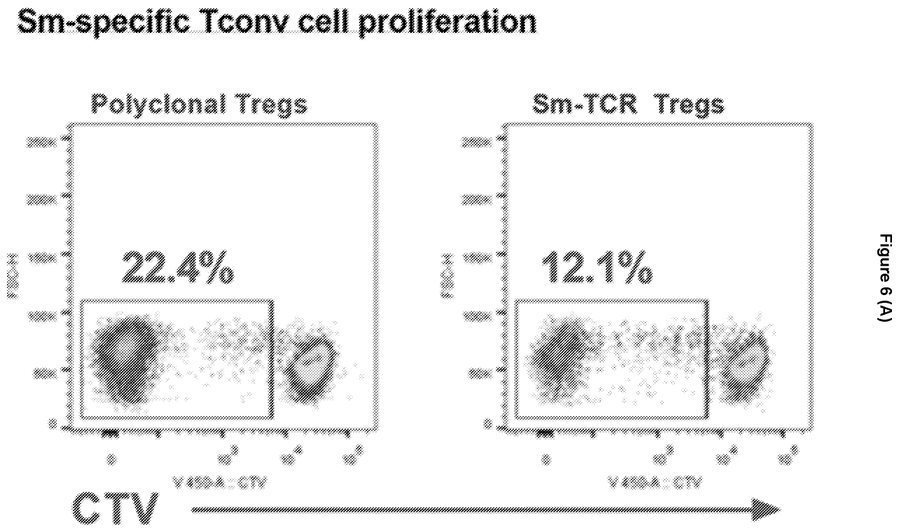

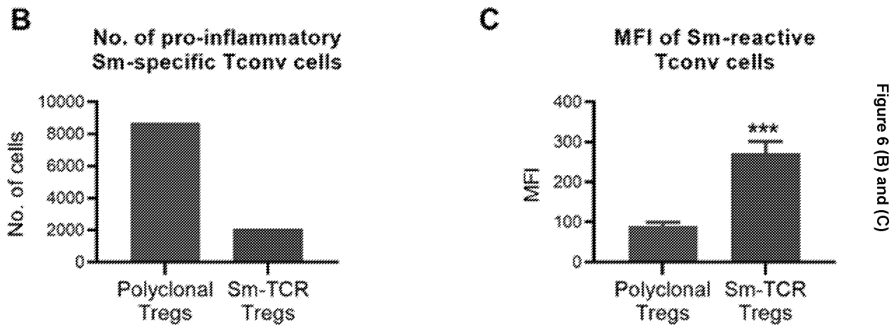

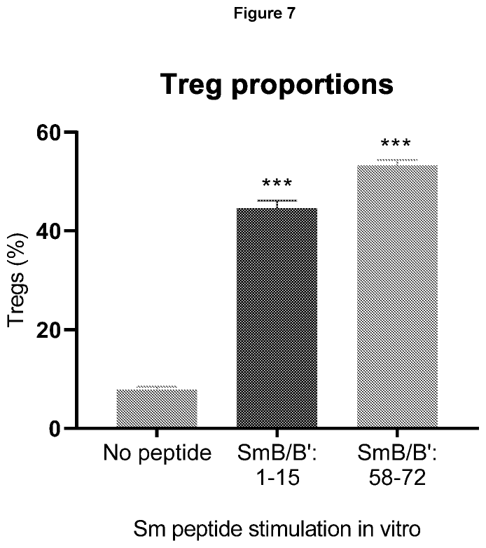

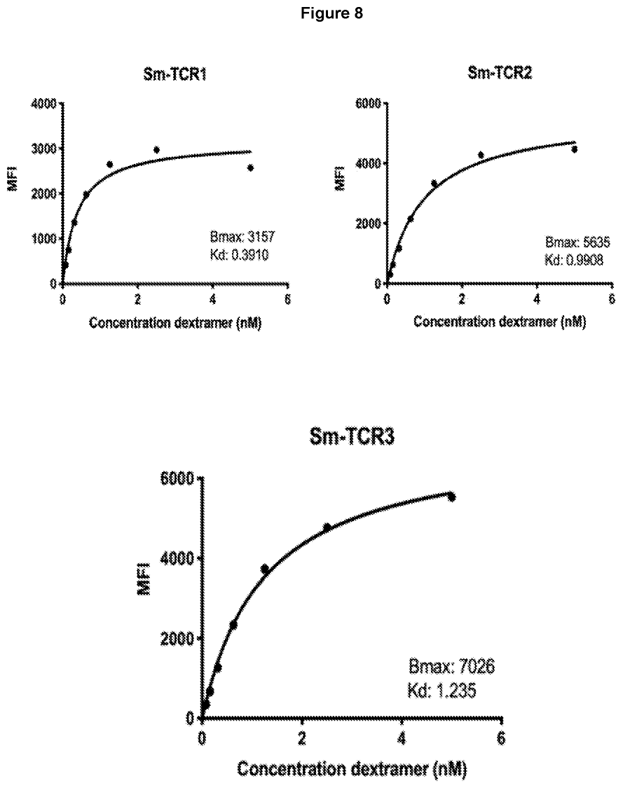

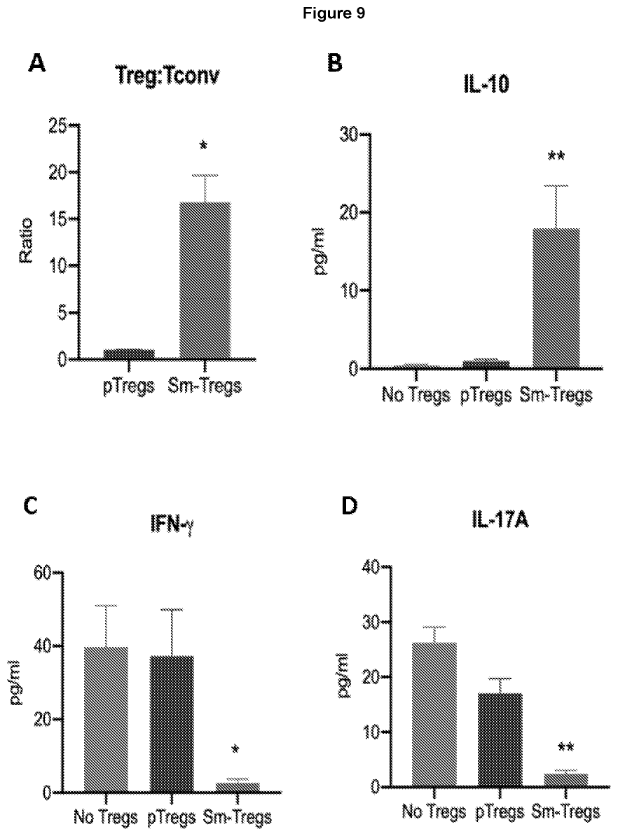

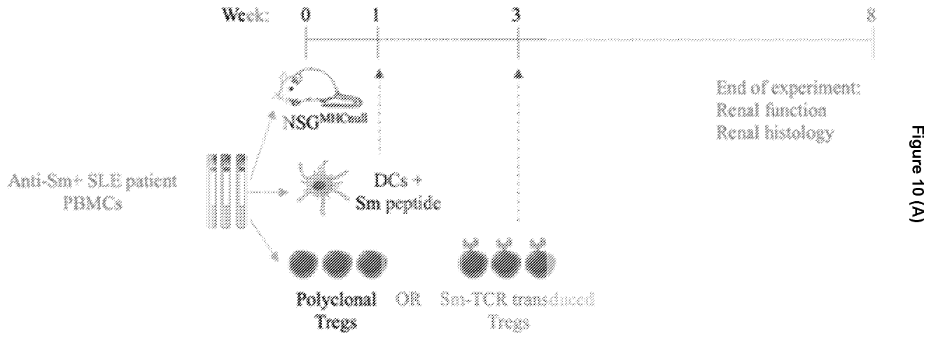

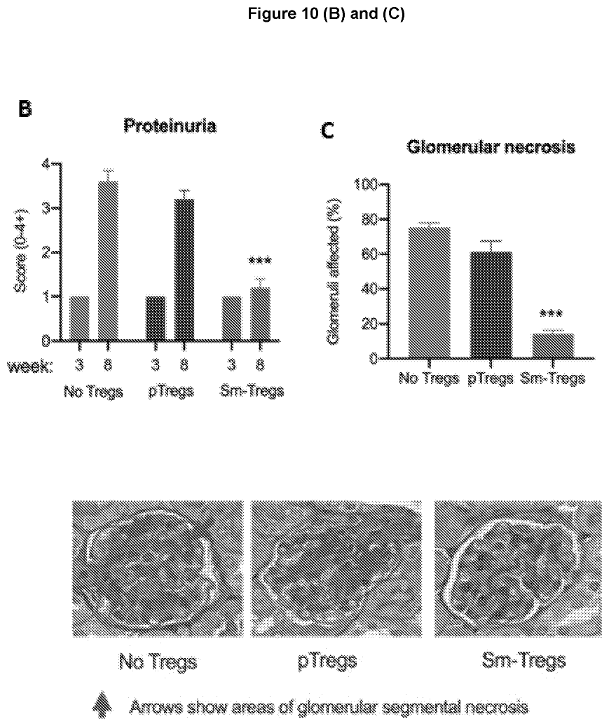

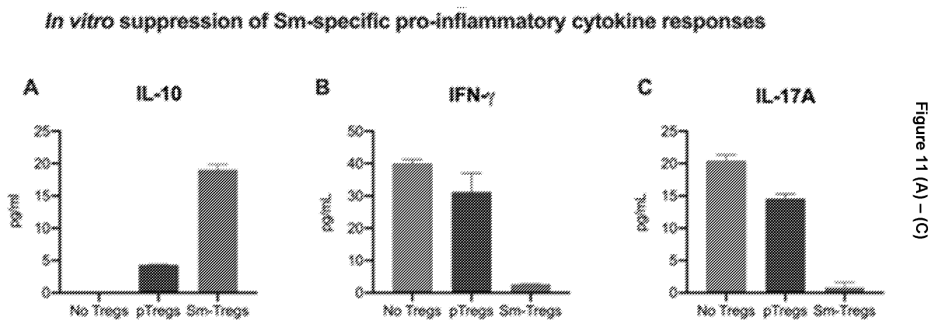

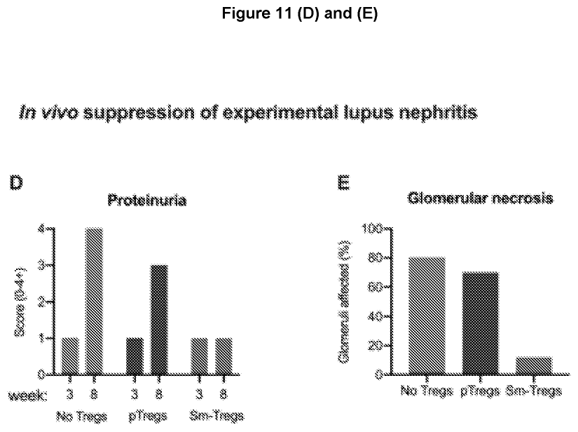

FIG. 5: TCR transduction of TCRs onto human Tregs.
A. Timeline of TCR transduction protocol. Human Tregs (CD4+CD25hi CD127lo) are first sorted by flow cytometry then stimulated with anti-CD3 and anti-CD28 beads followed by transduction on day 2 of the lentiviral construct of FIG. 4. After two rounds of re-stimulation (days 9 and 26), Tregs are harvested and analysed for TCR expression and stability of Treg phenotype.
B. Analysis of TCR expression in human Tregs at day 20 show that greater than 90% of transduced Tregs express the GFP tag.
C. Intracellular cytokine staining for the pro-inflammatory cytokine IFN-g, shows that the transduced Tregs do not switch into pro-inflammatory cells (staining for IL-17A was also negative).
D. Transduced TCRs are functional. To determine if this protocol leads to TCRs that are functional, transduced a Jurkat T cell line and stimulated the transduced Jurkat T cells with an antigen-presenting cell line (HLA-DR15+B-LCLs) pulsed with the TCRs cognate peptide. Here the inventors show upregulation of the early activation marker CD69 following stimulation demonstrating that the TCRs transduced using this protocol lead to functional TCRs on the surface of T cells.

FIG. 6: Compared to polyclonal Tregs, Sm-TCR transduced are more potent suppressors of Sm-specific Tconv cell reactivity.
A. In vitro T cell proliferation assay. HLA-DR15+PBMCs isolated from SLE patients were stimulated with the dominant Sm peptide SmB/B′:58-72 and co-cultured with either polyclonal Tregs (left FACS plot) or Sm-TCR transduced Tregs (right plot). Proliferation of pro-inflammatory T conventional cells (Tconv) cells was assessed by Cell Trace Violet (CTV) dilution. Sm-TCR transduced Tregs more potently inhibited Tconv cell proliferation 12.1% versus 22.4%.
B. Enumeration of proliferating cells showed that there were more Sm-specific Tconv cells in the polyclonal group compared to the Sm-TCR group (8659 versus 2053).
C. Mean Fluorescence Intensity (MFI) of Sm-reactive Tconv cells reflect the number of cell divisions. The lower the MFI the more cell divisions the Tconv cells undergo. The MFI of Sm-reactive Tconv cells in the group that received polyclonal Tregs were lower than in the Sm-TCR Treg group (89.1 versus 271). Error bars are SEM. *** P<0.001 by t-test.

FIG. 7: Expansion of regulatory T cells (Tregs) after stimulation with either peptide SmB/B′:1-15 or peptide SmB/B′:58-72. The proportions of Tregs, relative to total CD4+ T cells, was determined in the absence of peptide stimulation or after in vitro stimulation with either SmB/B′:1-15 or SmB/B′:58-72. The use of either SmB/B′:1-15 or SmB/B′:58-72 was found to selectively enhance the expansion of Tregs demonstrated by significant increases in the proportions of Tregs after peptide stimulation. Data shown is mean±SD of two independent experiments. *** P<0.001 by One-way ANOVA with Tukey's post-test compared to No peptide group.

FIG. 8: The dominant HLA-DR15 restricted Sm-TCR binds with high affinity to HLA-DR15 dextramers presenting SmB/B′:58-72. The TCR-binding affinity of Sm-specific TCRs derived by the inventors was determined using a dextramer based flow cytometry binding assay. The inventors cloned the top 3 TCRs (i.e. TCR1, TCR2 and TCR3 as identified in Table 1) into a Jurkat T cell line. The inventors the measured the mean fluorescence intensity (MFI) by flow cytometry and expressed the data using Scatchard plots. Relative Bmax and dissociation constants (Kd) are shown in each plot.

FIG. 9: HLA-DR15 restricted Sm-TCR Tregs suppress anti-Sm specific pro-inflammatory responses and restore tolerance. PBMCs from HLA-DR15+, anti-Sm+ SLE patients with lupus nephritis were co-cultured with the dominant HLA-DR15 restricted Sm peptide (SmB/B′:58-72) and either no Tregs, polyclonal Tregs (pTregs) or Tregs transduced with the HLA-DR15 restricted Sm-specific TCR 1 (Sm-Tregs). (A) In the presence of Sm-Tregs, the numbers of Sm-specific Tregs to Tconv cells is significantly increased. (B-D) In the presence of Sm-Tregs an anti-inflammatory response, i.e. high IL-10, low IFN-gamma and IL-17A, was dominant (similarly to a healthy persons), whereas without Tregs or with only pTregs, a pro-inflammatory response was dominant, i.e. low IL-10, high IFN-gamma and IL-17A (as is expected in autoimmune disease patients). These data demonstrate that Sm-Tregs have the capability to reset the aberrant immune response and restore tolerance to the targeted autoepitope. Results are expressed as mean+/−SEM using samples from four SLE patients, * P<0.05, ** P<0.01 when compared to No Tregs group and pTregs group.

FIG. 10: HLA-DR15 restricted Sm-Tregs halt the progression of nephritis. (A) NSG^{MHCnull} mice received PBMCs from SLE patients with lupus nephritis who were also positive for anti-Sm antibodies and HLA-DR15+. At the onset of functional renal injury (measured by an increase in proteinuria), week 3, mice were administered either no Tregs, polyclonal Tregs (pTregs) or HLA-DR15 restricted Sm-Tregs (transduced with HLA-DR15 TCR 1). (B and C) Mice that received no Tregs or pTregs progressed to severe nephritis (i.e high levels of proteinuria and >50% of glomeruli with necrosis), however, mice treated with Sm-Tregs did not progress. Results are expressed as mean+/−SEM of five SLE patient samples. *** P<0.001 compared to No Tregs and pTregs groups.

FIG. 11: HLA-DR3 restricted Sm-Tregs suppress anti-Sm pro-inflammatory cytokine responses and halt the progression of lupus nephritis. (A-C) PBMCs from a HLA-DR3+, anti-Sm+ SLE patient with lupus nephritis was co-cultured with the dominant HLA-DR3 restricted T cell epitope (SmD1:78-92) and either No Tregs, polyclonal Tregs (pTregs) or Tregs transduced with the HLA-DR3 restricted TCR (HLA-DR3 TCR 1, as identified in Table 2) (Sm-Tregs). Cytokine responses were measured at day 8. (D and E) NSG^{MHcnull} mice received PBMCs from a SLE patient with lupus nephritis who were also positive for anti-Sm antibodies and HLA-DR3+. At the onset of functional renal injury (measured by an increase in proteinuria), week 3, mice were administered either no Tregs, polyclonal Tregs (pTregs) or HLA-DR3 restricted Sm-Tregs (transduced with HLA-DR3 TCR 1).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have identified peptides derived from Smith proteins that bind to HLA molecules DR15 and DR3 which are prevalent in individuals having SLE. Those peptides, when bound to HLA molecules, result in CD4+T helper cell proliferation and have allowed identification of Smith protein specific T cell receptors. The invention therefore relates to the use of peptide immunotherapy to treat SLE, or adoptive cell therapy with T regulatory cells engineered to express a Smith protein specific TCR.

An advantage of an aspect of the invention is that both the peptides and TCRs identified are involved in interactions with HLA-DR subtypes common in lupus patients. Further, antigen-specific T regulatory cell therapy has a typically more potent immunosuppressive effect than polyclonal T regulatory cell therapy. Finally, antigen-specific T regulatory cell therapy has a typically more limited immunosuppressive effect on protective T cell immunity, for example that use to respond to viral infection and/or cancer.

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, T cell receptors and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 1 to 15” will be understood in an inclusive manner, i.e., the region comprises a sequence of amino acids as numbered 1, 2, 3, 4, 5, 6, 7, 8, 9, 10, 11, 12, 13, 14 and 15 in a specified sequence.

The term “consisting essentially of limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “nucleic acid” or “nucleic acid molecule” refers to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., a-enantiomeric forms of naturally-occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

As used herein, the term “recombinant” refers to a cell, microorganism, nucleic acid molecule, or vector that has been genetically engineered by human intervention—that is, modified by introduction of an exogenous or heterologous nucleic acid molecule, or refers to a cell or microorganism that has been altered such that expression of an endogenous nucleic acid molecule or gene is controlled, deregulated or constitutive. Human generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

A “conservative substitution” is recognized in the art as a substitution of one amino acid for another amino acid that has similar properties. Exemplary conservative substitutions are well known in the art (see, e.g., WO 97/09433 at page 10; Lehninger, Biochemistry, 2nd Edition; Worth Publishers, Inc. NY, N.Y., pp. 71-77, 1975; Lewin, Genes IV, Oxford University Press, NY and Cell Press, Cambridge, Mass., p. 8, 1990).

Binding Proteins

A “binding protein” as used herein, refers to a proteinaceous molecule or portion thereof (e.g., peptide, oligopeptide, polypeptide, protein) that possesses the ability to specifically and non-covalently associate, unite, or combine with a target (e.g., Smith protein or fragment thereof, Smith protein fragment:MHC complex). A binding protein may be purified, substantially purified, synthetic or recombinant. Exemplary binding proteins include single chain immunoglobulin variable regions (e.g., scTCR, scFv).

In certain embodiments, any of the binding proteins of the invention are each a T cell receptor (TCR), a chimeric antigen receptor or an antigen-binding fragment of a TCR, any of which can be chimeric, humanized or human. In further embodiments, an antigen-binding fragment of the TCR comprises a single chain TCR (scTCR) or a chimeric antigen receptor (CAR). In certain embodiments, a binding protein is a TCR.

“T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α (alpha) and β(beta) chains (also known as TCRα and TCRβ, respectively), or γ and δ chains (also known as TCRγ and TORδ, respectively). Like immunoglobulins, the extracellular portion of TCR chains (e.g., α-chain, β-chain) contain two immunoglobulin domains, a variable domain (e.g., α-chain variable domain or Vα, β-chain variable domain or Vβ; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or Cβ, typically amino acids 117 to 295 based on Kabat) adjacent to the cell membrane. Also like immunoglobulins, the variable domains contain complementary determining regions (CDRs) separated by framework regions (FRs) (see, e.g., Jores et al., Proc. Nat′lAcad. Sci. U.S.A. 57:9138, 1990; Chothia et al, EMBO J. 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal.

In any of the aforementioned embodiments, the present disclosure provides a high affinity engineered T cell receptor (TCR), comprising an alpha-chain (α-chain) and a beta-chain (β-chain), wherein the TCR binds to a complex of a fragment of a Smith protein and an HLA-DR15 molecule, preferably, the HLA-DR15 molecule is an HLA-DRA*01:01 and HLA-DRB1*15:01 molecule. In certain embodiments, a V beta chain comprises or is derived from a TRBV3, TRBV4, TRBV5, TRBV6, TRBV7, TRBV11, TRBV19, TRBV20, TRBV24, or TRBV28 allele. In further embodiments, a V alpha chain comprises or is derived from a TRAV1, TRAV2, TRAV3, TRAV4, TRAV8, TRAV9, TRAV12, TRAV14, TRAV17, TRAV21, TRAV23, TRAV25, TRAV26, TRAV27, TRAV29, TRAV38, TRAV39, or TRAV40 allele. In particular embodiments, a binding protein of the invention comprises: (a) a V beta chain that comprises or is derived from a TRBV11 allele (preferably TRBV11-2) and a V alpha chain that comprises or is derived from a TRAV9 allele (preferably TRAV9-2); (b) a V beta chain that comprises or is derived from a TRBV6 allele (preferably TRBV6-1) and a V alpha chain that comprises or is derived from a TRAV25 allele; (c) a V beta chain that comprises or is derived from a TRBV7 allele (preferably TRBV7-9) and a V alpha chain that comprises or is derived from a TRAV29 allele; (d) a V beta chain that comprises or is derived from a TRBV28 allele and a V alpha chain that comprises or is derived from a TRAV23 allele; (e) a V beta chain that comprises or is derived from a TRBV7 allele (preferably TRVB7-9) and a V alpha chain that comprises or is derived from a TRAV26 allele (preferably TRAV26-1); (f) a V beta chain that comprises or is derived from a TRBV7 allele (preferably TRBV7-3) and a V alpha chain that comprises or is derived from a TRAV8 allele (preferably TRAV8-6); (g) a V beta chain that comprises or is derived from a TRBV20 allele (preferably TRBV20-1) and a V alpha chain that comprises or is derived from a TRAV9 allele (preferably TRAV9-2); (h) a V beta chain that comprises or is derived from a TRBV3 allele (preferably TRBV3-1) and a V alpha chain that comprises or is derived from a TRAV2 allele; (i) a V beta chain that comprises or is derived from a TRBV4 allele (preferably TRBV4-2) and a V alpha chain that comprises or is derived from a TRAV17 allele; (j) a V beta chain that comprises or is derived from a TRBV4 allele (preferably TRBV4-2) and a V alpha chain that comprises or is derived from a TRAV27 allele; (k) a V beta chain that comprises or is derived from a TRBV6 allele (preferably TRBV6-5) and a V alpha chain that comprises or is derived from a TRAV2 allele.

In further embodiments, a binding protein of the invention comprises: (a) a V beta chain that comprises or is derived from a TRBV20 allele (preferably TRBV20-1) and a V alpha chain that comprises or is derived from a TRAV38 allele (preferably TRAV38-1); (b) a V beta chain that comprises or is derived from a TRBV6 allele (preferably TRBV6-4) and a V alpha chain that comprises or is derived from a TRAV1 allele (preferably TRAV1-2); (c) a V beta chain that comprises or is derived from a TRBV6 allele (preferably TRBV6-4) and a V alpha chain that comprises or is derived from a TRAV4 allele; (d) a V beta chain that comprises or is derived from a TRBV4 allele (preferably TRBV4-1) and a V alpha chain that comprises or is derived from a TRAV17 allele; (e) a V beta chain that comprises or is derived from a TRBV5 allele (preferably TRBV5-4) and a V alpha chain that comprises or is derived from a TRAV21 allele; (f) a V beta chain that comprises or is derived from a TRBV28 allele and a V alpha chain that comprises or is derived from a TRAV27 allele; (g) a V beta chain that comprises or is derived from a TRBV24 allele (preferably TRBV24-1) and a V alpha chain that comprises or is derived from a TRAV1 allele (preferably TRAV1-1).

In any aspect or embodiment, the binding protein of the invention comprises (a) a Vbeta chain that comprises or is derived from a TRBJ2 allele (preferably TRBJ2-7) and a Valpha chain that comprises or is derived from a TRAJ47 allele; (b) a Vbeta chain that comprises or is derived from a TRBJ2 allele (preferably TRBJ2-3) and a Valpha chain that comprises or is derived from a TRAJ54 allele; (c) a Vbeta chain that comprises or is derived from a TRBJ1 allele (preferably TRBJ1-1) and a Valpha chain that comprises or is derived from a TRAJ48 allele; (d) a Vbeta chain that comprises or is derived from a TRBJ2 allele (preferably TRBJ2-1) and a Valpha chain that comprises or is derived from a TRAJ44 allele; (e) a Vbeta chain that comprises or is derived from a TRBJ1 allele (preferably TRBJ1-5) and a Valpha chain that comprises or is derived from a TRAJ38 allele; (f) Vbeta chain that comprises or is derived from a TRBJ2 allele (preferably TRBJ2-1) and a Valpha chain that comprises or is derived from a TRAJ22 allele; (g) a Vbeta chain that comprises or is derived from a TRBJ2 allele (preferably TRBJ2-7) and a Valpha chain that comprises or is derived from a TRAJ11 allele; (h) a Vbeta chain that comprises or is derived from a TRBJ1 allele (preferably TRBJ1-1) and a Valpha chain that comprises or is derived from a TRAJ8 allele; (i) a Vbeta chain that comprises or is derived from a TRBJ2 allele (preferably TRBJ2-3) and a Valpha chain that comprises or is derived from a TRAJ45 allele; (j) a Vbeta chain that comprises or is derived from a TRBJ2 allele (preferably TRBJ2-3) and a Valpha chain that comprises or is derived from a TRAJ49 allele; (k) a Vbeta chain that comprises or is derived from a TRBJ1 allele (preferably TRBJ1-2) and a Valpha chain that comprises or is derived from a TRAJ7 allele.

In any aspect or embodiment, the binding protein of the invention comprises (a) a Vbeta chain that comprises or is derived from a TRBJ1 allele (preferably TRBJ1-4) and a Valpha chain that comprises or is derived from a TRAJ48 allele; (b) a Vbeta chain that comprises or is derived from a TRBJ1 (preferably TRBJ1-5) allele and a Valpha chain that comprises or is derived from a TRAJ48 allele; (c) a Vbeta chain that comprises or is derived from a TRBJ2 allele (preferably TRBJ2-1) and a Valpha chain that comprises or is derived from a TRAJ48 allele; (d) a Vbeta chain that comprises or is derived from a TRBJ2 allele (preferably TRBJ2-7) and a Valpha chain that comprises or is derived from a TRAJ48 allele; (e) a Vbeta chain that comprises or is derived from a TRBJ1 allele (preferably TRBJ1-1) and a Valpha chain that comprises or is derived from a TRAJ88 allele; (f) a Vbeta chain that comprises or is derived from a TRBJ1 allele (preferably TRBJ1-2) and a Valpha chain that comprises or is derived from a TRAJ12 allele; (g) a Vbeta chain that comprises or is derived from a TRBJ1 allele (preferably TRBJ1-2) and a Valpha chain that comprises or is derived from a TRAJ3 allele; (h) Vbeta chain that comprises or is derived from a TRBJ1 allele (preferably TRBJ1-3) and a Valpha chain that comprises or is derived from a TRAJ9 allele; (i) a Vbeta chain that comprises or is derived from a TRBJ2 allele (preferably TRBJ2-1) and a Valpha chain that comprises or is derived from a TRAJ28 allele; (j) a Vbeta chain that comprises or is derived from a TRBJ1 allele (preferably TRBJ1-2) and a Valpha chain that comprises or is derived from a TRAJ41 allele; (k) a Vbeta chain that comprises or is derived from a TRBJ2 allele (preferably TRBJ2-1) and a Valpha chain that comprises or is derived from a TRAJ9 allele.

In any aspect or embodiment, the binding protein of the invention comprises a V beta chain that comprises or is derived from a TRBD1 or TRBD2 allele.

In any aspect or embodiment, the binding protein of the invention comprises a V beta chain that comprises or is derived from a TRC1 or TRBC2 allele and a V alpha chain that comprises or is derived from a TRAC allele.

In any of the aforementioned embodiments, the present disclosure provides a high affinity engineered T cell receptor (TCR), comprising an alpha-chain (α-chain) and a beta-chain (β-chain), wherein the TCR binds to a complex of a fragment of a Smith protein and an HLA-DR3 molecule, preferably, the HLA-DR3 molecule is an HLA-DRA*01:01 and HLA-DRB1*03:01 molecule. In certain embodiments, a V beta chain comprises or is derived from a TRB2, TRBV4, TRBV5, TRB6, TRB7, TRBV9, TRB10, TRBV11, TRB12, TRBV20, TRBV24, TRB27 or TRBV29 allele. In further embodiments, a V alpha chain comprises or is derived from a TRAV1, TRAV2, TRAV8, TRAV9, TRAV10, TRAV12, TRAV20, TRAV26, TRAV30 or TRAV36 allele. In particular embodiments, a binding protein of the invention comprises: (a) a V beta chain that comprises or is derived from a TRBV5 allele (preferably TRBV5-1) and a V alpha chain that comprises or is derived from a TRAV20 allele; (b) a V beta chain that comprises or is derived from a TRBV29 allele (preferably TRBV29-1) and a V alpha chain that comprises or is derived from a TRAV12 allele (preferably TRAV12-1); (c) a V beta chain that comprises or is derived from a TRBV4 allele (preferably TRBV4-1) and a V alpha chain that comprises or is derived from a TRAV26 allele (preferably TRAV26-2); (d) a V beta chain that comprises or is derived from a TRBV4 allele (preferably TRBV4-1) and a V alpha chain that comprises or is derived from a TRAV30 allele; (e) a V beta chain that comprises or is derived from a TRBV4 allele (preferably TRVB4-1) and a V alpha chain that comprises or is derived from a TRAV36 allele (preferably TRAV36DV7); (f) a V beta chain that comprises or is derived from a TRBV24 allele (preferably TRBV24-1) and a V alpha chain that comprises or is derived from a TRAV12 allele (preferably TRAV12-1); (g) a V beta chain that comprises or is derived from a TRBV11 allele (preferably TRBV11-2) and a V alpha chain that comprises or is derived from a TRAV12 allele (preferably TRAV12-3); (h) a V beta chain that comprises or is derived from a TRBV20 allele (preferably TRBV20-1) and a V alpha chain that comprises or is derived from a TRAV9 allele (preferably TRAV9-2); (i) a V beta chain that comprises or is derived from a TRBV9 allele and a V alpha chain that comprises or is derived from a TRAV9 allele (preferably TRAV9-2); (j) a V beta chain that comprises or is derived from a TRBV20 allele (preferably TRBV20-1) and a V alpha chain that comprises or is derived from a TRAV12 allele (preferably TRAV12-1).

In further embodiments, a binding protein of the invention comprises: (a) a V beta chain that comprises or is derived from a TRBV27 allele and a V alpha chain that comprises or is derived from a TRAV12 allele (preferably TRAV12-1); (b) a V beta chain that comprises or is derived from a TRBV6 allele (preferably TRBV6-1) and a V alpha chain that comprises or is derived from a TRAV1 allele (preferably TRAV1-2); (c) a V beta chain that comprises or is derived from a TRBV7 allele (preferably TRBV7-9) and a V alpha chain that comprises or is derived from a TRAV12 allele (preferably TRAV12-2); (d) a V beta chain that comprises or is derived from a TRBV2 allele and a V alpha chain that comprises or is derived from a TRAV8 allele (TRAV8-3); (e) a V beta chain that comprises or is derived from a TRBV8 allele (preferably TRBV8-3) and a V alpha chain that comprises or is derived from a TRAV5 allele (preferably TRAV5-1); (f) a V beta chain that comprises or is derived from a TRBV7 allele (preferably TRBV7-9) and a V alpha chain that comprises or is derived from a TRAV10 allele; (g) a V beta chain that comprises or is derived from a TRBV7 allele (preferably TRBV7-9) and a V alpha chain that comprises or is derived from a TRAV19 allele; (h) a V beta chain that comprises or is derived from a TRBV10 allele (preferably TRBV10-3) and a V alpha chain that comprises or is derived from a TRAV2 allele; (i) a V beta chain that comprises or is derived from a TRBV12 allele (preferably TRBV12-4) and a V alpha chain that comprises or is derived from a TRAV20 allele.

In certain embodiments, a V beta chain comprises or is derived from a TRBJ1 or TRBJ2 allele. In further embodiments, a V alpha chain comprises or is derived from a TRAJ3, TRAJ6, TRAJ9, TRAJ13, TRAJ17, TRAJ23, TRAJ27, TRAJ28, TRAJ31, TRAJ33, TRAJ37, TRAJ42, TRAJ45, TRAJ47, TRAJ48, TRAV49 or TRAV54 allele.

In particular embodiments, the binding protein of the invention comprises (a) a Vbeta chain that comprises or is derived from a TRBJ1 allele (preferably TRBJ1-1) and a Valpha chain that comprises or is derived from a TRAJ6 allele; (b) a Vbeta chain that comprises or is derived from a TRBJ1 allele (preferably TRBJ1-5) and a Valpha chain that comprises or is derived from a TRAJ45 allele; (c) a Vbeta chain that comprises or is derived from a TRBJ2 allele (preferably TRBJ2-2) and a Valpha chain that comprises or is derived from a TRAJ54 allele; (d) a Vbeta chain that comprises or is derived from a TRBJ2 allele (preferably TRBJ2-1) and a Valpha chain that comprises or is derived from a TRAJ28 allele; (e) a Vbeta chain that comprises or is derived from a TRBJ2 allele (preferably TRBJ2-1) and a Valpha chain that comprises or is derived from a TRAJ49 allele; (f) Vbeta chain that comprises or is derived from a TRBJ2 allele (preferably TRBJ2-2) and a Valpha chain that comprises or is derived from a TRAJ48 allele; (g) a Vbeta chain that comprises or is derived from a TRBJ1 allele (preferably TRBJ1-2) and a Valpha chain that comprises or is derived from a TRAJ17 allele; (h) a Vbeta chain that comprises or is derived from a TRBJ2 allele (preferably TRBJ2-7) and a Valpha chain that comprises or is derived from a TRAJ27 allele; (i) a Vbeta chain that comprises or is derived from a TRBJ2 allele (preferably TRBJ2-1) and a Valpha chain that comprises or is derived from a TRAJ37 allele; (j) a Vbeta chain that comprises or is derived from a TRBJ2 allele (preferably TRBJ2-1) and a Valpha chain that comprises or is derived from a TRAJ3 allele.

In particular embodiments, the binding protein of the invention comprises (a) a Vbeta chain that comprises or is derived from a TRBJ1 allele (preferably TRBJ1-2) and a Valpha chain that comprises or is derived from a TRAJ9 allele; (b) a Vbeta chain that comprises or is derived from a TRBJ2 (preferably TRBJ2-7) allele and a Valpha chain that comprises or is derived from a TRAJ33 allele; (c) a Vbeta chain that comprises or is derived from a TRBJ2 allele (preferably TRBJ2-1) and a Valpha chain that comprises or is derived from a TRAJ49 allele; (d) a Vbeta chain that comprises or is derived from a TRBJ2 allele (preferably TRBJ2-6) and a Valpha chain that comprises or is derived from a TRAJ13 allele; (e) a Vbeta chain that comprises or is derived from a TRBJ1 allele (preferably TRBJ1-1) and a Valpha chain that comprises or is derived from a TRAJ23 allele; (f) a Vbeta chain that comprises or is derived from a TRBJ2 allele (preferably TRBJ2-1) and a Valpha chain that comprises or is derived from a TRAJ47allele; (g) a Vbeta chain that comprises or is derived from a TRBJ2 allele (preferably TRBJ2-7) and a Valpha chain that comprises or is derived from a TRAJ42 allele; (h) Vbeta chain that comprises or is derived from a TRBJ2 allele (preferably TRBJ2-1) and a Valpha chain that comprises or is derived from a TRAJ47 allele; (i) a Vbeta chain that comprises or is derived from a TRBJ2 allele (preferably TRBJ2-5) and a Valpha chain that comprises or is derived from a TRAJ31 allele; (j) a Vbeta chain that comprises or is derived from a TRBJ1 allele (preferably TRBJ1-4) and a Valpha chain that comprises or is derived from a TRAJ47 allele.

In any aspect or embodiment, the binding protein of the invention comprises a V beta chain that comprises or is derived from a TRBD1 or TRBD2 allele.

In any aspect or embodiment, the binding protein of the invention comprises a V beta chain that comprises or is derived from a TRC1 or TRBC2 allele and a V alpha chain that comprises or is derived from a TRAC allele.

In any aspect of the present invention, the binding protein comprises a Vα chain comprising the Vα domain and a Vβ chain comprising a Vβ domain. Preferably, the Vα chain and Vβ chain are modified to include a cysteine residue that allows formation of an additional interchain disulfide bond. The cysteine introduced into each of the Vα chain and Vβ chains allows preferential pairing of the Vα and Vβ chain when expressed in a cell that expresses endogenous TCR Vα and Vβ chains. Preferably, the residue at, or equivalent to, Thr48 on the TCR α chain and the residue at, or equivalent to, Ser57 on the TCR β chain are replaced with cysteines to facilitate the creation of an additional disulfide bond between the TCR constant regions. This modification allows preferential pairing of the introduced TCRs and reduces mispairing with endogenous TCRs. This is particularly beneficial for adoptive cell therapies where T regulatory cells are modified to express exogenous TCRs.

Methods useful for isolating and purifying recombinantly produced soluble TCR, by way of example, may include obtaining supernatants from suitable host cell/vector systems that secrete the recombinant soluble TCR into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble TCR described herein include batch cell culture, which is monitored and controlled to maintain appropriate culture conditions. Purification of the soluble TCR may be performed according to methods described herein and known in the art.

The SmB/B′-specific binding proteins or domains as described herein (e.g., SEQ ID NOS.:6-257, and variants thereof), may be functionally characterized according to any of a large number of art accepted methodologies for assaying T cell activity, including determination of T cell binding, activation or induction and also including determination of T cell responses that are antigen-specific. Examples include determination of T cell proliferation, T cell cytokine release, antigen specific T cell stimulation, MHC restricted T cell stimulation, CTL activity (e.g., by detecting Cr release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

As used herein, SmB/B′ refers to the ribonucleoprotein termed “Smith protein” or “small nuclear ribonucleoprotein-associated protein B and B”, a protein that in humans is encoded by the SNRPB gene. SmB/B′ may also be referred to by the aliases: COD, SNRPB1, snRNP-B, CCMS and small nuclear ribonucleoprotein polypeptides B and B1.

The protein encoded by the SNRPB gene is one of several nuclear proteins that are found in common among U1, U2, U4/U6, and U5 small ribonucleoprotein particles (snRNPs). These snRNPs are involved in pre-mRNA splicing, and the encoded protein may also play a role in pre-mRNA splicing or snRNP structure. Two transcript variants encoding different isoforms (B and B′) have been found for this gene.

The Sm and nuclear ribonucleoprotein (RNP) antigens are a particulate complex composed of small nuclear RNAs (U-RNAs) and proteins. This complex has also been referred to as extractable nuclear antigens (ENA), since it is soluble in saline. Autoantibodies to these antigens occur in systemic lupus erythematosis and mixed connective tissue disease.

The Sm (Smith) and related nuclear ribonucleoproteins (nRNPs) are targets for autoantibodies in SLE. These antigens are present in subcellular organelles called spliceosomes that are composed of peptide containing small RNAs. Anti-Sm antibodies are present in 15 to 30% of the patients with SLE, but they are highly specific for SLE. They occur more frequently (60%) in young black females with SLE. They almost never occur in healthy individuals or patients with other diseases. Anti-Sm antibodies are not to be confused with anti-smooth muscle antibodies detected in autoimmune liver disease.

Systemic lupus erythematosus (SLE) is characterized by the presence of various autoantibodies directed against a large number of intracellular antigens. Among the different autoantigenic candidates that are recognized by autoantibodies in SLE, the Sm antigens of the U-1 small nuclear ribonucleoprotein complex are considered pathognomonic of SLE. Antibodies to these autoantigens are sufficiently discriminating to be part of the American College of Rheumatology (ACR) classification criteria for SLE.

Adoptive Cell Therapy

The present invention provides methods of preparing cells for adoptive cell therapy, methods of treating subjects with those cells and the cells per se.

In certain embodiments, nucleic acid molecules encoding a binding protein of the invention are used to transfect/transduce a host cell (e.g., Treg cells) for use in adoptive transfer therapy.

In alternative embodiments, one or more peptides of the invention are used to activate and//or expand a population of T cells, in order to generate T cells (e.g., Treg cells) having specificity for the peptide.

Advances in TCR sequencing have been described (e.g., Robins et al, Blood 114:4099, 2009; Robins et al, Sci. Translat. Med. 2:47ra64, 2010; Robins et al, (September 10) J. Imm. Meth. Epub ahead of print, 2011; Warren et al, Genome Res. 2 1:790, 2011) and may be employed in the course of practicing the embodiments according to the present disclosure. Similarly, methods for transfecting/transducing T cells with desired nucleic acids have been described (e.g., U.S. Patent Application Pub. No. US 2004/0087025) as have adoptive transfer procedures using T-cells of desired antigen-specificity (e.g., Schmitt et al, Hum. Gen. 20:1240, 2009; Dossett et al, Mol. Ther. 77:742, 2009; Till et al, Blood 772:2261, 2008; Wang et al, Hum. Gene Ther. 75:712, 2007; Kuball et al, Blood 709:2331, 2007; US 2011/0243972; US 2011/0189141; e n et al, Ann. Rev. Immunol. 25:243, 2007), such that adaptation of these methodologies to the presently disclosed embodiments is contemplated, based on the teachings herein, including those directed to binding proteins of the invention.

A population of cells comprising regulatory T (Treg) cells may be derived from any source in which Treg cells exist, such as peripheral blood, the thymus, lymph nodes, spleen, and bone marrow.

A population of cells comprising Treg cells may also be derived from a mixed population of T cells, or from a population of conventional T cells. As described herein, the mixed population or conventional T cells may be contacted with a peptide of the invention to enrich Sm antigen specificity in the T cells. Alternatively, the mixed population or conventional T cells may be transduced with a nucleic acid encoding a binding protein of the invention. The T cells may then be converted into Treg cells using standard techniques known to the skilled person for generation of Treg cells. In certain embodiments, the mixed population of T cells, or conventional T cells are cultured in conditions to allow for increased expression of TGF-beta, Foxp3. This includes culturing cells with anti-CD3/anti-CD28 antibodies, inhibition of CDK8/19high doses of IL-2, TGF-beta, and rapamiycin In further embodiments, the converted or enriches population of Treg cells are stabilised (for example, by contacting the cells with Vitamin C or other agent for stabilising the Tregs).

The Treg cells used for infusion (or indeed the Tconv or mixed population of T cells used to generate the Tregs) can be isolated from an allogenic donor, preferably HLA matched, or from the subject diagnosed with a condition associated with the aberrant, unwanted or otherwise inappropriate immune response to a Smith protein. Preferably, the condition is SLE.

The T cells may also be generated from differentiation of induced pluripotent cells (iPSCs) or embryonic stem cells, preferably an embryonic stem cell line. The skilled person will be familiar with standard techniques for generating Treg cells from a stem cells, including an iPSC. Examples of these techniques are described in: Hague et al., (2012) J. Immunol., 189: 2338-36; and Hague et al., (2019) JCI Insight, 4: pii 126471).

Further still, in the context of a mixed population of T cells, the skilled person will be familiar with standard techniques for isolating the subpopulation of the T cells which are CD4+CD25+ T cells (Treg cells). For example, CD4+CD25+ T cells (Treg cells) can be obtained from a biological sample from a subject by negative and positive immuno-selection and cell sorting.

In any method of the invention the Treg cells that have been cultured in the presence of a nucleic acid or vector can be transferred into the same subject from which cells were obtained. In other words, the cells used in a method of the invention can be an autologous cell, i.e., can be obtained from the subject in which the medical condition is treated or prevented. Alternatively, the cell can be allogenically transferred into another subject. Preferably, the cell is autologous to the subject in a method of treating or preventing a medical condition in the subject.

As used herein, the term “ex vivo” or “ex vivo therapy” refers to a therapy where cells are obtained from a patient or a suitable alternate source, such as, a suitable allogenic donor, and are modified, such that the modified cells can be used to treat a disease which will be improved by the therapeutic benefit produced by the modified cells. Treatment includes the administration or re-introduction of the modified cells into the patient. A benefit of ex vivo therapy is the ability to provide the patient the benefit of the treatment, without exposing the patient to undesired collateral effects from the treatment.

The term “administered” means administration of a therapeutically effective dose of the aforementioned composition including the respective cells to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

An “enriched” or “purified” population of cells is an increase in the ratio of particular cells to other cells, for example, in comparison to the cells as found in a subject's body, or in comparison to the ratio prior to exposure to a peptide, nucleic acid or vector of the invention. In some embodiments, in an enriched or purified population of cells, the particular cells include at least 20%, 30%, 40%, 50%, 60%, 70%, 75%, 80%, 90%, 95% or 99% of the total cell population. A population of cells may be defined by one or more cell surface markers and/or properties.

Treg cells that express a binding protein of the invention can be administered to the subject by any method including, for example, injection, infusion, deposition, implantation, oral ingestion, or topical administration, or any combination thereof. Injections can be, e.g., intravenous, intramuscular, intradermal, subcutaneous or intraperitoneal, preferably intravenous. Single or multiple doses can be administered over a given time period, depending upon the condition, the severity thereof and the overall health of the subject, as can be determined by one skilled in the art without undue experimentation. The injections can be given at multiple locations.

Administration of the Treg cells can be alone or in combination with other therapeutic agents. Each dose can include about 10×10³ CD8+ T cells, 20×10³ cells, 50×10³ cells, 100×10³ cells, 200×10³ cells, 500×10³ cells, 1×10⁶ cells, 2×10⁶ cells, 20×10⁶ cells, 50×10⁶ cells, 100×10⁶ cells, 200×10⁶, 500×10⁶, 1×10⁹ cells, 2×10⁹ cells, 5×10⁹ cells, 10×10⁹ cells, and the like. Administration frequency can be, for example, once per week, twice per week, once every two weeks, once every three weeks, once every four weeks, once per month, once every two months, once every three months, once every four months, once every five months, once every six months, and so on. The total number of days where administration occurs can be one day, on 2 days, or on 3, 4, 5, 6, 7, 8, 9, 10, 11, 12, 13, 14, 15, 16, 17, 18, 19, or 20 days, and so on. It is understood that any given administration might involve two or more injections on the same day. For administration, at least 20%, at least 30%, at least 40%, at least 50%, at least 60%, at least 70%, at least 80%, at least 90%, at least 95%, at least 99%, of the Treg cells that are administered exhibit at least one property of a Treg cell.

Peptides

The present invention provides peptides derived from the Smith protein which can bind to HLA-DR15, specifically HLA-DRA*01:01 and HLA-DRB1*15:01 molecule, and induce CD4+ T cell proliferation. These peptides find particular application in immunotherapy to treat a condition associated with an aberrant, unwanted or otherwise inappropriate immune response to a Smith protein. Preferably, the condition is SLE.

In another aspect, the present invention provides a peptide comprising, consisting essentially of or consisting of an amino acid sequence of or equivalent to residues 1 to 15, or 58-72 of a SmB/B′ protein. In one embodiment, the SmB′ protein comprises the amino acid sequence of SEQ ID NO: 5. In a further embodiment, the peptide comprises or consists or consists essentially of the amino acid sequence set forth in any one of SEQ ID NOs: 1, 2, 3 or 4.

In any aspect, a peptide of the invention is capable of binding to, or forming a complex with, a HLA-DR15 molecule, preferably the HLA-DR15 molecule is HLA-DRA*01:01 and HLA-DRB115:01 molecule.

Further, the present invention provides peptides derived from the Smith protein which can bind to HLA-DR3, specifically HLA-DRA*01:01 and HLA-DRB1*03:01 molecule, and induce CD4+ T cell proliferation. These peptides find particular application in immunotherapy to treat a condition associated with an aberrant, unwanted or otherwise inappropriate immune response to a Smith protein. Preferably, the condition is SLE.

In another aspect, the present invention provides a peptide comprising, consisting essentially of or consisting of an amino acid sequence of or equivalent to residues 7-21 of a SmB/B′ protein or a peptide comprising, consisting essentially of or consisting of an amino acid sequence of or equivalent to residues 78-92 of an SmD1 protein. In one embodiment, the SmB′ protein comprises the amino acid sequence of SEQ ID NO: 5 wherein preferably, the peptide comprises or consists or consists essentially of the amino acid sequence set forth in SEQ ID NO: 259. In one embodiment the SmD1 protein comprises the amino acid sequence of SEQ ID NO: 260, wherein preferably, the peptide comprises or consists or consists essentially of the amino acid sequence set forth in SEQ ID NO: 258.

In any aspect, a peptide of the invention is capable of binding to, or forming a complex with, a HLA-DR3 molecule, preferably the HLA-DR3 molecule is HLA-DRA*01:01 and HLA-DRB1*03:01 molecule.

Reference to a “peptide” includes reference to a peptide, polypeptide or protein or parts thereof. The peptide may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “peptide” includes a peptide comprising a sequence of amino acids as well as a peptide associated with other molecules such as amino acids, lipids, carbohydrates or other peptides, polypeptides or proteins.

“Derivatives” include fragments, parts, portions and variants from natural, synthetic or recombinant sources including fusion proteins. Parts or fragments include, for example, active regions of the subject peptide. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence.

Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins. In one embodiment, cysteine residues are substituted with serine, as exemplified herein.

Chemical and functional equivalents of the subject peptide should be understood as molecules exhibiting any one or more of the functional activities of these molecules and may be derived from any source such as being chemically synthesized or identified via screening processes such as natural product screening.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH 4.

The guanidine group of arginine residues may be modified by the formation of heterocyclic condensation products with reagents such as 2,3-butanedione, phenylglyoxal and glyoxal. The carboxyl group may be modified by carbodiimide activation via O-acylisourea formation followed by subsequent derivatisation, for example, to a corresponding amide. Sulphydryl groups may be modified by methods such as carboxymethylation with iodoacetic acid or iodoacetamide; performic acid oxidation to cysteic acid; formation of mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Crosslinkers can be used, for example, to stabilise 3D conformations, using homo-bifunctional crosslinkers such as the bifunctional imido esters having (CH₂)n spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

It is possible to modify the structure of a peptide according to the invention for various purposes such as for increasing solubility, enhancing therapeutic or preventative efficacy, enhancing stability or increasing resistance to proteolytic degradation. A modified peptide may be produced in which the amino acid sequence has been altered, such as by amino acid substitution, deletion or addition, to modify immunogenicity. Similarly components may be added to peptides of the invention to produce the same result.

For example, a peptide can be modified so that it exhibits the ability to induce T cell anergy. In this instance, critical binding residues for the T cell receptor can be determined using known techniques (for example substitution of each residue and determination of the presence or absence of T cell reactivity) In one example, those residues shown to be essential to interact with the T cell receptor can be modified by replacing the essential amino acid with another, preferably similar amino acid residue (a conservative substitution) whose presence is shown to alter T cell reactivity or T cell functioning. In addition, those amino acid residues which are not essential for T cell receptor interaction can be modified by being replaced by another amino acid whose incorporation may then alter T cell reactivity or T cell functioning but does not, for example, eliminate binding to relevant MHC proteins.

Exemplary conservative substitutions are detailed, below, and include:

| Original Residue | Exemplary Substitutions |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Such modifications will result in the production of molecules falling within the scope of “mutants” of the subject peptide as herein defined. “Mutants” should be understood as a reference to peptides which exhibit one or more structural features or functional activities which are distinct from those exhibited by the non-mutated peptide counterpart.

Peptides of the invention may also be modified to incorporate one or more polymorphisms resulting from natural allelic variation and D-amino acids, non-natural amino acids or amino acid analogues may be substituted into the peptides to produce modified peptides which fall within the scope of the invention. Peptides may also be modified by conjugation with polyethylene glycol (PEG) by known techniques. Reporter groups may also be added to facilitate purification and potentially increase solubility of the peptides according to the invention. Other well-known types of modification including insertion of specific endoprotease cleavage sites, addition of functional groups or replacement of hydrophobic residues with less hydrophobic residues as well as site-directed mutagenesis of DNA encoding the peptides of the invention may also be used to introduce modifications which could be useful for a wide range of purposes. The various modifications to peptides according to the invention which have been mentioned above are mentioned by way of example only and are merely intended to be indicative of the broad range of modifications which can be effected.

The peptides of the present invention may be prepared by recombinant or chemical synthetic means. According to a preferred aspect of the present invention, there is provided a recombinant peptide or mutant thereof which is preferentially immunologically reactive with T cells from individuals with Smith protein autoreactivity, which is expressed by the expression of a host cell transformed with a vector coding for the peptide sequence of the present invention. The peptide may be fused to another peptide, polypeptide or protein. Alternatively, the peptide may be prepared by chemical synthetic techniques, such as by the Merrifield solid phase synthesis procedure. Furthermore, although synthetic peptides of the sequence given above represent a preferred embodiment, the present invention also extends to biologically pure preparations of the naturally occurring peptides or fragments thereof. By “biologically pure” is meant a preparation comprising at least about 60%, preferably at least about 70%, or preferably at least about 80% and still more preferably at least about 90% or greater as determined by weight, activity or other suitable means.

Nucleic Acids and Vectors

In another aspect, the present invention provides a nucleic acid molecule composition comprising one or more nucleic acid molecules encoding or complementary to a sequence encoding the binding proteins and peptides of the invention or a derivative, homologue or analogue thereof. The nucleic acid molecules of the invention may be used to produce a binding protein or peptide of the invention, or used for cell therapy to treat a disease or condition described herein.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In any aspect, a vector of the invention may comprise any one of more, or all, of the following:
(i) an EF1α (alpha) promoter;
(ii) a 2A ribosome skipping sequence;
(iii) a woodchuck hepatitis virus post-transcriptional regulatory element (WPRE);
(iv) arrangement to translate TCR β-chain variable (Vβ or Vbeta) domain prior to the (TCR) α-chain variable (Vα or Valpha) domain; or
(v) arrangement to translate TCR β-chain variable (Vβ or Vbeta) chain prior to the (TCR) α-chain variable (Vα or Valpha) chain.

Preferably, the vector is a lentiviral vector. Even more preferably the lentiviral vector has any one or more, or all, of the features shown in FIG. 4.

The term “operably-linked” refers to the association of two or more nucleic acid molecules on a single nucleic acid fragment so that the function of one is affected by the other. For example, a promoter is operably-linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

The term “introduced” in the context of inserting a nucleic acid molecule into a cell, means “transfection”, or ‘transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by an exogenous nucleic acid molecule introduced into the host cell, even if the host cell encodes a homologous protein or activity.

As described herein, more than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous or exogenous nucleic acid molecules encoding desired TCR specific for a WT-1 antigen peptide (e.g., TCRα and TCR-β). When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

The term “homologous” or “homolog” refers to a molecule or activity found in or derived from a host cell, species or strain. For example, a heterologous or exogenous nucleic acid molecule may be homologous to a native host cell gene, and may optionally have an altered expression level, a different sequence, an altered activity, or any combination thereof.

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values can be generated using the NCBI BLAST2.0 software as defined by Altschul et al. (1997) “Gapped BLAST and PSI-BLAST: a new generation of protein database search programs”, Nucleic Acids Res. 25:3389-3402, with the parameters set to default values.

As used herein, the term “host” refers to a cell (e.g., Treg cell) or microorganism targeted for genetic modification with a heterologous or exogenous nucleic acid molecule to produce a polypeptide of interest (e.g., high or enhanced affinity anti-WT-1 TCR). In certain embodiments, a host cell may optionally already possess or be modified to include other genetic modifications that confer desired properties related or unrelated to biosynthesis of the heterologous or exogenous protein (e.g., inclusion of a detectable marker; deleted, altered or truncated endogenous TCR; increased co-stimulatory factor expression). In some embodiments, host cells are genetically modified to express a protein or fusion protein that modulates immune signaling in a host cell to, for example, promote survival and/or expansion advantage to the modified cell (e.g., see immunomodulatory fusion proteins of WO 2016/141357, which are herein incorporated by reference in their entirety).

The nucleic acid molecule may be ligated to an expression vector capable of expression in a prokaryotic cell (e.g., E. coli) or a eukaryotic cell (e.g., yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of the binding protein or peptide of the present invention.

Such nucleic acids may be useful for recombinant production of binding proteins or peptides of the invention or proteins comprising them by insertion into an appropriate vector and transfection into a suitable cell line. Such expression vectors and host cell lines also form an aspect of the invention.

In producing peptides by recombinant techniques, host cells transformed with a nucleic acid having a sequence encoding a binding protein or peptide according to the invention or a functional equivalent of the nucleic acid sequence are cultured in a medium suitable for the particular cells concerned. Binding proteins or peptides can then be purified from cell culture medium, the host cells or both using techniques well known in the art such as ion exchange chromatography, gel filtration chromatography, ultrafiltration, electrophoresis or immunopurification with antibodies specific for the binding protein or peptide.

Nucleic acids encoding binding proteins or peptides of the invention may be expressed in bacterial cells such as E. coli, insect cells, yeast or mammalian cells such as Chinese hamster ovary cells (CHO). Suitable expression vectors, promoters, enhancers and other expression control elements are referred to in Sambruck et al (1989). Other suitable expression vectors, promoters, enhancers and other expression elements are well known to those skilled in the art. Examples of suitable expression vectors in yeast include Yep Sec 1 (Balderi et al., 1987, Embo J., 6:229-234); pMFa (Kurjan and Herskowitz., 1982, Cell., 30:933-943); JRY88 (Schultz et al., 1987, Gene., 54:113-123) and pYES2 (Invitrogen Corporation, San Diego, Calif.). These vectors are freely available as are baculovirus and mammalian expression systems. For example, a baculovirus system is commercially available (ParMingen, San Diego, Calif.) for expression in insect cells while the pMsg vector is commercially available (Pharmacia, Piscataway, N.J.) for expression in mammalian cells.

For expression in E. coli suitable expression vectors include among others, pTrc (Amann et al., 1998, Gene., 69201-315) pGex (Amrad Corporation, Melbourne, Australia); pMal (N.E. Biolabs, Beverley, Mass.); pRit5 (Pharmacia, Piscataway, N.J.); pEt-11d (Novagen, Maddison, Wis.) (Jameel et al., 1990, J. Virol., 64:3963-3966) and pSem (Knapp et al., 1990, Bio Techniques., 8.280-281). The use of pTRC, and pEt-11d, for example, will lead to the expression of unfused protein. The use of pMal, pRit5, pSem and pGex will lead to the expression of a protein or peptide fused to maltose E binding protein (pMal), protein A (pRit5), truncated-galactosidase (PSEM) or glutathione S-transferase (pGex). When a binding protein or peptide is expressed as a fusion protein, it is particularly advantageous to introduce an enzymatic cleavage site at the fusion junction between the carrier protein and the peptide concerned. The binding protein or peptide of the invention may then be recovered from the fusion protein through enzymatic cleavage at the enzymatic site and biochemical purification using conventional techniques for purification of proteins and peptides. The different vectors also have different promoter regions allowing constitutive or inducible expression or temperature induction. It may additionally be appropriate to express recombinant peptides in different E. coli hosts that have an altered capacity to degrade recombinantly expressed proteins. Alternatively, it may be advantageous to alter the nucleic acid sequence to use codons preferentially utilised by E. coli, where such nucleic acid alteration would not affect the amino acid sequence of the expressed proteins.

Host cells can be transformed to express the nucleic acids of the invention using conventional techniques such as calcium phosphate or calcium chloride co-precipitation, DEAE-dextran-mediated transfection or electroporation. Suitable methods for transforming the host cells may be found in Sambruck et al. (1989), and other laboratory texts. The nucleic acid sequence of the invention may also be chemically synthesised using standard techniques.

In addition to recombinant production of peptides according to the invention, the nucleic acids may be utilised as probes for experimental or purification purposes.

Conditions for Treatment

Identification and synthesis of the binding proteins, peptides, cells, nucleic acids, vectors and compositions of the invention as disclosed herein now facilitates the development of a range of prophylactic and therapeutic treatment protocols for use with respect to Smith protein related immune conditions. Also facilitated is the development of reagents for use therein. Accordingly, the present invention should be understood to extend to the use of the peptides or functional derivatives, homologues or analogues thereof in the therapeutic and/or prophylactic treatment of patients. Such methods of treatment include, but are not limited to:

Administration of the subject peptides or cell expressing binding proteins of the invention to a patient as a means of desensitising or inducing immunological tolerance. This may be achieved, for example, by inducing Smith protein directed Th2 anergy or apoptosis. One may utilise treatment protocols which are based on the administration of specific concentrations of a given cell expressing a binding protein or administration of a peptide in accordance with a specific regimen in order to induce tolerance. Such methodology may eliminate Smith protein hypersensitivity or it may reduce the severity of Smith protein hypersensitivity or sensitivity.

Preferably such treatment regimens are capable of modifying the T cell response or both the B and T cell response of the individual concerned. As used herein, modification of the autoimmune response of the subject can be defined as inducing either non-responsiveness or diminution in immunity to a Smith protein or other autoantigen, as determined by standard clinical procedures. In particular, it is expected that the use of Sm-specific Tregs may induce immune tolerance towards autoantigens beyond Smith protein, since immunosuppressive cells recruited as a result of Sm-specific Treg therapy (e.g., Tregs and myeloid derived suppressor cells), exhibit non-antigen-specific immunosuppressive capacity, creating a tolerant environment for multiple autoantigens

Exposure of an individual to the binding proteins, peptides, cells, nucleic acids, vectors and compositions of the invention may tolerise or anergise appropriate T cell subpopulations such that they become unresponsive to Smith protein and other autoantigens and do not participate in stimulating an immune response upon such exposure.

In one embodiment, said method desensitises or induces immunological tolerance to a Smith protein.

In another embodiment, said desensitization or tolerance is achieved by inducing T cell anergy or apoptosis.

In still another embodiment, said desensitisation or tolerance is achieved by inducing Smith-specific Treg cells.

The phrase “therapeutically effective amount” generally refers to an amount of a cell expressing a binding protein, or peptide of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a individual that may be exposed to or predisposed to the disease but does not yet experience or display symptoms of the disease). Biological and physiological parameters for identifying such patients are provided herein and are also well known by physicians.

In particularly preferred embodiments, the methods of the present invention can be to prevent or reduce the severity, or inhibit or minimise progression, of a flare-up or symptom of a disease or condition as described herein. As such, the methods of the present invention have utility as treatments as well as prophylaxes.

The terms “treatment” or “treating” of a subject includes the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of SLE and associated conditions as herein described, including any objective or subjective parameter such as abatement; remission; lessening of the rate of worsening; lessening severity of the condition; stabilization, diminishing of symptoms or making the condition more tolerable to the individual; slowing in the rate of degeneration or decline; making the final point of degeneration less debilitating; or improving a subject's physical or mental well-being.

It will also be understood that the methods described herein can be used in combination with existing standard of care treatments/therapies for SLE. The skilled person will be familiar with existing standard of care approaches to treatment of SLE including but not limited to the use of steroids, anti-malarials (hydroxychloroquine, cholorquine), immunosuppressants (azathioprine, methotrexate, mycophenolate mofeti, mucophenolic acid, tacrolimus, voclosporin, ciclosporin), kinase inibitors (baricitinib, tofacitinib, upaticitinib) and biologics (belimumab, rituximab, anifrolumab, ustekinumab, obinotuzumab). The present invention includes combinations of existing standard of care approaches with the specific methods of the present invention.

A “subject” herein is preferably human subject. Although the invention finds application in humans, the invention is also useful for veterinary purposes. The invention is useful for domestic or farm animals such as cattle, sheep, horses and poultry; for companion animals such as cats and dogs; and for zoo animals. It will be understood that the terms “subject” and “individual” are interchangeable in relation to an individual requiring treatment according to the present invention.

Systemic lupus erythematosus (SLE) is a multi-system autoimmune disease. At least 5 million people worldwide have SLE; 90% of those diagnosed are female and most develop the disease between the ages of 15-44. In Australia, SLE is diagnosed in ˜1 in 1000 people and is more prevalent and severe in Indigenous Australians and Asian Australians. SLE patients suffer chronic immune-mediated inflammatory damage in the brain, kidneys, heart, lungs, joints, skin, and other organs, resulting in a marked loss of life expectancy, exemplified by a standardized mortality ratio above 3. In a British cohort, the average age of death of the 14% of patients who died during follow-up was only 52 years. Most often the clinical course is characterised by episodic flares, which are associated with accrual of irreversible organ damage and thereby mortality.

Other forms of lupus include discoid, drug-induced and neonatal lupus. Of these, systemic lupus erythematosus (also known as SLE) is the most common and serious form. A more thorough categorization of lupus includes the following types: acute cutaneous lupus erythematosus, subacute cutaneous lupus erythematosus, discoid lupus erythematosus (chronic cutaneous), childhood discoid lupus erythematosus, generalized discoid lupus erythematosus, localized discoid lupus erythematosus, chilblain lupus erythematosus (Hutchinson), lupus erythematosus-lichen planus overlap syndrome, lupus erythematosus panniculitis (lupus erythematosus profundus), tumid lupus erythematosus, verrucous lupus erythematosus (hypertrophic lupus erythematosus), cutaneous lupus mucinosis, complement deficiency syndromes, drug-induced lupus erythematosus, neonatal lupus erythematosus, systemic lupus erythematosus.

Cutaneous lupus erythematosus (CLE) is seen in the majority of SLE cases and is most often observed in skin exposed to the sun, appearing as a variety of severe and in some cases disfiguring skin rashes. Lupus may also manifest as a purely cutaneous form, also known as incomplete lupus erythematosus. While all the factors leading to the development of SLE, and its pattern of intermittent flares, are not known, it is clear that sunlight exposure is important in systemic as well as cutaneous disease exacerbation.

Of the symptoms common to those diagnosed with lupus, almost all patients have joint pain and/or swelling (i.e., arthritis). Frequently affected joints are the fingers, hands, wrists, and knees. Other common symptoms include: pleuritic chest pain, oral and nasal ulcers, fatigue, fever with no other cause, general discomfort, uneasiness, or ill feeling (malaise), hair loss, sensitivity to sunlight, skin rash—a “butterfly” rash in about half people with SLE and also scarring “discoid” lesions, and swollen lymph nodes. The skilled person will be familiar with various other important manifestations of lupus, including but not limited to: nephritis, CNS involvement, haematological involvement, gastrointestinal involvement, and vasculitis.

As used herein, photosensitivity or abnormal light sensitivity in an individual with CLE or SLE includes skin rashes that result of unusual reaction to sunlight. Beyond skin rashes that can develop, exposure to the sun can cause those living with lupus to experience increased disease activity with symptoms such as joint pains, weakness, fatigue and fever. Two-thirds of people with lupus have increased sensitivity to ultraviolet rays, either from sunlight or from artificial inside light, such as fluorescent light—or both.

Compositions

Administration of a composition of the present invention (herein referred to as “agent”) in the form of a pharmaceutical composition, may be performed by any convenient means. In certain embodiments, the agent is a peptide as described herein, preferably a peptide comprising, consisting of or consisting essentially of the sequence set forth in any one of SEQ ID NOs: 1-4. The agent of the pharmaceutical composition is contemplated to exhibit therapeutic activity when administered in an amount which depends on the particular case. The variation depends, for example, on the human or animal and the agent chosen. A broad range of doses may be applicable. Considering a patient, for example, from about 0.01 μg to about 1 mg of an agent may be administered per dose. Dosage regimens may be adjusted to provide the optimum therapeutic response. For example, several divided doses may be administered daily, weekly, monthly or other suitable time intervals or the dose may be proportionally reduced as indicated by the exigencies of the situation. In another example, said composition is administered initially to induce tolerance and then, if necessary, booster administrations of the composition are administered to maintain tolerance. These boosters may be administered monthly, for example, and may be administered for any period of time, including the life of the patient.

The agent may be administered in a convenient manner such as by the oral, intravenous (where water soluble), intraperitoneal, intramuscular, subcutaneous, intradermal (with or without using a traditional needle or other transdermal delivery device), transdermal, intranasal, sublingual or suppository routes or implanting (e.g. using slow release molecules). Preferably, said composition is administered intradermally. The agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate. In the context of a peptide for administration, the composition comprising said peptide may be in the form of a liposome or conjugated to nanoparticles. The skilled person will be familiar with standard techniques for formulating peptides for administration to a subject in need thereof.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. Tonicity adjusting agents are useful to keep the preparation isotonic with human plasma and thus avoid tissue damage. Commonly used tonicity agents include Dextrose, Trehalose, Glycerin and Mannitol. Glycerol and sodium chloride are other options but are less commonly used. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 1000 μg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Routes of administration include, but are not limited to, respiratorally (eg. intranasally or orally via aerosol), intratracheally, nasopharyngeally, intravenously, intraperitoneally, subcutaneously, intracranially, intradermally, transdermally, intramuscularly, intraoccularly, intrathecally, intracereberally, intranasally, infusion, orally, rectally, via IV drip patch, implant and sublingual. Preferably, said route of administration is intravenously, subcutaneously, intradermally, transdermally or intranasally, more preferably, intravenously.

Yet another aspect of the present invention relates to the compositions, as hereinbefore defined, when used in any method of the present invention.

### Summary of Amino Acid and Nucleotide Sequences

TABLE 1
HLA-DR15 restricted peptides and TCRs

| Peptide/ | | | |
|---|---|---|---|
| protein | Description | SEQ ID NO: | Amino acid or nucleotide sequence |
| SmB/B′ | Epitope | 1 | KMLQHIDYRMRCILQ |
| 1-15 | | | |
| SmB/B′ | Epitope | 2 | RVLGLVLLRGENLVS |
| 58-72 | | | |
| SmB/B′ | Core epitope | 3 | IDYRMRCIL |
| 6-14 | | | |
| SmB/B′ | Core epitope | 4 | LVLLRGENL |
| 62-70 | | | |
| SmB′ protein | Mature protein | 5 | KMLQHIDYRMRCILQDGRIFIGTFKAFDKHMNLILCDCDE |
| | sequence | | FRKIKPKNSKQAEREEKRVLGLVLLRGENLVSMTVEGPP |
| | | | PKDTGIARVPLAGAAGGPGIGRAAGRGIPAGVPMPQAPA |
| | | | GLAGPVRGVGGPSQQVMTPQGRGTVAAAAAAATASIAG |
| | | | APTQYPPGRGGPPPPMGRGAPPPGMMGPPPGMRPPM |
| | | | GPPMGIPPGRGTPMGMPPPGMRPPPPGMRGPPPPGM |
| | | | RPPRP (underlined sequence corresponds to epitope). |
| | | | Note: The sequence above is SmB′, it is an |
| | | | alternatively spliced variant of SmB that only |
| | | | differs at the end (from residue 223: it is |
| | | | “LL” for SmB and “PPPPGMRPPRP” for SmB′. |
| TCR | Description | SEQ ID NO: | Amino acid or nucleotide sequence |
| 1 | CDR1 alpha | 6 | ATGYPS |
| | (protein) | | |
| | CDR2 alpha | 7 | ATKADDK |
| | (protein) | | |
| | CDR3 alpha | 8 | CALSSYGNKLVF |
| | (protein) | | |
| | CDR1 beta | 9 | SGHAT |
| | (protein) | | |
| | CDR2 beta | 10 | FQNNGV |
| | (protein) | | |
| | CDR3 beta | 11 | CASSSLSGSSYEQYF |
| | (protein) | | |
| | CDR1 alpha | 12 | GCCACAGGATACCCTTCC |
| | (DNA) | | |
| | CDR2 alpha | 13 | GCCACGAAGGCTGATGACAAG |
| | (DNA) | | |
| | CDR3 alpha | 14 | TGTGCTCTGAGTAGCTATGGAAACAAGCTGGTCTTT |
| | (DNA) | | |
| | CDR1 beta | 15 | TCTGGCCATGCTACC |
| | (DNA) | | |
| | CDR2 beta | 16 | TTTCAGAATAACGGTGTA |
| | (DNA) | | |
| | CDR3 beta | 17 | TGTGCCAGCAGCTCGCTTAGCGGGAGCTCCTACGAGCAGTACTTC |
| | (DNA) | | |
| 2 | CDR1 alpha | 18 | TTLSN |
| | (protein) | | |
| | CDR2 alpha | 19 | LVKSGEV |
| | (protein) | | |
| | CDR3 alpha | 20 | CAGHAGAQKLVF |
| | (protein) | | |
| | CDR1 beta | 21 | MNHNS |
| | (protein) | | |
| | CDR2 beta | 22 | SASEGT |
| | (protein) | | |
| | CDR3 beta | 23 | CASSEVWVGSTDTQYF |
| | (protein) | | |
| | CDR1 alpha | 24 | ACTACTTTAAGCAAT |
| | (DNA) | | |
| | CDR2 alpha | 25 | TTAGTGAAGAGTGGAGAAGTG |
| | (DNA) | | |
| | CDR3 alpha | 26 | TGTGCAGGGCACGCAGGAGCCCAGAAGCTGGTATTT |
| | (DNA) | | |
| | CDR1 beta | 27 | ATGAACCATAACTCC |
| | (DNA) | | |
| | CDR2 beta | 28 | TCAGCTTCTGAGGGTACC |
| | (DNA) | | |
| | CDR3 beta | 29 | TGTGCCAGCAGTGAGGTATGGGTCGGTAGCACAGATACGCAGTATTTT |
| | (DNA) | | |
| 3 | CDR1 alpha | 30 | NSMFDY |
| | (protein) | | |
| | CDR2 alpha | 31 | ISSIKDK |
| | (protein) | | |
| | CDR3 alpha | 32 | CAASRRFGNEKLTF |
| | (protein) | | |
| | CDR1 beta | 33 | SEHNR |
| | (protein) | | |
| | CDR2 beta | 34 | FQNEAQ |
| | (protein) | | |
| | CDR3 beta | 35 | CASRLWSRGRTTEAFF |
| | (protein) | | |
| | CDR1 alpha | 36 | AACAGCATGTTTGATTAT |
| | (DNA) | | |
| | CDR2 alpha | 37 | ATAAGTTCCATTAAGGATAAA |
| | (DNA) | | |
| | CDR3 alpha | 38 | TGTGCAGCAAGCAGGCGGTTTGGAAATGAGAAATTAACCTTT |
| | (DNA) | | |
| | CDR1 beta | 39 | TCTGAACACAACCGC |
| | (DNA) | | |
| | CDR2 beta | 40 | TTCCAGAATGAAGCTCAA |
| | (DNA) | | |
| | CDR3 beta | 41 | TGTGCCAGCAGGCTCTGGAGCAGGGGGCGGACCACTGAAGCTTTCTTT |
| | (DNA) | | |
| 4 | CDR1 alpha | 42 | NTAFDY |
| | (protein) | | |
| | CDR2 alpha | 43 | IRPDVSE |
| | (protein) | | |
| | CDR3 alpha | 44 | CAASKTGTASKLTF |
| | (protein) | | |
| | CDR1 beta | 45 | MDHEN |
| | (protein) | | |
| | CDR2 beta | 46 | SYDVKM |
| | (protein) | | |
| | CDR3 beta | 47 | CASSVNEQFF |
| | (protein) | | |
| | CDR1 alpha | 48 | AACACTGCGTTTGACTAC |
| | (DNA) | | |
| | CDR2 alpha | 49 | ATACGTCCAGATGTGAGTGAA |
| | (DNA) | | |
| | CDR3 alpha | 50 | TGTGCAGCAAGCAAGACCGGCACTGCCAGTAAACTCACCTTT |
| | (DNA) | | |
| | CDR1 beta | 51 | ATGGACCATGAAAAT |
| | (DNA) | | |
| | CDR2 beta | 52 | TCATATGATGTTAAAATG |
| | (DNA) | | |
| | CDR3 beta | 53 | TGTGCCAGCAGTGTGAATGAGCAGTTCTTC |
| | (DNA) | | |
| 5 | CDR1 alpha | 54 | TISGNEY |
| | (protein) | | |
| | CDR2 alpha | 55 | GLKNN |
| | (protein) | | |
| | CDR3 alpha | 56 | CIVKSWNNRKLIW |
| | (protein) | | |
| | CDR1 beta | 57 | SEHNR |
| | (protein) | | |
| | CDR2 beta | 58 | FQNEAQ |
| | (protein) | | |
| | CDR3 beta | 59 | CASLKRTGGHNQPQHF |
| | (protein) | | |
| | CDR1 alpha | 60 | ACCATCAGTGGAAATGAGTAT |
| | (DNA) | | |
| | CDR2 alpha | 61 | GGTCTAAAAAACAAT |
| | (DNA) | | |
| | CDR3 alpha | 62 | TGCATCGTCAAAAGTTGGAACAACCGTAAGCTGATTTGG |
| | (DNA) | | |
| | CDR1 beta | 63 | TCTGAACACAACCGC |
| | (DNA) | | |
| | CDR2 beta | 64 | TTCCAGAATGAAGCTCAA |
| | (DNA) | | |
| | CDR3 beta | 65 | TGTGCCAGCCTTAAGAGAACAGGGGGCCACAATCAGCCCCAGCATTTT |
| | (DNA) | | |
| 6 | CDR1 alpha | 66 | SSVSVY |
| | (protein) | | |
| | CDR2 alpha | 67 | YLSGSTQV |
| | (protein) | | |
| | CDR3 alpha | 68 | CAVSVSGSARQLTF |
| | (protein) | | |
| | CDR1 beta | 69 | SGHTA |
| | (protein) | | |
| | CDR2 beta | 70 | FQGTGA |
| | (protein) | | |
| | CDR3 beta | 71 | CSARDPGTLYEQYF |
| | (protein) | | |
| | CDR1 alpha | 72 | TCGTCTGTTTCAGTGTAT |
| | (DNA) | | |
| | CDR2 alpha | 73 | TATTTATCAGGATCCACCCAGGTT |
| | (DNA) | | |
| | CDR3 alpha | 74 | TGTGCTGTGAGTGTTTCTGGTTCTGCAAGGCAACTGACCTTT |
| | (DNA) | | |
| | CDR1 beta | 75 | TCAGGTCATACTGCC |
| | (DNA) | | |
| | CDR2 beta | 76 | TTCCAAGGCACGGGTGCG |
| | (DNA) | | |
| | CDR3 beta | 77 | TGTGCCAGCAGCCACCGTCTAGCGGGAGCTGAGCAGTTCTTC |
| | (DNA) | | |
| 7 | CDR1 alpha | 78 | ATGYPS |
| | (protein) | | |
| | CDR2 alpha | 79 | ATKADDK |
| | (protein) | | |
| | CDR3 alpha | 80 | CALTLNSGYSTLTF |
| | (protein) | | |
| | CDR1 beta | 81 | DFQATT |
| | (protein) | | |
| | CDR2 beta | 82 | SNEGSKA |
| | (protein) | | |
| | CDR3 beta | 83 | CSARDPGTLYEQYF |
| | (protein) | | |
| | CDR1 alpha | 84 | GCCACAGGATACCCTTCC |
| | (DNA) | | |
| | CDR2 alpha | 85 | GCCACGAAGGCTGATGACAAG |
| | (DNA) | | |
| | CDR3 alpha | 86 | TGTGCTCTGACCCTGAATTCAGGATACAGCACCCTCACCTTT |
| | (DNA) | | |
| | CDR1 beta | 87 | GACTTTCAGGCCACAACT |
| | (DNA) | | |
| | CDR2 beta | 88 | TCCAATGAGGGCTCCAAGGCC |
| | (DNA) | | |
| | CDR3 beta | 89 | TGCAGTGCTAGAGACCCAGGAACCTTATACGAGCAGTACTTC |
| | (DNA) | | |
| 8 | CDR1 alpha | 90 | VSNAYN |
| | (protein) | | |
| | CDR2 alpha | 91 | GSKP |
| | (protein) | | |
| | CDR3 alpha | 92 | CAVERPGFQKLVF |
| | (protein) | | |
| | CDR1 beta | 93 | LGHDT |
| | (protein) | | |
| | CDR2 beta | 94 | YNNKEL |
| | (protein) | | |
| | CDR3 beta | 95 | CASRTTGSGTEAFF |
| | (protein) | | |
| | CDR1 alpha | 96 | GTGTCCAATGCTTACAAC |
| | (DNA) | | |
| | CDR2 alpha | 97 | GGCTCAAAGCCT |
| | (DNA) | | |
| | CDR3 alpha | 98 | TGTGCTGTGGAGCGGCCAGGCTTTCAGAAACTTGTATTT |
| | (DNA) | | |
| | CDR1 beta | 99 | CTGGGCCATGATACT |
| | (DNA) | | |
| | CDR2 beta | 100 | TACAATAATAAGGAGCTC |
| | (DNA) | | |
| | CDR3 beta | 101 | TGTGCCAGCAGGACGACAGGGTCGGGGACTGAAGCTTTCTTT |
| | (DNA) | | |
| 9 | CDR1 alpha1 | 102 | TSINN |
| | (protein) | | |
| | CDR2 alpha1 | 103 | IRSNERE |
| | (protein) | | |
| | CDR3 alpha1 | 104 | CATPRRGADGLTF |
| | (protein) | | |
| | CDR1 alpha2 | 105 | SVFSS |
| | (protein) | | |
| | CDR2 alpha2 | 106 | VVTGGEV |
| | (protein) | | |
| | CDR3 alpha2 | 107 | CAFCNQFYF |
| | (protein) | | |
| | CDR1 beta | 108 | LGHNA |
| | (protein) | | |
| | CDR2 beta | 109 | YNFKEQ |
| | (protein) | | |
| | CDR3 beta | 110 | CASSPGGSGRLGDTQYF |
| | (protein) | | |
| | CDR1 alpha1 | 111 | ACTAGTATAAACAAT |
| | (DNA) | | |
| | CDR2 alpha1 | 112 | ATACGTTCAAATGAAAGAGAG |
| | (DNA) | | |
| | CDR3 alpha1 | 113 | TGTGCTACGCCGAGGCGAGGTGCTGACGGACTCACCTTT |
| | (DNA) | | |
| | CDR1 alpha2 | 114 | AGTGTTTTTTCCAGC |
| | (DNA) | | |
| | CDR2 alpha2 | 115 | GTAGTTACGGGTGGAGAAGTG |
| | (DNA) | | |
| | CDR3 alpha2 | 116 | TGTGCCTTTTGTAACCAGTTCTATTTT |
| | (DNA) | | |
| | CDR1 beta | 117 | CTGGGGCATAACGCT |
| | (DNA) | | |
| | CDR2 beta | 118 | TACAACTTTAAAGAACAG |
| | (DNA) | | |
| | CDR3 beta | 119 | TGTGCCAGCAGCCCGGGGGGTAGCGGGAGGTTGGGAGATACGCAGTATTTT |
| | (DNA) | | |
| 10 | CDR1 alpha | 120 | VSNAYN |
| | (protein) | | |
| | CDR2 alpha | 121 | GSKP |
| | (protein) | | |
| | CDR3 alpha | 122 | CAVEDSRGNNRLAF |
| | (protein) | | |
| | CDR1 beta | 123 | MNHEY |
| | (protein) | | |
| | CDR2 beta | 124 | SVGAGI |
| | (protein) | | |
| | CDR3 beta | 125 | CASGGTGGNYGYTF |
| | (protein) | | |
| | CDR1 alpha | 126 | GTGTCCAATGCTTACAAC |
| | (DNA) | | |
| | CDR2 alpha | 127 | GGCTCAAAGCCT |
| | (DNA) | | |
| | CDR3 alpha | 128 | TGTGCTGTGGAGGATTCGCGGGGGAACAACAGACTCGCTTTT |
| | (DNA) | | |
| | CDR1 beta | 129 | ATGAACCATGAATAC |
| | (DNA) | | |
| | CDR2 beta | 130 | TCAGTTGGTGCTGGTATC |
| | (DNA) | | |
| | CDR3 beta | 131 | TGTGCCAGCGGAGGGACAGGGGGGAACTATGGCTACACCTTC |
| | (DNA) | | |
| 11 | CDR1 alpha | 132 | TSENNYY |
| | (protein) | | |
| | CDR2 alpha | 133 | QEAYKQQN |
| | (protein) | | |
| | CDR3 alpha | 134 | CAFMKSLFGNEKLTF |
| | (protein) | | |
| | CDR1 beta | 135 | DFQATT |
| | (protein) | | |
| | CDR2 beta | 136 | SNEGSKA |
| | (protein) | | |
| | CDR3 beta | 137 | CSARSGEKLFF |
| | (protein) | | |
| | CDR1 alpha | 138 | ACCAGTGAGAATAATTATTAT |
| | (DNA) | | |
| | CDR2 alpha | 139 | CAAGAAGCTTATAAGCAACAGAAT |
| | (DNA) | | |
| | CDR3 alpha | 140 | TGTGCTTTCATGAAGTCGCTATTTGGAAATGAGAAATTAACCTTT |
| | (DNA) | | |
| | CDR1 beta | 141 | GACTTTCAGGCCACAACT |
| | (DNA) | | |
| | CDR2 beta | 142 | TCCAATGAGGGCTCCAAGGCC |
| | (DNA) | | |
| | CDR3 beta | 143 | TGCAGTGCTAGGTCGGGCGAAAAACTGTTTTTT |
| | (DNA) | | |
| 12 | CDR1 alpha | 144 | TSENNYY |
| | (protein) | | |
| | CDR2 alpha | 145 | QEAYKQQN |
| | (protein) | | |
| | CDR3 alpha | 146 | CAFMKSLFGNEKLTF |
| | (protein) | | |
| | CDR1 beta | 147 | DFQATT |
| | (protein) | | |
| | CDR2 beta | 148 | SNEGSKA |
| | (protein) | | |
| | CDR3 beta | 149 | CSARWGDQPQHF |
| | (protein) | | |
| | CDR1 alpha | 150 | ACCAGTGAGAATAATTATTAT |
| | (DNA) | | |
| | CDR2 alpha | 151 | CAAGAAGCTTATAAGCAACAGAAT |
| | (DNA) | | |
| | CDR3 alpha | 152 | TGTGCTTTCATGAAGTCTCCCTTTGGAAATGAGAAATTAACCTTT |
| | (DNA) | | |
| | CDR1 beta | 153 | GACTTTCAGGCCACAACT |
| | (DNA) | | |
| | CDR2 beta | 154 | TCCAATGAGGGCTCCAAGGCC |
| | (DNA) | | |
| | CDR3 beta | 155 | TGCAGTGCTAGGTGGGGTGATCAGCCCCAGCATTTT |
| | (DNA) | | |
| 13 | CDR1 alpha | 156 | TSENNYY |
| | (protein) | | |
| | CDR2 alpha | 157 | QEAYKQQN |
| | (protein) | | |
| | CDR3 alpha | 158 | CAFMKTNFGNEKLTF |
| | (protein) | | |
| | CDR1 beta | 159 | DFQATT |
| | (protein) | | |
| | CDR2 beta | 160 | SNEGSKA |
| | (protein) | | |
| | CDR3 beta | 161 | CSARSGTSKFF |
| | (protein) | | |
| | CDR1 alpha | 162 | ACCAGTGAGAATAATTATTAT |
| | (DNA) | | |
| | CDR2 alpha | 163 | CAAGAAGCTTATAAGCAACAGAAT |
| | (DNA) | | |
| | CDR3 alpha | 164 | TGTGCTTTCATGAAGACTAACTTTGGAAATGAGAAATTAACCTTT |
| | (DNA) | | |
| | CDR1 beta | 165 | GACTTTCAGGCCACAACT |
| | (DNA) | | |
| | CDR2 beta | 166 | TCCAATGAGGGCTCCAAGGCC |
| | (DNA) | | |
| | CDR3 beta | 167 | TGCAGTGCTAGGTCCGGGACTAGCAAATTCTTC |
| | (DNA) | | |
| 14 | CDR1 alpha | 168 | TSENNYY |
| | (protein) | | |
| | CDR2 alpha | 169 | QEAYKQQN |
| | (protein) | | |
| | CDR3 alpha | 170 | CAFTKSNFGNEKLTF |
| | (protein) | | |
| | CDR1 beta | 171 | DFQATT |
| | ( protein) | | |
| | CDR2 beta | 172 | SNEGSKA |
| | (protein) | | |
| | CDR3 beta | 173 | CSARTGDEQYF |
| | (protein) | | |
| | CDR1 alpha | 174 | ACCAGTGAGAATAATTATTAT |
| | (DNA) | | |
| | CDR2 alpha | 175 | CAAGAAGCTTATAAGCAACAGAAT |
| | (DNA) | | |
| | CDR3 alpha | 176 | TGTGCTTTCACGAAGTCTAACTTTGGAAATGAGAAATTAACCTTT |
| | (DNA) | | |
| | CDR1 beta | 177 | GACTTTCAGGCCACAACT |
| | (DNA) | | |
| | CDR2 beta | 178 | TCCAATGAGGGCTCCAAGGCC |
| | (DNA) | | |
| | CDR3 beta | 179 | TGCAGTGCTCGGACAGGAGACGAGCAGTACTTC |
| | (DNA) | | |
| 15 | CDR1 alpha | 180 | TSENNYY |
| | (protein) | | |
| | CDR2 alpha | 181 | QEAYKQQN |
| | (protein) | | |
| | CDR3 alpha | 182 | CAFIKSNFGNEKLTF |
| | (protein) | | |
| | CDR1 beta | 183 | DFQATT |
| | (protein) | | |
| | CDR2 beta | 184 | SNEGSKA |
| | (protein) | | |
| | CDR3 beta | 185 | CSARRGTEAFF |
| | (protein) | | |
| | CDR1 alpha | 186 | ACCAGTGAGAATAATTATTAT |
| | (DNA) | | |
| | CDR2 alpha | 187 | CAAGAAGCTTATAAGCAACAGAAT |
| | (DNA) | | |
| | CDR3 alpha | 188 | TGTGCTTTCATCAAATCTAACTTTGGAAATGAGAAATTAACCTTT |
| | (DNA) | | |
| | CDR1 beta | 189 | GACTTTCAGGCCACAACT |
| | (DNA) | | |
| | CDR2 beta | 190 | TCCAATGAGGGCTCCAAGGCC |
| | (DNA) | | |
| | CDR3 beta | 191 | TGCAGTGCCCGGAGGGGGACTGAAGCTTTCTTT |
| | (DNA) | | |
| 16 | CDR1 alpha1 | 192 | TSGFNG |
| | (protein) | | |
| | CDR2 alpha1 | 193 | NVLDGL |
| | (protein) | | |
| | CDR3 alpha1 | 194 | CAVMDSSYKLIF |
| | (protein) | | |
| | CDR1 alpha2 | 195 | NIATNDY |
| | (protein) | | |
| | CDR2 alpha2 | 196 | GYKTK |
| | (protein) | | |
| | CDR3 alpha2 | 197 | CLVKIIF |
| | (protein) | | |
| | CDR1 beta | 198 | MRHNA |
| | (protein) | | |
| | CDR2 beta | 199 | SNTAGT |
| | (protein) | | |
| | CDR3 beta | 200 | CASSEREHYGYTF |
| | (protein) | | |
| | CDR1 alpha1 | 201 | ACATCTGGGTTCAACGGG |
| | (DNA) | | |
| | CDR2 alpha1 | 202 | AATGTTCTGGATGGTTTG |
| | (DNA) | | |
| | CDR3 alpha1 | 203 | TGTGCTGTGATGGATAGCAGCTATAAATTGATCTTC |
| | (DNA) | | |
| | CDR1 alpha2 | 204 | AACATTGCTACAAATGATTAT |
| | (DNA) | | |
| | CDR2 alpha2 | 205 | GGATACAAGACAAAA |
| | (DNA) | | |
| | CDR3 alpha2 | 206 | TGCCTCGTGAAGATAATCTTT |
| | (DNA) | | |
| | CDR1 beta | 207 | ATGAGACATAATGCC |
| | (DNA) | | |
| | CDR2 beta | 208 | TCAAATACTGCAGGTACC |
| | (DNA) | | |
| | CDR3 beta | 209 | TGTGCCAGCAGTGAAAGGGAGCACTATGGCTACACCTTC |
| | (DNA) | | |
| 17 | CDR1 alpha | 210 | TSINN |
| | (protein) | | |
| | CDR2 alpha | 211 | IRSNERE |
| | (protein) | | |
| | CDR3 alpha | 212 | CATPYTGGFKTIF |
| | (protein) | | |
| | CDR1 beta | 213 | MGHRA |
| | (protein) | | |
| | CDR2 beta | 214 | YSYEKL |
| | (protein) | | |
| | CDR3 beta | 215 | CASSQLGSGNTIYF |
| | (protein) | | |
| | CDR1 alpha | 216 | ACTAGTATAAACAAT |
| | (DNA) | | |
| | CDR2 alpha | 217 | ATACGTTCAAATGAAAGAGAG |
| | (DNA) | | |
| | CDR3 alpha | 218 | TGTGCTACGCCATATACTGGAGGCTTCAAAACTATCTTT |
| | (DNA) | | |
| | CDR1 beta | 219 | ATGGGGCACAGGGCT |
| | (DNA) | | |
| | CDR2 beta | 220 | TACAGCTATGAGAAACTC |
| | (DNA) | | |
| | CDR3 beta | 221 | TGCGCCAGCAGCCAACTTGGCTCTGGAAACACCATATATTTT |
| | (DNA) | | |
| 18 | CDR1 alpha | 222 | DSAIYN |
| | (protein) | | |
| | CDR2 alpha | 223 | IQSSQRE |
| | (protein) | | |
| | CDR3 alpha | 224 | CAVKAQGGAGSYQLTF |
| | (protein) | | |
| | CDR1 beta | 225 | SGHNT |
| | (protein) | | |
| | CDR2 beta | 226 | YYREEE |
| | (protein) | | |
| | CDR3 beta | 227 | CGSGRERTDTQYF |
| | (protein) | | |
| | CDR1 alpha | 228 | GATAGCGCTATTTACAAC |
| | (DNA) | | |
| | CDR2 alpha | 229 | ATTCAGTCAAGTCAGAGAGAG |
| | (DNA) | | |
| | CDR3 alpha | 230 | TGTGCTGTGAAGGCCCAGGGTGGGGCTGGGAGTTACCAACTCACTTTC |
| | (DNA) | | |
| | CDR1 beta | 231 | TCTGGGCACAACACT |
| | (DNA) | | |
| | CDR2 beta | 232 | TATTATAGGGAGGAAGAG |
| | (DNA) | | |
| | CDR3 beta | 233 | TGTGGGTCCGGGCGGGAACGCACAGATACGCAGTATTTT |
| | (DNA) | | |
| 19 | CDR1 alpha | 234 | SVFSS |
| | (protein) | | |
| | CDR2 alpha | 235 | VVTGGEV |
| | (protein) | | |
| | CDR3 alpha | 236 | CAGANSGYALNF |
| | (protein) | | |
| | CDR1 beta | 237 | MDHEN |
| | (protein) | | |
| | CDR2 beta | 238 | SYDVKM |
| | (protein) | | |
| | CDR3 beta | 239 | CASSSLPGQLYGYTF |
| | (protein) | | |
| | CDR1 alpha | 240 | AGTGTTTTTTCCAGC |
| | (DNA) | | |
| | CDR2 alpha | 241 | GTAGTTACGGGTGGAGAAGTG |
| | (DNA) | | |
| | CDR3 alpha | 242 | TGTGCAGGAGCTAATTCCGGGTATGCACTCAACTTC |
| | (DNA) | | |
| | CDR1 beta | 243 | ATGGACCATGAAAAT |
| | (DNA) | | |
| | CDR2 beta | 244 | TCATATGATGTTAAAATG |
| | (DNA) | | |
| | CDR3 beta | 245 | TGTGCCAGCAGTTCCCTACCGGGACAGCTTTATGGCTACACCTTC |
| | (DNA) | | |
| 20 | CDR1 alpha | 246 | TSGFYG |
| | (protein) | | |
| | CDR2 alpha | 247 | NALDGL |
| | (protein) | | |
| | CDR3 alpha | 248 | CAEGGFKTIF |
| | (protein) | | |
| | CDR1 beta | 249 | KGHDR |
| | (protein) | | |
| | CDR2 beta | 250 | SFDVKD |
| | (protein) | | |
| | CDR3 beta | 251 | CATSGPLTGPPEQFF |
| | (protein) | | |
| | CDR1 alpha | 252 | ACATCTGGGTTTTATGGG |
| | (DNA) | | |
| | CDR2 alpha | 253 | AATGCTCTGGATGGTTTG |
| | (DNA) | | |
| | CDR3 alpha | 254 | TGCGCTGAGGGAGGCTTCAAAACTATCTTT |
| | (DNA) | | |
| | CDR1 beta | 255 | AAGGGTCATGATAGA |
| | (DNA) | | |
| | CDR2 beta | 256 | TCCTTTGATGTCAAAGAT |
| | (DNA) | | |
| | CDR3 beta | 257 | TGTGCCACCAGTGGCCCCCTCACAGGGCCCCCCGAGCAGTTCTTC |
| | (DNA) | | |

TABLE 2
HLA-DR3 restricted peptides and TCRs

| Peptide/ | | SEQ | |
|---|---|---|---|
| protein | Description | ID NO: | Amino acid sequence |
| SmD1 | Epitope | 258 | TLLVDVEPKVKSKKR |
| 78-92 | | | |
| SmB/B′ | Epitope | 259 | DYRMRCILQDGRIFI |
| 7-21 | | | |
| SmD1 | Full protein | 260 | MKLVRFLMKLSHETVTIELKNGTQVHGTITGVDVSMNTHL |
| protein | | | KAVKMTLKNREPVQLETLSIRGNNIRYFILPDSLPLDTLLV |
| | | | DVEPKVKSKKREAVAGRGRGRGRGRGRGRGRGRGGP |
| | | | RR |
| | | SEQ | |
| TCR | Description | ID NO: | Amino acid or nucleotide sequence |
| 1 | CDR1 alpha | 261 | VSGLRG |
| | (protein) | | |
| | CDR2 alpha | 262 | LYSAGEE |
| | (protein) | | |
| | CDR3 alpha | 263 | CAAAIGSYIPTF |
| | (protein) | | |
| | CDR1 beta | 264 | SGHRS |
| | (protein) | | |
| | CDR2 beta | 265 | YFSETQ |
| | (protein) | | |
| | CDR3 beta | 266 | CASSLGNSNTEAFF |
| | (protein) | | |
| | CDR1 alpha | 267 | GTCAGCGGTTTAAGAGGG |
| | (DNA) | | |
| | CDR2 alpha | 268 | CTGTATTCAGCTGGGGAAGAA |
| | (DNA) | | |
| | CDR3 alpha | 269 | TGTGCTGCGGCCATAGGAAGCTACATACCTACATTT |
| | (DNA) | | |
| | CDR1 beta | 270 | TCTGGGCATAGGAGT |
| | (DNA) | | |
| | CDR2 beta | 271 | TACTTCAGTGAGACACAG |
| | (DNA) | | |
| | CDR3 beta | 272 | TGCGCCAGCAGCTTGGGGAATTCGAACACTGAAGCTT |
| | (DNA) | | TCTTT |
| 2 | CDR1 alpha | 273 | DRGSQS |
| | (protein) | | |
| | CDR2 alpha | 274 | IYSNGD |
| | (protein) | | |
| | CDR3 alpha | 275 | CAVRESGGGADGLTF |
| | (protein) | | |
| | CDR1 beta | 276 | SQVTM |
| | (protein) | | |
| | CDR2 beta | 277 | ANQGSEA |
| | (protein) | | |
| | CDR3 beta | 278 | CSVGEQGMTQPQHF |
| | (protein) | | |
| | CDR1 alpha | 279 | GACCGAGGTTCCCAGTCC |
| | (DNA) | | |
| | CDR2 alpha | 280 | ATATACTCCAATGGTGAC |
| | (DNA) | | |
| | CDR3 alpha | 281 | TGTGCCGTGAGGGAGTCAGGAGGAGGTGCTGACGGA |
| | (DNA) | | CTCACCTTT |
| | CDR1 beta | 282 | AGCCAAGTCACCATG |
| | (DNA) | | |
| | CDR2 beta | 283 | GCAAATCAGGGCTCTGAGGCC |
| | (DNA) | | |
| | CDR3 beta | 284 | TGCAGCGTTGGGGAACAGGGTATGACGCAGCCCCAG |
| | (DNA) | | CATTTT |
| 3 | CDR1 alpha | 285 | TISGTDY |
| | (protein) | | |
| | CDR2 alpha | 286 | GLTSN |
| | (protein) | | |
| | CDR3 alpha | 287 | CILRYQGAQKLVF |
| | (protein) | | |
| | CDR1 beta | 288 | MGHRA |
| | (protein) | | |
| | CDR2 beta | 289 | YSYEKL |
| | (protein) | | |
| | CDR3 beta | 290 | CASSQVRERKNTGELFF |
| | (protein) | | |
| | CDR1 alpha | 291 | ACAATCAGTGGAACTGATTAC |
| | (DNA) | | |
| | CDR2 alpha | 292 | GGTCTTACAAGCAAT |
| | (DNA) | | |
| | CDR3 alpha | 293 | TGCATCCTGAGATATCAGGGAGCCCAGAAGCTGGTAT |
| | (DNA) | | TT |
| | CDR1 beta | 294 | ATGGGGCACAGGGCT |
| | (DNA) | | |
| | CDR2 beta | 295 | TACAGCTATGAGAAACTC |
| | (DNA) | | |
| | CDR3 beta | 296 | TGCGCCAGCAGCCAAGTAAGGGAGCGCAAGAACACC |
| | (DNA) | | GGGGAGCTGTTTTTT |
| 4 | CDR1 alpha | 297 | KALYS |
| | (protein) | | |
| | CDR2 alpha | 298 | LLKGGEQ |
| | (protein) | | |
| | CDR3 alpha | 299 | CGTGAGSYQLTF |
| | (protein) | | |
| | CDR1 beta | 300 | MGHRA |
| | (protein) | | |
| | CDR2 beta | 301 | YSYEKL |
| | (protein) | | |
| | CDR3 beta | 302 | CASSHPGLRAGASYNEQFF |
| | (protein) | | |
| | CDR1 alpha | 303 | AAGGCTTTATATTCT |
| | (DNA) | | |
| | CDR2 alpha | 304 | TTACTGAAGGGTGGAGAACAG |
| | (DNA) | | |
| | CDR3 alpha | 305 | TGCGGCACTGGGGCTGGGAGTTACCAACTCACTTTC |
| | (DNA) | | |
| | CDR1 beta | 306 | ATGGGGCACAGGGCT |
| | (DNA) | | |
| | CDR2 beta | 307 | TACAGCTATGAGAAACTC |
| | (DNA) | | |
| | CDR3 beta | 308 | TGCGCCAGCAGCCACCCCGGCTTAAGGGCGGGAGCC |
| | (DNA) | | TCCTACAATGAGCAGTTCTTC |
| 5 | CDR1 alpha | 309 | VTNFRS |
| | (protein) | | |
| | CDR2 alpha | 310 | LTSSGIE |
| | (protein) | | |
| | CDR3 alpha | 311 | CAVLNTGNQFYF |
| | (protein) | | |
| | CDR1 beta | 312 | MGHRA |
| | (protein) | | |
| | CDR2 beta | 313 | YSYEKL |
| | (protein) | | |
| | CDR3 beta | 314 | CASSRFPGGGYNEQFF |
| | (protein) | | |
| | CDR1 alpha | 315 | GTGACTAACTTTCGAAGC |
| | (DNA) | | |
| | CDR2 alpha | 316 | CTAACTTCAAGTGGAATTGAA |
| | (DNA) | | |
| | CDR3 alpha | 317 | TGTGCTGTCTTGAACACCGGTAACCAGTTCTATTTT |
| | (DNA) | | |
| | CDR1 beta | 318 | ATGGGGCACAGGGCT |
| | (DNA) | | |
| | CDR2 beta | 319 | TACAGCTATGAGAAACTC |
| | (DNA) | | |
| | CDR3 beta | 320 | TGCGCCAGCAGTCGGTTTCCGGGCGGGGGCTACAAT |
| | (DNA) | | GAGCAGTTCTTC |
| 6 | CDR1 alpha | 321 | NSASQS |
| | (protein) | | |
| | CDR2 alpha | 322 | VYSSGN |
| | (protein) | | |
| | CDR3 alpha | 323 | CVVNWSGNEKLTF |
| | (protein) | | |
| | CDR1 beta | 324 | KGHDR |
| | (protein) | | |
| | CDR2 beta | 325 | SFDVKD |
| | (protein) | | |
| | CDR3 beta | 326 | CATRGQGVGELFF |
| | (protein) | | |
| | CDR1 alpha | 327 | AACAGTGCTTCTCAGTCT |
| | (DNA) | | |
| | CDR2 alpha | 328 | GTATACTCCAGTGGTAAT |
| | (DNA) | | |
| | CDR3 alpha | 329 | TGTGTGGTGAACTGGTCCGGAAATGAGAAATTAACCTT |
| | (DNA) | | T |
| | CDR1 beta | 330 | AAGGGTCATGATAGA |
| | (DNA) | | |
| | CDR2 beta | 331 | TCCTTTGATGTCAAAGAT |
| | (DNA) | | |
| | CDR3 beta | 332 | TGTGCCACCAGGGGACAGGGGGTCGGGGAGCTGTTT |
| | (DNA) | | TTT |
| 7 | CDR1 alpha | 333 | NSAFQY |
| | (protein) | | |
| | CDR2 alpha | 334 | TYSSGN |
| | (protein) | | |
| | CDR3 alpha | 335 | CAMSVQAAGNKLTF |
| | (protein) | | |
| | CDR1 beta | 336 | SGHAT |
| | (protein) | | |
| | CDR2 beta | 337 | FQNNGV |
| | (protein) | | |
| | CDR3 beta | 338 | CASSLDRNYGYTF |
| | (protein) | | |
| | CDR1 alpha | 339 | AACAGTGCTTTTCAATAC |
| | (DNA) | | |
| | CDR2 alpha | 340 | ACATACTCCAGTGGTAAC |
| | (DNA) | | |
| | CDR3 alpha | 341 | TGTGCAATGAGCGTACAGGCTGCAGGCAACAAGCTAA |
| | (DNA) | | CTTTT |
| | CDR1 beta | 342 | TCTGGCCATGCTACC |
| | (DNA) | | |
| | CDR2 beta | 343 | TTTCAGAATAACGGTGTA |
| | (DNA) | | |
| | CDR3 beta | 344 | TGTGCCAGCAGCTTAGACAGGAACTATGGCTACACCT |
| | (DNA) | | TC |
| 8 | CDR1 alpha | 345 | ATGYPS |
| | (protein) | | |
| | CDR2 alpha | 346 | ATKADDK |
| | (protein) | | |
| | CDR3 alpha | 347 | CALSDNTNAGKSTF |
| | (protein) | | |
| | CDR1 beta | 348 | DFQATT |
| | (protein) | | |
| | CDR2 beta | 349 | SNEGSKA |
| | (protein) | | |
| | CDR3 beta | 350 | CSARTSGQYEQYF |
| | (protein) | | |
| | CDR1 alpha | 351 | GCCACAGGATACCCTTCC |
| | (DNA) | | |
| | CDR2 alpha | 352 | GCCACGAAGGCTGATGACAAG |
| | (DNA) | | |
| | CDR3 alpha | 353 | TGTGCTCTGAGTGATAACACCAATGCAGGCAAATCAAC |
| | (DNA) | | CTTT |
| | CDR1 beta | 354 | GACTTTCAGGCCACAACT |
| | (DNA) | | |
| | CDR2 beta | 355 | TCCAATGAGGGCTCCAAGGCC |
| | (DNA) | | |
| | CDR3 beta | 356 | TGCAGTGCTAGGACTAGCGGTCAATACGAGCAGTACT |
| | (DNA) | | TC |
| 9 | CDR1 alpha | 357 | ATGYPS |
| | (protein) | | |
| | CDR2 alpha | 358 | ATKADDK |
| | (protein) | | |
| | CDR3 alpha | 359 | CALSDLLDSGNTGKLIF |
| | (protein) | | |
| | CDR1 beta | 360 | SGDLS |
| | (protein) | | |
| | CDR2 beta | 361 | YYNGEE |
| | (protein) | | |
| | CDR3 beta | 362 | CASRGSYNEQFF |
| | (protein) | | |
| | CDR1 alpha | 363 | GCCACAGGATACCCTTCC |
| | (DNA) | | |
| | CDR2 alpha | 364 | GCCACGAAGGCTGATGACAAG |
| | (DNA) | | |
| | CDR3 alpha | 365 | TGTGCTCTGAGTGATCTGTTGGACTCTGGCAACACAG |
| | (DNA) | | GCAAACTAATCTTT |
| | CDR1 beta | 366 | TCTGGAGACCTCTCT |
| | (DNA) | | |
| | CDR2 beta | 367 | TATTATAATGGAGAAGAG |
| | (DNA) | | |
| | CDR3 beta | 368 | TGTGCCAGTAGAGGATCCTACAATGAGCAGTTCTTC |
| | (DNA) | | |
| 10 | CDR1 alpha | 369 | NSASQS |
| | (protein) | | |
| | CDR2 alpha | 370 | VYSSGN |
| | (protein) | | |
| | CDR3 alpha | 371 | CVVNTPSSKIIF |
| | (protein) | | |
| | CDR1 beta | 372 | DFQATT |
| | (protein) | | |
| | CDR2 beta | 373 | SNEGSKA |
| | (protein) | | |
| | CDR3 beta | 374 | CSARSGQGQFF |
| | (protein) | | |
| | CDR1 alpha | 375 | AACAGTGCTTCTCAGTCT |
| | (DNA) | | |
| | CDR2 alpha | 376 | GTATACTCCAGTGGTAAT |
| | (DNA) | | |
| | CDR3 alpha | 377 | TGTGTGGTGAACACTCCCTCTTCCAAGATAATCTTT |
| | (DNA) | | |
| | CDR1 beta | 378 | GACTTTCAGGCCACAACT |
| | (DNA) | | |
| | CDR2 beta | 379 | TCCAATGAGGGCTCCAAGGCC |
| | (DNA) | | |
| | CDR3 beta | 380 | TGCAGTGCTAGATCGGGACAGGGGCAGTTCTTC |
| | (DNA) | | |
| 11 | CDR1 alpha | 381 | NSASQS |
| | (protein) | | |
| | CDR2 alpha | 382 | VYSSGN |
| | (protein) | | |
| | CDR3 alpha | 383 | CVVPPGGGFKTIF |
| | (protein) | | |
| | CDR1 beta | 384 | MNHEY |
| | (protein) | | |
| | CDR2 beta | 385 | SMNVEV |
| | (protein) | | |
| | CDR3 beta | 386 | CASRTRTASYGYTF |
| | (protein) | | |
| | CDR1 alpha | 387 | AACAGTGCTTCTCAGTCT |
| | (DNA) | | |
| | CDR2 alpha | 388 | GTATACTCCAGTGGTAAT |
| | (DNA) | | |
| | CDR3 alpha | 389 | TGTGTGGTGCCTCCTGGGGGAGGCTTCAAAACTATCT |
| | (DNA) | | TT |
| | CDR1 beta | 390 | ATGAACCATGAGTAT |
| | (DNA) | | |
| | CDR2 beta | 391 | TCAATGAATGTTGAGGTG |
| | (DNA) | | |
| | CDR3 beta | 392 | TGTGCCAGCAGGACCAGGACAGCCAGTTATGGCTACA |
| | (DNA) | | CCTTC |
| 12 | CDR1 alpha | 393 | TSGFNG |
| | (protein) | | |
| | CDR2 alpha | 394 | NVLDGL |
| | (protein) | | |
| | CDR3 alpha | 395 | CAVLDSNYQLIW |
| | (protein) | | |
| | CDR1 beta | 396 | MNHNS |
| | (protein) | | |
| | CDR2 beta | 397 | SASEGT |
| | (protein) | | |
| | CDR3 beta | 398 | CASSEEASGSYEQYF |
| | (protein) | | |
| | CDR1 alpha | 399 | ACATCTGGGTTCAACGGG |
| | (DNA) | | |
| | CDR2 alpha | 400 | AATGTTCTGGATGGTTTG |
| | (DNA) | | |
| | CDR3 alpha | 401 | TGTGCTGTCCTGGATAGCAACTATCAGTTAATCTGG |
| | (DNA) | | |
| | CDR1 beta | 402 | ATGAACCATAACTCC |
| | (DNA) | | |
| | CDR2 beta | 403 | TCAGCTTCTGAGGGTACC |
| | (DNA) | | |
| | CDR3 beta | 404 | TGTGCCAGCAGTGAAGAGGCTAGCGGGTCCTACGAG |
| | (DNA) | | CAGTACTTC |
| 13 | CDR1 alpha | 405 | DRGSQS |
| | (protein) | | |
| | CDR2 alpha | 406 | IYSNGD |
| | (protein) | | |
| | CDR3 alpha | 407 | CAVSGNQFYF |
| | (protein) | | |
| | CDR1 beta | 408 | SEHNR |
| | (protein) | | |
| | CDR2 beta | 409 | FQNEAQ |
| | (protein) | | |
| | CDR3 beta | 410 | CASSPLGSGRYNEQFF |
| | (protein) | | |
| | CDR1 alpha | 411 | GACCGAGGTTCCCAGTCC |
| | (DNA) | | |
| | CDR2 alpha | 412 | ATATACTCCAATGGTGAC |
| | (DNA) | | |
| | CDR3 alpha | 413 | TGTGCCGTCTCAGGGAACCAGTTCTATTTT |
| | (DNA) | | |
| | CDR1 beta | 414 | TCTGAACACAACCGC |
| | (DNA) | | |
| | CDR2 beta | 415 | TTCCAGAATGAAGCTCAA |
| | (DNA) | | |
| | CDR3 beta | 416 | TGTGCCAGCAGCCCCCTGGGTAGCGGGAGGTACAAT |
| | (DNA) | | GAGCAGTTCTTC |
| 14 | CDR1 alpha | 417 | YGATPY |
| | (protein) | | |
| | CDR2 alpha | 418 | YFSGDTLV |
| | (protein) | | |
| | CDR3 alpha | 419 | CAVGPFSGGYQKVTF |
| | (protein) | | |
| | CDR1 beta | 420 | SNHLY |
| | (protein) | | |
| | CDR2 beta | 421 | FYNNEI |
| | (protein) | | |
| | CDR3 beta | 422 | CASREVHSGANVLTF |
| | (protein) | | |
| | CDR1 alpha | 423 | TATGGGGCAACACCTTAT |
| | (DNA) | | |
| | CDR2 alpha | 424 | TACTTTTCAGGAGACACTCTGGTT |
| | (DNA) | | |
| | CDR3 alpha | 425 | TGTGCTGTGGGTCCCTTTTCTGGGGGTTACCAGAAAG |
| | (DNA) | | TTACCTTT |
| | CDR1 beta | 426 | TCTAATCACTTATAC |
| | (DNA) | | |
| | CDR2 beta | 427 | TTTTATAATAATGAAATC |
| | (DNA) | | |
| | CDR3 beta | 428 | TGTGCCAGCAGAGAGGTACACTCTGGGGCCAACGTCC |
| | (DNA) | | TGACTTTC |
| 15 | CDR1 alpha | 429 | YGATPY |
| | (protein) | | |
| | CDR2 alpha | 430 | YFSGDTLV |
| | (protein) | | |
| | CDR3 alpha | 431 | CAFYNQGGKLIF |
| | (protein) | | |
| | CDR1 beta | 432 | SNHLY |
| | (protein) | | |
| | CDR2 beta | 433 | FYNNEI |
| | (protein) | | |
| | CDR3 beta | 434 | CASRVMNTEAFF |
| | (protein) | | |
| | CDR1 alpha | 435 | TATGGGGCAACACCTTAT |
| | (DNA) | | |
| | CDR2 alpha | 436 | TACTTTTCAGGAGACACTCTGGTT |
| | (DNA) | | |
| | CDR3 alpha | 437 | TGTGCTTTTTATAACCAGGGAGGAAAGCTTATCTTC |
| | (DNA) | | |
| | CDR1 beta | 438 | TCTAATCACTTATAC |
| | (DNA) | | |
| | CDR2 beta | 439 | TTTTATAATAATGAAATC |
| | (DNA) | | |
| | CDR3 beta | 440 | TGTGCCAGCAGGGTCATGAACACTGAAGCTTTCTTT |
| | (DNA) | | |
| 16 | CDR1 alpha | 441 | YGATPY |
| | (protein) | | |
| | CDR2 alpha | 442 | YFSGDTLV |
| | (protein) | | |
| | CDR3 alpha | 443 | CAVGFLEYGNKLVF |
| | (protein) | | |
| | CDR1 beta | 444 | SGHRS |
| | (protein) | | |
| | CDR2 beta | 445 | YFSETQ |
| | (protein) | | |
| | CDR3 beta | 446 | CASSSLAGANEQFF |
| | (protein) | | |
| | CDR1 alpha | 447 | TATGGGGCAACACCTTAT |
| | (DNA) | | |
| | CDR2 alpha | 448 | TACTTTTCAGGAGACACTCTGGTT |
| | (DNA) | | |
| | CDR3 alpha | 449 | TGTGCTGTGGGCTTCTTGGAATATGGAAACAAACTGGT |
| | (DNA) | | CTTT |
| | CDR1 beta | 450 | TCTGGGCATAGGAGT |
| | (DNA) | | |
| | CDR2 beta | 451 | TACTTCAGTGAGACACAG |
| | (DNA) | | |
| | CDR3 beta | 452 | TGCGCCAGCAGCTCCCTAGCGGGAGCCAATGAGCAG |
| | (DNA) | | TTCTTC |
| 17 | CDR1 alpha | 453 | VSPFSN |
| | (protein) | | |
| | CDR2 alpha | 454 | MTFSENT |
| | (protein) | | |
| | CDR3 alpha | 455 | CVVSAGYGGSQGNLIF |
| | (protein) | | |
| | CDR1 beta | 456 | SEHNR |
| | (protein) | | |
| | CDR2 beta | 457 | FQNEAQ |
| | (protein) | | |
| | CDR3 beta | 458 | CASTPGGSSREQYF |
| | (protein) | | |
| | CDR1 alpha | 459 | GTGAGCCCCTTCAGCAAC |
| | (DNA) | | |
| | CDR2 alpha | 460 | ATGACTTTCAGTGAGAACACA |
| | (DNA) | | |
| | CDR3 alpha | 461 | TGTGTGGTGAGCGCGGGATATGGAGGAAGCCAAGGA |
| | (DNA) | | AATCTCATCTTT |
| | CDR1 beta | 462 | TCTGAACACAACCGC |
| | (DNA) | | |
| | CDR2 beta | 463 | TTCCAGAATGAAGCTCAA |
| | (DNA) | | |
| | CDR3 beta | 464 | TGTGCCAGCACCCCTGGGGGCTCCTCCCGCGAGCAG |
| | (DNA) | | TACTTC |
| 18 | CDR1 alpha | 465 | TRDTTYY |
| | (protein) | | |
| | CDR2 alpha | 466 | RNSFDEQN |
| | (protein) | | |
| | CDR3 alpha | 467 | CALSTHGNKLVF |
| | (protein) | | |
| | CDR1 beta | 468 | SEHNR |
| | (protein) | | |
| | CDR2 beta | 469 | FQNEAQ |
| | (protein) | | |
| | CDR3 beta | 470 | CASSLDRENEQFF |
| | (protein) | | |
| | CDR1 alpha | 471 | ACCCGTGATACTACTTATTAC |
| | (DNA) | | |
| | CDR2 alpha | 472 | CGGAACTCTTTTGATGAGCAAAAT |
| | (DNA) | | |
| | CDR3 alpha | 473 | TGTGCTCTGAGTACCCATGGAAACAAACTGGTCTTT |
| | (DNA) | | |
| | CDR1 beta | 474 | TCTGAACACAACCGC |
| | (DNA) | | |
| | CDR2 beta | 475 | TTCCAGAATGAAGCTCAA |
| | (DNA) | | |
| | CDR3 beta | 476 | TGTGCCAGCAGCTTAGACAGGGAAAATGAGCAGTTCT |
| | (DNA) | | TC |
| 19 | CDR1 alpha | 477 | VSNAYN |
| | (protein) | | |
| | CDR2 alpha | 478 | GSKP |
| | (protein) | | |
| | CDR3 alpha | 479 | CAVEDRDNNARLMF |
| | (protein | | |
| | CDR1 beta | 480 | ENHRY |
| | (protein) | | |
| | CDR2 beta | 481 | SYGVKD |
| | (protein) | | |
| | CDR3 beta | 482 | CAISERPVGEQETQYF |
| | (protein) | | |
| | CDR1 alpha | 483 | GTGTCCAATGCTTACAAC |
| | (DNA) | | |
| | CDR2 alpha | 484 | GGCTCAAAGCCT |
| | (DNA) | | |
| | CDR3 alpha | 485 | TGTGCTGTGGAGGATAGGGATAACAATGCCAGACTCA |
| | (DNA) | | TGTTT |
| | CDR1 beta | 486 | GAGAACCACCGCTAT |
| | (DNA) | | |
| | CDR2 beta | 487 | TCATATGGTGTTAAAGAT |
| | (DNA) | | |
| | CDR3 beta | 488 | TGTGCCATCAGTGAACGGCCGGTCGGGGAGCAAGAG |
| | (DNA) | | ACCCAGTACTTC |
| 20 | CDR1 alpha | 489 | VSGLRG |
| | (protein) | | |
| | CDR2 alpha | 490 | LYSAGEE |
| | (protein) | | |
| | CDR3 alpha | 491 | CAVQIGGYGNKLVF |
| | (protein) | | |
| | CDR1 beta | 492 | SGHDY |
| | (protein) | | |
| | CDR2 beta | 493 | FNNNVP |
| | (protein) | | |
| | CDR3 beta | 494 | CASSPRPSEKLFF |
| | (protein) | | |
| | CDR1 alpha | 495 | GTCAGCGGTTTAAGAGGG |
| | (DNA) | | |
| | CDR2 alpha | 496 | CTGTATTCAGCTGGGGAAGAA |
| | (DNA) | | |
| | CDR3 alpha | 497 | TGTGCTGTGCAGATCGGAGGGTATGGAAACAAACTGG |
| | (DNA) | | TCTTT |
| | CDR1 beta | 498 | TCAGGACACGACTAC |
| | (DNA) | | |
| | CDR2 beta | 499 | TTTAACAACAACGTTCCG |
| | (DNA) | | |
| | CDR3 beta | 500 | TGTGCCAGCAGCCCCCGGCCGAGTGAAAAACTGTTTT |
| | (DNA) | | TT |

TABLE 3
HLA-DR15 restricted TCRs

| | | SEQ | |
|---|---|---|---|
| TCR | Description | ID NO: | Amino acid sequence |
| 1 | TRA | 501 | MNYSPGLVSLILLLLGRTRGNSVTQMEGPVTLSEEAFLTI |
| | | | NCTYTATGYPSLFWYVQYPGEGLQLLLKATKADDKGSN |
| | | | KGFEATYRKETTSFHLEKGSVQVSDSAVYFCALSSYGNK |
| | | | LVFGAGTILRVKSYIQNPDPAVYQLRDSKSSDKSVCLF |
| | TRB | 502 | MRGQGRPSSSAFAHSDPDWAKLPSFPDPAMGTRLLCW |
| | | | AALCLLGAELTEAGVAQSPRYKIIEKRQSVAFWCNPISGH |
| | | | ATLYWYQQILGQGPKLLIQFQNNGVVDDSQLPKDRFSAE |
| | | | RLKGVDSTLKIQPAKLEDSAVYLCASSSLSGSSYEQYFG |
| | | | PGTRLTVTEDLKNVFPPEVAVFEPSEAEISHTQKATLVCL |
| | | | ATGFYPDHVELSWWVNGKEVHSGVSTDPQPLKEQPALN |
| | | | DSRYCL |
| 2 | TRA | 503 | MHTSTFQNRPQLFLLIWKKLVPGNPFRRSWMKREREML |
| | | | LITSMLVLWMQLSQVNGQQVMQIPQYQHVQEGEDFTTY |
| | | | CNSSTTLSNIQWYKQRPGGHPVFLIQLVKSGEVKKQKRL |
| | | | TFQFGEAKKNSSLHITATQTTDVGTYFCAGHAGAQKLVF |
| | | | GQGTRLTINPNIQNPDPAVYQLRD |
| | TRB | 504 | MKSQNDPLESTVPLSPMHRPRRPLHPVAPAMSIGLLCCV |
| | | | AFSLLWASPVNAGVTQTPKFQVLKTGQSMTLQCAQDMN |
| | | | HNSMYWYRQDPGMGLRLIYYSASEGTTDKGEVPNGYNV |
| | | | SRLNKREFSLRLESAAPSQTSVYFCASSEVWVGSTDTQY |
| | | | FGPGTRLTVLEDLKNVFPPEVAVFEPSEAEISHTQKATLV |
| | | | CLATGFYPDHVELSWWVNGKEVHSGVSTDPPLKEQPAL |
| | | | NDSRYCL |
| 3 | TRA | 505 | MAMLLGASVLILWLQPDWVNSQQKNDDQQVKQNSPSLS |
| | | | VQEGRISILNCDYTNSMFDYFLWYKKYPAEGPTFLISISSI |
| | | | KDKNEDGRFTVFLNKSAKHLSLHIVPSQPGDSAVYFCAA |
| | | | SRRFGNEKLTFGTGTRLTIIPNIQNPDPAVYQLRD |
| | TRB | 506 | MVHIRVAAILDLTHQYQVILRLSDVTVGTALWRQGRPSSS |
| | | | APAHSDPDLVKLPSCPDPAMGTSLLCWMALCLLGADHA |
| | | | DTGVSQNPRHKITKRGQNVTFRCDPISEHNRLYWYRQTL |
| | | | GQGPEFLTYFQNEAQLEKSRLLSDRFSAERPKGSFSTLEI |
| | | | QRTEQGDSAMYLCASRLWSRGRTTEAFFGQGTRLTVVE |
| | | | DLNKVFPPEVAVFEPSEAEISHTQKATLVCLATGFYPDHV |
| | | | ELSWWVNGKEVHSGVSTDPQPLKEQPALNDSRYCL |
| 4 | TRA | 507 | MDKILGASFLVLWLQLCWVSGQQKEKSDQQQVKQSPQS |
| | | | LIVQKGGISIINCAYENTAFDYFPWYQQFPGKGPALLIAIR |
| | | | PDVSEKKEGRFTISFNKSAKQFSLHIMDSQPGDSATYFC |
| | | | AASKTGTASKLTFGTGTRLQVTLDIQNPDPAVYQLRD |
| | TRB | 508 | MRVGRDNDITDHQPTAWSWEKTYSFFKAAMGIRLLCRV |
| | | | AFCFLAVGLVDVKVTQSSRYLVKRTGEKVFLECVQDMDH |
| | | | ENMFWYRQDPGLGLRLIYFSYDVKMKEKGDIPEGYSVSR |
| | | | EKKERFSLILESASTNQTSMYLCASSVNEQFFGPGTRLTV |
| | | | LEDLKNVFPPEVAVFEPSEAEISHTQKATLVCLATGF |
| 5 | TRA | 509 | MRLVARVTVFLTFGTIIDAKTTQPPSMDCAEGRAANLPCN |
| | | | HSTISGNEYVYWYRQIHSQGPQYIIHGLKNNETNEMASLII |
| | | | TEDRKSSTLILPHATLRDTAVYYCIVKSWNNRKLIWGLGT |
| | | | SLAVNPNIQNPDPAVYQLRD |
| | TRB | 510 | MGTSLLCWMALCLLGADHADTGVSQNPRHKITKRGQNV |
| | | | TFRCDPISEHNRLYWYRQTLGQGPEFLTYFQNEAQLEKS |
| | | | RLLSDRFSAERPKGSFSTLEIQRTEQGDSAMYLCASLKR |
| | | | TGGHNQPQHFGDGTRLSILEDLNKVFPPEVAVFEPSEAEI |
| | | | SHTQKATLVCLATGFYPDHVELSWWVNGKEVHSGVSTD |
| | | | PQPLKEQPALNDSRYCL |
| 6 | TRA | 511 | MLLLLVPAFQVIFTLGGTRAQSVTQLDSQVPVFEEAPVEL |
| | | | RCNYSSSVSVYLFWYVQYPNQGLQLLLKYLSGSTLVKGI |
| | | | NGFEAEFNKSQTSFHLRKPSVHISDTAEYFCAVSVSGSA |
| | | | RQLTFGSGTQLTVLPDIQNPDPAVYQLRDSKSSDKSVCL |
| | | | FTDFDSQTNVSQSKDSDVYITDKTVLDMRSMDFKSNSAV |
| | | | AWSNKSDFACANAF |
| | TRB | 512 | MGTRLLCWAALCLLGADHTGAGVSTPSNKVTEKGKYV |
| | | | ELRCDPISGHTALYWYRQSLGQGPEFLIYFQGTGAADDS |
| | | | GLPNDRFFAVRPEGSVSTLKIQRTERGDSAVYLCASSHR |
| | | | LAGAEQFFGPGTRLTVLEDLKNVFPPEVAVFEPSEAEISH |
| | | | TQKATLVCLATGFYPDHVELSWWVNGKEVHSGVSTDPQ |
| | | | PLKEQPALNDSRYCL |
| 7 | TRA | 513 | MNYSPGLVSLILLLLGRTRGNSVTQMEGPVTLSEEAFLTI |
| | | | NCTYTATGYPSLFWYVQYPGEGLQLLLKATKADDKGSN |
| | | | KGFEATYRKETTSFHLEKGSVQVSDSAVYFCALTLNSGY |
| | | | STLTFGKGTMLLVSPDIQNPDPAVYQLRDSKSSDKSVCL |
| | | | FTDFD |
| | TRB | 514 | MEAVVTTLPREGGVRPSRKMLLLLLLLGPGSGLGAVVSQ |
| | | | HPSWVICKSGTSVKIECRSLDFQATTMFWYRQFPKQSLM |
| | | | LMATSNEGSKATYEQGVEKDKFLINHASLTLSTLTVTSAH |
| | | | PEDSSFYICSARDPGTLYEQYFGPGTRLTVTEDLKNVFP |
| | | | PEVAVFEPS |
| 8 | TRA | 515 | MALQSTLGAVWLGLLLNSLWKVAESKDQVFQPSTVASS |
| | | | EGAVVEIFCNHSVSNAYNFFWYLHFPGCAPRLLVKGSKP |
| | | | SQQGRYNMTYERFSSSLLILQVREADAAVYYCAVERPGF |
| | | | QKLVFGTGTRLLVSPNIQNPDPAVYQLRD |
| | TRB | 516 | MGCRLLCCVVFCLLQAGPLDTAVSQTPKYLVTQMGNDK |
| | | | SIKCEQNLGHDTMYWYKQDSKKFLKIMFSYNNKELIINET |
| | | | VPNRFSPKSPDKAHLNLHINSLELGDSAVYFCASRTTGS |
| | | | GTEAFFGQGTRLTVVEDLNKVFPPEVAVFEPSEAEISHT |
| | | | QKATLVCLATGFFPDHVELSWWVNGKEVHSGVSTDPQP |
| | | | LKEQPALNDSRYCL |
| 9 | TRA | 517 | METLLGVSLVILWLQLARVNSQQGEEDPQALSIQEGENA |
| | | | TMNCSYKTSINNLQWYRQNSGRGLVHLILIRSNEREKHS |
| | | | GRLRVTLDTSKKSSSLLITASRAADTASYFCATPRRGADG |
| | | | LTFGKGTHLIIQPYIQNPDPAVYQLRD |
| | TRA | 518 | MVLKFSVSILWIQLAWVSTQLLEQSPQFLSIQEGENLTVY |
| | | | CNSSSVFSSLQWYRQEPGEGPVLLVTVVTGGEVKKLKR |
| | | | LTFQFGDARKDSSLHITAAQPGDTGLYLCAFCNQFYFGT |
| | | | GTSLTVIPNIQNPDPAVYQLRD |
| | TRB | 519 | MGCRLLCCAVLCLLGAVPMETGVTQTPRHLVMGMTNKK |
| | | | SLKCEQHLGHNAMYWYKQSAKKPLELMFVYNFKEQTEN |
| | | | NSVPSRFSPECPNSSHLFLHLHTLQPEDSALYLCASSPG |
| | | | GSGRLGDTQYFGPGTRLTVLEDLKNVFPPEVAVFEPSEA |
| | | | EISHTQKATLVCLATGFFPDHVELSWWVNGKEVHSGVST |
| | | | DPQPLKEQPALNDSRYCL |
| 10 | TRA | 520 | MALQSTLGAVWLGLLLNSLWKVAESKDQVFQPSTVASS |
| | | | EGAVVEIFCNHSVSNAYNFFWYLHFPGCAPRLLVKGSKP |
| | | | SQQGRYNMTYERFSSSLLILQVREADAAVYYCAVEDSRG |
| | | | NNRLAFGKGNQVVVIPNIQNPDPAVYQLRDSKSSDKSV |
| | TRB | 521 | MSQNDFLESPAPLSSMHRYRRPLRHAASAMSIGLLCCAA |
| | | | LSLLWAGPVNAGVTQTPKFQVLKTGQSMTLQCAQDMNH |
| | | | EYMSWYRQDPGMGLRLIHYSVGAGITDQGEVPNGYNVS |
| | | | RSTTEDFPLRLLSAAPSQTSVYFCASGGTGGNYGYTFGS |
| | | | GTRLTVVEDLNKVFPPEVAVFEPSEAEISHTQKATLVCLA |
| | | | TGFFPDHVELSWWVNGKEVHSGVSTDPQPLKEQPALND |
| | | | SRYCL |
| 11 | TRA | 522 | MTRVSLLWAVVVSTCLESGMAQTVTQSQPEMSVQEAET |
| | | | VTLSCTYDTSESNYYLFWYKQPPSRQMILVIRQEAYKQQ |
| | | | NATENRFSVNFQKAAKSFSLKISDSQLGDTAMYFCAFMK |
| | | | SLFGNEKLTFGTGTRLTIIPNIQNPDPAVYQLRDSKSSDK |
| | | | SVCLFTDFDSQ |
| | TRB | 523 | MEAVVTTLPREGGVRPSRKMLLLLLLLGPGSGLGAVVSQ |
| | | | HPSRVICKSGTSVKIECRSLDFQATTMFWYRQFPKQSLM |
| | | | LMATSNEGSKATYEQGVEKDKFLINHASLTLSTLTVTSAH |
| | | | PEDSSFYICSARSGEKLFFGSGTQLSVLEDLNKVFPPEVA |
| | | | VFEPSEAEISHTQKATLVCLATGFFPDHVELSWWVNGKE |
| | | | VHSGVSTDPQPLKEQPALNDSRYCL |
| 12 | TRA | 524 | MTRVSLLWAVVVSTCLESGMAQTVTQSQPEMSVQEAET |
| | | | VTLSCTYDTSESNYYLFWYKQPPSRQMILVIRQEAYKQQ |
| | | | NATENRFSVNFQKAAKSFSLKISDSQLGDTAMYFCAFMK |
| | | | SPFGNEKLTFGTGTRLTIIPNIQNPDPAVYQLRDSKSSDK |
| | | | SVCLFTDFDSQTNVSQSKDSDVYITDKTVLDMRSMDFKS |
| | | | NSAVAWSNKSDFACANAF |
| | TRB | 525 | MEAVVTTLPREGGVRPSRKMLLLLLLLGPGSGLGAVVSQ |
| | | | HPSWVICKSGTSVKIECRSLDFQATTMFWYRQFPKQSLM |
| | | | LMATSNEGSKATYEQGVEKDKFLINHASLTLSTLTVTSAH |
| | | | PEDSSFYICSARWGDQPQHFGDGTRLSILEDLNKVFPPE |
| | | | VAVFEPSEAEISHTQKATLVCLATGFYPDHVELSWWVNG |
| | | | KEVHSGVSTDPQPLKEQPALNDSRYCL |
| 13 | TRA | 526 | MTRVSLLWAVVVSTCLESGMAQTVTQSQPEMSVQEAET |
| | | | VTLSCTYDTSESNYYLFWYKQPPSRQMILVIRQEAYKQQ |
| | | | NATENRFSVNFQKAAKSFSLKISDSQLGDTAMYFCAFMK |
| | | | TNFGNEKLTFGTGTRLTIIPNIQNPDPAVYQLRD |
| | TRB | 527 | MEAVVTTLPREGGVRPSRKMLLLLLLLGPGSGLGAVVSQ |
| | | | HPSWVICKSGTSVKIECRSLDFQATTMFWYRQFPKQSLM |
| | | | LMATSNEGSKATYEQGVEKDKFLINHASLTLSTLTVTSAH |
| | | | PEDSSFYICSARSGTSKFFGPGTRLTVLEDLKNVFPPEVA |
| | | | VFEPSEAEISHTQKATLVCLATGFFPDHVELSWWVNGKE |
| | | | VHSGVST |
| 14 | TRA | 528 | MTRVSLLWAVVVSTCLESGMAQTVTQSQPEMSVQEAET |
| | | | VTLSCTYDTSESNYYLFWYKQPPSRQMILVIRQEAYKQQ |
| | | | NATENRFSVNFQKAAKSFSLKISDSQLGDTAMYFCAFTK |
| | | | SNFGNEKLTFGTGTRLTIIPNIQNPDPAVYQLRD |
| | TRB | 529 | MEAVVTTLPREGGVRPSRKMLLLLLLLGPGSGLGAVVSQ |
| | | | HPSWVICKSGTSVKIECRSLDFQATTMFWYRQFPKQSLM |
| | | | LMATSNEGSKATYEQGVEKDKFLINHASLTLSTLTVTSAH |
| | | | PEDSSFYICSARTGDEQYFGPGTRLTVTEDLKNVFPPEV |
| | | | AVFEPSEAEISHTQKATLVCLATGFYPDHVELSWWVNGK |
| | | | EVHSGVSTDPQPLKEQPALNDSRYCL |
| 15 | TRA | 530 | MGIAFPALKENSHQAQRRTHQSRRLFTLQGSAVSMTRV |
| | | | SLLWAVVVSTCLESGMAQTVTQSQPEMSVQEAETVTLS |
| | | | CTYDTSESNYYLFWYKQPPSRQMILVIRQEAYKQQNATE |
| | | | NRFSVNFQKAAKSFSLKISDSQLGDTAMYFCAFIKSNFGN |
| | | | EKLTFGTGTRLTIIPNIQNPDPAVYQLRDSKSSDKSVC |
| | TRB | 531 | MEAVVTTLPREGGVRPSRKMLLLLLLLGPGSGLGAVVSQ |
| | | | HPSRVICKSGTSVKIECRSLDFQATTMFWYRQFPKQSLM |
| | | | LMATSNEGSKATYEQGVEKDKFLINHASLTLSTLTVTSAH |
| | | | PEDSSFYICSARRGTEAFFGQGTRLTVVEDLNKVFPPEV |
| | | | AVFEPSEAEISHTQKATLVCLATGFFPDHVELSWWVNGK |
| | | | EVHSGVSTDPQPLKEQPALNDSRYCL |
| 16 | TRA | 532 | MAQELGMQCQARGILQQMWGVFLLYVSMKMGGTTGQN |
| | | | IDQPTEMTATEGAIVQINCTYQTSGFNGLFWYQQHAGEA |
| | | | PTFLSYNVLDGLEEKGRFSSFLSRSKGYSYLLLKELQMK |
| | | | DSASYLCAVMDSSYKLIFGSGTRLLVRPDIQNPDPAVYQL |
| | | | RDSKSSDKSVCLFTDF |
| | TRA | 533 | MRQVARVIVFLTLSTLSLAKTTQPISMDSYEGQEVNITCS |
| | | | HNNIATNDYITWYQQFPSQGPRFIIQGYKTKVTNEVASLFI |
| | | | PADRKSSTLSLPRVSLSDTAVYYCLVKIIFGSGTRLSIRPNI |
| | | | QNPDPAVYQLRDSKSSDKSVCLFTDF |
| | TRB | 534 | MRSQDDFLDSPVRLSSTHRPRRPLCLVASAMRIRLLCCV |
| | | | AFSLLWAGPVIAGITQAPTSQILAAGRRMTLRCTQDMRH |
| | | | NAMYWYRQDLGLGLRLIHYSNTAGTTGKGEVPDGYSVS |
| | | | RANTDDFPLTLASAVPSQTSVYFCASSEREHYGYTFGSG |
| | | | TRLTVVEDLNKVFPPEVAVFEPSEAEISHTQKATLVCLAT |
| | | | GFYPDHVELSWWVNGKEVHSGVSTDPQPLKEQPALND |
| | | | SRYCL |
| 17 | TRA | 535 | METLLGVSLVILWLQLARVNSQQGEEDPQALSIQEGENA |
| | | | TMNCSYKTSINNLQWYRQNSGRGLVHLILIRSNEREKHS |
| | | | GRLRVTLDTSKKSSSLLITASRAADTASYFCATPYTGGFK |
| | | | TIFGAGTRLFVKANIQNPDPAVYQLRDSKSSDKSVCLFTD |
| | | | FD |
| | TRB | 536 | MGCRLLCCAVLCLLGAVPIDTEVTQTPKHLVMGMTNKKS |
| | | | LKCEQHMGHRAMYWYKQKAKKPPELMFVYSYEKLSINE |
| | | | SVPSRFSPECPNSSLLNLHLHALQPEDSALYLCASSQLG |
| | | | SGNTIYFGEGSWLTVVEDLNKVFPPEVAVFEPSEAEISHT |
| | | | QKATLVCLATGFYPDHVELSWWVNGKEVHSGVSTDPQP |
| | | | LKEQPALNDSRYCL |
| 18 | TRA | 537 | METLLGLLILWLQLQWVSSKQEVTQIPAALSVPEGENLVL |
| | | | NCSFTDSAIYNLQWFRQDPGKGLTSLLLIQSSQREQTSG |
| | | | RLNASLDKSSGRSTLYIAASQPGDSATYLCAVKAQGGAG |
| | | | SYQLTFGKGTKLSVIPNIQNPDPAVYQLRDSKSSDKSVCL |
| | | | FTD |
| | TRB | 538 | MGPGLLCWVLLCLLGAGSVETGVTQSPTHLIKTRGQQVT |
| | | | LRCSSQSGHNTVSWYQQALGQGPQFIFQYYREEENGR |
| | | | GNFPPRFSGLQFPNYSSELNVNALELDDSALYLCGSGRE |
| | | | RTDTQYFGPGTRLTVLEDLKNVFPPEVAVFEPSEAEISHT |
| | | | QKATLVCLATGFYPDHV |
| 19 | TRA | 539 | MVLKFSVSILWIQLAWVSTQLLEQSPQFLSIQEGENLTVY |
| | | | CNSSSVFSSLQWYRQEPGEGPVLLVTVVTGGEVKKLKR |
| | | | LTFQFGDARKDSSLHITAAQPGDTGLYLCAGANSGYALN |
| | | | FGKGTSLLVTPHIQNPDPAVYQLRD |
| | TRB | 540 | MRVGRDNDITDHQPTAWSWEKTYSFFKAAMGIRLLCRV |
| | | | AFCFLAVGLVDVKVTQSSRYLVKRTGEKVFLECVQDMDH |
| | | | ENMFWYRQDPGLGLRLIYFSYDVKMKEKGDIPEGYSVSR |
| | | | EKKERFSLILESASTNQTSMYLCASSSLPGQLYGYTFGS |
| | | | GTRLTVVEDLNKVFPPEVAVFEPSEAEISHTQKATLVCLA |
| | | | TGFYPDHVELSWWVNGKEVHSGVSTDPQPLKEQPALND |
| | | | SRYCL |
| 20 | TRA | 541 | MQCQAHGILQQMWGAFLLYVSMKMGGTAGQSLEQPSE |
| | | | VTAVEGAIVQINCTYQTSGFYGLSWYQQHDGGAPTFLSY |
| | | | NALDGLEETGRFSSFLSRSDSYGYLLLQELQMKDSASYF |
| | | | CAEGGFKTIFGAGTRLFVKANIQNPDPAVYQLRD |
| | TRB | 542 | MASLLFFCGAFYLLGTGSMDADVTQTPRNRITKTGKRIML |
| | | | ECSQTKGHDRMYWYRQDPGLGLRLIYYSFDVKDINKGEI |
| | | | SDGYSVSRQAQAKFSLSLESAIPNQTALYFCATSGPLTG |
| | | | PPEQFFGPGTRLTVLEDLKNVFPPEVAVFEPSEAEISHTQ |
| | | | KATLVCLATGFYPDHVELSWWVNGKEVHSGVSTDPQPL |
| | | SEQ | |
| TCR | Description | ID NO: | Nucleotide sequence |
| 1 | TRA | 543 | ACTGCGCGGTCACCCTTTTCTTATATGGGGACTGTGAT |
| | | | TTCTTCATGTTAAGGATCAAGACCATTATTTGGGTAACA |
| | | | CACTAAAGATGAACTATTCTCCAGGCTTAGTATCTCTG |
| | | | ATACTCTTACTGCTTGGAAGAACCCGTGGAAATTCAGT |
| | | | GACCCAGATGGAAGGGCCAGTGACTCTCTCAGAAGAG |
| | | | GCCTTCCTGACTATAAACTGCACGTACACAGCCACAG |
| | | | GATACCCTTCCCTTTTCTGGTATGTCCAATATCCTGGA |
| | | | GAAGGTCTACAGCTCCTCCTGAAAGCCACGAAGGCTG |
| | | | ATGACAAGGGAAGCAACAAAGGTTTTGAAGCCACATA |
| | | | CCGTAAAGAAACCACTTCTTTCCACTTGGAGAAAGGCT |
| | | | CAGTTCAAGTGTCAGACTCAGCGGTGTACTTCTGTGCT |
| | | | CTGAGTAGCTATGGAAACAAGCTGGTCTTTGGCGCAG |
| | | | GAACCATTCTGAGAGTCAAGTCCTATATCCAGAACCCT |
| | | | GACCCTGCCGTGTACCAGCTGAGAGACTCTAAATCCA |
| | | | GTGACAAGTCTGTCTGCCTATTCAC |
| | TRB | 544 | GCACTAACTGATATCTCTGTTTCGAGTTGCCTTCAACT |
| | | | CGAAACATCCAGCAGAGGATGGGCCTAGAGATGGAGT |
| | | | AGGAGATCCAGTCCCCAAGCCCTAGAGATGTGAGTGA |
| | | | GGACACTTGGCTGAACTTACACAGTCTGTGAATACAGT |
| | | | AAAGCAACTTCGTGCTCTCTTTTAGCTTATAATAATTGG |
| | | | TTTCTAACAATGTAGGCATTTGTGGAGGCAATGATGTC |
| | | | ACTGTGGGAACTGCCATGAGAGGACAGGGACGTCCCT |
| | | | CCTCCTCTGCTTTTGCTCACAGTGACCCTGATTGGGCA |
| | | | AAGCTCCCATCCTTCCCTGACCCTGCCATGGGCACCA |
| | | | GGCTCCTCTGCTGGGCGGCCCTCTGTCTCCTGGGAG |
| | | | CAGAACTCACAGAAGCTGGAGTTGCCCAGTCTCCCAG |
| | | | ATATAAGATTATAGAGAAAAGGCAGAGTGTGGCTTTTT |
| | | | GGTGCAATCCTATATCTGGCCATGCTACCCTTTACTGG |
| | | | TACCAGCAGATCCTGGGACAGGGCCCAAAGCTTCTGA |
| | | | TTCAGTTTCAGAATAACGGTGTAGTGGATGATTCACAG |
| | | | TTGCCTAAGGATCGATTTTCTGCAGAGAGGCTCAAAG |
| | | | GAGTAGACTCCACTCTCAAGATCCAGCCTGCAAAGCTT |
| | | | GAGGACTCGGCCGTGTATCTCTGTGCCAGCAGCTCGC |
| | | | TTAGCGGGAGCTCCTACGAGCAGTACTTCGGGCCGG |
| | | | GCACCAGGCTCACGGTCACAGAGGACCTGAAAAACGT |
| | | | GTTCCCACCCGAGGTCGCTGTGTTTGAGCCATCAGAA |
| | | | GCAGAGATCTCCCACACCCAAAAGGCCACACTGGTGT |
| | | | GCCTGGCCACAGGCTTCTACCCCGACCACGTGGAGCT |
| | | | GAGCTGGTGGGTGAATGGGAAGGAGGTGCACAGTGG |
| | | | GGTCAGCACAGACCCGCAGCCCCTCAAGGAGCAGCC |
| | | | CGCCCTCAATGACTCCAGATACTGCCTGA |
| 2 | TRA | 545 | AAAACATAGACTCTCCTCTGAGAGGGTGGGCAAAATA |
| | | | GTGTGATTGGTTTTCTCAACTTGCAGTCACCTCTTTCC |
| | | | CTTTCTTTCCAAAATGCACACATCTACTTTCCAAAATAG |
| | | | ACCGCAACTATTTCTATTGATCTGGAAGAAGCTTGTAC |
| | | | CAGGCAACCCATTTAGGAGAAGTTGGATGAAGAGGGA |
| | | | GAGGGAGATGCTACTCATCACATCAATGTTGGTCTTAT |
| | | | GGATGCAATTGTCACAGGTGAATGGACAACAGGTAAT |
| | | | GCAAATTCCTCAGTACCAGCATGTACAAGAAGGAGAG |
| | | | GACTTCACCACGTACTGCAATTCCTCAACTACTTTAAG |
| | | | CAATATACAGTGGTATAAGCAAAGGCCTGGTGGACAT |
| | | | CCCGTTTTTTTGATACAGTTAGTGAAGAGTGGAGAAGT |
| | | | GAAGAAGCAGAAAAGACTGACATTTCAGTTTGGAGAA |
| | | | GCAAAAAAGAACAGCTCCCTGCACATCACAGCCACCC |
| | | | AGACTACAGATGTAGGAACCTACTTCTGTGCAGGGCA |
| | | | CGCAGGAGCCCAGAAGCTGGTATTTGGCCAAGGAACC |
| | | | AGGCTGACTATCAACCCAAATATCCAGAACCCTGACCC |
| | | | TGCCGTGTACCAGCTGAGAGACTAGATCGGAAG |
| | TRB | 546 | GGATTCTTCCCATTCAAACGGTTCACCGGTGCATGAAT |
| | | | CTTGAATTTGACCATCTGGGGAAGGGGCGTGGCCTCT |
| | | | CCTGACAGGAAGGCTCTGGGGCCCAGGCAGGGAGAA |
| | | | TGAAGTCTCAGAATGACCCCCTTGAGAGTACTGTTCCC |
| | | | CTATCACCGATGCACAGACCCAGAAGACCCCTCCATC |
| | | | CTGTAGCACCTGCCATGAGCATCGGGCTCCTGTGCTG |
| | | | TGTGGCCTTTTCTCTCCTGTGGGCAAGTCCAGTGAATG |
| | | | CTGGTGTCACTCAGACCCCAAAATTCCAGGTCCTGAA |
| | | | GACAGGACAGAGCATGACACTGCAGTGTGCCCAGGAT |
| | | | ATGAACCATAACTCCATGTACTGGTATCGACAAGACCC |
| | | | AGGCATGGGACTGAGGCTGATTTATTACTCAGCTTCTG |
| | | | AGGGTACCACTGACAAAGGAGAAGTCCCCAATGGCTA |
| | | | CAATGTCTCCAGATTAAACAAACGGGAGTTCTCGCTCA |
| | | | GGCTGGAGTCGGCTGCTCCCTCCCAGACATCTGTGTA |
| | | | CTTCTGTGCCAGCAGTGAGGTATGGGTCGGTAGCACA |
| | | | GATACGCAGTATTTTGGCCCAGGCACCCGGCTGACAG |
| | | | TGCTCGAGGACCTGAAAAACGTGTTCCCACCCGAGGT |
| | | | CGCTGTGTTTGAGCCATCAGAAGCAGAGATCTCCCAC |
| | | | ACCCAAAAGGCCACACTGGTGTGCCTGGCCACAGGCT |
| | | | TCTACCCCGACCACGTGGAGCTGAGCTGGTGGGTGAA |
| | | | TGGGAAGGAGGTGCACAGTGGGGTCAGCACAGACCC |
| | | | GCAGCCCCTCAAGGAGCAGCCCGCCCTCAATGACTCC |
| | | | AGATACTGCCTGA |
| 3 | TRA | 547 | TTATATGGGGGTTCATATGTAAAATGAAGGGTCTGTGG |
| | | | AAGGACATGAATAAAGCACAGGAGGTTGAAGTCAGAT |
| | | | TTGCAGCTTTCTAGGCAGGAGATAAGACAATCTGCATC |
| | | | TTCACAGGAGGGATGGCCATGCTCCTGGGGGCATCAG |
| | | | TGCTGATTCTGTGGCTTCAGCCAGACTGGGTAAACAG |
| | | | TCAACAGAAGAATGATGACCAGCAAGTTAAGCAAAATT |
| | | | CACCATCCCTGAGCGTCCAGGAAGGAAGAATTTCTATT |
| | | | CTGAACTGTGACTATACTAACAGCATGTTTGATTATTTC |
| | | | CTATGGTACAAAAAATACCCTGCTGAAGGTCCTACATT |
| | | | CCTGATATCTATAAGTTCCATTAAGGATAAAAATGAAGA |
| | | | TGGAAGATTCACTGTTTTCTTAAACAAAAGTGCCAAGC |
| | | | ACCTCTCTCTGCACATTGTGCCCTCCCAGCCTGGAGA |
| | | | CTCTGCAGTGTACTTCTGTGCAGCAAGCAGGCGGTTT |
| | | | GGAAATGAGAAATTAACCTTTGGGACTGGAACAAGACT |
| | | | CACCATCATACCCAATATCCAGAACCCTGACCCTGCC |
| | | | GTGTACCAGCTGAGAGACTAGAT |
| | TRB | 548 | CCACTTCCCAAACACCTCCCACTCCTACCCAGACCGT |
| | | | GGATGGGCAGGAAATGCAGGAACAGAGCCAGAAACA |
| | | | GGAGATCTCCAAGGAAGGTTGACAGTCAGCACTGGGA |
| | | | TCGTCTGTGTAAAGTGCTGCTGAAGCAGCCAGGTGGC |
| | | | ATGTCCAGCCGACAATGCGAAAGGAAAAATGGTGCAC |
| | | | ATCAGAGTTGCTGCCATCTTAGACTTAACTCATCAGTA |
| | | | TCAGGTGATCCTGAGGCTCAGTGATGTCACTGTGGGA |
| | | | ACTGCTCTGTGGCGACAAGGACGTCCCTCATCCTCTG |
| | | | CTCCTGCTCACAGTGACCCTGATCTGGTAAAGCTCCC |
| | | | ATCCTGCCCTGACCCTGCCATGGGCACCAGCCTCCTC |
| | | | TGCTGGATGGCCCTGTGTCTCCTGGGGGCAGATCACG |
| | | | CAGATACTGGAGTCTCCCAGAACCCCAGACACAAGAT |
| | | | CACAAAGAGGGGACAGAATGTAACTTTCAGGTGTGAT |
| | | | CCAATTTCTGAACACAACCGCCTTTATTGGTACCGACA |
| | | | GACCCTGGGGCAGGGCCCAGAGTTTCTGACTTACTTC |
| | | | CAGAATGAAGCTCAACTAGAAAAATCAAGGCTGCTCAG |
| | | | TGATCGGTTCTCTGCAGAGAGGCCTAAGGGATCTTTCT |
| | | | CCACCTTGGAGATCCAGCGCACAGAGCAGGGGGACT |
| | | | CGGCCATGTATCTCTGTGCCAGCAGGCTCTGGAGCAG |
| | | | GGGGCGGACCACTGAAGCTTTCTTTGGACAAGGCACC |
| | | | AGACTCACAGTTGTAGAGGACCTGAACAAGGTGTTCC |
| | | | CACCCGAGGTCGCTGTGTTTGAGCCATCAGAAGCAGA |
| | | | GATCTCCCACACCCAAAAGGCCACACTGGTGTGCCTG |
| | | | GCCACAGGCTTCTACCCCGACCACGTGGAGCTGAGCT |
| | | | GGTGGGTGAATGGGAAGGAGGTGCACAGTGGGGTCA |
| | | | GCACAGACCCGCAGCCCCTCAAGGAGCAGCCCGCCC |
| | | | TCAATGACTCCAGATACTGCCTG |
| 4 | TRA | 549 | TTCTAAATTTGGACAAAGTGGGGAAGTGCTTCCTTTGA |
| | | | CAGAGACAGCTTTAAGTGAAAGCACTTGTGAAAGGGC |
| | | | GGGGCCTGCTGAAAGAATTCAGTTGAGGGTGAATTTA |
| | | | CAGAGTTTCAGCTGGTTGGGAAGACTGGAAGACCACC |
| | | | TGGGCTGTCATTGAGCTCTGGTGCCAGGAGGAATGGA |
| | | | CAAGATCTTAGGAGCATCATTTTTAGTTCTGTGGCTTC |
| | | | AACTATGCTGGGTGAGTGGCCAACAGAAGGAGAAAAG |
| | | | TGACCAGCAGCAGGTGAAACAAAGTCCTCAATCTTTGA |
| | | | TAGTCCAGAAAGGAGGGATTTCAATTATAAACTGTGCT |
| | | | TATGAGAACACTGCGTTTGACTACTTTCCATGGTACCA |
| | | | ACAATTCCCTGGGAAAGGCCCTGCATTATTGATAGCCA |
| | | | TACGTCCAGATGTGAGTGAAAAGAAAGAAGGAAGATT |
| | | | CACAATCTCCTTCAATAAAAGTGCCAAGCAGTTCTCAT |
| | | | TGCATATCATGGATTCCCAGCCTGGAGACTCAGCCAC |
| | | | CTACTTCTGTGCAGCAAGCAAGACCGGCACTGCCAGT |
| | | | AAACTCACCTTTGGGACTGGAACAAGACTTCAGGTCAC |
| | | | GCTCGATATCCAGAACCCTGACCCTGCCGTGTACCAG |
| | | | CTGAGAGACTAG |
| | TRB | 550 | GGGAGTCTAGACACACTAAGCCATGCTTACCAGTTACT |
| | | | ATAGTGGGAACACTCCCAAAATTCAAGTTCGCAGATGC |
| | | | AAATCAAGAGCCAACCATGCAAGCAGTCCCTTCTAAG |
| | | | GCCAGCAGTCTCAGGCCCGCTGTGTCAACTCTTTTCTA |
| | | | TACGTGCACATACACACAGTGACACTTCCATGAGCACA |
| | | | AGGGATGTCCTTCACTCTCTAGCACCTCTAGACCCTTT |
| | | | CTAGTCTAGACAATGGCTGAGGGATTATGAGAGTGGG |
| | | | CCGGGACAATGACATCACAGACCATCAACCCACTGCC |
| | | | TGGTCCTGGGAGAAGACCTATTCTTTCTTCAAAGCAGC |
| | | | CATGGGAATCAGGCTCCTCTGTCGTGTGGCCTTTTGTT |
| | | | TCCTGGCTGTAGGCCTCGTAGATGTGAAAGTAACCCA |
| | | | GAGCTCGAGATATCTAGTCAAAAGGACGGGAGAGAAA |
| | | | GTTTTTCTGGAATGTGTCCAGGATATGGACCATGAAAA |
| | | | TATGTTCTGGTATCGACAAGACCCAGGTCTGGGGCTA |
| | | | CGGCTGATCTATTTCTCATATGATGTTAAAATGAAAGAA |
| | | | AAAGGAGATATTCCTGAGGGGTACAGTGTCTCTAGAG |
| | | | AGAAGAAGGAGCGCTTCTCCCTGATTCTGGAGTCCGC |
| | | | CAGCACCAACCAGACATCTATGTACCTCTGTGCCAGC |
| | | | AGTGTGAATGAGCAGTTCTTCGGGCCAGGGACACGGC |
| | | | TCACCGTGCTAGAGGACCTGAAAAACGTGTTCCCACC |
| | | | CGAGGTCGCTGTGTTTGAGCCATCAGAAGCAGAGATC |
| | | | TCCCACACCCAAAAGGCCACACTGGTGTGCCTGGCCA |
| | | | CAGGCTTC |
| 5 | TRA | 551 | GACAGAGCTCACTATAGGCTTCTGGGATTTAATGACAC |
| | | | TTTCCACAAGCTCCATGGAGCAGAGGAAACTTTATTTG |
| | | | GTACATTGAGGAAGAAATGATGGAAGAAAAGGTCTGG |
| | | | ATAGATACATCCTTATGATCAGTTTTCCTCATTCTGTGC |
| | | | CAGCCTCTTAGACTTTAAACCTCATAAATGATTTCAGTA |
| | | | GACACCCCCTGAGTTGAGGATGTCTGGTTGATATCCT |
| | | | CACCGTCTTCTCAGGAACATGACTAAACCTGGGGAGG |
| | | | AAGACATAGAAAAGCAACTGCTCTATCATTGGCTGACA |
| | | | AGATCCTGACAACAACACAATGAGAGCTGCACTCCTTG |
| | | | CAAGTCTTTACTAGTAGCTGTTGCTTCTGTTCTCTGGA |
| | | | AATTCTTTAAACCTAGAATCAGACACAAAAACTGAACTC |
| | | | TGGGTCCACAATCCTCATTTGTCCTTGAAGTATGAGGC |
| | | | TGGTGGCAAGAGTAACTGTGTTTCTGACCTTTGGAACT |
| | | | ATAATTGATGCTAAGACCACCCAGCCCCCCTCCATGG |
| | | | ATTGCGCTGAAGGAAGAGCTGCAAACCTGCCTTGTAA |
| | | | TCACTCTACCATCAGTGGAAATGAGTATGTGTATTGGT |
| | | | ATCGACAGATTCACTCCCAGGGGCCACAGTATATCATT |
| | | | CATGGTCTAAAAAACAATGAAACCAATGAAATGGCCTC |
| | | | TCTGATCATCACAGAAGACAGAAAGTCCAGCACCTTGA |
| | | | TCCTGCCCCACGCTACGCTGAGAGACACTGCTGTGTA |
| | | | CTATTGCATCGTCAAAAGTTGGAACAACCGTAAGCTGA |
| | | | TTTGGGGATTGGGAACAAGCCTGGCAGTAAATCCGAA |
| | | | TATCCAGAACCCTGACCCTGCCGTGTACCAGCTGAGA |
| | | | GACTAGA |
| | TRB | 552 | GCCATCTTAGACTTAACTCATCAGTATCAGGTGATCCT |
| | | | GAGGCTCAGTGATGTCACTGTGGGAACTGCTCTGTGG |
| | | | CGACAAGGACGTCCCTCATCCTCTGCTCCTGCTCACA |
| | | | GTGACCCTGATCTGGTAAAGCTCCCATCCTGCCCTGA |
| | | | CCCTGCCATGGGCACCAGCCTCCTCTGCTGGATGGCC |
| | | | CTGTGTCTCCTGGGGGCAGATCACGCAGATACTGGAG |
| | | | TCTCCCAGAACCCCAGACACAAGATCACAAAGAGGGG |
| | | | ACAGAATGTAACTTTCAGGTGTGATCCAATTTCTGAAC |
| | | | ACAACCGCCTTTATTGGTACCGACAGACCCTGGGGCA |
| | | | GGGCCCAGAGTTTCTGACTTACTTCCAGAATGAAGCTC |
| | | | AACTAGAAAAATCAAGGCTGCTCAGTGATCGGTTCTCT |
| | | | GCAGAGAGGCCTAAGGGATCTTTCTCCACCTTGGAGA |
| | | | TCCAGCGCACAGAGCAGGGGGACTCGGCCATGTATCT |
| | | | CTGTGCCAGCCTTAAGAGAACAGGGGGCCACAATCAG |
| | | | CCCCAGCATTTTGGTGATGGGACTCGACTCTCCATCCT |
| | | | AGAGGACCTGAACAAGGTGTTCCCACCCGAGGTCGCT |
| | | | GTGTTTGAGCCATCAGAAGCAGAGATCTCCCACACCC |
| | | | AAAAGGCCACACTGGTGTGCCTGGCCACAGGCTTCTA |
| | | | CCCCGACCACGTGGAGCTGAGCTGGTGGGTGAATGG |
| | | | GAAGGAGGTGCACAGTGGGGTCAGCACAGACCCGCA |
| | | | GCCCCTCAAGGAGCAGCCCGCCCTCAATGACTCCAGA |
| | | | TACTGCCTGA |
| 6 | TRA | 553 | GAGTCAGATTCTTGCTATGCTGCCCGGGCTGGTCTTG |
| | | | AACCCCTGGCTTCAAGCAATCTTCCAGCTTCAGCCACA |
| | | | CAAAGTGTGGGGATTACAGAAGGTAACATCATCACAG |
| | | | ATTCAGGAGGAAAGAAACGCTTTCTGTTGAAGTTTTGA |
| | | | TATCATGTCTGAAATGAGAAGGAAACAGCTTGCTCCCT |
| | | | TGGATCCGTGGTTCCGCCTGTTGCCTCTAGAGGGCGA |
| | | | TGTTGCATCAAGTAGGCATCTGTTTTCATCGCAGACCA |
| | | | TCCTCATCCATTGAGCCTCCTCCCTGTAGCTGGCTGAT |
| | | | GTAGCTCGTTGATATCTGTGTGGATAGGGAGCTGTGA |
| | | | CGAGGGCAAGAGGTCAGAACACATCCAGGCTCCTTAA |
| | | | GGGAAAGCCTCTTTCTGTTTCTGAAACTTTTCAAAGCC |
| | | | AGGGACTTGTCCAATCCAACCTCCTCACAGTTCCTAGC |
| | | | TCCTGAGGCTCAGCGCCCTTGGCTTCTGTCCGCCCAG |
| | | | CTCAAGGTCCTGCAGCATTGCCACTGCTCAGCCATGC |
| | | | TCCTGCTGCTCGTCCCAGCGTTCCAGGTGATTTTTACC |
| | | | CTGGGAGGAACCAGAGCCCAGTCTGTGACCCAGCTTG |
| | | | ACAGCCAAGTCCCTGTCTTTGAAGAAGCCCCTGTGGA |
| | | | GCTGAGGTGCAACTACTCATCGTCTGTTTCAGTGTATC |
| | | | TCTTCTGGTATGTGCAATACCCCAACCAAGGACTCCAG |
| | | | CTTCTCCTGAAGTATTTATCAGGATCCACCCTGGTTAA |
| | | | AGGCATCAACGGTTTTGAGGCTGAATTTAACAAGAGTC |
| | | | AAACTTCCTTCCACTTGAGGAAACCCTCAGTCCATATA |
| | | | AGCGACACGGCTGAGTACTTCTGTGCTGTGAGTGTTT |
| | | | CTGGTTCTGCAAGGCAACTGACCTTTGGATCTGGGAC |
| | | | ACAATTGACTGTTTTACCTGATATCCAGAACCCTGACC |
| | | | CTGCCGTGTACCAGCTGAGAGACTCTAAATCCAGTGA |
| | | | CAAGTCTGTCTGCCTATTCACCGATTTTGATTCTAAA |
| | | | CAAATGTGTCACAAAGTAAGGATTCTGATGTGTATATC |
| | | | ACAGACAAAACTGTGCTAGACATGAGGTCTATGGACTT |
| | | | CAAGAGCAACAGTGCTGTGGCCTGGAGCAACAAATCT |
| | | | GACTTTGCATGTGCAAACGCCTTCA |
| | TRB | 554 | GTGGTGAGGAGATAAACTCAGAAATGCAGCATGAGGC |
| | | | CTGTAGCTCCAGACAGCTCTAGAGCACAAAGTGAAGA |
| | | | CCGATGCATTGATGTTGTTTAAAAGGAGCTAATAAATA |
| | | | TCTAAAGCAGTCATCCAAGTGTGTTCTAATATAAATCCT |
| | | | GTGTTCCTGAGGTTGTGGGGATTGAGAGAGGAAGTGA |
| | | | TGTCACTGTGGGTACTGTTCTGTGTCAGGACAAGGAC |
| | | | GTCCCTCCTCCTCTGCTCCTGCTCACAGTGACCCTGAT |
| | | | CTGGTAAAGCTCCCATCCTGCCCTGACTCTGTCATGG |
| | | | GCACCAGGCTCCTCTGCTGGGCAGCCCTGTGCCTCCT |
| | | | GGGGGCAGATCACACAGGTGCTGGAGTCTCCCAGAC |
| | | | CCCCAGTAACAAGGTCACAGAGAAGGGAAAATATGTA |
| | | | GAGCTCAGGTGTGATCCAATTTCAGGTCATACTGCCCT |
| | | | TTACTGGTACCGACAAAGCCTGGGGCAGGGCCCAGA |
| | | | GTTTCTAATTTACTTCCAAGGCACGGGTGCGGCAGAT |
| | | | GACTCAGGGCTGCCCAACGATCGGTTCTTTGCAGTCA |
| | | | GGCCTGAGGGATCCGTCTCTACTCTGAAGATCCAGCG |
| | | | CACAGAGCGGGGGGACTCAGCCGTGTATCTCTGTGCC |
| | | | AGCAGCCACCGTCTAGCGGGAGCTGAGCAGTTCTTCG |
| | | | GGCCAGGGACACGGCTCACCGTGCTAGAGGACCTGA |
| | | | AAAACGTGTTCCCACCCGAGGTCGCTGTGTTTGAGCC |
| | | | ATCAGAAGCAGAGATCTCCCACACCCAAAAGGCCACA |
| | | | CTGGTGTGCCTGGCCACAGGCTTCTACCCCGACCACG |
| | | | TGGAGCTGAGCTGGTGGGTGAATGGGAAGGAGGTGC |
| | | | ACAGTGGGGTCAGCACAGACCCGCAGCCCCTCAAGG |
| | | | AGCAGCCCGCCCTCAATGACTCCAGATACTGCCTGA |
| 7 | TRA | 555 | TATGGGGACTGTGATTTCTTCATGTTAAGGATCAAGAC |
| | | | CATTATTTGGGTAACACACTAAAGATGAACTATTCTCCA |
| | | | GGCTTAGTATCTCTGATACTCTTACTGCTTGGAAGAAC |
| | | | CCGTGGAAATTCAGTGACCCAGATGGAAGGGCCAGTG |
| | | | ACTCTCTCAGAAGAGGCCTTCCTGACTATAAACTGCAC |
| | | | GTACACAGCCACAGGATACCCTTCCCTTTTCTGGTATG |
| | | | TCCAATATCCTGGAGAAGGTCTACAGCTCCTCCTGAAA |
| | | | GCCACGAAGGCTGATGACAAGGGAAGCAACAAAGGTT |
| | | | TTGAAGCCACATACCGTAAAGAAACCACTTCTTTCCAC |
| | | | TTGGAGAAAGGCTCAGTTCAAGTGTCAGACTCAGCGG |
| | | | TGTACTTCTGTGCTCTGACCCTGAATTCAGGATACAGC |
| | | | ACCCTCACCTTTGGGAAGGGGACTATGCTTCTAGTCTC |
| | | | TCCAGATATCCAGAACCCTGACCCTGCCGTGTACCAG |
| | | | CTGAGAGACTCTAAATCCAGTGACAAGTCTGTCTGCCT |
| | | | ATTCACCGATTTTGATT |
| | TRB | 556 | GAGACTAGGGCATCCTTGGGATTCTGTGATCAGTCAT |
| | | | CCCTCCTCGCTGGTGAATGGAGGCAGTGGTCACAACT |
| | | | CTCCCCAGAGAAGGTGGTGTGAGGCCATCACGGAAGA |
| | | | TGCTGCTGCTTCTGCTGCTTCTGGGGCCAGGCTCCGG |
| | | | GCTTGGTGCTGTCGTCTCTCAACATCCGAGCTGGGTT |
| | | | ATCTGTAAGAGTGGAACCTCTGTGAAGATCGAGTGCC |
| | | | GTTCCCTGGACTTTCAGGCCACAACTATGTTTTGGTAT |
| | | | CGTCAGTTCCCGAAACAGAGTCTCATGCTGATGGCAA |
| | | | CTTCCAATGAGGGCTCCAAGGCCACATACGAGCAAGG |
| | | | CGTCGAGAAGGACAAGTTTCTCATCAACCATGCAAGC |
| | | | CTGACCTTGTCCACTCTGACAGTGACCAGTGCCCATC |
| | | | CTGAAGACAGCAGCTTCTACATCTGCAGTGCTAGAGA |
| | | | CCCAGGAACCTTATACGAGCAGTACTTCGGGCCGGGC |
| | | | ACCAGGCTCACGGTCACAGAGGACCTGAAAAACGTGT |
| | | | TCCCACCCGAGGTCGCTGTGTTTGAGCCATCAGA |
| 8 | TRA | 557 | GTCATTACTAAGCAGCCTCACAACTGTAGCAACCCTTC |
| | | | TAAAGGTTGTAGATTCTGGCTGATGATGTCACTGACAC |
| | | | AAAGGAAAAAATGCAAAACAGGTAGTCTTAAATAAGCA |
| | | | TTCTGGTGAGACCAACTGCATTTTGGCCATGGCTTTGC |
| | | | AGAGCACTCTGGGGGCGGTGTGGCTAGGGCTTCTCCT |
| | | | CAACTCTCTCTGGAAGGTTGCAGAAAGCAAGGACCAA |
| | | | GTGTTTCAGCCTTCCACAGTGGCATCTTCAGAGGGAG |
| | | | CTGTGGTGGAAATCTTCTGTAATCACTCTGTGTCCAAT |
| | | | GCTTACAACTTCTTCTGGTACCTTCACTTCCCGGGATG |
| | | | TGCACCAAGACTCCTTGTTAAAGGCTCAAAGCCTTCTC |
| | | | AGCAGGGACGATACAACATGACCTATGAACGGTTCTC |
| | | | TTCATCGCTGCTCATCCTCCAGGTGCGGGAGGCAGAT |
| | | | GCTGCTGTTTACTACTGTGCTGTGGAGCGGCCAGGCT |
| | | | TTCAGAAACTTGTATTTGGAACTGGCACCCGACTTCTG |
| | | | GTCAGTCCAAATATCCAGAACCCTGACCCTGCCGTGT |
| | | | ACCAGCTGAGAGACT |
| | TRB | 558 | GGCTCACTGGCGCAGCACCTCTCAGCGGCAGTGGAA |
| | | | ACCACAGCCTAGTCCTCTCACCACTGCAGACCAGAAT |
| | | | CCTGCCCTGGGCCTTGCCTGGTCTGCCTCACTCTGCC |
| | | | ATGGGCTGCAGGCTCCTCTGCTGTGTGGTCTTCTGCC |
| | | | TCCTCCAAGCAGGTCCCTTGGACACAGCTGTTTCCCA |
| | | | GACTCCAAAATACCTGGTCACACAGATGGGAAACGAC |
| | | | AAGTCCATTAAATGTGAACAAAATCTGGGCCATGATAC |
| | | | TATGTATTGGTATAAACAGGACTCTAAGAAATTTCTGAA |
| | | | GATAATGTTTAGCTACAATAATAAGGAGCTCATTATAAA |
| | | | TGAAACAGTTCCAAATCGCTTCTCACCTAAATCTCCAG |
| | | | ACAAAGCTCACTTAAATCTTCACATCAATTCCCTGGAG |
| | | | CTTGGTGACTCTGCTGTGTATTTCTGTGCCAGCAGGAC |
| | | | GACAGGGTCGGGGACTGAAGCTTTCTTTGGACAAGGC |
| | | | ACCAGACTCACAGTTGTAGAGGACCTGAACAAGGTGT |
| | | | TCCCACCCGAGGTCGCTGTGTTTGAGCCATCAGAAGC |
| | | | AGAGATCTCCCACACCCAAAAGGCCACACTGGTGTGC |
| | | | CTGGCCACAGGCTTCTTCCCTGACCACGTGGAGCTGA |
| | | | GCTGGTGGGTGAATGGGAAGGAGGTGCACAGTGGGG |
| | | | TCAGCACAGACCCGCAGCCCCTCAAGGAGCAGCCCG |
| | | | CCCTCAATGACTCCAGATACTGCCTGA |
| 9 | TRA | 559 | GGTAATTTCTTCTGAGTCAGCTCTGAACACACGTCCTG |
| | | | ATGGCAGCAGGATGATGTTACGATCACAAGAGGGCAG |
| | | | TGCCTCCCTCTCCTCAGACTAACTTGGCTTTAAATCAG |
| | | | CCTATCTGCATTGAAAGGAAAGGACTGAGCTTGCCTGT |
| | | | GACTGGCTAGGGAGGAACCTGAGACTAGGGGACAGA |
| | | | AAGACTAGGGATTCACCCAGTAAAGAGAGCTCATCTGT |
| | | | GACTGAGGAGCCTTGCTCCATTTCAGGTCTTCTGTGAT |
| | | | TTCAATAAGGAAGAAGAATGGAAACTCTCCTGGGAGT |
| | | | GTCTTTGGTGATTCTATGGCTTCAACTGGCTAGGGTGA |
| | | | ACAGTCAACAGGGAGAAGAGGATCCTCAGGCCTTGAG |
| | | | CATCCAGGAGGGTGAAAATGCCACCATGAACTGCAGT |
| | | | TACAAAACTAGTATAAACAATTTACAGTGGTATAGACAA |
| | | | AATTCAGGTAGAGGCCTTGTCCACCTAATTTTAATACG |
| | | | TTCAAATGAAAGAGAGAAACACAGTGGAAGATTAAGAG |
| | | | TCACGCTTGACACTTCCAAGAAAAGCAGTTCCTTGTTG |
| | | | ATCACGGCTTCCCGGGCAGCAGACACTGCTTCTTACT |
| | | | TCTGTGCTACGCCGAGGCGAGGTGCTGACGGACTCAC |
| | | | CTTTGGCAAAGGGACTCATCTAATCATCCAGCCCTATA |
| | | | TCCAGAACCCTGACCCTGCCGTGTACCAGCTGAGAGA |
| | | | CT |
| | TRA | 560 | GCCTCCTGCCTTTCTATGGAACTCAAAGTGGGTGGCA |
| | | | GCAAGCACTCTTCTAGCCCAGAGAAGTCTGTTCCAGG |
| | | | ACGGGCTCTTTCAGGAGCAGCTAAAGTCAGGGGCCAT |
| | | | GTCCACCATGTGATAGAAAGACAAGATGGTCCTGAAAT |
| | | | TCTCCGTGTCCATTCTTTGGATTCAGTTGGCATGGGTG |
| | | | AGCACCCAGCTGCTGGAGCAGAGCCCTCAGTTTCTAA |
| | | | GCATCCAAGAGGGAGAAAATCTCACTGTGTACTGCAA |
| | | | CTCCTCAAGTGTTTTTTCCAGCTTACAATGGTACAGAC |
| | | | AGGAGCCTGGGGAAGGTCCTGTCCTCCTGGTGACAGT |
| | | | AGTTACGGGTGGAGAAGTGAAGAAGCTGAAGAGACTA |
| | | | ACCTTTCAGTTTGGTGATGCAAGAAAGGACAGTTCTCT |
| | | | CCACATCACTGCGGCCCAGCCTGGTGATACAGGCCTC |
| | | | TACCTCTGTGCCTTTTGTAACCAGTTCTATTTTGGGAC |
| | | | AGGGACAAGTTTGACGGTCATTCCAAATATCCAGAACC |
| | | | CTGACCCTGCCGTGTACCAGCTGAGAGACT |
| | TRB | 561 | AGCCCATTCATCCCAGCCCAGACAATTCAAATCTGCCT |
| | | | TCTATCAGGACCTAGAAAGGATGTAAAACGGCCGGGT |
| | | | ATAAATATCCCCTGGGTCTGGGGAAACTGTCAGGAGC |
| | | | AGTGACATCACAGGAAAAACCACCAACCAAGGCCAAG |
| | | | GAGACCAGAGCCCAGCACCTCACCCAGAGGACCCCA |
| | | | GTCAGAGGCCCCATCTCAGACCCGAGGCTAGCATGG |
| | | | GCTGCAGGCTGCTCTGCTGTGCGGTTCTCTGTCTCCT |
| | | | GGGAGCGGTCCCCATGGAAACGGGAGTTACGCAGAC |
| | | | ACCAAGACACCTGGTCATGGGAATGACAAATAAGAAG |
| | | | TCTTTGAAATGTGAACAACATCTGGGGCATAACGCTAT |
| | | | GTATTGGTACAAGCAAAGTGCTAAGAAGCCACTGGAG |
| | | | CTCATGTTTGTCTACAACTTTAAAGAACAGACTGAAAA |
| | | | CAACAGTGTGCCAAGTCGCTTCTCACCTGAATGCCCC |
| | | | AACAGCTCTCACTTATTCCTTCACCTACACACCCTGCA |
| | | | GCCAGAAGACTCGGCCCTGTATCTCTGTGCCAGCAGC |
| | | | CCGGGGGGTAGCGGGAGGTTGGGAGATACGCAGTAT |
| | | | TTTGGCCCAGGCACCCGGCTGACAGTGCTCGAGGAC |
| | | | CTGAAAAACGTGTTCCCACCCGAGGTCGCTGTGTTTG |
| | | | AGCCATCAGAAGCAGAGATCTCCCACACCCAAAAGGC |
| | | | CACACTGGTGTGCCTGGCCACAGGCTTCTTCCCTGAC |
| | | | CACGTGGAGCTGAGCTGGTGGGTGAATGGGAAGGAG |
| | | | GTGCACAGTGGGGTCAGCACGGACCCGCAGCCCCTC |
| | | | AAGGAGCAGCCCGCCCTCAATGACTCCAGATACTGCC |
| | | | TGA |
| 10 | TRA | 562 | CAACTGTAGCAACCCTTCTAAAGGTTGTAGATTCTGGC |
| | | | TGATGATGTCACTGACACAAAGGAAAAAATGCAAAACA |
| | | | GGTAGTCTTAAATAAGCATTCTGGTGAGACCAACTGCA |
| | | | TTTTGGCCATGGCTTTGCAGAGCACTCTGGGGGCGGT |
| | | | GTGGCTAGGGCTTCTCCTCAACTCTCTCTGGAAGGTT |
| | | | GCAGAAAGCAAGGACCAAGTGTTTCAGCCTTCCACAG |
| | | | TGGCATCTTCAGAGGGAGCTGTGGTGGAAATCTTCTG |
| | | | TAATCACTCTGTGTCCAATGCTTACAACTTCTTCTGGTA |
| | | | CCTTCACTTCCCGGGATGTGCACCAAGACTCCTTGTTA |
| | | | AAGGCTCAAAGCCTTCTCAGCAGGGACGATACAACAT |
| | | | GACCTATGAACGGTTCTCTTCATCGCTGCTCATCCTCC |
| | | | AGGTGCGGGAGGCAGATGCTGCTGTTTACTACTGTGC |
| | | | TGTGGAGGATTCGCGGGGGAACAACAGACTCGCTTTT |
| | | | GGGAAGGGGAACCAAGTGGTGGTCATACCAAATATCC |
| | | | AGAACCCTGACCCTGCCGTGTACCAGCTGAGAGACTC |
| | | | TAAATCCAGTGACAAGTCTGTC |
| | TRB | 563 | GGGGTGTGGCCTCTCCTGGCCTCTCCCTCCCTGGGG |
| | | | CCCAGGCAGGGAGAATGTCTCAGAATGACTTCCTTGA |
| | | | GAGTCCTGCTCCCCTTTCATCAATGCACAGATACAGAA |
| | | | GACCCCTCCGTCATGCAGCATCTGCCATGAGCATCGG |
| | | | CCTCCTGTGCTGTGCAGCCTTGTCTCTCCTGTGGGCA |
| | | | GGTCCAGTGAATGCTGGTGTCACTCAGACCCCAAAAT |
| | | | TCCAGGTCCTGAAGACAGGACAGAGCATGACACTGCA |
| | | | GTGTGCCCAGGATATGAACCATGAATACATGTCCTGGT |
| | | | ATCGACAAGACCCAGGCATGGGGCTGAGGCTGATTCA |
| | | | TTACTCAGTTGGTGCTGGTATCACTGACCAAGGAGAA |
| | | | GTCCCCAATGGCTACAATGTCTCCAGATCAACCACAGA |
| | | | GGATTTCCCGCTCAGGCTGCTGTCGGCTGCTCCCTCC |
| | | | CAGACATCTGTGTACTTCTGTGCCAGCGGAGGGACAG |
| | | | GGGGGAACTATGGCTACACCTTCGGTTCGGGGACCAG |
| | | | GTTAACCGTTGTAGAGGACCTGAACAAGGTGTTCCCA |
| | | | CCCGAGGTCGCTGTGTTTGAGCCATCAGAAGCAGAGA |
| | | | TCTCCCACACCCAAAAGGCCACACTGGTGTGCCTGGC |
| | | | CACAGGCTTCTTCCCTGACCACGTGGAGCTGAGCTGG |
| | | | TGGGTGAATGGGAAGGAGGTGCACAGTGGGGTCAGC |
| | | | ACAGACCCGCAGCCCCTCAAGGAGCAGCCCGCCCTC |
| | | | AATGACTCCAGATACTGCCTGA |
| 11 | TRA | 564 | TGGGGACCTATCATAGCATTTCCTGCCCTGAAGGAGA |
| | | | ATTCTCACCAAGCACAGAGGAGAACCCATCAGAGCAG |
| | | | GAGACTTTTCACTCTGCAGGGGAGCGCTGTCAGCATG |
| | | | ACACGTGTTAGCTTGCTGTGGGCAGTCGTGGTCTCCA |
| | | | CCTGTCTTGAATCCGGCATGGCCCAGACAGTCACTCA |
| | | | GTCTCAACCAGAGATGTCTGTGCAGGAGGCAGAGACT |
| | | | GTGACCCTGAGTTGCACATATGACACCAGTGAGAGTA |
| | | | ATTATTATTTGTTCTGGTACAAACAGCCTCCCAGCAGG |
| | | | CAGATGATTCTCGTTATTCGCCAAGAAGCTTATAAGCA |
| | | | ACAGAATGCAACGGAGAATCGTTTCTCTGTGAACTTCC |
| | | | AGAAAGCAGCCAAATCCTTCAGTCTCAAGATCTCAGAC |
| | | | TCACAGCTGGGGGACACTGCGATGTATTTCTGTGCTTT |
| | | | CATGAAGTCGCTATTTGGAAATGAGAAATTAACCTTTG |
| | | | GGACTGGAACAAGACTCACCATCATACCCAATATCCAG |
| | | | AACCCTGACCCTGCCGTGTACCAGCTGAGAGACTCTA |
| | | | AATCCAGTGACAAGTCTGTCTGCCTATTCACCGATTTT |
| | | | GATTCTCAAAC |
| | TRB | 565 | AGTCATGGGCAAAGATTACCACCAGGGGGCAGACTAG |
| | | | GGCATCCTTGGGATTCTGTGATCAGTCATCCCTCCTCG |
| | | | CTGGTGAATGGAGGCAGTGGTCACAACTCTCCCCAGA |
| | | | GAAGGTGGTGTGAGGCCATCACGGAAGATGCTGCTGC |
| | | | TTCTGCTGCTTCTGGGGCCAGGCTCCGGGCTTGGTGC |
| | | | TGTCGTCTCTCAACATCCGAGCAGGGTTATCTGTAAGA |
| | | | GTGGAACCTCTGTGAAGATCGAGTGCCGTTCCCTGGA |
| | | | CTTTCAGGCCACAACTATGTTTTGGTATCGTCAGTTCC |
| | | | CGAAACAGAGTCTCATGCTGATGGCAACTTCCAATGA |
| | | | GGGCTCCAAGGCCACATACGAGCAAGGCGTCGAGAA |
| | | | GGACAAGTTTCTCATCAACCATGCAAGCCTGACCTTGT |
| | | | CCACTCTGACAGTGACCAGTGCCCATCCTGAAGACAG |
| | | | CAGCTTCTACATCTGCAGTGCTAGGTCGGGCGAAAAA |
| | | | CTGTTTTTTGGCAGTGGAACCCAGCTCTCTGTCTTGGA |
| | | | GGACCTGAACAAGGTGTTCCCACCCGAGGTCGCTGTG |
| | | | TTTGAGCCATCAGAAGCAGAGATCTCCCACACCCAAAA |
| | | | GGCCACACTGGTGTGCCTGGCCACAGGCTTCTTCCCT |
| | | | GACCACGTGGAGCTGAGCTGGTGGGTGAATGGGAAG |
| | | | GAGGTGCACAGTGGGGTCAGCACAGACCCGCAGCCC |
| | | | CTCAAGGAGCAGCCCGCCCTCAATGACTCCAGATACT |
| | | | GCCTGA |
| 12 | TRA | 566 | ATATGGGGAGCATTTCCTGCCCTGAAGGAGAATTCTCA |
| | | | CCAAGCACAGAGGAGAACCCATCAGAGCAGGAGACTT |
| | | | TTCACTCTGCAGGGGAGCGCTGTCAGCATGACACGTG |
| | | | TTAGCTTGCTGTGGGCAGTCGTGGTCTCCACCTGTCTT |
| | | | GAATCCGGCATGGCCCAGACAGTCACTCAGTCTCAAC |
| | | | CAGAGATGTCTGTGCAGGAGGCAGAGACTGTGACCCT |
| | | | GAGTTGCACATATGACACCAGTGAGAGTAATTATTATT |
| | | | TGTTCTGGTACAAACAGCCTCCCAGCAGGCAGATGAT |
| | | | TCTCGTTATTCGCCAAGAAGCTTATAAGCAACAGAATG |
| | | | CAACGGAGAATCGTTTCTCTGTGAACTTCCAGAAAGCA |
| | | | GCCAAATCCTTCAGTCTCAAGATCTCAGACTCACAGCT |
| | | | GGGGGACACTGCGATGTATTTCTGTGCTTTCATGAAGT |
| | | | CTCCCTTTGGAAATGAGAAATTAACCTTTGGGACTGGA |
| | | | ACAAGACTCACCATCATACCCAATATCCAGAACCCTGA |
| | | | CCCTGCCGTGTACCAGCTGAGAGACTCTAAATCCAGT |
| | | | GACAAGTCTGTCTGCCTATTCACCGATTTTGATTCTCA |
| | | | AACAAATGTGTCACAAAGTAAGGATTCTGATGTGTATA |
| | | | TCACAGACAAAACTGTGCTAGACATGAGGTCTATGGAC |
| | | | TTCAAGAGCAACAGTGCTGTGGCCTGGAGCAACAAAT |
| | | | CTGACTTTGCATGTGCAAACGCCTTCA |
| | TRB | 567 | GGGGGGGGATTCTGTGATCAGTCATCCCTCCTCGCTG |
| | | | GTGAATGGAGGCAGTGGTCACAACTCTCCCCAGAGAA |
| | | | GGTGGTGTGAGGCCATCACGGAAGATGCTGCTGCTTC |
| | | | TGCTGCTTCTGGGGCCAGGCTCCGGGCTTGGTGCTGT |
| | | | CGTCTCTCAACATCCGAGCTGGGTTATCTGTAAGAGTG |
| | | | GAACCTCTGTGAAGATCGAGTGCCGTTCCCTGGACTT |
| | | | TCAGGCCACAACTATGTTTTGGTATCGTCAGTTCCCGA |
| | | | AACAGAGTCTCATGCTGATGGCAACTTCCAATGAGGG |
| | | | CTCCAAGGCCACATACGAGCAAGGCGTCGAGAAGGAC |
| | | | AAGTTTCTCATCAACCATGCAAGCCTGACCTTGTCCAC |
| | | | TCTGACAGTGACCAGTGCCCATCCTGAAGACAGCAGC |
| | | | TTCTACATCTGCAGTGCTAGGTGGGGTGATCAGCCCC |
| | | | AGCATTTTGGTGATGGGACTCGACTCTCCATCCTAGAG |
| | | | GACCTGAACAAGGTGTTCCCACCCGAGGTCGCTGTGT |
| | | | TTGAGCCATCAGAAGCAGAGATCTCCCACACCCAAAA |
| | | | GGCCACACTGGTGTGCCTGGCCACAGGCTTCTACCCC |
| | | | GACCACGTGGAGCTGAGCTGGTGGGTGAATGGGAAG |
| | | | GAGGTGCACAGTGGGGTCAGCACAGACCCGCAGCCC |
| | | | CTCAAGGAGCAGCCCGCCCTCAATGACTCCAGATACT |
| | | | GCCTGA |
| 13 | TRA | 568 | TGGGGCATTTCCTGCCCTGAAGGAGAATTCTCACCAA |
| | | | GCACAGAGGAGAACCCATCAGAGCAGGAGACTTTTCA |
| | | | CTCTGCAGGGGAGCGCTGTCAGCATGACACGTGTTAG |
| | | | CTTGCTGTGGGCAGTCGTGGTCTCCACCTGTCTTGAA |
| | | | TCCGGCATGGCCCAGACAGTCACTCAGTCTCAACCAG |
| | | | AGATGTCTGTGCAGGAGGCAGAGACTGTGACCCTGAG |
| | | | TTGCACATATGACACCAGTGAGAGTAATTATTATTTGTT |
| | | | CTGGTACAAACAGCCTCCCAGCAGGCAGATGATTCTC |
| | | | GTTATTCGCCAAGAAGCTTATAAGCAACAGAATGCAAC |
| | | | GGAGAATCGTTTCTCTGTGAACTTCCAGAAAGCAGCCA |
| | | | AATCCTTCAGTCTCAAGATCTCAGACTCACAGCTGGGG |
| | | | GACACTGCGATGTATTTCTGTGCTTTCATGAAGACTAA |
| | | | CTTTGGAAATGAGAAATTAACCTTTGGGACTGGAACAA |
| | | | GACTCACCATCATACCCAATATCCAGAACCCTGACCCT |
| | | | GCCGTGTACCAGCTGAGAGACTAGATC |
| 14 | TRB | 569 | AGTCATGGGCAAAGATTACCACCAGGGGGCAGACTAG |
| | | | GGCATCCTTGGGATTCTGTGATCAGTCATCCCTCCTCG |
| | | | CTGGTGAATGGAGGCAGTGGTCACAACTCTCCCCAGA |
| | | | GAAGGTGGTGTGAGGCCATCACGGAAGATGCTGCTGC |
| | | | TTCTGCTGCTTCTGGGGCCAGGCTCCGGGCTTGGTGC |
| | | | TGTCGTCTCTCAACATCCGAGCTGGGTTATCTGTAAGA |
| | | | GTGGAACCTCTGTGAAGATCGAGTGCCGTTCCCTGGA |
| | | | CTTTCAGGCCACAACTATGTTTTGGTATCGTCAGTTCC |
| | | | CGAAACAGAGTCTCATGCTGATGGCAACTTCCAATGA |
| | | | GGGCTCCAAGGCCACATACGAGCAAGGCGTCGAGAA |
| | | | GGACAAGTTTCTCATCAACCATGCAAGCCTGACCTTGT |
| | | | CCACTCTGACAGTGACCAGTGCCCATCCTGAAGACAG |
| | | | CAGCTTCTACATCTGCAGTGCTAGGTCCGGGACTAGC |
| | | | AAATTCTTCGGGCCAGGGACACGGCTCACCGTGCTAG |
| | | | AGGACCTGAAAAACGTGTTCCCACCCGAGGTCGCTGT |
| | | | GTTTGAGCCATCAGAAGCAGAGATCTCCCACACCCAA |
| | | | AAGGCCACACTGGTGTGCCTGGCCACAGGCTTCTTCC |
| | | | CTGACCACGTGGAGCTGAGCTGGTGGGTGAATGGGA |
| | | | AGGAGGTGCACAGTGGGGTCAGCACG |
| | TRA | 570 | GAGAAATGTTTCCCTCCTTGAAACCTAGTCACCCACCC |
| | | | ACCTATCATAGCATTTCCTGCCCTGAAGGAGAATTCTC |
| | | | ACCAAGCACAGAGGAGAACCCATCAGAGCAGGAGACT |
| | | | TTTCACTCTGCAGGGGAGCGCTGTCAGCATGACACGT |
| | | | GTTAGCTTGCTGTGGGCAGTCGTGGTCTCCACCTGTC |
| | | | TTGAATCCGGCATGGCCCAGACAGTCACTCAGTCTCA |
| | | | ACCAGAGATGTCTGTGCAGGAGGCAGAGACTGTGACC |
| | | | CTGAGTTGCACATATGACACCAGTGAGAGTAATTATTA |
| | | | TTTGTTCTGGTACAAACAGCCTCCCAGCAGGCAGATG |
| | | | ATTCTCGTTATTCGCCAAGAAGCTTATAAGCAACAGAA |
| | | | TGCAACGGAGAATCGTTTCTCTGTGAACTTCCAGAAAG |
| | | | CAGCCAAATCCTTCAGTCTCAAGATCTCAGACTCACAG |
| | | | CTGGGGGACACTGCGATGTATTTCTGTGCTTTCACGAA |
| | | | GTCTAACTTTGGAAATGAGAAATTAACCTTTGGGACTG |
| | | | GAACAAGACTCACCATCATACCCAATATCCAGAACCCT |
| | | | GACCCTGCCGTGTACCAGCTGAGAGACTAGATCGG |
| 15 | TRB | 571 | TGGGGCTGTATGGTTTTTTGACAGGCAGAGATGGACG |
| | | | TAACATCAGTCATGGGCAAAGATTACCACCAGGGGGC |
| | | | AGACTAGGGCATCCTTGGGATTCTGTGATCAGTCATCC |
| | | | CTCCTCGCTGGTGAATGGAGGCAGTGGTCACAACTCT |
| | | | CCCCAGAGAAGGTGGTGTGAGGCCATCACGGAAGAT |
| | | | GCTGCTGCTTCTGCTGCTTCTGGGGCCAGGCTCCGGG |
| | | | CTTGGTGCTGTCGTCTCTCAACATCCGAGCTGGGTTAT |
| | | | CTGTAAGAGTGGAACCTCTGTGAAGATCGAGTGCCGT |
| | | | TCCCTGGACTTTCAGGCCACAACTATGTTTTGGTATCG |
| | | | TCAGTTCCCGAAACAGAGTCTCATGCTGATGGCAACTT |
| | | | CCAATGAGGGCTCCAAGGCCACATACGAGCAAGGCGT |
| | | | CGAGAAGGACAAGTTTCTCATCAACCATGCAAGCCTG |
| | | | ACCTTGTCCACTCTGACAGTGACCAGTGCCCATCCTG |
| | | | AAGACAGCAGCTTCTACATCTGCAGTGCTCGGACAGG |
| | | | AGACGAGCAGTACTTCGGGCCGGGCACCAGGCTCAC |
| | | | GGTCACAGAGGACCTGAAAAACGTGTTCCCACCCGAG |
| | | | GTCGCTGTGTTTGAGCCATCAGAAGCAGAGATCTCCC |
| | | | ACACCCAAAAGGCCACACTGGTGTGCCTGGCCACAGG |
| | | | CTTCTACCCCGACCACGTGGAGCTGAGCTGGTGGGTG |
| | | | AATGGGAAGGAGGTGCACAGTGGGGTCAGCACAGAC |
| | | | CCGCAGCCCCTCAAGGAGCAGCCCGCCCTCAATGACT |
| | | | CCAGATACTGCCTGA |
| | TRA | 572 | ATATGGGGATAGCATTTCCTGCCCTGAAGGAGAATTCT |
| | | | CACCAAGCACAGAGGAGAACCCATCAGAGCAGGAGAC |
| | | | TTTTCACTCTGCAGGGGAGCGCTGTCAGCATGACACG |
| | | | TGTTAGCTTGCTGTGGGCAGTCGTGGTCTCCACCTGT |
| | | | CTTGAATCCGGCATGGCCCAGACAGTCACTCAGTCTC |
| | | | AACCAGAGATGTCTGTGCAGGAGGCAGAGACTGTGAC |
| | | | CCTGAGTTGCACATATGACACCAGTGAGAGTAATTATT |
| | | | ATTTGTTCTGGTACAAACAGCCTCCCAGCAGGCAGAT |
| | | | GATTCTCGTTATTCGCCAAGAAGCTTATAAGCAACAGA |
| | | | ATGCAACGGAGAATCGTTTCTCTGTGAACTTCCAGAAA |
| | | | GCAGCCAAATCCTTCAGTCTCAAGATCTCAGACTCACA |
| | | | GCTGGGGGACACTGCGATGTATTTCTGTGCTTTCATCA |
| | | | AATCTAACTTTGGAAATGAGAAATTAACCTTTGGGACT |
| | | | GGAACAAGACTCACCATCATACCCAATATCCAGAACCC |
| | | | TGACCCTGCCGTGTACCAGCTGAGAGACTCTAAATCC |
| | | | AGTGACAAGTCTGTCTGCCT |
| | TRB | 573 | GGGGATCCTTGGGATTCTGTGATCAGTCATCCCTCCT |
| | | | CGCTGGTGAATGGAGGCAGTGGTCACAACTCTCCCCA |
| | | | GAGAAGGTGGTGTGAGGCCATCACGGAAGATGCTGCT |
| | | | GCTTCTGCTGCTTCTGGGGCCAGGCTCCGGGCTTGGT |
| | | | GCTGTCGTCTCTCAACATCCGAGCAGGGTTATCTGTAA |
| | | | GAGTGGAACCTCTGTGAAGATCGAGTGCCGTTCCCTG |
| | | | GACTTTCAGGCCACAACTATGTTTTGGTATCGTCAGTT |
| | | | CCCGAAACAGAGTCTCATGCTGATGGCAACTTCCAAT |
| | | | GAGGGCTCCAAGGCCACATACGAGCAAGGCGTCGAG |
| | | | AAGGACAAGTTTCTCATCAACCATGCAAGCCTGACCTT |
| | | | GTCCACTCTGACAGTGACCAGTGCCCATCCTGAAGAC |
| | | | AGCAGCTTCTACATCTGCAGTGCCCGGAGGGGGACTG |
| | | | AAGCTTTCTTTGGACAAGGCACCAGACTCACAGTTGTA |
| | | | GAGGACCTGAACAAGGTGTTCCCACCCGAGGTCGCTG |
| | | | TGTTTGAGCCATCAGAAGCAGAGATCTCCCACACCCA |
| | | | AAAGGCCACACTGGTGTGCCTGGCCACAGGCTTCTTC |
| | | | CCTGACCACGTGGAGCTGAGCTGGTGGGTGAATGGG |
| | | | AAGGAGGTGCACAGTGGGGTCAGCACGGACCCGCAG |
| | | | CCCCTCAAGGAGCAGCCCGCCCTCAATGACTCCAGAT |
| | | | ACTGCCTGA |
| 16 | TRA | 574 | CGGAGTCTCACTTTGTCGCCCAGGCTGGAGTGCAGTG |
| | | | GTGCGATCTCTGCTCACTGCAAACTCCGCCTCCCGGG |
| | | | TTCCCGCCATTCTCCTGCCTCAGCCTCTCGAGTAGCT |
| | | | GGGACTACAGGCGCCCGCCACAGCGCCCAGCTAATTT |
| | | | TTTTGTATTTTTTGGTAGAGACGGGGTTTCACCGTGTT |
| | | | AGCCAGGATGGTCTCGATCTCCTGACCTCGTGATCCG |
| | | | CCCACCTCGGCCTCCCAAAGCGCTGGGATTAAAGGCG |
| | | | TGAGCCACCGCGCCCGGCCTACTTTGTCTTATGTTATT |
| | | | CCCATTTGCCGTCTCTGTTCCTTATACATTATGCTTTTT |
| | | | CAACTTTACCAGAATCACTTGGATTAAAACCCGTGGAT |
| | | | TTCTCAGTAGGAAATGTTCATGTGAAGACACTTCTGTA |
| | | | GTAACAGAACCTACAGCTGCTCCTGTAGAAGGAAGTT |
| | | | GAAAGTCATCCCTTCAAGAAAGGGGCTCCTCCCCTTG |
| | | | TAATTCTACTGGGTTTTGCATCCAGACTGAGTTTCCTT |
| | | | CCCTCACCCACATGAAGTGTCTACCTTCTGCAGACTCC |
| | | | AATGGCTCAGGAACTGGGAATGCAGTGCCAGGCTCGT |
| | | | GGTATCCTGCAGCAGATGTGGGGAGTTTTCCTTCTTTA |
| | | | TGTTTCCATGAAGATGGGAGGCACTACAGGACAAAAC |
| | | | ATTGACCAGCCCACTGAGATGACAGCTACGGAAGGTG |
| | | | CCATTGTCCAGATCAACTGCACGTACCAGACATCTGG |
| | | | GTTCAACGGGCTGTTCTGGTACCAGCAACATGCTGGC |
| | | | GAAGCACCCACATTTCTGTCTTACAATGTTCTGGATGG |
| | | | TTTGGAGGAGAAAGGTCGTTTTTCTTCATTCCTTAGTC |
| | | | GGTCTAAAGGGTACAGTTACCTCCTTTTGAAGGAGCTC |
| | | | CAGATGAAAGACTCTGCCTCTTACCTCTGTGCTGTGAT |
| | | | GGATAGCAGCTATAAATTGATCTTCGGGAGTGGGACC |
| | | | AGACTGCTGGTCAGGCCTGATATCCAGAACCCTGACC |
| | | | CTGCCGTGTACCAGCTGAGAGACTCTAAATCCAGTGA |
| | | | CAAGTCTGTCTGCCTATTCACCGATTTT |
| | TRA | 575 | TATATGGGGAGTACCAGCAGTTTTCACTCCCTACTCTT |
| | | | CTTGGGGCTTTAGCTGAACTTTGAGGGGAGCCCTGCT |
| | | | CTGAGGAGCTGCTGCCCTGCTATTGGCTGCTGTACTG |
| | | | GGGCAGCCTGAGTGACAGCTGCTGGTGTGGGCCCTG |
| | | | GCAGTTGCTGCTGGGCTCATTGCAGCTCAGACACAGC |
| | | | AAAAGAGCCTAGAACCTGGGTCCTAGTTTGCACCTAG |
| | | | AATATGAGGCAAGTGGCGAGAGTGATCGTGTTCCTGA |
| | | | CCCTGAGTACTTTGAGCCTTGCTAAGACCACCCAGCC |
| | | | CATCTCCATGGACTCATATGAAGGACAAGAAGTGAACA |
| | | | TAACCTGTAGCCACAACAACATTGCTACAAATGATTAT |
| | | | ATCACGTGGTACCAACAGTTTCCCAGCCAAGGACCAC |
| | | | GATTTATTATTCAAGGATACAAGACAAAAGTTACAAAC |
| | | | GAAGTGGCCTCCCTGTTTATCCCTGCCGACAGAAAGT |
| | | | CCAGCACTCTGAGCCTGCCCCGGGTTTCCCTGAGCGA |
| | | | CACTGCTGTGTACTACTGCCTCGTGAAGATAATCTTTG |
| | | | GATCAGGGACCAGACTCAGCATCCGGCCAAATATCCA |
| | | | GAACCCTGACCCTGCCGTGTACCAGCTGAGAGACTCT |
| | | | AAATCCAGTGACAAGTCTGTCTGCCTATTCACCGATTT |
| | | | T |
| | TRB | 576 | GTCGGCTGAAGGGAATGCTTGGCTGCGTGCTGGCATC |
| | | | AGGAGGCGGTTGGAATTCAGAGTAGGTCCTGGGACTG |
| | | | CCCAGGGCAAGGAATAAAATTTATATTGAATTACAAAC |
| | | | TGTTTGCACAAAAGTCAAGAAAAACTTGAAAGCCTTAT |
| | | | ATCAAGAAGCATTCTTCCCATCCAAACTTCAGTGGTGC |
| | | | ATTTATTTTGGATTTGACCCTCTGGGGAAGGGGCATGG |
| | | | CCTCTCTCGACAAGAAGGTTCTGGGGACCAGGCAGAG |
| | | | AGAATGAGGTCTCAGGATGACTTCCTTGACAGCCCTG |
| | | | TTCGCCTTTCATCAACACACAGACCCAGAAGACCTCTC |
| | | | TGTCTTGTAGCATCTGCCATGAGAATCAGGCTCCTGTG |
| | | | CTGTGTGGCCTTTTCTCTCCTGTGGGCAGGTCCAGTG |
| | | | ATTGCTGGGATCACCCAGGCACCAACATCTCAGATCC |
| | | | TGGCAGCAGGACGGCGCATGACACTGAGATGTACCCA |
| | | | GGATATGAGACATAATGCCATGTACTGGTATAGACAAG |
| | | | ATCTAGGACTGGGGCTAAGGCTCATCCATTATTCAAAT |
| | | | ACTGCAGGTACCACTGGCAAAGGAGAAGTCCCTGATG |
| | | | GTTATAGTGTCTCCAGAGCAAACACAGATGATTTCCCC |
| | | | CTCACGTTGGCGTCTGCTGTACCCTCTCAGACATCTGT |
| | | | GTACTTCTGTGCCAGCAGTGAAAGGGAGCACTATGGC |
| | | | TACACCTTCGGTTCGGGGACCAGGTTAACCGTTGTAG |
| | | | AGGACCTGAACAAGGTGTTCCCACCCGAGGTCGCTGT |
| | | | GTTTGAGCCATCAGAAGCAGAGATCTCCCACACCCAA |
| | | | AAGGCCACACTGGTGTGCCTGGCCACAGGCTTCTACC |
| | | | CCGACCACGTGGAGCTGAGCTGGTGGGTGAATGGGA |
| | | | AGGAGGTGCACAGTGGGGTCAGCACGGACCCGCAGC |
| | | | CCCTCAAGGAGCAGCCCGCCCTCAATGACTCCAGATA |
| | | | CTGCCTGA |
| 17 | TRA | 577 | GAACTTGGCTTTAAATCAGCCTATCTGCATTGAAAGGA |
| | | | AAGGACTGAGCTTGCCTGTGACTGGCTAGGGAGGAAC |
| | | | CTGAGACTAGGGGACAGAAAGACTAGGGATTCACCCA |
| | | | GTAAAGAGAGCTCATCTGTGACTGAGGAGCCTTGCTC |
| | | | CATTTCAGGTCTTCTGTGATTTCAATAAGGAAGAAGAA |
| | | | TGGAAACTCTCCTGGGAGTGTCTTTGGTGATTCTATGG |
| | | | CTTCAACTGGCTAGGGTGAACAGTCAACAGGGAGAAG |
| | | | AGGATCCTCAGGCCTTGAGCATCCAGGAGGGTGAAAA |
| | | | TGCCACCATGAACTGCAGTTACAAAACTAGTATAAACA |
| | | | ATTTACAGTGGTATAGACAAAATTCAGGTAGAGGCCTT |
| | | | GTCCACCTAATTTTAATACGTTCAAATGAAAGAGAGAA |
| | | | ACACAGTGGAAGATTAAGAGTCACGCTTGACACTTCCA |
| | | | AGAAAAGCAGTTCCTTGTTGATCACGGCTTCCCGGGC |
| | | | AGCAGACACTGCTTCTTACTTCTGTGCTACGCCATATA |
| | | | CTGGAGGCTTCAAAACTATCTTTGGAGCAGGAACAAG |
| | | | ACTATTTGTTAAAGCAAATATCCAGAACCCTGACCCTG |
| | | | CCGTGTACCAGCTGAGAGACTCTAAATCCAGTGACAA |
| | | | GTCTGTCTGCCTATTCACCGATTTTGATTC |
| | TRB | 578 | AGAACTTAGAAAGGATGTAAAGCAGTCAGGAAGAAAC |
| | | | ATCCCCTGGGTCTGGGGAAACTATCAGGAGCAGTGAC |
| | | | ATCACAAGAAAAACCACCAACCAGGGCCAAGGAGACC |
| | | | AGAGCCCAGCACCTCGCCCAAAGGACCCCAGTCAGA |
| | | | GGCCCCATCTCAGACCCGAGGCTAGCATGGGCTGCA |
| | | | GGCTGCTCTGCTGTGCGGTTCTCTGTCTCCTGGGAGC |
| | | | AGTTCCCATAGACACTGAAGTTACCCAGACACCAAAAC |
| | | | ACCTGGTCATGGGAATGACAAATAAGAAGTCTTTGAAA |
| | | | TGTGAACAACATATGGGGCACAGGGCTATGTATTGGT |
| | | | ACAAGCAGAAAGCTAAGAAGCCACCGGAGCTCATGTT |
| | | | TGTCTACAGCTATGAGAAACTCTCTATAAATGAAAGTG |
| | | | TGCCAAGTCGCTTCTCACCTGAATGCCCCAACAGCTCT |
| | | | CTCTTAAACCTTCACCTACACGCCCTGCAGCCAGAAGA |
| | | | CTCAGCCCTGTATCTCTGCGCCAGCAGCCAACTTGGC |
| | | | TCTGGAAACACCATATATTTTGGAGAGGGAAGTTGGCT |
| | | | CACTGTTGTAGAGGACCTGAACAAGGTGTTCCCACCC |
| | | | GAGGTCGCTGTGTTTGAGCCATCAGAAGCAGAGATCT |
| | | | CCCACACCCAAAAGGCCACACTGGTGTGCCTGGCCAC |
| | | | AGGCTTCTACCCCGACCACGTGGAGCTGAGCTGGTGG |
| | | | GTGAATGGGAAGGAGGTGCACAGTGGGGTCAGCACA |
| | | | GACCCGCAGCCCCTCAAGGAGCAGCCCGCCCTCAAT |
| | | | GACTCCAGATACTGCCTGA |
| 18 | TRA | 579 | AGAGAGAGAGAGAGAGAGAGAGAGAGAGAGAGACTT |
| | | | GTCTCAGATTGGCTGGGCCAACTCTGTGTTTCCTGTAG |
| | | | AAATGTTTGAACATATGAGAGAGCAAAGCAGAGTGTTT |
| | | | ATCTTGTGAGCCATTCTCCATATTTCAGATATAAGATTT |
| | | | CAGTTCTCAGTGAGTCTAAGTGACAGAAGGAATGGAG |
| | | | ACCCTCTTGGGCCTGCTTATCCTTTGGCTGCAGCTGC |
| | | | AATGGGTGAGCAGCAAACAGGAGGTGACGCAGATTCC |
| | | | TGCAGCTCTGAGTGTCCCAGAAGGAGAAAACTTGGTT |
| | | | CTCAACTGCAGTTTCACTGATAGCGCTATTTACAACCT |
| | | | CCAGTGGTTTAGGCAGGACCCTGGGAAAGGTCTCACA |
| | | | TCTCTGTTGCTTATTCAGTCAAGTCAGAGAGAGCAAAC |
| | | | AAGTGGAAGACTTAATGCCTCGCTGGATAAATCATCAG |
| | | | GACGTAGTACTTTATACATTGCAGCTTCTCAGCCTGGT |
| | | | GACTCAGCCACCTACCTCTGTGCTGTGAAGGCCCAGG |
| | | | GTGGGGCTGGGAGTTACCAACTCACTTTCGGGAAGGG |
| | | | GACCAAACTCTCGGTCATACCAAATATCCAGAACCCTG |
| | | | ACCCTGCCGTGTACCAGCTGAGAGACTCTAAATCCAG |
| | | | TGACAAGTCTGTCTGCCTATTCACCGATTT |
| | TRB | 580 | GAGAGGGCAATGGGGCAGCCTGTGAGCTGGGGCAAC |
| | | | GTAGGCAGAGAAGGAACTGTGTCACCACAGAAACTTC |
| | | | TGCCTTCACCCATCCCTTCAGCTCTGCAGGACAGGTA |
| | | | GAGACTCCAGGATCATCCACTGAGCACTGGACATAAG |
| | | | GAAGGCTGCATGGGGAGGACTCAGGACAGTGACATCA |
| | | | CAGGATACCCCTCCTATTAGGAAAATCAAGGCCCAGA |
| | | | ATTCACTCGGCTCTTCCCCAGGAGGACCAAGCCCTGA |
| | | | ATCAGGTGCAGTGCTGCCTGCCCCACTGTGCCATGGG |
| | | | CCCTGGGCTCCTCTGCTGGGTGCTGCTTTGTCTCCTG |
| | | | GGAGCAGGCTCAGTGGAGACTGGAGTCACCCAAAGTC |
| | | | CCACACACCTGATCAAAACGAGAGGACAGCAAGTGAC |
| | | | TCTGAGATGCTCTTCTCAGTCTGGGCACAACACTGTGT |
| | | | CCTGGTACCAACAGGCCCTGGGTCAGGGGCCCCAGT |
| | | | TTATCTTTCAGTATTATAGGGAGGAAGAGAATGGCAGA |
| | | | GGAAACTTCCCTCCTAGATTCTCAGGTCTCCAGTTCCC |
| | | | TAATTATAGCTCTGAGCTGAATGTGAACGCCTTGGAGC |
| | | | TGGACGACTCGGCCCTGTATCTCTGTGGGTCCGGGCG |
| | | | GGAACGCACAGATACGCAGTATTTTGGCCCAGGCACC |
| | | | CGGCTGACAGTGCTCGAGGACCTGAAAAACGTGTTCC |
| | | | CACCCGAGGTCGCTGTGTTTGAGCCATCAGAAGCAGA |
| | | | GATCTCCCACACCCAAAAGGCCACACTGGTGTGCCTG |
| | | | GCCACAGGCTTCTACCCCGACCACGTGG |
| 19 | TRA | 581 | TGGGGGGCCTTTCTATGGAACTCAAAGTGGGTGGCAG |
| | | | CAAGCACTCTTCTAGCCCAGAGAAGTCTGTTCCAGGA |
| | | | CGGGCTCTTTCAGGAGCAGCTAAAGTCAGGGGCCATG |
| | | | TCCACCATGTGATAGAAAGACAAGATGGTCCTGAAATT |
| | | | CTCCGTGTCCATTCTTTGGATTCAGTTGGCATGGGTGA |
| | | | GCACCCAGCTGCTGGAGCAGAGCCCTCAGTTTCTAAG |
| | | | CATCCAAGAGGGAGAAAATCTCACTGTGTACTGCAACT |
| | | | CCTCAAGTGTTTTTTCCAGCTTACAATGGTACAGACAG |
| | | | GAGCCTGGGGAAGGTCCTGTCCTCCTGGTGACAGTAG |
| | | | TTACGGGTGGAGAAGTGAAGAAGCTGAAGAGACTAAC |
| | | | CTTTCAGTTTGGTGATGCAAGAAAGGACAGTTCTCTCC |
| | | | ACATCACTGCGGCCCAGCCTGGTGATACAGGCCTCTA |
| | | | CCTCTGTGCAGGAGCTAATTCCGGGTATGCACTCAAC |
| | | | TTCGGCAAAGGCACCTCGCTGTTGGTCACACCCCATA |
| | | | TCCAGAACCCTGACCCTGCCGTGTACCAGCTGAGAGA |
| | | | CT |
| | TRB | 582 | AGACACACTAAGCCATGCTTACCAGTTACTATAGTGGG |
| | | | AACACTCCCAAAATTCAAGTTCGCAGATGCAAATCAAG |
| | | | AGCCAACCATGCAAGCAGTCCCTTCTAAGGCCAGCAG |
| | | | TCTCAGGCCCGCTGTGTCAACTCTTTTCTATACGTGCA |
| | | | CATACACACAGTGACACTTCCATGAGCACAAGGGATG |
| | | | TCCTTCACTCTCTAGCACCTCTAGACCCTTTCTAGTCT |
| | | | AGACAATGGCTGAGGGATTATGAGAGTGGGCCGGGA |
| | | | CAATGACATCACAGACCATCAACCCACTGCCTGGTCCT |
| | | | GGGAGAAGACCTATTCTTTCTTCAAAGCAGCCATGGG |
| | | | AATCAGGCTCCTCTGTCGTGTGGCCTTTTGTTTCCTGG |
| | | | CTGTAGGCCTCGTAGATGTGAAAGTAACCCAGAGCTC |
| | | | GAGATATCTAGTCAAAAGGACGGGAGAGAAAGTTTTTC |
| | | | TGGAATGTGTCCAGGATATGGACCATGAAAATATGTTC |
| | | | TGGTATCGACAAGACCCAGGTCTGGGGCTACGGCTGA |
| | | | TCTATTTCTCATATGATGTTAAAATGAAAGAAAAAGGAG |
| | | | ATATTCCTGAGGGGTACAGTGTCTCTAGAGAGAAGAA |
| | | | GGAGCGCTTCTCCCTGATTCTGGAGTCCGCCAGCACC |
| | | | AACCAGACATCTATGTACCTCTGTGCCAGCAGTTCCCT |
| | | | ACCGGGACAGCTTTATGGCTACACCTTCGGTTCGGGG |
| | | | ACCAGGTTAACCGTTGTAGAGGACCTGAACAAGGTGT |
| | | | TCCCACCCGAGGTCGCTGTGTTTGAGCCATCAGAAGC |
| | | | AGAGATCTCCCACACCCAAAAGGCCACACTGGTGTGC |
| | | | CTGGCCACAGGCTTCTACCCCGACCACGTGGAGCTGA |
| | | | GCTGGTGGGTGAATGGGAAGGAGGTGCACAGTGGGG |
| | | | TCAGCACAGACCCGCAGCCCCTCAAGGAGCAGCCCG |
| | | | CCCTCAATGACTCCAGATACTGCCTGA |
| 20 | TRA | 583 | TTCTACCTCAGCGTCTTGAGTAGCTGGGATTACAGGCA |
| | | | TAAGCCACTGTGCCCAGCTTAAAACCTGTGGATTTATC |
| | | | AGTAGAAAATGTTCATGTAAAGATACTCCTGTAAGAGA |
| | | | AACCATAGCTGCTCCAGTGGAAGGAAGCTTAAACTCAT |
| | | | CCCTTCAAGAAAGAAGCTCCTCCCTTTGTATTTCTACT |
| | | | GGGTTTTGCATCCGGACTGATCTTCCTTCCCTCACCCA |
| | | | CATGAAGTGTCTACCTTCTGCAGACTACAGTGGCTCAG |
| | | | GAACCGGGGATGCAGTGCCAGGCTCATGGTATCCTGC |
| | | | AGCAGATGTGGGGAGCTTTCCTTCTCTATGTTTCCATG |
| | | | AAGATGGGAGGCACTGCAGGACAAAGCCTTGAGCAGC |
| | | | CCTCTGAAGTGACAGCTGTGGAAGGAGCCATTGTCCA |
| | | | GATAAACTGCACGTACCAGACATCTGGGTTTTATGGGC |
| | | | TGTCCTGGTACCAGCAACATGATGGCGGAGCACCCAC |
| | | | ATTTCTTTCTTACAATGCTCTGGATGGTTTGGAGGAGA |
| | | | CAGGTCGTTTTTCTTCATTCCTTAGTCGCTCTGATAGTT |
| | | | ATGGTTACCTCCTTCTACAGGAGCTCCAGATGAAAGAC |
| | | | TCTGCCTCTTACTTCTGCGCTGAGGGAGGCTTCAAAAC |
| | | | TATCTTTGGAGCAGGAACAAGACTATTTGTTAAAGCAA |
| | | | ATATCCAGAACCCTGACCCTGCCGTGTACCAGCTGAG |
| | | | AGACTT |
| | TRB | 584 | ACAACTCTCTGGTGGTTGCCAGTAAAGCGCCAGAAAA |
| | | | ACAGAAGAGAAGCAAGTAGAAGAATTTCAGCATCTGCT |
| | | | AAGAACATATAATACATAAGCCAACATGGCATCTTTCTT |
| | | | TAAGACCCAGTACCTTGGGAGGGACAATGACATCACT |
| | | | TCCTGAATCCTCCCAGTTTTCTATTTCCATGCCCTGCTT |
| | | | CCCTCAACATCCAGAGCTGGAAACACCTCCATCCTGC |
| | | | CTCTTCATGCCATGGCCTCCCTGCTCTTCTTCTGTGGG |
| | | | GCCTTTTATCTCCTGGGAACAGGGTCCATGGATGCTG |
| | | | ATGTTACCCAGACCCCAAGGAATAGGATCACAAAGAC |
| | | | AGGAAAGAGGATTATGCTGGAATGTTCTCAGACTAAG |
| | | | GGTCATGATAGAATGTACTGGTATCGACAAGACCCAG |
| | | | GACTGGGCCTACGGTTGATCTATTACTCCTTTGATGTC |
| | | | AAAGATATAAACAAAGGAGAGATCTCTGATGGATACAG |
| | | | TGTCTCTCGACAGGCACAGGCTAAATTCTCCCTGTCCC |
| | | | TAGAGTCTGCCATCCCCAACCAGACAGCTCTTTACTTC |
| | | | TGTGCCACCAGTGGCCCCCTCACAGGGCCCCCCGAG |
| | | | CAGTTCTTCGGGCCAGGGACACGGCTCACCGTGCTAG |
| | | | AGGACCTGAAAAACGTGTTCCCACCCGAGGTCGCTGT |
| | | | GTTTGAGCCATCAGAAGCAGAGATCTCCCACACCCAA |
| | | | AAGGCCACACTGGTGTGCCTGGCCACAGGCTTCTACC |
| | | | CCGACCACGTGGAGCTGAGCTGGTGGGTGAATGGGA |
| | | | AGGAGGTGCACAGTGGGGTCAGCACAGACCCGCAGC |
| | | | CCCTCA |

TABLE 4
HLA-DR3 restricted TCRs

| | | SEQ | |
|---|---|---|---|
| | Descrip- | ID | |
| TCR | tion | NO: | Amino acid sequence |
| 1 | TRA | 585 | MEKMLECAFIVLWLQLGWLSGEDQVTQSPEALRLQEGE |
| | | | SSSLNCSYTVSGLRGLFWYRQDPGKGPEFLFTLYSAGE |
| | | | EKEKERLKATLTKKESFLHITAPKPEDSATYLCAAAIG |
| | | | SYIPTFGRGTSLIVHPYIQNPDPAVYQLRD |
| | TRB | 586 | MGSRLLCWVLLCLLGAGPVKAGVTQTPRYLIKTRGQQVT |
| | | | LSCSPISGHRSVSWYQQTPGQGLQFLFEYFSETQRNKG |
| | | | NFPGRFSGRQFSNSRSEMNVSTLELGDSALYLCASSLGN |
| | | | SNTEAFFGQGTRLTVVEDLNKVFPPEVAVFEPSEAEISH |
| | | | TQKATLVCLATGFYPDHVELSWWVNGKEVHSGVSTDPQ |
| | | | PLKEQPALNDSRYCL |
| 2 | TRA | 587 | MMKSLRVLLVILWLQLSWVWSQQKEVEQNSGPLSVPEG |
| | | | AIASLNCTYSDRGSQSFFWYRQYSGKSPELIMSIYSNGD |
| | | | KEDGRFTAQLNKASQYVSLLIRDSQPSDSATYLCAVRES |
| | | | GGGADGLTFGKGTHLIIQPYIQNPDPAVYQLRDSKSSD |
| | TRB | 588 | MLSLLLLLLGLGSVFSAVISQKPSRDICQRGTSLTIQCQ |
| | | | VDSQVTMMFWYRQQPGQSLTLIATANQGSEATYESGFVI |
| | | | DKFPISRPNLTFSTLTVSNMSPEDSSIYLCSVGEQGMTQ |
| | | | PQHFGDGTRLSILEDLNKVFPPEVAVFEPSEAEISHTQK |
| | | | ATLVCLATGF |
| 3 | TRA | 589 | MKLVTSITVLLSLGIMGDAKTTQPNSMESNEEEPVHLPCN |
| | | | HSTISGTDYIHWYRQLPSQGPEYVIHGLTSNVNNRMASL |
| | | | AIAEDRKSSTLILHRATLRDAAVYYCILRYQGAQKLVFGQ |
| | | | GTRLTINPNIQNPDPAVYQLRD |
| | TRB | 590 | MGCRLLCCAVLCLLGAVPIDTEVTQTPKHLVMGMTNKKS |
| | | | LKCEQHMGHRAMYWYKQKAKKPPELMFVYSYEKLSINE |
| | | | SVPSRFSPECPNSSLLNLHLHALQPEDSALYLCASSQVR |
| | | | ERKNTGELFFGEGSRLTVLEDLKNVFPPEVAVFEPSEAEI |
| | | | SHTQKATLVCLATGFYPDHVELSWWVNGKEVHSGVSTD |
| | | | PQPLKEQPALNDSRYCL |
| 4 | TRA | 591 | METLLKVLSGTLLWQLTWVRSQQPVQSPQAVILREGEDA |
| | | | VINCSSSKALYSVHWYRQKHGEAPVFLMILLKGGEQKGH |
| | | | EKISASFNEKKQQSSLYLTASQLSYSGTYFCGTGAGSYQ |
| | | | LTFGKGTKLSVIPNIQNPDPAVYQLRDSKSSDKSVCLFT |
| | | | DFDS |
| | TRB | 592 | MGCRLLCCAVLCLLGAVPIDTEVTQTPKHLVMGMTNKKS |
| | | | LKCEQHMGHRAMYWYKQKAKKPPELMFVYSYEKLSINES |
| | | | VPSRFSPECPNSSLLNLHLHALQPEDSALYLCASSHPGL |
| | | | RAGASYNEQFFGPGTRLTVLEDLKNVFPPEVAVFEPSEA |
| | | | EISHTQKATLVCLATGFYPDHVELSWWVNGKEVHSGVS |
| 5 | TRA | 593 | MMKCPQALLAIFWLLLSWVSSEDKVVQSPLSLVVHEGDT |
| | | | VTLNCSYEVTNFRSLLWYKQEKKAPTFLFMLTSSGIEKK |
| | | | SGRLSSILDKKELFSILNITATQTGDSAVYLCAVLNTGN |
| | | | QFYFGTGTSLTVIPNIQNPDPAVYQLRDSKSSDK |
| | TRB | 594 | MGCRLLCCAVLCLLGAVPIDTEVTQTPKHLVMGMTNKKS |
| | | | LKCEQHMGHRAMYWYKQKAKKPPELMFVYSYEKLSINE |
| | | | SVPSRFSPECPNSSLLNLHLHALQPEDSALYLCASSRFP |
| | | | GGGYNEQFFGPGTRLTVLEDLKNVFPPEVAVFEPSEAEI |
| | | | SHTQKATLVCLATGFYPDHVELSWWVN |
| 6 | TRA | 595 | MMISLRVLLVILWLQLSWVWSQRKEVEQDPGPFNVPEG |
| | | | ATVAFNCTYSNSASQSFFWYRQDCRKEPKLLMSVYSSG |
| | | | NEDGRFTAQLNRASQYISLLIRDSKLSDSATYLCVVNW |
| | | | SGNEKLTFGTGTRLTIIPNIQNPDPAVYQLRDLKTTQT |
| | | | RVF |
| | TRB | 596 | MASLLFFCGAFYLLGTGSMDADVTQTPRNRITKTGKRIM |
| | | | LECSQTKGHDRMYWYRQDPGLGLRLIYYSFDVKDINKGE |
| | | | ISDGYSVSRQAQAKFSLSLESAIPNQTALYFCATRGQGV |
| | | | GELFFGEGSRLTVLEDLKNVFPPEVAVFEPSEAEISHTQ |
| | | | KATLVCLA |
| 7 | TRA | 597 | MMKSLRVLLVILWLQLSWVWSQQKEVEQDPGPLSVPEG |
| | | | AIVSLNCTYSNSAFQYFMWYRQYSRKGPELLMYTYSSG |
| | | | NKEDGRFTAQVDKSSKYISLFIRDSQPSDSATYLCAMSV |
| | | | QAAGNKLTFGGGTRVLVKPNIQNPDPAVYQLRDYDGP |
| | TRB | 598 | MRGQGRPSSFAVAHSDPDWAKLPSFPDPAMGTRLLCW |
| | | | AALCLLGAELTEAGVAQSPRYKIIEKRQSVAFWCNPISGH |
| | | | ATLYWYQQILGQGPKLLIQFQNNGVVDDSQLPKDRFSAE |
| | | | RLKGVDSTLKIQPAKLEDSAVYLCASSLDRNYGYTFGSG |
| | | | TRLTVVEDLNKVFPPEVAVFEPSEAEISHTQKATLVCLAT |
| | | | GFYPDHVELSWWVNGKEVHSGVSTDPQPLKEQPALND |
| | | | SRYCL |
| 8 | TRA | 599 | MNYSPGLVSLILLLLGRTRGNSVTQMEGPVTLSEEAFLTI |
| | | | NCTYTATGYPSLFWYVQYPGEGLQLLLKATKADDKGSN |
| | | | KGFEATYRKETTSFHLEKGSVQVSDSAVYFCALSDNTNA |
| | | | GKSTFGDGTTLTVKPNIQNPDPAVYQLRDSKSSDKSVCL |
| | | | FTDFDSQTNVSQSKDSDVYITDKTVLDMRSMDFKSNSAV |
| | | | AW |
| | TRB | 600 | MEAVVTTLPREGGVRPSRKMLLLLLLGPGSGLGAVVSQ |
| | | | HPSWVICKSGTSVKIECRSLDFQATTMFWYRQFPKQSLM |
| | | | LMATSNEGSKATYEQGVEKDKFLINHASLTLSTLTVTSAH |
| | | | PEDSSFYICSARTSGQYEQYFGPGTRLTVTEDLKNVFPP |
| | | | EVAVFEPSEAEISHTQKATLVCLATGFYPDHVELSWWVN |
| | | | GKEVHSGVSTDPQPLKEQPALNDSRYCL |
| 9 | TRA | 601 | MNYSPGLVSLILLLLGRTRGDSVTQMEGPVTLSEEACLTI |
| | | | NCTYTATGYPSLFWYVQYPGEGLQLLLKATKADDKGSN |
| | | | KGFEATYRKETTSFHLEKGSVQVSDSAVYFCALSDLLDS |
| | | | GNTGKLIFGQGTTLQVKPDIQNPDPAVYQLRD |
| | TRB | 602 | MGFRLLCCVAFCLLGAGPVDSGVTQTPKHLITATGQRVT |
| | | | LRCSPRSGDLSVYWYQQSLDQGLQFLIQYYNGEERAKG |
| | | | NILERFSAQQFPDLHSELNLSSLELGDSALYFCASRGSYN |
| | | | EQFFGPGTRLTVLEDLKNVFPPEVAVFEPSEAEISHTQKA |
| | | | TLVCLATGFYPDHVELSWWVNGK |
| 10 | TRA | 603 | MMISLRVLLVILWLQLSWVWSQRKEVEQDPGPFNVPEG |
| | | | ATVAFNCTYSNSASQSFFWYRQDCRKEPKLLMSVYSSG |
| | | | NEDGRFTAQLNRASQYISLLIRDSKLSDSATYLCVVNTPS |
| | | | SKIIFGSGTRLSIRPNIQNPDPAVYQLRD |
| | TRB | 604 | MEAVVTTLPREGGVRPSRKMLLLLLLLGPGSGLGAVVSQ |
| | | | HPSWVICKSGTSVKIECRSLDFQATTMFWYRQFPKQSLM |
| | | | LMATSNEGSKATYEQGVEKDKF |
| | | | LINHASLTLSTLTVTSAHPEDSSFYICSARSGQGQFFGPG |
| | | | TRLTVLEDLKNVFPPEVAVFEPSEAEISHTQKATLVCLAT |
| | | | GFYPDHVELSWWVNGKEVHSGVSTDPQPLKEQPALND |
| | | | SRYCL |
| 11 | TRA | 605 | MMISLRVLLVILWLQLSWVWSQRKEVEQDPGPFNVPEG |
| | | | ATVAFNCTYSNSASQSFFWYRQDCRKEPKLLMSVYSSG |
| | | | NEDGRFTAQLNRASQYISLLIRDSKLSDSATYLCVVPPGG |
| | | | GFKTIFGAGTRLFVKANIQNPDPAVYQLRDSKSSDKSVCL |
| | | | FTDFDSQTNVSQSKDSDVYITDKTVLDMRSMDFKSNSAV |
| | | | AWSNKSDFACANAF |
| | TRB | 606 | MGPQLLGYVVLCLLGAGPLEAQVTQNPRYLITVTGKKLT |
| | | | VTCSQNMNHEYMSWYRQDPGLGLRQIYYSMNVEVTDK |
| | | | GDVPEGYKVSRKEKRNFPLILESPSPNQTSLYFCASRTR |
| | | | TASYGYTFGSGTRLTVVEDLNKVFPPEVAVFEPSEAEISH |
| | | | TQKATLVCLATGFFPDHVELSWWVNGKEVHSGVSTDPQ |
| | | | PLKEQPALNDSRYCL |
| 12 | TRA | 607 | MAQELGMQCQARGILQQMWGVFLLYVSMKMGGTTGQN |
| | | | IDQPTEMTATEGAIVQINCTYQTSGFNGLFWYQQHAGEA |
| | | | PTFLSYNVLDGLEEKGRFSSFLSRSKGYSYLLLKELQMK |
| | | | DSASYLCAVLDSNYQLIWGAGTKLIIKPDIQNPDPAVYQL |
| | | | RD |
| | TRB | 608 | MKSQNDPLESTVPLSPMHRPRRPLHPVAPAMSIGLLCCV |
| | | | AFSLLWASPVNAGVTQTPKFQVLKTGQSMTLQCAQDMN |
| | | | HNSMYWYRQDPGMGLRLIYYSASEGTTDKGEVPNGYNV |
| | | | SRLNKREFSLRLESAAPSQTSVYFCASSEEASGSYEQYF |
| | | | GPGTRLTVTEDLKNVFPPEVAVFEPS |
| 13 | TRA | 609 | MMKSLRVLLVILWLQLSWVWSQQKEVEQNSGPLSVPEG |
| | | | AIASLNCTYSDRVSQSFFWYRQYSGKSPELIMSIYSNGD |
| | | | KEDGRFTAQLNKASQYVSLLIRDSQPSDSATYLCAVSGN |
| | | | QFYFGTGTSLTVIPNIQNPDPAVYQLRD |
| | TRB | 610 | MGTSLLCWMALCLLGADHADTGVSQDPRHKITKRGQNV |
| | | | TFRCDPISEHNRLYWYRQTLGQGPEFLTYFQNEAQLEKS |
| | | | RLLSDRFSAERPKGSFSTLEIQRTEQGDSAMYLCASSPL |
| | | | GSGRYNEQFFGPGTRLTVLEDLKNVFPPEVAVFEPSEAE |
| | | | ISHTQKATLVCLATGFYPDHVELSWWVNGKEVHSGVST |
| | | | DPQPLKEQPALNDSRYCL |
| 14 | TRA | 611 | MLLELIPLLGIHFVLRTARAQSVTQPDIHITVSEGASLE |
| | | | LRCNYSYGATPYLFWYVQSPGQGLQLLLKYFSGDTLVQG |
| | | | IKGFEAEFKRSQSSFNLRKPSVHWSDAAEYFCAVGPFSG |
| | | | GYQKVTFGTGTKLQVIPNIQNPDPAVYQLRD |
| | TRB | 612 | MDTWLVCWAIFSLLKAGLTEPEVTQTPSHQVTQMGQEVIL |
| | | | RCVPISNHLYFYWYRQILGQKVEFLVSFYNNEISEKSEIF |
| | | | DDQFSVERPDGSNFTLKIRSTKLEDSAMYFCASREVHSG |
| | | | ANVLTFGAGSRLTVLEDLKNVFPPEVAVFEPSEAEISHTQ |
| | | | KATLVCLATGFYPDHVELSWWVNGKEVHSGVSTDPQPL |
| | | | KEQPALNDSRYCL |
| 15 | TRA | 613 | MLLELIPLLGIHFVLRTARAQSVTQPDIHITVSEGASLEL |
| | | | RCNYSYGATPYLFWYVQSPGQGLQLLLKYFSGDTLVQGIK |
| | | | GFEAEFKRSQSSFNLRKPSVHWSDAAEYFCAFYNQGGKLI |
| | | | FGQGTELSVKPNIQNPDPAVYQLRDSKSSDKSVCLFTDF |
| | | | DSQTNVSQSKDSDV |
| | TRB | 614 | MDTWLVCWAIFSLLKAGLTEPEVTQTPSHQVTQMGQEVIL |
| | | | RCVPISNHLYFYWYRQILGQKVEFLVSFYNNEISEKSEIF |
| | | | DDQFSVERPDGSNFTLKIRSTKLEDSAMYFCASRVMNTE |
| | | | AFFGQGTRLTVVEDLNKVFPPEVAVFEPSEAEISHTQKAT |
| | | | LVCLATGFFPDHVELSWWVNGKEVHSGVSTDPQPLKEQ |
| | | | PALNDSRYCL |
| 16 | TRA | 615 | MLLELIPLLGIHFVLRTARAQSVTQPDIHITVSEGASLELRC |
| | | | NYSYGATPYLFWYVQSPGQGLQLLLKYFSGDTLVQGIKG |
| | | | FEAEFKRSQSSFNLRKPSVHWSDAAEYFCAVGFLEYGN |
| | | | KLVFGAGTILRVKSYIQNPDPAVYQLRDSKSSDKSVCLFT |
| | | | DFDSQTNVSQSKDSDVYITDKTVLDMRSMDFKSNSAVA |
| | | | WSNKS |
| | TRB | 616 | MGSRLLCWVLLCLLGAGPVKAGVTQTPRYLIKTRGQQVT |
| | | | LSCSPISGHRSVSWYQQTPGQGLQFLFEYFSETQRNKG |
| | | | NFPGRFSGRQFSNSRSEMNVSTLELGDSALYLCASSSLA |
| | | | GANEQFFGPGTRLTVLEDLKNVFPPEVAVFEPSEAEISHT |
| | | | QKATLVCLATGFYPDHVEL |
| 17 | TRA | 617 | MKKHLTTFLVILWLYFYRGNGKNQVEQSPQSLIILEGKNC |
| | | | TLQCNYTVSPFSNLRWYKQDTGRGPVSLTIMTFSENTKS |
| | | | NRRYTATLDADTKQSSLHITASQLSDSASYICVVSAGYG |
| | | | GSQGNLIFGKGTKLSVKPNIQNPDPAVYQLRD |
| | TRB | 618 | MGTSLLCWMALCLLGADHADTGVSQDPRHKITKRGQNV |
| | | | TFRCDPISEHNRLYWYRQTLGQGPEFLTYFQNEAQLEKS |
| | | | RLLSDRFSAERPKGSFSTLEIQRTEQGDSAMYLCASTPG |
| | | | GSSREQYFGPGTRLTVTEDLKNVFPPEVAVFEPSEAEIS |
| | | | HTQKATLVCLATGF |
| 18 | TRA | 619 | MNMLTASLLRAVIASICVVSSMAQKVTQAQTEISVVEKED |
| | | | VTLDCVYETRDTTYYLFWYKQPPSGELVFLIRRNSFDEQ |
| | | | NEISGRYSWNFQKSTSSFNFTITASQVVDSAVYFCALSTH |
| | | | GNKLVFGAGTILRVKSYIQNPDPAVYQLRD |
| | TRB | 620 | MGDVTVGTALWRQGRPSSCAPAHSDPDLVKLPSCPDPA |
| | | | MGTSLLCWMALCLLGADHADTGVSQDPRHKITKRGQNV |
| | | | TFRCDPISEHNRLYWYRQTLGQGPEFLTYFQNEAQLEKS |
| | | | RLLSDRFSAERPKGSFSTLEIQRTEQGDSAMYLCASSLD |
| | | | RENEQFFGPGTRLTVLEDLKNVFPPEVAVFEPSEAEISHT |
| | | | QKATLVCL |
| 19 | TRA | 621 | MALQSTLGAVWLGLLLNSLWKVAESKDQVFQPSTVASS |
| | | | EGAVVEIFCNHSVSNAYNFFWYLHFPGCAPRLLVKGSKP |
| | | | SQQGRYNMTYERFSSSLLILQVREADAAVYYCAVEDRDN |
| | | | NARLMFGDGTQLVVKPNIQNPDPAVYQLRD |
| | TRB | 622 | MRSWPGPEMGTRLFFYVALCLLWTGHMDAGITQSPRHK |
| | | | VTETGTPVTLRCHQTENHRYMYWYRQDPGHGLRLIHYS |
| | | | YGVKDTDKGEVSDGYSVSRSKTEDFLLTLESATSSQTSV |
| | | | YFCAISERPVGEQETQYFGPGTRLLVLEDLKNVFPPEVA |
| | | | VFEPSEAEISHTQKATLVCLATGF |
| 20 | TRA | 623 | MEKMLECAFIVLWLQLGWLSGEDQVTQSPEALRLQEGE |
| | | | SSSLNCSYTVSGLRGLFWYRQDPGKGPEFLFTLYSAGE |
| | | | EKEKERLKATLTKKESFLHITAPKPEDSATYLCAVQIG |
| | | | GYGNKLVFGAGTILRVKSYIQNPDPAVYQLRD |
| | TRB | 624 | MDSWTLCCVSLCILVAKHTDAGVIQSPRHEVTEMGQEVT |
| | | | LRCKPISGHDYLFWYRQTMMRGLELLIYFNNNVPIDDSG |
| | | | MPEDRFSAKMPNASFSTLKIQPSEPRDSAVYFCASSPRP |
| | | | SEKLFFGSGTQLSVLEDLNKVFPPEVAVFEPSEAEISHT |
| | | | QKATLVCLATGFYPDHVELSWWVNGKEVHSGVSTDPQPL |
| | | | KEQPALNDSRYCL |
| | | SEQ | |
| | Descrip- | ID | |
| TCR | tion | NO: | Nucleotide sequence |
| 1 | TRA | 625 | ATTTCAAAAGCATGAAAATTACTAGAGGACTTGCATTT |
| | | | GATTGGTTGAGCAGCCAACAGAAAGGCTCTTTCCTGC |
| | | | ATTACAGAATATGAACTAAGATACAGAAGTGGCGCCTC |
| | | | TGAGAAAAGAAGGTTGGAATTATCGTAATTTGTTTCTA |
| | | | GGCTGAGATACCAGCATGGAGAAAATGTTGGAGTGTG |
| | | | CATTCATAGTCTTGTGGCTTCAGCTTGGCTGGTTGAGT |
| | | | GGAGAAGACCAGGTGACGCAGAGTCCCGAGGCCCTG |
| | | | AGACTCCAGGAGGGAGAGAGTAGCAGTCTCAACTGCA |
| | | | GTTACACAGTCAGCGGTTTAAGAGGGCTGTTCTGGTAT |
| | | | AGGCAAGATCCTGGGAAAGGCCCTGAATTCCTCTTCA |
| | | | CCCTGTATTCAGCTGGGGAAGAAAAGGAGAAAGAAAG |
| | | | GCTAAAAGCCACATTAACAAAGAAGGAAAGCTTTCTGC |
| | | | ACATCACAGCCCCTAAACCTGAAGACTCAGCCACTTAT |
| | | | CTCTGTGCTGCGGCCATAGGAAGCTACATACCTACATT |
| | | | TGGAAGAGGAACCAGCCTTATTGTTCATCCGTATATCC |
| | | | AGAACCCTGACCCTGCCGTGTACCAGCTGAGAGACTA |
| | | | GATCGGG |
| | TRB | 626 | AGAGTAGTGATATCACAGCCTAACATCCGATCAGAGAT |
| | | | GCAATGCCCAAAACCCCAGCTCTCAGAGGACCAGTAT |
| | | | CCCTCACAGGGTGACACCTGACCAGCTCTGTCCCACC |
| | | | TGGCCATGGGCTCCAGGTACCTCTGATGGGAAGACCT |
| | | | TTGTCTCTTGGGAACAAGTGAATCCTTGGCACAGGCC |
| | | | CAGTGGATTCTGCTGTGCAGAACAGAGAGCAGTGGAC |
| | | | CTCAGGAGGCCTGCAAGGGGAGGACATAGGACAGTG |
| | | | ACATCACAGTATGCCCCTCCCACCAGGAAAAGCAAGG |
| | | | CTGAGAATTTAGCTCTTTCCCAGGAGGACCAAGCCCT |
| | | | GAGCACAGACACAGTGCTGCCTGCCCCTTTGTGCCAT |
| | | | GGGCTCCAGGCTGCTCTGTTGGGTGCTGCTTTGTCTC |
| | | | CTGGGAGCAGGCCCAGTAAAGGCTGGAGTCACTCAAA |
| | | | CTCCAAGATATCTGATCAAAACGAGAGGACAGCAAGT |
| | | | GACACTGAGCTGCTCCCCTATCTCTGGGCATAGGAGT |
| | | | GTATCCTGGTACCAACAGACCCCAGGACAGGGCCTTC |
| | | | AGTTCCTCTTTGAATACTTCAGTGAGACACAGAGAAAC |
| | | | AAAGGAAACTTCCCTGGTCGATTCTCAGGGCGCCAGT |
| | | | TCTCTAACTCTCGCTCTGAGATGAATGTGAGCACCTTG |
| | | | GAGCTGGGGGACTCGGCCCTTTATCTTTGCGCCAGCA |
| | | | GCTTGGGGAATTCGAACACTGAAGCTTTCTTTGGACAA |
| | | | GGCACCAGACTCACAGTTGTAGAGGACCTGAACAAGG |
| | | | TGTTCCCACCCGAGGTCGCTGTGTTTGAGCCATCAGA |
| | | | AGCAGAGATCTCCCACACCCAAAAGGCCACACTGGTG |
| | | | TGCCTGGCCACAGGCTTCTACCCCGACCACGTGGAGC |
| | | | TGAGCTGGTGGGTGAATGGGAAGGAGGTGCACAGTG |
| | | | GGGTCAGCACAGACCCGCAGCCCCTCAAGGAGCAGC |
| | | | CCGCCCTCAATGACTCCAGATACTGCCTGA |
| 2 | TRA | 627 | GAGAGGAGGAGGAGGAGGAGGAGGAAGGATGAGGG |
| | | | GAGGGGCTTAGTGGTGAATTAGGGGTGTTAAAAAGAG |
| | | | CATCATTTTTTTGAACTGGTAAAGCAGATTCTTTTTATG |
| | | | ATTTTTAAAGTAGAAATATCCATTCCAGGTGCATTTTTT |
| | | | AAGGGTTTAAAATTTGAATCCTCAGTGAACCAGGGCAG |
| | | | AGAAGAATGATGAAATCCTTGAGAGTTTTACTAGTGAT |
| | | | CCTGTGGCTTCAGTTGAGCTGGGTTTGGAGCCAACAG |
| | | | AAGGAGGTGGAGCAGAATTCTGGACCCCTCAGTGTTC |
| | | | CAGAGGGAGCCATTGCCTCTCTCAACTGCACTTACAG |
| | | | TGACCGAGGTTCCCAGTCCTTCTTCTGGTACAGACAAT |
| | | | ATTCTGGGAAAAGCCCTGAGTTGATAATGTCCATATAC |
| | | | TCCAATGGTGACAAAGAAGATGGAAGGTTTACAGCAC |
| | | | AGCTCAATAAAGCCAGCCAGTATGTTTCTCTGCTCATC |
| | | | AGAGACTCCCAGCCCAGTGATTCAGCCACCTACCTCT |
| | | | GTGCCGTGAGGGAGTCAGGAGGAGGTGCTGACGGAC |
| | | | TCACCTTTGGCAAAGGGACTCATCTAATCATCCAGCCC |
| | | | TATATCCAGAACCCTGACCCTGCCGTGTACCAGCTGA |
| | | | GAGACTCTAAATCCAGTGAC |
| | TRB | 628 | GACCTGTTAGAGGAGGGAAACAGAGTGGGAAGGAACT |
| | | | ACGCTGTAGGAAGGAAAGATGAACTTGAGTTTCACTTC |
| | | | TTAGTGCCTTTTCTCAGGGGAGAGGCCATCACTTGAA |
| | | | GATGCTGAGTCTTCTGCTCCTTCTCCTGGGACTAGGCT |
| | | | CTGTGTTCAGTGCTGTCATCTCTCAAAAGCCAAGCAGG |
| | | | GATATCTGTCAACGTGGAACCTCCCTGACGATCCAGT |
| | | | GTCAAGTCGATAGCCAAGTCACCATGATGTTCTGGTAC |
| | | | CGTCAGCAACCTGGACAGAGCCTGACACTGATCGCAA |
| | | | CTGCAAATCAGGGCTCTGAGGCCACATATGAGAGTGG |
| | | | ATTTGTCATTGACAAGTTTCCCATCAGCCGCCCAAACC |
| | | | TAACATTCTCAACTCTGACTGTGAGCAACATGAGCCCT |
| | | | GAAGACAGCAGCATATATCTCTGCAGCGTTGGGGAAC |
| | | | AGGGTATGACGCAGCCCCAGCATTTTGGTGATGGGAC |
| | | | TCGACTCTCCATCCTAGAGGACCTGAACAAGGTGTTC |
| | | | CCACCCGAGGTCGCTGTGTTTGAGCCATCAGAAGCAG |
| | | | AGATCTCCCACACCCAAAAGGCCACACTGGTGTGCCT |
| | | | GGCCACAGGCTTCT |
| 3 | TRA | 629 | GAGGAAGCTGCTCTATCTTTAGCTGACGATTTCTGGG |
| | | | GAGCACTCAAATTGAAACCTGCCTGATGTGGGATGTG |
| | | | CTGTGGCTGCTGCTTTGTTGCTTGGGACCTCCTCTGA |
| | | | CCTAGGATCAGACACAGAGTCTGAGTTCTGGGGCCTG |
| | | | GAACCTCAATGTGCACTTGAACAATGAAGTTGGTGACA |
| | | | AGCATTACTGTACTCCTATCTTTGGGTATTATGGGTGA |
| | | | TGCTAAGACCACACAGCCAAATTCAATGGAGAGTAAC |
| | | | GAAGAAGAGCCTGTTCACTTGCCTTGTAACCACTCCAC |
| | | | AATCAGTGGAACTGATTACATACATTGGTATCGACAGC |
| | | | TTCCCTCCCAGGGTCCAGAGTACGTGATTCATGGTCTT |
| | | | ACAAGCAATGTGAACAACAGAATGGCCTCTCTGGCAAT |
| | | | CGCTGAAGACAGAAAGTCCAGTACCTTGATCCTGCAC |
| | | | CGTGCTACCTTGAGAGATGCTGCTGTGTACTACTGCAT |
| | | | CCTGAGATATCAGGGAGCCCAGAAGCTGGTATTTGGC |
| | | | CAAGGAACCAGGCTGACTATCAACCCAAATATCCAGAA |
| | | | CCCTGACCCTGCCGTGTACCAGCTGAGAGACTAGAT |
| | TRB | 630 | GATTCATCCTAGCCCAGCAAATTCAAATCTACCTTCTAT |
| | | | CAGAACTTAGAAAGGATGTAAAGCAGTCAGGAAGAAA |
| | | | CATCCCCTGGGTCTGGGGAAACTATCAGGAGCAGTGA |
| | | | CATCACAAGAAAAACCACCAACCAGGGCCAAGGAGAC |
| | | | CAGAGCCCAGCACCTCGCCCAAAGGACCCCAGTCAGA |
| | | | GGCCCCATCTCAGACCCGAGGCTAGCATGGGCTGCA |
| | | | GGCTGCTCTGCTGTGCGGTTCTCTGTCTCCTGGGAGC |
| | | | AGTTCCCATAGACACTGAAGTTACCCAGACACCAAAAC |
| | | | ACCTGGTCATGGGAATGACAAATAAGAAGTCTTTGAAA |
| | | | TGTGAACAACATATGGGGCACAGGGCTATGTATTGGT |
| | | | ACAAGCAGAAAGCTAAGAAGCCACCGGAGCTCATGTT |
| | | | TGTCTACAGCTATGAGAAACTCTCTATAAATGAAAGTG |
| | | | TGCCAAGTCGCTTCTCACCTGAATGCCCCAACAGCTCT |
| | | | CTCTTAAACCTTCACCTACACGCCCTGCAGCCAGAAGA |
| | | | CTCAGCCCTGTATCTCTGCGCCAGCAGCCAAGTAAGG |
| | | | GAGCGCAAGAACACCGGGGAGCTGTTTTTTGGAGAAG |
| | | | GCTCTAGGCTGACCGTACTGGAGGACCTGAAAAACGT |
| | | | GTTCCCACCCGAGGTCGCTGTGTTTGAGCCATCAGAA |
| | | | GCAGAGATCTCCCACACCCAAAAGGCCACACTGGTGT |
| | | | GCCTGGCCACAGGCTTCTACCCCGACCACGTGGAGCT |
| | | | GAGCTGGTGGGTGAATGGGAAGGAGGTGCACAGTGG |
| | | | GGTCAGCACAGACCCGCAGCCCCTCAAGGAGCAGCC |
| | | | CGCCCTCAATGACTCCAGATACTGCCTGA |
| 4 | TRA | 631 | CATTCCTCAGTCTGGCCACAGGGGAGAGCTGTTTAAA |
| | | | AAGTATTCTAGGTAGATGCATTTAGTTCTCTGCAGTTAA |
| | | | ACTGTTGTGATGTGTCTCTGATTGGTTGCCCCCAAAGT |
| | | | TGCAATATCCTGGAAGCAATTGCAACAGGCCTCATTCT |
| | | | GAGTTCAAAGCAACTCCTGTTAAGGAAGCCCATTCAGA |
| | | | AGCTGACTGGATATTCTGGCAGGCCAAGGATGGAGAC |
| | | | TCTCCTGAAAGTGCTTTCAGGCACCTTGTTGTGGCAGT |
| | | | TGACCTGGGTGAGAAGCCAACAACCAGTGCAGAGTCC |
| | | | TCAAGCCGTGATCCTCCGAGAAGGGGAAGATGCTGTC |
| | | | ATCAACTGCAGTTCCTCCAAGGCTTTATATTCTGTACA |
| | | | CTGGTACAGGCAGAAGCATGGTGAAGCACCCGTCTTC |
| | | | CTGATGATATTACTGAAGGGTGGAGAACAGAAGGGTC |
| | | | ATGAAAAAATATCTGCTTCATTTAATGAAAAAAAGCAGC |
| | | | AAAGCTCCCTGTACCTTACGGCCTCCCAGCTCAGTTAC |
| | | | TCAGGAACCTACTTCTGCGGCACTGGGGCTGGGAGTT |
| | | | ACCAACTCACTTTCGGGAAGGGGACCAAACTCTCGGT |
| | | | CATACCAAATATCCAGAACCCTGACCCTGCCGTGTACC |
| | | | AGCTGAGAGACTCTAAATCCAGTGACAAGTCTGTCTGC |
| | | | CTATTCACCGATTTTGATTCTC |
| | TRB | 632 | GCAGAACTTAGAAAGGATGTAAAGCAGTCAGGAAGAA |
| | | | ACATCCCCTGGGTCTGGGGAAACTATCAGGAGCAGTG |
| | | | ACATCACAAGAAAAACCACCAACCAGGGCCAAGGAGÅ |
| | | | CCAGAGCCCAGCACCTCGCCCAAAGGACCCCAGTCA |
| | | | GAGGCCCCATCTCAGACCCGAGGCTAGCATGGGCTG |
| | | | CAGGCTGCTCTGCTGTGCGGTTCTCTGTCTCCTGGGA |
| | | | GCAGTTCCCATAGACACTGAAGTTACCCAGACACCAAA |
| | | | ACACCTGGTCATGGGAATGACAAATAAGAAGTCTTTGA |
| | | | AATGTGAACAACATATGGGGCACAGGGCTATGTATTG |
| | | | GTACAAGCAGAAAGCTAAGAAGCCACCGGAGCTCATG |
| | | | TTTGTCTACAGCTATGAGAAACTCTCTATAAATGAAAGT |
| | | | GTGCCAAGTCGCTTCTCACCTGAATGCCCCAACAGCT |
| | | | CTCTCTTAAACCTTCACCTACACGCCCTGCAGCCAGAA |
| | | | GACTCAGCCCTGTATCTCTGCGCCAGCAGCCACCCCG |
| | | | GCTTAAGGGCGGGAGCCTCCTACAATGAGCAGTTCTT |
| | | | CGGGCCAGGGACACGGCTCACCGTGCTAGAGGACCT |
| | | | GAAAAACGTGTTCCCACCCGAGGTCGCTGTGTTTGAG |
| | | | CCATCAGAAGCAGAGATCTCCCACACCCAAAAGGCCA |
| | | | CACTGGTGTGCCTGGCCACAGGCTTCTACCCCGACCA |
| | | | CGTGGAGCTGAGCTGGTGGGTGAATGGGAAGGAGGT |
| | | | GCACAGTGGGGTCAGCAC |
| 5 | TRA | 633 | CTATTTCCCTTTTTCTGAGTGTGTGTCTGTGTGCATGT |
| | | | GTGTGTGCACGCACGCGCACACAGGCAGACGCTAGG |
| | | | GGTAGAGTGTGATGGTTCAAAAAGAAAAGGAAACGCT |
| | | | TAAAGGTAGTGAATCACGTTTTGCCCAGGAAAACACAC |
| | | | TTGATAACTGAAGGATGATGAAGTGTCCACAGGCTTTA |
| | | | CTAGCTATCTTTTGGCTTCTACTGAGCTGGGTGAGCAG |
| | | | TGAAGACAAGGTGGTACAAAGCCCTCTATCTCTGGTTG |
| | | | TCCACGAGGGAGACACTGTAACTCTCAATTGCAGTTAT |
| | | | GAAGTGACTAACTTTCGAAGCCTACTATGGTACAAGCA |
| | | | GGAAAAGAAAGCTCCCACATTTCTATTTATGCTAACTT |
| | | | CAAGTGGAATTGAAAAGAAGTCAGGAAGACTAAGTAG |
| | | | CATATTAGATAAGAAAGAACTTTTCAGCATCCTGAACAT |
| | | | CACAGCCACCCAGACCGGAGACTCGGCCGTCTACCTC |
| | | | TGTGCTGTCTTGAACACCGGTAACCAGTTCTATTTTGG |
| | | | GACAGGGACAAGTTTGACGGTCATTCCAAATATCCAGA |
| | | | ACCCTGACCCTGCCGTGTACCAGCTGAGAGACTCTAA |
| | | | ATCCAGTGACAAGT |
| | TRB | 634 | AGAAAGGATGTAAAGCAGTCAGGAAGAAACATCCCCT |
| | | | GGGTCTGGGGAAACTATCAGGAGCAGTGACATCACAA |
| | | | GAAAAACCACCAACCAGGGCCAAGGAGACCAGAGCC |
| | | | CAGCACCTCGCCCAAAGGACCCCAGTCAGAGGCCCC |
| | | | ATCTCAGACCCGAGGCTAGCATGGGCTGCAGGCTGCT |
| | | | CTGCTGTGCGGTTCTCTGTCTCCTGGGAGCAGTTCCC |
| | | | ATAGACACTGAAGTTACCCAGACACCAAAACACCTGGT |
| | | | CATGGGAATGACAAATAAGAAGTCTTTGAAATGTGAAC |
| | | | AACATATGGGGCACAGGGCTATGTATTGGTACAAGCA |
| | | | GAAAGCTAAGAAGCCACCGGAGCTCATGTTTGTCTAC |
| | | | AGCTATGAGAAACTCTCTATAAATGAAAGTGTGCCAAG |
| | | | TCGCTTCTCACCTGAATGCCCCAACAGCTCTCTCTTAA |
| | | | ACCTTCACCTACACGCCCTGCAGCCAGAAGACTCAGC |
| | | | CCTGTATCTCTGCGCCAGCAGTCGGTTTCCGGGCGGG |
| | | | GGCTACAATGAGCAGTTCTTCGGGCCAGGGACACGGC |
| | | | TCACCGTGCTAGAGGACCTGAAAAACGTGTTCCCACC |
| | | | CGAGGTCGCTGTGTTTGAGCCATCAGAAGCAGAGATC |
| | | | TCCCACACCCAAAAGGCCACACTGGTGTGCCTGGCCA |
| | | | CAGGCTTCTACCCCGACCACGTGGAGCTGAGCTGGTG |
| | | | GGTGAATG |
| 6 | TRA | 635 | CGTGTGTGTGTGTGTCTGTGTGTGTGTGTGTGTGCTTG |
| | | | AGAGAGAGAGAAGGAGAGAAAGAGAGAGAATGGGAA |
| | | | GGGCGATAAGAGGAGGGGTTAGTGATATACCAGGGGT |
| | | | TGTGAAAATAACCTCTTTTTTCTAATTGGTAGGACAGAT |
| | | | TCTTTTTACGATTCCTAAAGTGGAAGAAATAAAGTATCT |
| | | | CTGCTATGTTCATTTCTTTTTGGATTGAAAATTTTAATC |
| | | | CTCAGTGAACCAGGGCAGAAAAGAATGATGATATCCTT |
| | | | GAGAGTTTTACTGGTGATCCTGTGGCTTCAGTTAAGCT |
| | | | GGGTTTGGAGCCAACGGAAGGAGGTGGAGCAGGATC |
| | | | CTGGACCCTTCAATGTTCCAGAGGGAGCCACTGTCGC |
| | | | TTTCAACTGTACTTACAGCAACAGTGCTTCTCAGTCTTT |
| | | | CTTCTGGTACAGACAGGATTGCAGGAAAGAACCTAAG |
| | | | TTGCTGATGTCCGTATACTCCAGTGGTAATGAAGATGG |
| | | | AAGGTTTACAGCACAGCTCAATAGAGCCAGCCAGTATA |
| | | | TTTCCCTGCTCATCAGAGACTCCAAGCTCAGTGATTCA |
| | | | GCCACCTACCTCTGTGTGGTGAACTGGTCCGGAAATG |
| | | | AGAAATTAACCTTTGGGACTGGAACAAGACTCACCATC |
| | | | ATACCCAATATCCAGAACCCTGACCCTGCCGTGTACCA |
| | | | GCTGAGAGACTTAAAGACAACCCAAACCAGAGTCTTTT |
| | | | GACAGGATATATTCAGACAGATCTGCGTGGGATGA |
| 7 | TRB | 636 | GCATCTTTCTTTAAGACCCAGTACCTTGGGAGGGACAA |
| | | | TGACATCACTTCCTGAATCCTCCCAGTTTTCTATTTCCA |
| | | | TGCCCTGCTTCCCTCAACATCCAGAGCTGGAAACACC |
| | | | TCCATCCTGCCTCTTCATGCCATGGCCTCCCTGCTCTT |
| | | | CTTCTGTGGGGCCTTTTATCTCCTGGGAACAGGGTCC |
| | | | ATGGATGCTGATGTTACCCAGACCCCAAGGAATAGGA |
| | | | TCACAAAGACAGGAAAGAGGATTATGCTGGAATGTTCT |
| | | | CAGACTAAGGGTCATGATAGAATGTACTGGTATCGACA |
| | | | AGACCCAGGACTGGGCCTACGGTTGATCTATTACTCC |
| | | | TTTGATGTCAAAGATATAAACAAAGGAGAGATCTCTGA |
| | | | TGGATACAGTGTCTCTCGACAGGCACAGGCTAAATTCT |
| | | | CCCTGTCCCTAGAGTCTGCCATCCCCAACCAGACAGC |
| | | | TCTTTACTTCTGTGCCACCAGGGGACAGGGGGTCGGG |
| | | | GAGCTGTTTTTTGGAGAAGGCTCTAGGCTGACCGTAC |
| | | | TGGAGGACCTGAAAAACGTGTTCCCACCCGAGGTCGC |
| | | | TGTGTTTGAGCCATCAGAAGCAGAGATCTCCCACACC |
| | | | CAAAAGGCCACACTGGTGTGCCTGGCC |
| | TRA | 637 | ATGTGCACTTGCAAGTGCACACACACACATGAATAAGA |
| | | | GCAAGACAGAGGGAGAGATGAGGGAGGAGCTTAATG |
| | | | ATGGAGCAGAGGTGTTAAAAAGAACATCCTTTTTCTAA |
| | | | TTGGTAGGACAGATTTCTTTTATGATTCCTACAGCAGA |
| | | | AAAATGAGAAACGTTTGTTATTATTTTTTTTTCGTGTTTA |
| | | | AGGTTTGAATCCTCAGTGAACCAGGGCAGAAAAGAAT |
| | | | GATGAAATCCTTGAGAGTTTTACTGGTGATCCTGTGGC |
| | | | TTCAGTTAAGCTGGGTTTGGAGCCAACAGAAGGAGGT |
| | | | GGAGCAGGATCCTGGACCACTCAGTGTTCCAGAGGGA |
| | | | GCCATTGTTTCTCTCAACTGCACTTACAGGAACAGTGC |
| | | | TTTTCAATACTTCATGTGGTACAGACAGTATTCCAGAAÅ |
| | | | AGGCCCTGAGTTGCTGATGTACACATACTCCAGTGGT |
| | | | AACAAAGAAGATGGAAGGTTTACAGCACAGGTCGATA |
| | | | AATCCAGCAAGTATATCTCCTTGTTCATCAGAGACTCA |
| | | | CAGCCCAGTGATTCAGCCACCTACCTCTGTGCAATGA |
| | | | GCGTACAGGCTGCAGGCAACAAGCTAACTTTTGGAGG |
| | | | AGGAACCAGGGTGCTAGTTAAACCAAATATCCAGAAC |
| | | | CCTGACCCTGCCGTGTACCAGCTGAGAGACTATGATG |
| | | | GACCATG |
| | TRB | 638 | AATGATGTCACTGTGGGAACTGCCATGAGAGGACAGG |
| | | | GACGTCCCTCCTCCTTTGCTGTTGCTCACAGTGACCCT |
| | | | GATTGGGCAAAGCTCCCATCCTTCCCTGACCCTGCCA |
| | | | TGGGCACCAGGCTCCTCTGCTGGGGGGCCCTCTGTCT |
| | | | CCTGGGAGCAGAACTCACAGAAGCTGGAGTTGCCCAG |
| | | | TCTCCCAGATATAAGATTATAGAGAAAAGGCAGAGTGT |
| | | | GGCTTTTTGGTGCAATCCTATATCTGGCCATGCTACCC |
| | | | TTTACTGGTACCAGCAGATCCTGGGACAGGGCCCAAA |
| | | | GCTTCTGATTCAGTTTCAGAATAACGGTGTAGTGGATG |
| | | | ATTCACAGTTGCCTAAGGATCGATTTTCTGCAGAGAGG |
| | | | CTCAAAGGAGTAGACTCCACTCTCAAGATCCAACCTGC |
| | | | AAAGCTTGAGGACTCGGCCGTGTATCTCTGTGCCAGC |
| | | | AGCTTAGACAGGAACTATGGCTACACCTTCGGTTCGG |
| | | | GGACCAGGTTAACCGTTGTAGAGGACCTGAACAAGGT |
| | | | GTTCCCACCCGAGGTCGCTGTGTTTGAGCCATCAGAA |
| | | | GCAGAGATCTCCCACACCCAAAAGGCCACACTGGTGT |
| | | | GCCTGGCCACAGGCTTCTACCCCGACCACGTGGAGCT |
| | | | GAGCTGGTGGGTGAATGGGAAGGAGGTGCACAGTGG |
| | | | GGTCAGCACAGACCCGCAGCCCCTCAAGGAGCAGCC |
| | | | CGCCCTCAATGACTCCAGATACTGCCTGA |
| 8 | TRA | 639 | GGGGACTGTGATTTCTTCATGTTAAGGATCAAGACCAT |
| | | | TATTTGGGTAACACACTAAAGATGAACTATTCTCCAGG |
| | | | CTTAGTATCTCTGATACTCTTACTGCTTGGAAGAACCC |
| | | | GTGGAAATTCAGTGACCCAGATGGAAGGGCCAGTGAC |
| | | | TCTCTCAGAAGAGGCCTTCCTGACTATAAACTGCACGT |
| | | | ACACAGCCACAGGATACCCTTCCCTTTTCTGGTATGTC |
| | | | CAATATCCTGGAGAAGGTCTACAGCTCCTCCTGAAAG |
| | | | CCACGAAGGCTGATGACAAGGGAAGCAACAAAGGTTT |
| | | | TGAAGCCACATACCGTAAAGAAACCACTTCTTTCCACT |
| | | | TGGAGAAAGGCTCAGTTCAAGTGTCAGACTCAGCGGT |
| | | | GTACTTCTGTGCTCTGAGTGATAACACCAATGCAGGCA |
| | | | AATCAACCTTTGGGGATGGGACTACGCTCACTGTGAA |
| | | | GCCAAATATCCAGAACCCTGACCCTGCCGTGTACCAG |
| | | | CTGAGAGACTCTAAATCCAGTGACAAGTCTGTCTGCCT |
| | | | ATTCACCGATTTTGATTCTCAAACAAATGTGTCACAAAG |
| | | | TAAGGATTCTGATGTGTATATCACAGACAAAACTGTGC |
| | | | TAGACATGAGGTCTATGGACTTCAAGAGCAACAGTGCT |
| | | | GTGGCCTGG |
| | TRB | 640 | GTCATGGGCAAAGATTACCACCAGGGGGCAGACTAGG |
| | | | GCATCCTTGGGATTCTGTGATCAGTCATCCCTCCTCGC |
| | | | TGGTGAATGGAGGCAGTGGTCACAACTCTCCCCAGAG |
| | | | AAGGTGGTGTGAGGCCATCACGGAAGATGCTGCTGCT |
| | | | TCTGCTGCTTCTGGGGCCAGGCTCCGGGCTTGGTGCT |
| | | | GTCGTCTCTCAACATCCGAGCTGGGTTATCTGTAAGAG |
| | | | TGGAACCTCTGTGAAGATCGAGTGCCGTTCCCTGGAC |
| | | | TTTCAGGCCACAACTATGTTTTGGTATCGTCAGTTCCC |
| | | | GAAACAGAGTCTCATGCTGATGGCAACTTCCAATGAG |
| | | | GGCTCCAAGGCCACATACGAGCAAGGCGTCGAGAAG |
| | | | GACAAGTTTCTCATCAACCATGCAAGCCTGACCTTGTC |
| | | | CACTCTGACAGTGACCAGTGCCCATCCTGAAGACAGC |
| | | | AGCTTCTACATCTGCAGTGCTAGGACTAGCGGTCAATA |
| | | | CGAGCAGTACTTCGGGCCGGGCACCAGGCTCACGGT |
| | | | CACAGAGGACCTGAAAAACGTGTTCCCACCCGAGGTC |
| | | | GCTGTGTTTGAGCCATCAGAAGCAGAGATCTCCCACA |
| | | | CCCAAAAGGCCACACTGGTGTGCCTGGCCACAGGCTT |
| | | | CTACCCCGACCACGTGGAGCTGAGCTGGTGGGTGAAT |
| | | | GGGAAGGAGGTGCACAGTGGGGTCAGCACAGACCCG |
| | | | CAGCCCCTCAAGGAGCAGCCCGCCCTCAATGACTCCA |
| | | | GATACTGCCTGA |
| 9 | TRA | 641 | AACACAAACTCATTTCCTGCTATCACTGTAGGTTTCACT |
| | | | GTGATTTCTTCATGTTAAGGATCAAGACCATTATTTGG |
| | | | GTAACACACTAAAGATGAACTATTCTCCAGGCTTAGTA |
| | | | TCTCTGATACTCTTACTGCTTGGAAGAACCCGTGGAGA |
| | | | TTCAGTGACCCAGATGGAAGGGCCAGTGACTCTCTCA |
| | | | GAAGAGGCCTGCCTGACTATAAACTGCACGTACACAG |
| | | | CCACAGGATACCCTTCCCTTTTCTGGTATGTCCAATAT |
| | | | CCTGGAGAAGGTCTACAGCTCCTCCTGAAAGCCACGÅ |
| | | | AGGCTGATGACAAGGGAAGCAACAAAGGTTTTGAAGC |
| | | | CACATACCGTAAGGAAACCACTTCTTTCCACTTGGAGA |
| | | | AAGGCTCAGTTCAAGTGTCAGACTCAGCGGTGTACTT |
| | | | CTGTGCTCTGAGTGATCTGTTGGACTCTGGCAACACA |
| | | | GGCAAACTAATCTTTGGGCAAGGGACAACTTTACAAGT |
| | | | AAAACCAGATATCCAGAACCCTGACCCTGCCGTGTAC |
| | | | CAGCTGAGAGACTAGA |
| | TRB | 642 | GTACTGTGTTCAGTCCTCCTCACCTTTAGCTTCTGGCC |
| | | | TGTGCAAGACAGGAATAGTACATGGAGTAGCAGAACC |
| | | | TGAGGCCTGCCAAAGAGTACAGTGGGCTGGAGTGGC |
| | | | CTGCAGGGGAAAGTATGTCAGTGTGGTGACATCACAG |
| | | | GTCAATGCTCCTATCAGGAAGAAGGGAGCTTAGGAAC |
| | | | TTCAGAATGCTTACTACAGAGACACCAGCCCCAAGCTA |
| | | | GGAGATCCTGCCATGGGCTTCAGGCTCCTCTGCTGTG |
| | | | TGGCCTTTTGTCTCCTGGGAGCAGGCCCAGTGGATTC |
| | | | TGGAGTCACACAAACCCCAAAGCACCTGATCACAGCA |
| | | | ACTGGACAGCGAGTGACGCTGAGATGCTCCCCTAGGT |
| | | | CTGGAGACCTCTCTGTGTACTGGTACCAACAGAGCCT |
| | | | GGACCAGGGCCTCCAGTTCCTCATTCAGTATTATAATG |
| | | | GAGAAGAGAGAGCAAAAGGAAACATTCTTGAACGATT |
| | | | CTCCGCACAACAGTTCCCTGACTTGCACTCTGAACTAA |
| | | | ACCTGAGCTCTCTGGAGCTGGGGGACTCAGCTTTGTA |
| | | | TTTCTGTGCCAGTAGAGGATCCTACAATGAGCAGTTCT |
| | | | TCGGGCCAGGGACACGGCTCACCGTGCTAGAGGACC |
| | | | TGAAAAACGTGTTCCCACCCGAGGTCGCTGTGTTTGA |
| | | | GCCATCAGAAGCAGAGATCTCCCACACCCAAAAGGCC |
| | | | ACACTGGTGTGCCTGGCCACAGGCTTCTACCCCGACC |
| | | | ACGTGGAGCTGAGCTGGTGGGTGAATGGGAAG |
| 10 | TRA | 643 | AGTGCTCGTGTGTGTGTGTGTCTGTGTGTGTGTGTGT |
| | | | GTGCTTGAGAGAGAGAGAAGGAGAGAAAGAGAGAGAA |
| | | | TGGGAAGGGCGATAAGAGGAGGGGTTAGTGATATACC |
| | | | AGGGGTTGTGAAAATAACCTCTTTTTTCTAATTGGTAG |
| | | | GACAGATTCTTTTTACGATTCCTAAAGTGGAAGAAATA |
| | | | AAGTATCTCTGCTATGTTCATTTCTTTTTGGATTGAAAA |
| | | | TTTTAATCCTCAGTGAACCAGGGCAGAAAAGAATGATG |
| | | | ATATCCTTGAGAGTTTTACTGGTGATCCTGTGGCTTCA |
| | | | GTTAAGCTGGGTTTGGAGCCAACGGAAGGAGGTGGA |
| | | | GCAGGATCCTGGACCCTTCAATGTTCCAGAGGGAGCC |
| | | | ACTGTCGCTTTCAACTGTACTTACAGCAACAGTGCTTC |
| | | | TCAGTCTTTCTTCTGGTACAGACAGGATTCCAGGAAAG |
| | | | AACCTAAGTTGCTGATGTCCGTATACTCCAGTGGTAAT |
| | | | GAAGATGGAAGGTTTACAGCACAGCTCAATAGAGCCA |
| | | | GCCAGTATATTTCCCTGCTCATCAGAGACTCCAAGCTC |
| | | | AGTGATTCAGCCACCTACCTCTGTGTGGTGAACACTCC |
| | | | CTCTTCCAAGATAATCTTTGGATCAGGGACCAGACTCA |
| | | | GCATCCGGCCAAATATCCAGAACCCTGACCCTGCCGT |
| | | | GTACCAGCTGAGAGACT |
| | TRB | 644 | GAGGGGGCAGACTAGGGCATCCTTGGGATTCTGTGAT |
| | | | CAGTCATCCCTCCTCGCTGGTGAATGGAGGCAGTGGT |
| | | | CACAACTCTCCCCAGAGAAGGTGGTGTGAGGCCATCA |
| | | | CGGAAGATGCTGCTGCTTCTGCTGCTTCTGGGGCCAG |
| | | | GCTCCGGGCTTGGTGCTGTCGTCTCTCAACATCCGAG |
| | | | CTGGGTTATCTGTAAGAGTGGAACCTCTGTGAAGATC |
| | | | GAGTGCCGTTCCCTGGACTTTCAGGCCACAACTATGTT |
| | | | TTGGTATCGTCAGTTCCCGAAACAGAGTCTCATGCTGA |
| | | | TGGCAACTTCCAATGAGGGCTCCAAGGCCACATACGA |
| | | | GCAAGGCGTCGAGAAGGACAAGTTTCTCATCAACCAT |
| | | | GCAAGCCTGACCTTGTCCACTCTGACAGTGACCAGTG |
| | | | CCCATCCTGAAGACAGCAGCTTCTACATCTGCAGTGCT |
| | | | AGATCGGGACAGGGGCAGTTCTTCGGGCCAGGGACA |
| | | | CGGCTCACCGTGCTAGAGGACCTGAAAAACGTGTTCC |
| | | | CACCCGAGGTCGCTGTGTTTGAGCCATCAGAAGCAGA |
| | | | GATCTCCCACACCCAAAAGGCCACACTGGTGTGCCTG |
| | | | GCCACAGGCTTCTACCCCGACCACGTGGAGCTGAGCT |
| | | | GGTGGGTGAATGGGAAGGAGGTGCACAGTGGGGTCA |
| | | | GCACAGACCCGCAGCCCCTCAAGGAGCAGCCCGCCC |
| | | | TCAATGACTCCAGATACTGCCTG |
| 11 | TRA | 645 | GGCTCGTGTGTATGTGTGTGTGTGTGTGTGTGTGTGT |
| | | | GTGTGTGCTTGAGAGAGAGAGAAGGAGAGAAAGAGAG |
| | | | AGAATGGGAAGGGCGATAAGAGGAGGGGTTAGTGATA |
| | | | TACCAGGGGTTGTGAAAATAACCTTTTTTTTCTAATTGG |
| | | | TAGGACAGATTCTTTTTATGATTCCTAAAGTGGAAGAÅ |
| | | | ATAAAGTATCTCTGCTATGTTCATTTCTTTTTGGATTGA |
| | | | AAATTTTAATCCTCAGTGAACCAGGGCAGAAAAGAATG |
| | | | ATGATATCCTTGAGAGTTTTACTGGTGATCCTGTGGCT |
| | | | TCAGTTAAGCTGGGTTTGGAGCCAACGGAAGGAGGTG |
| | | | GAGCAGGATCCTGGACCCTTCAATGTTCCAGAGGGAG |
| | | | CCACTGTCGCTTTCAACTGTACTTACAGCAACAGTGCT |
| | | | TCTCAGTCTTTCTTCTGGTACAGACAGGATTGCAGGAA |
| | | | AGAACCTAAGTTGCTGATGTCCGTATACTCCAGTGGTA |
| | | | ATGAAGATGGAAGGTTTACAGCACAGCTCAATAGAGC |
| | | | CAGCCAGTATATTTCCCTGCTCATCAGAGACTCCAAGC |
| | | | TCAGTGATTCAGCCACCTACCTCTGTGTGGTGCCTCCT |
| | | | GGGGGAGGCTTCAAAACTATCTTTGGAGCAGGAACAA |
| | | | GACTATTTGTTAAAGCAAATATCCAGAACCCTGACCCT |
| | | | GCCGTGTACCAGCTGAGAGACTCTAAATCCAGTGACA |
| | | | AGTCTGTCTGCCTATTCACCGATTTTGATTCTCAAACAA |
| | | | ATGTGTCACAAAGTAAGGATTCTGATGTGTATATCACA |
| | | | GACAAAACTGTGCTAGACATGAGGTCTATGGACTTCAA |
| | | | GAGCAACAGTGCTGTGGCCTGGAGCAACAAATCTGAC |
| | | | TTTGCATGTGCAAACGCCTTCA |
| | TRB | 646 | GGACATTGATTACAAATGTCCTCAGCCTGAGTTGGCTG |
| | | | TGTTGCGTTTGTACACTTCAGCCCTCTGAGCTGAAGTG |
| | | | GGAGTGGTTTCTCTCCTGAAAATGTTTCTGAGGCCCAA |
| | | | ATAGCTGAAGAGGTGGAGACGTTACAGAAACCACCTG |
| | | | GAGCCCCCAGAACTGGCAGACACCTGCCTGATGCTGC |
| | | | CATGGGCCCCCAGCTCCTTGGCTATGTGGTCCTTTGC |
| | | | CTTCTAGGAGCAGGCCCCCTGGAAGCCCAAGTGACCC |
| | | | AGAACCCAAGATACCTCATCACAGTGACTGGAAAGAA |
| | | | GTTAACAGTGACTTGTTCTCAGAATATGAACCATGAGT |
| | | | ATATGTCCTGGTATCGACAAGACCCAGGGCTGGGCTT |
| | | | AAGGCAGATCTACTATTCAATGAATGTTGAGGTGACTG |
| | | | ATAAGGGAGATGTTCCTGAAGGGTACAAAGTCTCTCG |
| | | | AAAAGAGAAGAGGAATTTCCCCCTGATCCTGGAGTCG |
| | | | CCCAGCCCCAACCAGACCTCTCTGTACTTCTGTGCCA |
| | | | GCAGGACCAGGACAGCCAGTTATGGCTACACCTTCGG |
| | | | TTCGGGGACCAGGTTAACCGTTGTAGAGGACCTGAAC |
| | | | AAGGTGTTCCCACCCGAGGTCGCTGTGTTTGAGCCAT |
| | | | CAGAAGCAGAGATCTCCCACACCCAAAAGGCCACACT |
| | | | GGTGTGCCTGGCCACAGGCTTCTTCCCTGACCACGTG |
| | | | GAGCTGAGCTGGTGGGTGAATGGGAAGGAGGTGCAC |
| | | | AGTGGGGTCAGCACAGACCCGCAGCCCCTCAAGGAG |
| | | | CAGCCCGCCCTCAATGACTCCAGATACTGCCTGA |
| 12 | TRA | 647 | AACTCCGCCTCCCGGGTTCCCGCCATTCTCCTGCCTC |
| | | | AGCCTCTCGAGTAGCTGGGACTACAGGCGCCCGCCA |
| | | | CAGCGCCCAGCTAATTTTTTTGTATTTTTTGGTAGAGA |
| | | | CGGGGTTTCACCGTGTTAGCCAGGATGGTCTCGATCT |
| | | | CCTGACCTCGTGATCCGCCCACCTCGGCCTCCCAAAG |
| | | | CGCTGGGATTAAAGGCGTGAGCCACCGCGCCCGGCC |
| | | | TACTTTGTCTTATGTTATTCCCATTTGCCGTCTCTGTTC |
| | | | CTTATACATTATGCTTTTTCAACTTTACCAGAATCACTT |
| | | | GGATTAAAACCCGTGGATTTCTCAGTAGGAAATGTTCA |
| | | | TGTGAAGACACTTCTGTAGTAACAGAACCTACAGCTGC |
| | | | TCCTGTAGAAGGAAGTTGAAAGTCATCCCTTCAAGAAA |
| | | | GGGGCTCCTCCCCTTGTAATTCTACTGGGTTTTGCATC |
| | | | CAGACTGAGTTTCCTTCCCTCACCCACATGAAGTGTCT |
| | | | ACCTTCTGCAGACTCCAATGGCTCAGGAACTGGGAAT |
| | | | GCAGTGCCAGGCTCGTGGTATCCTGCAGCAGATGTGG |
| | | | GGAGTTTTCCTTCTTTATGTTTCCATGAAGATGGGAGG |
| | | | CACTACAGGACAAAACATTGACCAGCCCACTGAGATG |
| | | | ACAGCTACGGAAGGTGCCATTGTCCAGATCAACTGCA |
| | | | CGTACCAGACATCTGGGTTCAACGGGCTGTTCTGGTA |
| | | | CCAGCAACATGCTGGCGAAGCACCCACATTTCTGTCTT |
| | | | ACAATGTTCTGGATGGTTTGGAGGAGAAAGGTCGTTTT |
| | | | TCTTCATTCCTTAGTCGGTCTAAAGGGTACAGTTACCT |
| | | | CCTTTTGAAGGAGCTCCAGATGAAAGACTCTGCCTCTT |
| | | | ACCTCTGTGCTGTCCTGGATAGCAACTATCAGTTAATC |
| | | | TGGGGCGCTGGGACCAAGCTAATTATAAAGCCAGATA |
| | | | TCCAGAACCCTGACCCTGCCGTGTACCAGCTGAGAGA |
| | | | CT |
| | TRB | 648 | TGGGGGAAGGGGCGTGGCCTCTCCTGACAGGAAGGC |
| | | | TCTGGGGCCCAGGCAGGGAGAATGAAGTCTCAGAATG |
| | | | ACCCCCTTGAGAGTACTGTTCCCCTATCACCGATGCAC |
| | | | AGACCCAGAAGACCCCTCCATCCTGTAGCACCTGCCA |
| | | | TGAGCATCGGGCTCCTGTGCTGTGTGGCCTTTTCTCT |
| | | | CCTGTGGGCAAGTCCAGTGAATGCTGGTGTCACTCAG |
| | | | ACCCCAAAATTCCAGGTCCTGAAGACAGGACAGAGCA |
| | | | TGACACTGCAGTGTGCCCAGGATATGAACCATAACTC |
| | | | CATGTACTGGTATCGACAAGACCCAGGCATGGGACTG |
| | | | AGGCTGATTTATTACTCAGCTTCTGAGGGTACCACTGA |
| | | | CAAAGGAGAAGTCCCCAATGGCTACAATGTCTCCAGA |
| | | | TTAAACAAACGGGAGTTCTCGCTCAGGCTGGAGTCGG |
| | | | CTGCTCCCTCCCAGACATCTGTGTACTTCTGTGCCAGC |
| | | | AGTGAAGAGGCTAGCGGGTCCTACGAGCAGTACTTCG |
| | | | GGCCGGGCACCAGGCTCACGGTCACAGAGGACCTGA |
| | | | AAAACGTGTTCCCACCCGAGGTCGCTGTGTTTGAGCC |
| | | | ATCAGA |
| 13 | TRA | 649 | TGTGTGTGTGTGTGTGTGTGTGTGTGAGAGAGAGAGA |
| | | | GAGAGAGAGAGGAGGAGGAGGAGGAGGAGGAAGGAT |
| | | | GAGGGGAGGGGCTTAGTGGTGAATTAGGGGTGTTAAA |
| | | | AAGAGCATCATTTTTTTGAACTGGTAAAGCAGATTCTTT |
| | | | TTATGATTTTTAAAGTAGAAATATCCATTCCAGGTGCAT |
| | | | TTTTTAAGGGTTTAAAATTTGAATCCTCAGTGAACCAG |
| | | | GGCAGAGAAGAATGATGAAATCCTTGAGAGTTTTACTA |
| | | | GTGATCCTGTGGCTTCAGTTGAGCTGGGTTTGGAGCC |
| | | | AACAGAAGGAGGTGGAGCAGAATTCTGGACCCCTCAG |
| | | | TGTTCCAGAGGGAGCCATTGCCTCTCTCAACTGCACTT |
| | | | ACAGTGACCGAGTTTCCCAGTCCTTCTTCTGGTACAGA |
| | | | CAATATTCTGGGAAAAGCCCTGAGTTGATAATGTCCAT |
| | | | ATACTCCAATGGTGACAAAGAAGATGGAAGGTTTACAG |
| | | | CACAGCTCAATAAAGCCAGCCAGTATGTTTCTCTGCTC |
| | | | ATCAGAGACTCCCAGCCCAGTGATTCAGCCACCTACC |
| | | | TCTGTGCCGTCTCAGGGAACCAGTTCTATTTTGGGACA |
| | | | GGGACAAGTTTGACGGTCATTCCAAATATCCAGAACCC |
| | | | TGACCCTGCCGTGTACCAGCTGAGAGACTA |
| | TRB | 650 | GGTCACTGTGGGAACTGCTCTGTGGCGACAAGGACGT |
| | | | CCCTCATCCTGTGCTCCTGCTCACAGTGACCCTGATCT |
| | | | GGTAAAGCTCCCATCCTGCCCTGACCCTGCCATGGGC |
| | | | ACCAGCCTCCTCTGCTGGATGGCCCTGTGTCTCCTGG |
| | | | GGGCAGATCACGCAGATACTGGAGTCTCCCAGGACCC |
| | | | CAGACACAAGATCACAAAGAGGGGACAGAATGTAACT |
| | | | TTCAGGTGTGATCCAATTTCTGAACACAACCGCCTTTA |
| | | | TTGGTACCGACAGACCCTGGGGCAGGGCCCAGAGTTT |
| | | | CTGACTTACTTCCAGAATGAAGCTCAACTAGAAAAATC |
| | | | AAGGCTGCTCAGTGATCGGTTCTCTGCAGAGAGGCCT |
| | | | AAGGGATCTTTCTCCACCTTGGAGATCCAGCGCACAG |
| | | | AGCAGGGGGACTCGGCCATGTATCTCTGTGCCAGCAG |
| | | | CCCCCTGGGTAGCGGGAGGTACAATGAGCAGTTCTTC |
| | | | GGGCCAGGGACACGGCTCACCGTGCTAGAGGACCTG |
| | | | AAAAACGTGTTCCCACCCGAGGTCGCTGTGTTTGAGC |
| | | | CATCAGAAGCAGAGATCTCCCACACCCAAAAGGCCAC |
| | | | ACTGGTGTGCCTGGCCACAGGCTTCTACCCCGACCAC |
| | | | GTGGAGCTGAGCTGGTGGGTGAATGGGAAGGAGGTG |
| | | | CACAGTGGGGTCAGCACAGACCCGCAGCCCCTCAAG |
| | | | GAGCAGCCCGCCCTCAATGACTCCAGATACTGCCTGA |
| 14 | TRA | 651 | ATGTTTTGATACATCTCAGAAATTAGGAGAAACACACT |
| | | | AATGAGCTCTCTCTGCTAAACTGGTTCTGCACTTGGCC |
| | | | TCCAGAGGGCGATGCTGCACACACTGGAGCTTTTGTT |
| | | | TCTGTTGAAGATCAATCCACTGCTCAGTTTCTTCTTCCT |
| | | | GCAGCTGGTTGAGTTCTTTCCAGACAAAGACAAGTGA |
| | | | CAAGAATTAGAGGTTTAAAAAGCAACCAGATTCATCTC |
| | | | AGCAGCTTTTGTAGTTTTAAATAAGCAAGGAGTTTCTC |
| | | | CAGCGAAACTTCCTCACACCTCTTGGTCTTGGTCTCTT |
| | | | CAGACACTTTCCTTCCTGTTCTCTGGAGATCTTGCAGA |
| | | | AAAGAGCCTGCAGTGTTTCCCTTGTTCAGCCATGCTCC |
| | | | TGGAGCTTATCCCACTGCTGGGGATACATTTTGTCCTG |
| | | | AGAACTGCCAGAGCCCAGTCAGTGACCCAGCCTGACA |
| | | | TCCACATCACTGTCTCTGAAGGAGCCTCACTGGAGTT |
| | | | GAGATGTAACTATTCCTATGGGGCAACACCTTATCTCT |
| | | | TCTGGTATGTCCAGTCCCCCGGCCAAGGCCTCCAGCT |
| | | | GCTCCTGAAGTACTTTTCAGGAGACACTCTGGTTCAAG |
| | | | GCATTAAAGGCTTTGAGGCTGAATTTAAGAGGAGTCAA |
| | | | TCTTCCTTCAATCTGAGGAAACCCTCTGTGCATTGGAG |
| | | | TGATGCTGCTGAGTACTTCTGTGCTGTGGGTCCCTTTT |
| | | | CTGGGGGTTACCAGAAAGTTACCTTTGGAACTGGAAC |
| | | | AAAGCTCCAAGTCATCCCAAATATCCAGAACCCTGACC |
| | | | CTGCCGTGTACCAGCTGAGAGACTAGATCGGAA |
| | TRB | 652 | TCCGTCCATTTCTTATATGGGAGACCTTGCCTGTGGGG |
| | | | CCATGGGAGCTCAAAATGCCCCTCCTTTCCTCCACAG |
| | | | GACCAGATGCCTGAGCTAGGAAAGGCCTCATTCCTGC |
| | | | TGTGATCCTGCCATGGATACCTGGCTCGTATGCTGGG |
| | | | CAATTTTTAGTCTCTTGAAAGCAGGACTCACAGÅACCT |
| | | | GAAGTCACCCAGACTCCCAGCCATCAGGTCACACAGA |
| | | | TGGGACAGGAAGTGATCTTGCGCTGTGTCCCCATCTC |
| | | | TAATCACTTATACTTCTATTGGTACAGACAAATCTTGGG |
| | | | GCAGAAAGTCGAGTTTCTGGTTTCCTTTTATAATAATGA |
| | | | AATCTCAGAGAAGTCTGAAATATTCGATGATCAATTCT |
| | | | CAGTTGAAAGGCCTGATGGATCAAATTTCACTCTGAAG |
| | | | ATCCGGTCCACAAAGCTGGAGGACTCAGCCATGTACT |
| | | | TCTGTGCCAGCAGAGAGGTACACTCTGGGGCCAACGT |
| | | | CCTGACTTTCGGGGCCGGCAGCAGGCTGACCGTGCT |
| | | | GGAGGACCTGAAAAACGTGTTCCCACCCGAGGTCGCT |
| | | | GTGTTTGAGCCATCAGAAGCAGAGATCTCCCACACCC |
| | | | AAAAGGCCACACTGGTGTGCCTGGCCACAGGCTTCTA |
| | | | CCCCGACCACGTGGAGCTGAGCTGGTGGGTGAATGG |
| | | | GAAGGAGGTGCACAGTGGGGTCAGCACAGACCCGCA |
| | | | GCCCCTCAAGGAGCAGCCCGCCCTCAATGACTCCAGA |
| | | | TACTGCCTG |
| 15 | TRA | 653 | GACACTGGAGCTTTTGTTTCTGTTGAAGATCAATCCAC |
| | | | TGCTCAGTTTCTTCTTCCTGCAGCTGGTTGAGTTCTTT |
| | | | CCAGACAAAGACAAGTGACAAGAATTAGAGGTTTAAAA |
| | | | AGCAACCAGATTCATCTCAGCAGCTTTTGTAGTTTTAA |
| | | | ATAAGCAAGGAGTTTCTCCAGCGAAACTTCCTCACACC |
| | | | TCTTGGTCTTGGTCTCTTCAGACACTTTCCTTCCTGTTC |
| | | | TCTGGAGATCTTGCAGAAAAGAGCCTGCAGTGTTTCC |
| | | | CTTGTTCAGCCATGCTCCTGGAGCTTATCCCACTGCTG |
| | | | GGGATACATTTTGTCCTGAGAACTGCCAGAGCCCAGT |
| | | | CAGTGACCCAGCCTGACATCCACATCACTGTCTCTGAA |
| | | | GGAGCCTCACTGGAGTTGAGATGTAACTATTCCTATGG |
| | | | GGCAACACCTTATCTCTTCTGGTATGTCCAGTCCCCCG |
| | | | GCCAAGGCCTCCAGCTGCTCCTGAAGTACTTTTCAGG |
| | | | AGACACTCTGGTTCAAGGCATTAAAGGCTTTGAGGCT |
| | | | GAATTTAAGAGGAGTCAATCTTCCTTCAATCTGAGGAA |
| | | | ACCCTCTGTGCATTGGAGTGATGCTGCTGAGTACTTCT |
| | | | GTGCTTTTTATAACCAGGGAGGAAAGCTTATCTTCGGA |
| | | | CAGGGAACGGAGTTATCTGTGAAACCCAATATCCAGA |
| | | | ACCCTGACCCTGCCGTGTACCAGCTGAGAGACTCTAA |
| | | | ATCCAGTGACAAGTCTGTCTGCCTATTCACCGATTTTG |
| | | | ATTCTCAAACAAATGTGTCACAAAGTAAGGATTCTGAT |
| | | | GTG |
| | TRB | 654 | TAAGGGGGGGCCAGACCTTGCCTGTGGGGCCATGGG |
| | | | AGCTCAAAATGCCCCTCCTTTCCTCCACAGGACCAGAT |
| | | | GCCTGAGCTAGGAAAGGCCTCATTCCTGCTGTGATCC |
| | | | TGCCATGGATACCTGGCTCGTATGCTGGGCAATTTTTA |
| | | | GTCTCTTGAAAGCAGGACTCACAGAACCTGAAGTCAC |
| | | | CCAGACTCCCAGCCATCAGGTCACACAGATGGGACAG |
| | | | GAAGTGATCTTGCGCTGTGTCCCCATCTCTAATCACTT |
| | | | ATACTTCTATTGGTACAGACAAATCTTGGGGCAGAAAG |
| | | | TCGAGTTTCTGGTTTCCTTTTATAATAATGAAATCTCAG |
| | | | AGAAGTCTGAAATATTCGATGATCAATTCTCAGTTGAA |
| | | | AGGCCTGATGGATCAAATTTCACTCTGAAGATCCGGTC |
| | | | CACAAAGCTGGAGGACTCAGCCATGTACTTCTGTGCC |
| | | | AGCAGGGTCATGAACACTGAAGCTTTCTTTGGACAAG |
| | | | GCACCAGACTCACAGTTGTAGAGGACCTGAACAAGGT |
| | | | GTTCCCACCCGAGGTCGCTGTGTTTGAGCCATCAGAA |
| | | | GCAGAGATCTCCCACACCCAAAAGGCCACACTGGTGT |
| | | | GCCTGGCCACAGGCTTCTTCCCTGACCACGTGGAGCT |
| | | | GAGCTGGTGGGTGAATGGGAAGGAGGTGCACAGTGG |
| | | | GGTCAGCACAGACCCGCAGCCCCTCAAGGAGCAGCC |
| | | | CGCCCTCAATGACTCCAGATACTGCCTG |
| 16 | TRA | 655 | GGCTTTTGTTTCTGTTGAAGATCAATCCACTGCTCAGT |
| | | | TTCTTCTTCCTGCAGCTGGTTGAGTTCTTTCCAGACAA |
| | | | AGACAAGTGACAAGAATTAGAGGTTTAAAAAGCAACCA |
| | | | GATTCATCTCAGCAGCTTTTGTAGTTTTAAATAAGCAAG |
| | | | GAGTTTCTCCAGCGAAACTTCCTCACACCTCTTGGTCT |
| | | | TGGTCTCTTCAGACACTTTCCTTCCTGTTCTCTGGAGA |
| | | | TCTTGCAGAAAAGAGCCTGCAGTGTTTCCCTTGTTCAG |
| | | | CCATGCTCCTGGAGCTTATCCCACTGCTGGGGATACA |
| | | | TTTTGTCCTGAGAACTGCCAGAGCCCAGTCAGTGACC |
| | | | CAGCCTGACATCCACATCACTGTCTCTGAAGGAGCCT |
| | | | CACTGGAGTTGAGATGTAACTATTCCTATGGGGCAACA |
| | | | CCTTATCTCTTCTGGTATGTCCAGTCCCCCGGCCAAG |
| | | | GCCTCCAGCTGCTCCTGAAGTACTTTTCAGGAGACACT |
| | | | CTGGTTCAAGGCATTAAAGGCTTTGAGGCTGAATTTAA |
| | | | GAGGAGTCAATCTTCCTTCAATCTGAGGAAACCCTCTG |
| | | | TGCATTGGAGTGATGCTGCTGAGTACTTCTGTGCTGTG |
| | | | GGCTTCTTGGAATATGGAAACAAACTGGTCTTTGGCGC |
| | | | AGGAACCATTCTGAGAGTCAAGTCCTATATCCAGAACC |
| | | | CTGACCCTGCCGTGTACCAGCTGAGAGACTCTAAATC |
| | | | CAGTGACAAGTCTGTCTGCCTATTCACCGATTTTGATT |
| | | | CTCAAACAAATGTGTCACAAAGTAAGGATTCTGATGTG |
| | | | TATATCACAGACAAAACTGTGCTAGACATGAGGTCTAT |
| | | | GGACTTCAAGAGCAACAGTGCTGTGGCCTGGAGCAAC |
| | | | AAATCTG |
| | TRB | 656 | GGATCAGAGATGCAATGCCCAAAACCCCAGCTCTCAG |
| | | | AGGACCAGTATCCCTCACAGGGTGACACCTGACCAGC |
| | | | TCTGTCCCACCTGGCCATGGGCTCCAGGTACCTCTGA |
| | | | TGGGAAGACCTTTGTCTCTTGGGAACAAGTGAATCCTT |
| | | | GGCACAGGCCCAGTGGATTCTGCTGTGCAGAACAGAG |
| | | | AGCAGTGGACCTCAGGAGGCCTGCAAGGGGAGGACA |
| | | | TAGGACAGTGACATCACAGTATGCCCCTCCCACCAGG |
| | | | AAAAGCAAGGCTGAGAATTTAGCTCTTTCCCAGGAGG |
| | | | ACCAAGCCCTGAGCACAGACACAGTGCTGCCTGCCCC |
| | | | TTTGTGCCATGGGCTCCAGGCTGCTCTGTTGGGTGCT |
| | | | GCTTTGTCTCCTGGGAGCAGGCCCAGTAAAGGCTGGA |
| | | | GTCACTCAAACTCCAAGATATCTGATCAAAACGAGAGG |
| | | | ACAGCAAGTGACACTGAGCTGCTCCCCTATCTCTGGG |
| | | | CATAGGAGTGTATCCTGGTACCAACAGACCCCAGGAC |
| | | | AGGGCCTTCAGTTCCTCTTTGAATACTTCAGTGAGACA |
| | | | CAGAGAAACAAAGGAAACTTCCCTGGTCGATTCTCAG |
| | | | GGCGCCAGTTCTCTAACTCTCGCTCTGAGATGAATGT |
| | | | GAGCACCTTGGAGCTGGGGGACTCGGCCCTTTATCTT |
| | | | TGCGCCAGCAGCTCCCTAGCGGGAGCCAATGAGCAG |
| | | | TTCTTCGGGCCAGGGACACGGCTCACCGTGCTAGAGG |
| | | | ACCTGAAAAACGTGTTCCCACCCGAGGTCGCTGTGTT |
| | | | TGAGCCATCAGAAGCAGAGATCTCCCACACCCAAAAG |
| | | | GCCACACTGGTGTGCCTGGCCACAGGCTTCTACCCCG |
| | | | ACCACGTGGAGCTGAG |
| 17 | TRA | 657 | ATATGGGGAGTGTTAAAAAAAAAGAGAAGATGTTGAAT |
| | | | ACACAAGTCAACTTCTGGGAGCAGATCTCTGCAGAATA |
| | | | AAAATGAAAAAGCATCTGACGACCTTCTTGGTGATTTT |
| | | | GTGGCTTTATTTTTATAGGGGGAATGGCAAAAACCAAG |
| | | | TGGAGCAGAGTCCTCAGTCCCTGATCATCCTGGAGGG |
| | | | AAAGAACTGCACTCTTCAATGCAATTATACAGTGAGCC |
| | | | CCTTCAGCAACTTAAGGTGGTATAAGCAAGATACTGGG |
| | | | AGAGGTCCTGTTTCCCTGACAATCATGACTTTCAGTGA |
| | | | GAACACAAAGTCGAACAGAAGATATACAGCAACTCTG |
| | | | GATGCAGACACAAAGCAAAGCTCTCTGCACATCACAG |
| | | | CCTCCCAGCTCAGCGATTCAGCCTCCTACATCTGTGT |
| | | | GGTGAGCGCGGGATATGGAGGAAGCCAAGGAAATCT |
| | | | CATCTTTGGAAAAGGCACTAAACTCTCTGTTAAACCAA |
| | | | ATATCCAGAACCCTGACCCTGCCGTGTACCAGCTGAG |
| | | | AGACT |
| | TRB | 658 | TGGGGTCACTGTGGGAACTGCTCTGTGGCGACAAGGA |
| | | | CGTCCCTCATCCTCTGCTCCTGCTCACAGTGACCCTG |
| | | | ATCTGGTAAAGCTCCCATCCTGCCCTGACCCTGCCAT |
| | | | GGGCACCAGCCTCCTCTGCTGGATGGCCCTGTGTCTC |
| | | | CTGGGGGCAGATCACGCAGATACTGGAGTCTCCCAGG |
| | | | ACCCCAGACACAAGATCACAAAGAGGGGACAGAATGT |
| | | | AACTTTCAGGTGTGATCCAATTTCTGAACACAACCGCC |
| | | | TTTATTGGTACCGACAGACCCTGGGGCAGGGCCCAGA |
| | | | GTTTCTGACTTACTTCCAGAATGAAGCTCAACTAGAAA |
| | | | AATCAAGGCTGCTCAGTGATCGGTTCTCTGCAGAGAG |
| | | | GCCTAAGGGATCTTTCTCCACCTTGGAGATCCAGCGC |
| | | | ACAGAGCAGGGGGACTCGGCCATGTATCTCTGTGCCA |
| | | | GCACCCCTGGGGGCTCCTCCCGCGAGCAGTACTTCG |
| | | | GGCCGGGCACCAGGCTCACGGTCACAGAGGACCTGA |
| | | | AAAACGTGTTCCCACCCGAGGTCGCTGTGTTTGAGCC |
| | | | ATCAGAAGCAGAGATCTCCCACACCCAAAAGGCCACA |
| | | | CTGGTGTGCCTGGCCACAGGCTTCT |
| 18 | TRA | 659 | GGGGGACTTCCTGTAAAAGGCAGAAGCCTCACACAGC |
| | | | CCAGTAACTTTGCTAGTACCTCTTGAGTGCAAGGTGGA |
| | | | GAATTAAGATCTGGATTTGAGACGGAGCACGGAACATT |
| | | | TCACTCAGGGGAAGAGCTATGAACATGCTGACTGCCA |
| | | | GCCTGTTGAGGGCAGTCATAGCCTCCATCTGTGTTGT |
| | | | ATCCAGCATGGCTCAGAAGGTAACTCAAGCGCAGACT |
| | | | GAAATTTCTGTGGTGGAGAAGGAGGATGTGACCTTGG |
| | | | ACTGTGTGTATGAAACCCGTGATACTACTTATTACTTAT |
| | | | TCTGGTACAAGCAACCACCAAGTGGAGAATTGGTTTTC |
| | | | CTTATTCGTCGGAACTCTTTTGATGAGCAAAATGAAAT |
| | | | AAGTGGTCGGTATTCTTGGAACTTCCAGAAATCCACCA |
| | | | GTTCCTTCAACTTCACCATCACAGCCTCACAAGTCGTG |
| | | | GACTCAGCAGTATACTTCTGTGCTCTGAGTACCCATGG |
| | | | AAACAAACTGGTCTTTGGCGCAGGAACCATTCTGAGA |
| | | | GTCAAGTCCTATATCCAGAACCCTGACCCTGCCGTGTA |
| | | | CCAGCTGAGAGACT |
| | TRB | 660 | ATGGGGGATGTCACTGTGGGAACTGCTCTGTGGCGAC |
| | | | AAGGACGTCCCTCATCCTGTGCTCCTGCTCACAGTGA |
| | | | CCCTGATCTGGTAAAGCTCCCATCCTGCCCTGACCCT |
| | | | GCCATGGGCACCAGCCTCCTCTGCTGGATGGCCCTGT |
| | | | GTCTCCTGGGGGCAGATCACGCAGATACTGGAGTCTC |
| | | | CCAGGACCCCAGACACAAGATCACAAAGAGGGGACAG |
| | | | AATGTAACTTTCAGGTGTGATCCAATTTCTGAACACAA |
| | | | CCGCCTTTATTGGTACCGACAGACCCTGGGGCAGGGC |
| | | | CCAGAGTTTCTGACTTACTTCCAGAATGAAGCTCAACT |
| | | | AGAAAAATCAAGGCTGCTCAGTGATCGGTTCTCTGCA |
| | | | GAGAGGCCTAAGGGATCTTTCTCCACCTTGGAGATCC |
| | | | AGCGCACAGAGCAGGGGGACTCGGCCATGTATCTCTG |
| | | | TGCCAGCAGCTTAGACAGGGAAAATGAGCAGTTCTTC |
| | | | GGGCCAGGGACACGGCTCACCGTGCTAGAGGACCTG |
| | | | AAAAACGTGTTCCCACCCGAGGTCGCTGTGTTTGAGC |
| | | | CATCAGAAGCAGAGATCTCCCACACCCAAAAGGCCAC |
| | | | ACTGGTGTGCCTGG |
| 19 | TRA | 661 | GTCACTGACACAAAGAAAAAAATGCAAAACAGGTAGTC |
| | | | TTAAATAAGCATTCTGGTGAGACCAACTGCATTTTGGC |
| | | | CATGGCTTTGCAGAGCACTCTGGGGGCGGTGTGGCTA |
| | | | GGGCTTCTCCTCAACTCTCTCTGGAAGGTTGCAGAAA |
| | | | GCAAGGACCAAGTGTTTCAGCCTTCCACAGTGGCATC |
| | | | TTCAGAGGGAGCTGTGGTGGAAATCTTCTGTAATCACT |
| | | | CTGTGTCCAATGCTTACAACTTCTTCTGGTACCTTCAC |
| | | | TTCCCGGGATGTGCACCAAGACTCCTTGTTAAAGGCT |
| | | | CAAAGCCTTCTCAGCAGGGACGATACAACATGACCTAT |
| | | | GAACGGTTCTCTTCATCGCTGCTCATCCTCCAGGTGC |
| | | | GGGAGGCAGATGCTGCTGTTTACTACTGTGCTGTGGA |
| | | | GGATAGGGATAACAATGCCAGACTCATGTTTGGAGAT |
| | | | GGAACTCAGCTGGTGGTGAAGCCCAATATCCAGAACC |
| | | | CTGACCCTGCCGTGTACCAGCTGAGAGACTC |
| | TRB | 662 | TATGGGGACTGAGAACCTAAGTTCTATTTCCCCAGGCA |
| | | | GGGCTGGGAGAGATGAGATCCTGGCCTGGACCTGAA |
| | | | ATGGGCACAAGGTTGTTCTTCTATGTGGCCCTTTGTCT |
| | | | CCTGTGGACAGGACACATGGATGCTGGAATCACCCAG |
| | | | AGCCCAAGACACAAGGTCACAGAGACAGGAACACCAG |
| | | | TGACTCTGAGATGTCATCAGACTGAGAACCACCGCTAT |
| | | | ATGTACTGGTATCGACAAGACCCGGGGCATGGGCTGA |
| | | | GGCTGATCCATTACTCATATGGTGTTAAAGATACTGAC |
| | | | AAAGGAGAAGTCTCAGATGGCTATAGTGTCTCTAGATC |
| | | | CCAGCTCCCAGACATCTGTGTACTTCTGTGCCATCAGT |
| | | | GAACGGCCGGTCGGGGAGCAAGAGACCCAGTACTTC |
| | | | GGGCCAGGCACGCGGCTCCTGGTGCTCGAGGACCTG |
| | | | AAAAACGTGTTCCCACCCGAGGTCGCTGTGTTTGAGC |
| | | | CATCAGAAGCAGAGATCTCCCACACCCAAAAGGCCAC |
| | | | ACTGGTGTGCCTGGCCACAGGCTTCT |
| 20 | TRA | 663 | CCTTATTTGGGGGCATTTGATTGGTTGAGCAGCCAACA |
| | | | GAAAGGCTCTTTCCTGCATTACAGAATATGAACTAAGA |
| | | | TACAGAAGTGGCGCCTCTGAGAAAAGAAGGTTGGAAT |
| | | | TATCGTAATTTGTTTCTAGGCTGAGATACCAGCATGGA |
| | | | GAAAATGTTGGAGTGTGCATTCATAGTCTTGTGGCTTC |
| | | | AGCTTGGCTGGTTGAGTGGAGAAGACCAGGTGACGCA |
| | | | GAGTCCCGAGGCCCTGAGACTCCAGGAGGGAGAGAG |
| | | | TAGCAGTCTCAACTGCAGTTACACAGTCAGCGGTTTAA |
| | | | GAGGGCTGTTCTGGTATAGGCAAGATCCTGGGAAAGG |
| | | | CCCTGAATTCCTCTTCACCCTGTATTCAGCTGGGGAAG |
| | | | AAAAGGAGAAAGAAAGGCTAAAAGCCACATTAACAAAG |
| | | | AAGGAAAGCTTTCTGCACATCACAGCCCCTAAACCTGA |
| | | | AGACTCAGCCACTTATCTCTGTGCTGTGCAGATCGGA |
| | | | GGGTATGGAAACAAACTGGTCTTTGGCGCAGGAACCA |
| | | | TTCTGAGAGTCAAGTCCTATATCCAGAACCCTGACCCT |
| | | | GCCGTGTACCAGCTGAGAGACTAGA |
| | TRB | 664 | GGTCACAAAATTCATTTCTTTGCTCATGTTCACAGAGG |
| | | | GCCTGGTCTGGAATATTCCACATCTGCTCTCACTCTGC |
| | | | CATGGACTCCTGGACCCTCTGCTGTGTGTCCCTTTGC |
| | | | ATCCTGGTAGCAAAGCACACAGATGCTGGAGTTATCC |
| | | | AGTCACCCCGGCACGAGGTGACAGAGATGGGACAAG |
| | | | AAGTGACTCTGAGATGTAAACCAATTTCAGGACACGAC |
| | | | TACCTTTTCTGGTACAGACAGACCATGATGCGGGGAC |
| | | | TGGAGTTGCTCATTTACTTTAACAACAACGTTCCGATA |
| | | | GATGATTCAGGGATGCCCGAGGATCGATTCTCAGCTA |
| | | | AGATGCCTAATGCATCATTCTCCACTCTGAAGATCCAG |
| | | | CCCTCAGAACCCAGGGACTCAGCTGTGTACTTCTGTG |
| | | | CCAGCAGCCCCCGGCCGAGTGAAAAACTGTTTTTTGG |
| | | | CAGTGGAACCCAGCTCTCTGTCTTGGAGGACCTGAAC |
| | | | AAGGTGTTCCCACCCGAGGTCGCTGTGTTTGAGCCAT |
| | | | CAGAAGCAGAGATCTCCCACACCCAAAAGGCCACACT |
| | | | GGTGTGCCTGGCCACAGGCTTCTACCCCGACCACGTG |
| | | | GAGCTGAGCTGGTGGGTGAATGGGAAGGAGGTGCAC |
| | | | AGTGGGGTCAGCACAGACCCGCAGCCCCTCAAGGAG |
| | | | CAGCCCGCCCTCAATGACTCCAGATACTGCCTGA |

### EXAMPLES

### Example 1

Epitope Mapping

To identify Sm-derived peptides that bind to HLA-DR15 the inventors employed ProImmune's REVEAL MHC-peptide Binding Assay. Briefly, 149 peptides (15-mers overlapping by 12 amino acids) spanning the three known immunogenic Sm proteins (SmB/B′, SmD1 and SmD3 (Migliorini, 2005, Autoimmunity, 38:47-54) were synthesized. The relative affinities of each peptide for HLA-DR15 complex (HLA-DRA1*01:01+HLA-DRB115:01) was measured against a known positive control. Peptides that bind with high affinity emit a high signal by forming a tertiary complex which can be detected by an antibody.

HLA Typing

Healthy human whole donor blood was HLA-typed at high resolution by the Victorian Transplant and Immunogenetics Service, Red Cross, Melbourne. The typing of common and well documented alleles (CWD) was performed using the IMGT/HLA reference database and SSO/SSP methods or using sequence based typing (SBT) using next-generation sequencing (NGS). (Mack, et al, 2013, Tissue Antigens, 81: 194-203).

Isolation of Primary Human Cells

Fully HLA-typed, fresh, human whole blood was collected and PBMCs were isolated using Lymphoprep density gradient medium in Sepmate-50 tubes following instructions of the manufacturer (Stemcell). The monocytes were purified from PBMCs using EasySep magnet and Human Monocyte Isolation Kit following the manufacturer's instructions (Stemcell). Monocytes were differentiated for 7 days into mature dendritic cells using ImmunoCult Dendritic Cell Culture Kit (Stemcell).

CD4+ cells were purified directly from whole blood with RosetteSep Human CD4+ T Cell Enrichment Cocktail following the manufacturer's instructions (Stemcell).

Naïve, T regulatory cells were purified by first enriching for CD4+ cells using with RosetteSep Human CD4+ T Cell Enrichment Cocktail then sorting the CD4+, CD25high, CD127low, CD45RA+, PI− cells on a FACS Aria Fusion flow cytometer (BD) using the following antibodies: anti-human CD4 Pacific Blue (Biolegend), anti-human CD25 APC (Biolegend), anti-human CD127 PE (Biolegend), anti-human CD45RA PE Cy7 (BD).

In Vitro Co-Culture

200,000 freshly isolated, CD4+ enriched cells were stained with 5 uM Cell Trace Violet (CTV) cell proliferation dye (Invitrogen) and co-cultured with 100,000 mature HLA-matched dendritic cells in the presence of 100 ug/mL of either peptide SmD178-92 (HLA-DRB1:0301), SmB/B′₇₋₂₁ (HLA-DRB1:0301), SmB/B′₁₋₁₅ (HLA-DRB1:1501), or SmB/B′₅₈₋₇₂ (HLA-DRB1:1501) (Mimotopes) in one well of a 96 well, flat bottom tissue culture plate (Corning) in RPMI 1640 medium (Gibco) supplemented with 10% human AB serum, 2 mM L-glutamine (Gibco) and 1% penicillin/streptomycin (Gibco). To determine the efficacy of the Sm-TCR transduced Tregs, 10⁵ PBMCs were cultured with either 10³ Sm-TCR transduced Tregs or 10⁴ control polyclonal Tregs. Replicate wells were plated and co-cultured for 5 days in a 5% CO₂ incubator at 37° C.

Flow Cytometry

After 5 days co-culture, cells were collected and stained with dCODE custom dextramer-PE following the manufacturer's instructions (Immudex). Cells were further stained with anti-human CD4 APC (eBioscience), anti-human CD8 Alexa Fluor 488 (Biolegend) and Propidium iodide (PI) (Sigma). CD8−, PI−, CD4+, CTVlow cells were sorted using a FACS Aria Fusion flow cytometer (BD), enumerated by trypan blue stain on a hemocytometer and immediately sent for 10× sequencing.

10× Sequencing

To generate cDNA libraries for single cell whole transcriptome sequencing with V(D)J and dextramer enrichment, the FACS sorted cells were resuspended at a concentration of 700-1200 cells/uL and loaded into a Chromium Controller (10× Genomics) following the manufacturer's protocol for the Chromium Single Cell Reagent Kit with the Chromium V(D)J human T cell enrichment kit and Chromium Single Cell Feature Barcode Library Kit (all 10× Genomics). The targeted cell recovery was set to 10,000 cells. The single cell cDNA libraries were sequenced with paired-end (V(D)J library) or single-end (transcriptome) 150-bp reads on the Illumina NextSeq Sequencer. Whole transcriptomic data along with paired V(D)J reads were processed using Cell Ranger Version 3.1 (10× Genomics) and Seurat R Package. After data processing, the data were visualised in Loupe Cell Browser and Loupe VDJ Browser (10× Genomics) and clonally expanded TCR sequences were selected for further analysis.

Plasmid Design

A lentiviral plasmid backbone (Creative Biolabs) containing an EF1alpha promoter 5′ of the EcoRI restriction site and a Woodchuck Hepatitis Virus Post-transcriptional Regulatory Element (WPRE) 3′ of the XbaI restriction site was used. These elements were flanked by 5′ and 3′ Long Terminal Repeat (LTR) sequences respectively. The TCR transgene sequences were designed in SnapGene (GSL Biotech LLC) to contain a 5′ EcoRI restriction site followed by the TCR beta chain, spaced with a P2A ribosome skipping sequence, followed by the TCR alpha chain, spaced by a T2A ribosome skipping sequence, followed by the enhanced green fluorescent protein (eGFP) sequence and lastly a 3′ XbaI restriction site. The TCR alpha and beta chains underwent minimal murinization (Sommermeyer, J Immunol, 2010) and cysteination (Cohen, Cancer Res, 2007) as well as codon and gene optimization for Homo sapiens using the GeneOptimiser tool (Invitrogen). The TCR transgene cassette was synthesised by GeneArt (Invitrogen) and ligated into the lentiviral backbone at the EcoRI and XbaI restriction sites.

Viral Production

Lentiviral particles encoding TCRs were prepared by transient transfection of HEK 293T cells using Lipofectamine 3000 reagent according to the manufacturer's instructions (Life Technologies). The lentiviral vector pLenti-TCR containing the alpha-beta TCR inserts and eGFP and the LentiArt Virus Packaging plasmids pHelp1, pHelp2 and pHelp3 (Creative Biolabs) were mixed at a 3:1:1:1 ratio (pLenti-TCR:pHelp1:pHelp2:pHelp3) and transfected at 25.9 μg per 55 cm2 petri dish. At 24 hr and 52 h after transfection, supernatants were collected, passed on a 0.45 μm PVDF Millex-HV filter, and concentrated by Lenti-X Concentrator reagent following the manufacturer's instructions (Clontech). Viral particles were resuspended in PBS and frozen in aliquots at −80° C. until use. HIV-1 Gag p24 antigen concentration was measured with the HIV-1 p24Antigen ELISA kit (Abcam).

Viral Transduction

To transduce primary human naïve T regulatory cells (Tregs), the sorted Tregs were placed in RPMI-1640 (Gibco) supplemented with 10% human AB serum, 2 mM L-glutamine (Gibco), 50 μM 2-mercaptoethanol and incubated with T Cell Activator aCD2, aCD3, aCD28 Microbeads (Miltenyi Biotech) at a bead-to-cell ratio of 1:2 and 300 IU/mL IL-2 (Stemcell) for 48 hr. Lentiviral particles (400 ng of HIV-1 p24 Gag/cell) were spinoculated for 2 hr at 32° C. and 1,500×g onto 24-well plates coated with 5 ug/cm² RetroNectin (Takara Bio). Activated Tregs (0.25×10⁶ cells/well) were added and spinoculated for 2 hr at 32° C. and 1,500×g then placed in a 5% CO2 incubator at 37° C. for 48 hr.

Transduced Treg Expansion and Phenotypic Analysis

48 hr after transduction and every subsequent 48 hr, 50% of cell culture medium was aspirated and replaced with fresh RPMI-1640 supplemented with 10% human AB serum, 2 mM L-glutamine, 50 μM 2-mercaptoethanol, 1% Penicillin/Streptomycin and 300 IU/mL IL-2. Cell culture was expanded upon 80% confluency. After two weeks expansion, to assess the phenotypic stability, the Treg cells were analysed on an LSR Fortessa X20 flow cytometer (BD) after staining with Live/Dead Fixable Near-IR (Invitrogen), CD4-BUV496 (BD), CD25-BUV395 (BD), CD127-PE CF594 (BD), TCRVbx-PE (where x is the antibody specific for the particular TCR clone), FoxP3-BV421 (Biolegend), Latency Associated Peptide (LAP)-APC (eBioscience), GARP-BV786 (BD), Helios-PE Cy7 (Biolegend), IL10-BV650 (BD), IFN gamma-BB700 (BD), IL17A-APCR700 (BD) and IL2-BV711 (BD).

### Example 2—Identification of Sm Derived Peptides that Bind to HLA-DR15

Identification of Sm derived peptides that bind to HLA-DR15 are shown in FIG. 1. Specifically, in FIG. 1A the MHC Class II Proimmune REVEAL assay was employed to identify Sm derived peptides (15-mers overlapping by 12 amino acids) that bind to HLA-DR15. The results of the assay are presented as percentage of binding relative to a positive control at 0 hours (blue bars) and 24 hours (red bars). Based on these scores a stability index (red bars) for each peptide was derived. The positive control scores are 100% at 0 hours, 6.4% at 24 hours; and had a stability index of 6.0. FIG. 1 B-D shows binding scores and stability indices of SmB/B′, SmD1 and SmD3 derived peptides.

### Example 3—Human T Cell Reactivity to the Top Three HLA-DR15 Restricted Sm-Peptides

Human T cell reactivity to the top three HLA-DR15 restricted Sm-peptides is shown in FIG. 2. Specifically, as shown in FIG. 2A, to determine if the HLA-DR15 restricted Sm-peptides could induce T cell reactivity, the top three high-binders: SmB/B′:1-15, SmB/B′:58-72, or SmD3:43-57 were cultured respectively, with human CD4+ T cells. T cell reactivity was determined by cell proliferation using Cell Trace Violet (CTV) assays.

FIG. 2B shows representative FACS plots showing the percentages of CTVlo CD4+ T cells. strong proliferative responses in CD4+ T cells cultured with SmB/B′:1-15 and SmB/B′:58-72 compared to No peptide and SmD3:43-57.

### Example 4—Human T Cell Reactivity to HLA-DR3 Restricted Sm-Peptides

Human T cell reactivity to HLA-DR3 restricted Sm-peptides is shown in FIG. 3. Specifically, as shown in FIG. 3A, to determine if human T cell reactivity to HLA-DR3 restricted Sm-peptides could be measured, the inventors tested the SmD1:78-92 peptide previously identified by Deshmukh U S et al, 2011, and the top in silico (IEDB) predicted binding peptide of SmB/B′, SmB/B′:7-21. These peptides were cultured individually with CD4+ T cells in co-culture cell proliferation assays and reactivity assessed by Cell Trace Violet (CTV) dilution.

FIG. 3B shows representative FACS plots showing the percentages of CTVlo CD4+ T cells. Strong proliferative responses were observed only in CD4+ T cells cultured with SmD1:78-92

### Example 5—Modified Lentiviral Construct

A map of the modified lentiviral construct used to transduce TCRs onto human regulatory T cells is shown in FIG. 4. The relative locations of the alpha and beta chains, P2A, T2A as well as the introduced murinised mutations and cysteination are shown.

### Example 6—TCR Transduction of TCRs onto Human Tregs

TCR transduction of TCRs onto human Tregs is shown in FIG. 5. Specifically, in FIG. 5A, a timeline of the TCR transduction protocol is shown. Human Tregs (CD4+CD25hi CD127lo) are first sorted by flow cytometry then stimulated with anti-CD3 and anti-CD28 beads followed by lentiviral TCR transduction on day 2. After two rounds of re-stimulation (days 9 and 16), Tregs are harvested and analysed for TCR expression and stability of Treg phenotype.

FIG. 5B shows an analysis of TCR expression in human Tregs at day 20 show that greater than 90% of transduced Tregs express the GFP tag.

Intracellular cytokine staining for the pro-inflammatory cytokine IFN-g is shown in FIG. 5C, revealing that the transduced Tregs do not switch into pro-inflammatory cells (staining for IL-17A was also negative).

The results in FIG. 5D show the transduced TCRs are functional. To determine if this protocol leads to TCRs that are functional, the inventors transduced a Jurkat T cell line and stimulated the transduced Jurkat T cells with an antigen-presenting cell line (HLA-DR15+ B-LCLs) pulsed with the TCRs cognate peptide. Here the inventors show upregulation of the early activation marker CD69 following stimulation demonstrating that the TCRs transduced using this protocol lead to functional TCRs on the surface of T cells.

### Example 7—Transduced Sm-TCRs are More Potent Suppressors of Sm-Specific T Conv Cell Reactivity

An in vitro T cell proliferation assay was performed wherein HLA-DR15+ PBMCs were stimulated with the dominant Sm peptide SmB/B′:58-72 and co-cultured with either polyclonal Tregs or Sm-TCR transduced Tregs (wherein the Tregs were transduced with the lentiviral vector as shown in FIG. 4 and wherein the TCR corresponds to HLA-DR15 TCR #1, having a CDR3a of CALSSYGNKLVF (SEQ ID NO: 8) and a CDR3β sequence of CASSSLSGSSYEQYF (SEQ ID NO:11). Proliferation of pro-inflammatory T conventional cells (Tconv) cells was assessed by Cell Trace Violet (CTV) dilution. Sm-TCR transduced Tregs more potently inhibited Tconv cell proliferation 12.1% versus 22.4%.

Enumeration of proliferating cells showed that there were more Sm-specific Tconv cells in the polyclonal group compared to the Sm-TCR group (8659 versus 2053).

Mean Fluorescence Intensity (MFI) of Sm-reactive Tconv cells reflect the number of cell divisions. The lower the MFI the more cell divisions the Tconv cells undergo. The MFI of Sm-reactive Tconv cells in the group that received polyclonal Tregs was lower than in the Sm-TCR Treg group (89.1 versus 271). Data are shown in FIG. 6. Error bars are SEM. *** P<0.001 by t-test.

These data demonstrate that Tregs transduced with the Sm-specific TCR more potently suppress autoreactive pro-inflammatory responses against the Sm antigen. The inventors believe that these data provide proof-of-concept that Tregs transduced with Sm-specific TCRs are better at suppressing autoreactive T cell responses against the Sm autoantigen and that fewer numbers of Tregs will be required to effect antigen-specific suppression. In addition, the ability to use fewer Tregs reduces the risk of the development of side effects in patients that receive Tregs.

### Example 8—Stimulation with Either Peptide SmB/B′:1-15 or Peptide SmB/B′:58-72 Causes Expansion of Regulatory T Cells

CD4+ T cells from a DR15 homozygous donor were co-cultured with autologous monocyte derived dendritic cells pulsed with peptide SmB/B′:1-15 or peptide SmB/B′:58-72 or no peptide (control). Eight days later, CD4+ cells were single cell sequenced using the 10× Genomics Human Immune Repertoire Single Cell Profiling kits. Cell clusters that expressed high Foxp3 and TIGIT as well as clusters with high expression of CD52 and LTB were labelled as Tregs.

The results, shown in FIG. 7, demonstrate that stimulation with either peptide SmB/B′:1-15 or peptide SmB/B′:58-72 results in selective expansion of T regs, as demonstrated by significant increases in the proportions of Tregs after peptide stimulation. Data shown is mean±SD of two independent experiments. *** P<0.001 by One-way ANOVA with Tukey's post-test compared to No peptide group.

### Example 9—the Dominant HLA-DR15 Restricted Sm-TCR Binds with High Affinity to HLA-DR15 Dextramers Presenting SmB/B′:58-72

To determine the binding affinity of dominant Sm-specific TCR, the inventors conducted dextramer based flow cytometry binding assays. Dextramers contain 10 peptide-MHC complexes bound together on a dextran backbone. These fluorochrome labelled dextramers allow for the detection of Sm-specific T cells and can be used to determine the relative affinities of Sm-specific TCRs.

First, using custom lentiviral vectors, the inventors cloned the top 3 TCRs obtained in Example 8 into a Jurkat T cell line. These TCRs are identified as TCRs 1-3 in Table 1.

Then, the inventors measured the mean fluorescence intensity (MFI) by flow cytometry and expressed the data using Scatchard plots. The results are shown in FIG. 8. The lower the concentration at which the MFI reaches a plateau, the lower the dissociation constant (Kd), which means the higher the affinity. The inventors found that the top Sm TCR binds with the highest affinity compared to the second and third highest Sm-specific TCRs. This result is important because it justifies this novel method of using high-throughput single cell TCR sequencing to identify high-affinity TCRs to treat disease.

### Example 10—Sm-TCR Tregs Suppress Anti-Sm Specific Pro-Inflammatory Responses and Restore Tolerance

To determine the efficacy of Sm-Tregs at suppressing anti-Sm specific pro-inflammatory responses the inventors generated Sm-Tregs, using SLE patient-derived Tregs, and tested them in in vitro co-cultures. The inventors compared the patient anti-Sm responses with either no Tregs or with polyclonal Tregs (pTregs).

Firstly, the inventors measured the effect of Sm-Tregs on the expansion of pro-inflammatory Sm-specific T conventional cells (Tconv) using a proliferation assay. The results (FIG. 9A) show that in the presence of Sm-Tregs, the numbers of Tregs to Sm-specific Tconv is markedly increased. This result means that the Sm-Tregs potently suppresses the expansion of Sm-specific Tconv cells. Furthermore, the ratio of >10 autoantigen specific Tregs to Tconv is similar to the inventors' other data showing that healthy individuals have 10 times as many autoantigen specific Tregs to Tconv

Next, the inventors measured cytokine production and found that in the presence of Sm-Tregs an anti-inflammatory response, i.e. high IL-10, low IFN-g and IL-17A, was dominant (like in healthy individuals), whereas without Tregs or with only pTregs, a pro-inflammatory response was dominant, i.e. low IL-10, high IFN-g and IL-17A (as is expected in autoimmune disease patients). These data (shown in FIGS. 9 B-D) demonstrate that Sm-Tregs have the capability to reset the aberrant immune response and restore tolerance to the targeted autoepitope. Results are expressed as mean+/−SEM using samples from four SLE patients, * P<0.05, ** P<0.01 when compared to No Tregs group and pTregs group.

### Example 11— HLA-DR15 Restricted Sm-Tregs Halt the Progression of Nephritis

To demonstrate the efficacy of Sm-Tregs at halting disease progression, the inventors devised a new humanised model of lupus nephritis.

In this model, the adoptive transfer of PBMCs from patients with lupus nephritis into immunocompromised NSGMHCnull mice lead to the development of functional renal injury (measured by an increase in urinary protein) and segmental glomerular necrosis (measured by histological staining of kidney sections). At the onset of functional renal injury, week 3, mice were given either no Tregs, polyclonal Tregs (pTregs) or Sm-Tregs. FIG. 10A provides a schematic of the experimental protocol.

Mice that received no Tregs or pTregs progressed to severe nephritis (i.e high levels of proteinuria and >50% of glomeruli with necrosis), however, the nephritis mice treated with Sm-Tregs did not display further progression of disease (see FIGS. 10B-C). Results are expressed as mean+/−SEM of five SLE patient samples. *** P<0.001 compared to No Tregs and pTregs groups.

### Example 12— HLA-DR3 Restricted Sm-Tregs Halt the Progression of Nephritis

Similarly to the approach outlined in Example 11, the inventors determined whether HLA-DR3 restricted Sm-Tregs also had therapeutic efficacy.

As shown in FIG. 11, HLA-DR3 restricted Sm-Tregs were shown to suppress anti-Sm pro-inflammatory cytokine responses and halt the progression of lupus nephritis. In brief, PBMCs from a HLA-DR3+, anti-Sm+ SLE patient with lupus nephritis was co-cultured with the dominant HLA-DR3 restricted T cell epitope (SmD1:78-92) and either No Tregs, polyclonal Tregs (pTregs) or Tregs transduced with the HLA-DR3 restricted TCR (HLA-DR3 TCR 1 as identified in Table 2) (Sm-Tregs). Cytokine responses were measured at day 8.

^{NSGMHC}null mice received PBMCs from a SLE patient with lupus nephritis who were also positive for anti-Sm antibodies and HLA-DR3+. At the onset of functional renal injury (measured by an increase in proteinuria), week 3, mice were administered either no Tregs, polyclonal Tregs (pTregs) or HLA-DR3 restricted Sm-Tregs (transduced with HLA-DR3 TCR 1).

### CONCLUSIONS

In summary, the results here demonstrate that the inventors have identified highly reactive T cell receptors specific for the Smith (Sm) antigen, a key target autoantigen in lupus. Also shown is that these T cell receptors can be transduced onto human Tregs which can be used to specifically suppress autoimmunity to the Sm antigen.

The inventors have demonstrated that both HLA-DR15 and HLA-DR3 restricted Sm TCRs are therapeutically effective and can be used to halt the progression of autoimmune disease.

Clinically, the present invention allows for a novel antigen-specific regulatory cell based treatment whereby autologous regulatory T cells specific for the Sm antigen are adoptively transferred into lupus patient to suppress their underlying cause of disease and halt disease progression.

Current treatments for lupus are non-specific, and have toxic side effects. The current standard of care for lupus is the use of corticosteroids which itself causes significant side effects including diabetes and osteoporosis, and the use of non-specific immunosuppressive drugs which have harmful side effects and have poor efficacy. The only approved new, add-on treatment for lupus in the last 50 years is the anti-BAFF antibody, belimumab. However, to date, it only has limited clinical efficacy; and its contraindications include active nephritis and central nervous system symptoms. Thus, with existing treatments, accrual of irreversible organ damage is the usual outcome over time, resulting in standardised mortality rates some 2-3-times greater than the healthy community. Thus, there is still a clear and unmet need for better treatments for lupus.

The treatments described herein, i.e. the use of Sm-specific Tregs and the peptides disclosed herein, are expected to enhance the potency of Tregs and induce enhanced immunosuppression with fewer suppressive effects on protective immunity. It is expected that the use of Sm-specific Tregs (and peptides to activate/expand such Tregs) may induce immune tolerance towards autoantigens beyond Smith protein, since immunosuppressive cells recruited as a result of Sm-specific Treg therapy (e.g., Tregs and myeloid derived suppressor cells), exhibit additional non-antigen-specific immunosuppressive capacity, creating a tolerant environment for multiple autoantigens.

It will be understood that the invention disclosed and defined in this specification extends to all alternative combinations of two or more of the individual features mentioned or evident from the text or drawings. All of these different combinations constitute various alternative aspects of the invention.

1. A binding protein comprising a T cell receptor (TCR) α-chain variable (Vα or Valpha) domain and a TCR β-chain variable (Vβ or Vbeta) domain, wherein the binding protein is capable of binding to a complex of a fragment of a Smith protein and an HLA-DR15 or HLA-DR3 molecule.

2. The binding protein according to claim 1, wherein the HLA-DR15 molecule is an HLA-DRA*01:01 and HLA-DRB1*15:01 molecule.

3-9. (canceled)

10. The binding protein according to claim 1, wherein the HLA-DR3 molecule is an HLA-DRA*01:01 and HLA-DRB1*03:01 molecule.

11-13. (canceled)

14. The binding protein according to claim 1, wherein
the Vα domain comprises a CDR3 comprising an amino acid sequence at least about 80%, at least 85%, at least 90%, at least 92%, at least 95%, at least 97%, at least 99% identical to a sequence of any one of SEQ ID NOs: 8, 20, 32, 44, 56, 68, 80, 92, 104, 107, 122, 134, 146, 158, 170, 182, 194, 197, 212, 224, 236 and 248; and/or wherein
the Vβ domain comprises a CDR3 comprising an amino acid sequence at least about 80%, at least 85%, at least 90%, at least 92%, at least 95%, at least 97%, at least 99% identical to a sequence of any one of SEQ ID NOs: 11, 23, 35, 47, 59, 71, 83, 95, 110, 125, 137, 149, 161, 173, 185, 200, 215, 227, 239 and 251; wherein
the binding protein is capable of binding to a complex of a fragment of a Smith protein and an HLA-DR15 molecule.

15. (canceled)

16. The binding protein according to claim 1, wherein
the T cell receptor (TCR) α-chain variable (Vα or Valpha) domain comprises:
(i) a complementarity determining region (CDR) 1 comprising a sequence at least about 80%, at least 85%, at least 90%, at least 92%, at least 95%, at least 97%, at least 99% identical to a sequence set forth in any one of SEQ ID NOs: 6, 18, 30, 42, 54, 66, 78, 90, 102, 105, 120, 132, 144, 156, 168, 180, 192, 195, 210, 222, 234 or 246; a CDR2 comprising a sequence at least about 80%, at least 85%, at least 90%, at least 92%, at least 95%, at least 97%, at least 99% identical to a sequence set in any one of SEQ ID NOs: 7, 19, 31, 43, 55, 67, 79, 91, 103, 106, 121, 133, 145, 157, 169, 181, 193, 196, 211, 223, 235 or 247; and a CDR3 comprising a sequence at least about 80%, at least 85%, at least 90%, at least 92%, at least 95%, at least 97%, at least 99% identical to a sequence set forth in any one of SEQ ID NOs: 8, 20, 32, 44, 56, 68, 80, 92, 104, 107, 122, 134, 146, 158, 170, 182, 194, 197, 212, 224, 236 or 248; or
(ii) a VH comprising a CDR1 comprising a sequence set forth in any one of SEQ ID NOs: 6, 18, 30, 42, 54, 66, 78, 90, 102, 105, 120, 132, 144, 156, 168, 180, 192, 195, 210, 222, 234 or 246, a CDR2 comprising a sequence set forth between in any one of SEQ ID NOs: 7, 19, 31, 43, 55, 67, 79, 91, 103, 106, 121, 133, 145, 157, 169, 181, 193, 196, 211, 223, 235 or 247 and a CDR3 comprising a sequence set forth in any one of SEQ ID NOs: 8, 20, 32, 44, 56, 68, 80, 92, 104, 107, 122, 134, 146, 158, 170, 182, 194, 197, 212, 224, 236 or 248; and wherein
the TCR β-chain variable (Vβ or Vbeta) domain comprises:
(i) a CDR1 comprising a sequence at least about 80%, at least 85%, at least 90%, at least 92%, at least 95%, at least 97%, at least 99% identical to a sequence set forth in any one of SEQ ID NOs: 9, 21, 33, 45, 57, 69, 81, 93, 108, 123, 135, 147, 159, 171, 183, 198, 213, 225, 237 or 249, a CDR2 comprising a sequence at least about 80%, at least 85%, at least 90%, at least 92%, at least 95%, at least 97%, at least 99% identical to a sequence set forth in any one of SEQ ID NOs: 10, 22, 34, 46, 58, 70, 82, 94, 109, 124, 136, 148, 160, 172, 184, 199, 214, 226, 238 or 250 and a CDR3 comprising a sequence at least about 80%, at least 85%, at least 90%, at least 92%, at least 95%, at least 97%, at least 99% identical to a sequence set forth in any one of SEQ ID NOs: 11, 23, 35, 47, 59, 71, 83, 95, 110, 125, 137, 149, 161, 173, 185, 200, 215, 227, 239 or 251; or
(ii) a CDR1 comprising a sequence set forth in any one of SEQ ID NOs: 9, 21, 33, 45, 57, 69, 81, 93, 108, 123, 135, 147, 159, 171, 183, 198, 213, 225, 237 or 249, a CDR2 comprising a sequence set forth in any one of SEQ ID NOs: 10, 22, 34, 46, 58, 70, 82, 94, 109, 124, 136, 148, 160, 172, 184, 199, 214, 226, 238 or 250 and a CDR3 comprising a sequence set forth in any one of SEQ ID NOs: 11, 23, 35, 47, 59, 71, 83, 95, 110, 125, 137, 149, 161, 173, 185, 200, 215, 227, 239 or 251.

17-20. (canceled)

21. The binding protein according to claim 1, wherein
the Vα domain comprises a CDR3 comprising an amino acid sequence at least about 80%, at least 85%, at least 90%, at least 92%, at least 95%, at least 97%, at least 99% identical to a sequence of any one of SEQ ID NOs: 263, 275, 287, 299, 311, 323, 335, 347, 359, 371, 383, 395, 407, 419, 431, 443, 455, 467, 479 and 491; and/or wherein
the Vβ domain comprises a CDR3 comprising an amino acid sequence at least about 80%, at least 85%, at least 90%, at least 92%, at least 95%, at least 97%, at least 99% identical to a sequence of any one of SEQ ID NOs: 266, 278, 290, 302, 314, 326, 338, 350, 362, 374, 386, 398, 410, 422, 434, 446, 458, 470, 482 and 494; wherein
the binding protein is capable of binding to a complex of a fragment of a Smith protein and a HLA-DR3 molecule.

22. (canceled)

23. The binding protein according to claim 1, wherein
the T cell receptor (TCR) α-chain variable (Vα or Valpha) domain comprises:
(i) a complementarity determining region (CDR) 1 comprising a sequence at least about 80%, at least 85%, at least 90%, at least 92%, at least 95%, at least 97%, at least 99% identical to a sequence set forth in any one of SEQ ID NOs: 261, 273, 285, 297, 309, 321, 333, 345, 357, 369, 381, 393, 405, 417, 429, 441, 453, 465, 477 or 489; a CDR2 comprising a sequence at least about 80%, at least 85%, at least 90%, at least 92%, at least 95%, at least 97%, at least 99% identical to a sequence set in any one of SEQ ID NOs: 262, 274, 286, 298, 310, 322, 334, 346, 358, 370, 382, 394, 406, 418, 430, 442, 454, 466, 478, or 490; and a CDR3 comprising a sequence at least about 80%, at least 85%, at least 90%, at least 92%, at least 95%, at least 97%, at least 99% identical to a sequence set forth in any one of SEQ ID NOs: 263, 275, 287, 299, 311, 323, 335, 347, 359, 371, 383, 395, 407, 419, 431, 443, 455, 467, 479 and 491; or
(ii) CDR 1 comprising a sequence set forth in any one of SEQ ID NOs: 261, 273, 285, 297, 309, 321, 333, 345, 357, 369, 381, 393, 405, 417, 429, 441, 453, 465, 477 or 489; a CDR2 comprising a sequence set forth in any one of SEQ ID NOs: 262, 274, 286, 298, 310, 322, 334, 346, 358, 370, 382, 394, 406, 418, 430, 442, 454, 466, 478, or 490; and a CDR3 comprising a sequence set forth in any one of SEQ ID NOs: 263, 275, 287, 299, 311, 323, 335, 347, 359, 371, 383, 395, 407, 419, 431, 443, 455, 467, 479 and 491; and wherein
the TCR β-chain variable (Vβ or Vbeta) domain comprises:
(i) a CDR1 comprising a sequence at least about 80%, at least 85%, at least 90%, at least 92%, at least 95%, at least 97%, at least 99% identical to a sequence set forth in any one of SEQ ID NOs: 264, 276, 288, 300, 312, 324, 336, 348, 360, 372, 384, 396, 408, 420, 432, 444, 456, 468, 480 or 492, a CDR2 comprising a sequence at least about 80%, at least 85%, at least 90%, at least 92%, at least 95%, at least 97%, at least 99% identical to a sequence set forth in any one of SEQ ID NOs: 265, 277, 289, 301, 313, 325, 337, 349, 361, 373, 385, 397, 409, 421, 433, 445, 457, 469, 481 or 493 and a CDR3 comprising a sequence at least about 80%, at least 85%, at least 90%, at least 92%, at least 95%, at least 97%, at least 99% identical to a sequence set forth in any one of SEQ ID NOs: 266, 278, 290, 302, 314, 326, 338, 350, 362, 374, 386, 398, 410, 422, 434, 446, 458, 470, 482 and 494; or
(ii) a CDR1 comprising a sequence set forth in any one of SEQ ID NOs: 264, 276, 288, 300, 312, 324, 336, 348, 360, 372, 384, 396, 408, 420, 432, 444, 456, 468, 480 or 492, a CDR2 comprising a sequence set forth in any one of SEQ ID NOs: 265, 277, 289, 301, 313, 325, 337, 349, 361, 373, 385, 397, 409, 421, 433, 445, 457, 469, 481 or 493 and a CDR3 comprising a sequence set forth in any one of SEQ ID NOs: 266, 278, 290, 302, 314, 326, 338, 350, 362, 374, 386, 398, 410, 422, 434, 446, 458, 470, 482 and 494.

24-25. (canceled)

26. The binding protein according to claim 1, wherein the TCRα chain comprises or consists of an amino acid sequence as set forth in any one of SEQ ID NOS.: 501, 503, 505, 507, 509, 511, 513, 515, 517, 518, 520, 522, 524, 526, 528, 530, 532, 533, 535, 537, 539, and 541; and/or a TCRβ chain that comprises or consists of an amino acid sequence as set forth in any one of SEQ ID Nos: 502, 504, 506, 508, 510, 512, 514, 516, 519, 521, 523, 525, 527, 529, 531, 534, 536, 538, 540, and 542 or any combination thereof.

27. The binding protein according to claim 21, wherein the TCRα chain comprises or consists of an amino acid sequence as set forth in any one of SEQ ID NOs: 585, 587, 589, 591, 593, 595, 597, 599, 601, 603, 605, 607, 609, 611, 613, 615, 617, 619, 621, and 623; and/or a TCRβ chain that comprises or consists of an amino acid sequence as set forth in any one of SEQ ID NOs: 586, 588, 590, 592, 594, 596, 598, 600, 602, 604, 606, 608, 610, 612, 614, 616, 618, 620, 622 and 624 or any combination thereof.

28. The binding protein according to claim 14, wherein the TCRα chain and TCRβ chain are modified to include a cysteine residue that allows formation of an additional interchain disulfide bond.

29. (canceled)

30. A peptide comprising, consisting essentially of or consisting of an amino acid sequence of or equivalent to residues 1 to 15 or 58 to 72 of a SmB/B′ protein.

31-37. (canceled)

38. A nucleic acid comprising, consisting essentially of or consisting of a nucleotide sequence encoding a binding protein according to claim 1.

39. A vector comprising a nucleic acid according to claim 38.

40-46. (canceled)

47. A cell comprising a nucleic acid according to claim 38.

48. (canceled)

49. A method of preparing a population of T regulatory cells for use in the treatment of systemic lupus erythematosus (SLE), the method comprising:
providing a population of T regulatory cells,
introducing a nucleic acid according to claim 38 into the population of T regulatory cells,
providing conditions to allow the expression of the binding protein on the surface of the T regulatory cells,
thereby preparing a population of T regulatory cells for use in the treatment of SLE.

50. A method of preparing a population of T regulatory cells for use in the treatment of systemic lupus erythematosus (SLE), the method comprising providing a mixed population of T cells or a population of T cells exhibiting at least one property of a T conventional cell,
introducing a nucleic acid according to claim 38 into the population of T cells,
providing conditions to allow the expression of the binding protein on the surface of the T cells,
isolating T regulatory cells from the mixed population of T cells, or
alternatively, culturing the cells in conditions for promoting conversion of the cells in the population to T regulatory cells,
optionally, stabilising the converted T regulatory cells,
thereby preparing a population of T regulatory cells for use in the treatment of SLE.

51. A method of preparing a population of T regulatory cells for use in the treatment of systemic lupus erythematosus (SLE), the method comprising:
culturing a population of T cells in the presence of a peptide according to claim 30 under conditions and for a sufficient time to allow expansion of a subpopulation of cells which are activated by the peptide,
optionally, where the population of T cells comprises a mixed population of cells, or comprises conventional T cells, isolating the T regulatory cells from the mixed population or converting the T cells into T regulatory cells,
thereby preparing a population of T regulatory cells for use in the treatment of SLE.

52-66. (canceled)

67. A composition comprising a binding protein according to claim 1 and a pharmaceutically acceptable carrier, diluent or excipient.

68. A method of treating or preventing a condition in a subject, wherein the condition is associated with an aberrant, unwanted or otherwise inappropriate immune response to a Smith protein, the method comprising:
providing a population of T cells exhibiting at least one property of a regulatory T cell,
introducing a nucleic acid according to claim 38 into the population of T cells,
providing conditions to allow the expression of the binding protein on the surface of the T cells,
administering the T cells expressing the binding protein on their surface,
thereby treating or preventing the condition in the subject.

69-70. (canceled)

71. The method according to claim 68, wherein the condition associated with an aberrant, unwanted or otherwise inappropriate immune response to a Smith protein is systemic lupus erythematosus (SLE) or lupus nephritis (LN).

72. (canceled)

73. The binding protein according to claim 21, wherein the TCRα chain and TCRβ chain are modified to include a cysteine residue that allows formation of an additional interchain disulfide bond.


SEQUENCE LISTING (664 sequences)

SEQ ID NO: 1 - protein, 15 aa, Homo sapiens
KMLQHIDYRMRCILQ

SEQ ID NO: 2 - protein, 15 aa, Homo sapiens
RVLGLVLLRGENLVS

SEQ ID NO: 3 - protein, 9 aa, Homo sapiens
IDYRMRCIL

SEQ ID NO: 4 - protein, 9 aa, Homo sapiens
LVLLRGENL

SEQ ID NO: 5 - protein, 233 aa, Homo sapiens
KMLQHIDYRMRCILQDGRIFIGTFKAFDKHMNLILCDCDEFRKIKPKNSKQAEREEKRVLGLVLLRGENLVSMTVEGPPPKDTGIARVPLAGAAGGPGIGRAAGRGIPAGVPMPQAPAGLAGPVRGVGGPSQQVMTPQGRGTVAAAAAAATASIAGAPTQYPPGRGGPPPPMGRGAPPPGMMGPPPGMRPPMGPPMGIPPGRGTPMGMPPPGMRPPPPGMRGPPPPGMRPPRP

SEQ ID NO: 6 - protein, 6 aa, Homo sapiens
ATGYPS

SEQ ID NO: 7 - protein, 7 aa, Homo sapiens
ATKADDK

SEQ ID NO: 8 - protein, 12 aa, Homo sapiens
CALSSYGNKLVF

SEQ ID NO: 9 - protein, 5 aa, Homo sapiens
SGHAT

SEQ ID NO: 10 - protein, 6 aa, Homo sapiens
FQNNGV

SEQ ID NO: 11 - protein, 15 aa, Homo sapiens
CASSSLSGSSYEQYF

SEQ ID NO: 12 - DNA, 18 bp, Homo sapiens
gccacaggatacccttcc

SEQ ID NO: 13 - DNA, 21 bp, Homo sapiens
gccacgaaggctgatgacaag

SEQ ID NO: 14 - DNA, 36 bp, Homo sapiens
tgtgctctgagtagctatggaaacaagctggtcttt

SEQ ID NO: 15 - DNA, 15 bp, Homo sapiens
tctggccatgctacc

SEQ ID NO: 16 - DNA, 18 bp, Homo sapiens
tttcagaataacggtgta

SEQ ID NO: 17 - DNA, 45 bp, Homo sapiens
tgtgccagcagctcgcttagcgggagctcctacgagcagtacttc

SEQ ID NO: 18 - protein, 5 aa, Homo sapiens
TTLSN

SEQ ID NO: 19 - protein, 7 aa, Homo sapiens
LVKSGEV

SEQ ID NO: 20 - protein, 12 aa, Homo sapiens
CAGHAGAQKLVF

SEQ ID NO: 21 - protein, 5 aa, Homo sapiens
MNHNS

SEQ ID NO: 22 - protein, 6 aa, Homo sapiens
SASEGT

SEQ ID NO: 23 - protein, 16 aa, Homo sapiens
CASSEVWVGSTDTQYF

SEQ ID NO: 24 - DNA, 15 bp, Homo sapiens
actactttaagcaat

SEQ ID NO: 25 - DNA, 21 bp, Homo sapiens
ttagtgaagagtggagaagtg

SEQ ID NO: 26 - DNA, 36 bp, Homo sapiens
tgtgcagggcacgcaggagcccagaagctggtattt

SEQ ID NO: 27 - DNA, 15 bp, Homo sapiens
atgaaccataactcc

SEQ ID NO: 28 - DNA, 18 bp, Homo sapiens
tcagcttctgagggtacc

SEQ ID NO: 29 - DNA, 48 bp, Homo sapiens
tgtgccagcagtgaggtatgggtcggtagcacagatacgcagtatttt

SEQ ID NO: 30 - protein, 6 aa, Homo sapiens
NSMFDY

SEQ ID NO: 31 - protein, 7 aa, Homo sapiens
ISSIKDK

SEQ ID NO: 32 - protein, 14 aa, Homo sapiens
CAASRRFGNEKLTF

SEQ ID NO: 33 - protein, 5 aa, Homo sapiens
SEHNR

SEQ ID NO: 34 - protein, 6 aa, Homo sapiens
FQNEAQ

SEQ ID NO: 35 - protein, 16 aa, Homo sapiens
CASRLWSRGRTTEAFF

SEQ ID NO: 36 - DNA, 18 bp, Homo sapiens
aacagcatgtttgattat

SEQ ID NO: 37 - DNA, 21 bp, Homo sapiens
ataagttccattaaggataaa

SEQ ID NO: 38 - DNA, 42 bp, Homo sapiens
tgtgcagcaagcaggcggtttggaaatgagaaattaaccttt

SEQ ID NO: 39 - DNA, 15 bp, Homo sapiens
tctgaacacaaccgc

SEQ ID NO: 40 - DNA, 18 bp, Homo sapiens
ttccagaatgaagctcaa

SEQ ID NO: 41 - DNA, 48 bp, Homo sapiens
tgtgccagcaggctctggagcagggggcggaccactgaagctttcttt

SEQ ID NO: 42 - protein, 6 aa, Homo sapiens
NTAFDY

SEQ ID NO: 43 - protein, 7 aa, Homo sapiens
IRPDVSE

SEQ ID NO: 44 - protein, 14 aa, Homo sapiens
CAASKTGTASKLTF

SEQ ID NO: 45 - protein, 5 aa, Homo sapiens
MDHEN

SEQ ID NO: 46 - protein, 6 aa, Homo sapiens
SYDVKM

SEQ ID NO: 47 - protein, 10 aa, Homo sapiens
CASSVNEQFF

SEQ ID NO: 48 - DNA, 18 bp, Homo sapiens
aacactgcgtttgactac

SEQ ID NO: 49 - DNA, 21 bp, Homo sapiens
atacgtccagatgtgagtgaa

SEQ ID NO: 50 - DNA, 42 bp, Homo sapiens
tgtgcagcaagcaagaccggcactgccagtaaactcaccttt

SEQ ID NO: 51 - DNA, 15 bp, Homo sapiens
atggaccatgaaaat

SEQ ID NO: 52 - DNA, 18 bp, Homo sapiens
tcatatgatgttaaaatg

SEQ ID NO: 53 - DNA, 30 bp, Homo sapiens
tgtgccagcagtgtgaatgagcagttcttc

SEQ ID NO: 54 - protein, 7 aa, Homo sapiens
TISGNEY

SEQ ID NO: 55 - protein, 5 aa, Homo sapiens
GLKNN

SEQ ID NO: 56 - protein, 13 aa, Homo sapiens
CIVKSWNNRKLIW

SEQ ID NO: 57 - protein, 5 aa, Homo sapiens
SEHNR

SEQ ID NO: 58 - protein, 6 aa, Homo sapiens
FQNEAQ

SEQ ID NO: 59 - protein, 16 aa, Homo sapiens
CASLKRTGGHNQPQHF

SEQ ID NO: 60 - DNA, 21 bp, Homo sapiens
accatcagtggaaatgagtat

SEQ ID NO: 61 - DNA, 15 bp, Homo sapiens
ggtctaaaaaacaat

SEQ ID NO: 62 - DNA, 39 bp, Homo sapiens
tgcatcgtcaaaagttggaacaaccgtaagctgatttgg

SEQ ID NO: 63 - DNA, 15 bp, Homo sapiens
tctgaacacaaccgc

SEQ ID NO: 64 - DNA, 18 bp, Homo sapiens
ttccagaatgaagctcaa

SEQ ID NO: 65 - DNA, 48 bp, Homo sapiens
tgtgccagccttaagagaacagggggccacaatcagccccagcatttt

SEQ ID NO: 66 - protein, 6 aa, Homo sapiens
SSVSVY

SEQ ID NO: 67 - protein, 8 aa, Homo sapiens
YLSGSTQV

SEQ ID NO: 68 - protein, 14 aa, Homo sapiens
CAVSVSGSARQLTF

SEQ ID NO: 69 - protein, 5 aa, Homo sapiens
SGHTA

SEQ ID NO: 70 - protein, 6 aa, Homo sapiens
FQGTGA

SEQ ID NO: 71 - protein, 14 aa, Homo sapiens
CSARDPGTLYEQYF

SEQ ID NO: 72 - DNA, 18 bp, Homo sapiens
tcgtctgtttcagtgtat

SEQ ID NO: 73 - DNA, 24 bp, Homo sapiens
tatttatcaggatccacccaggtt

SEQ ID NO: 74 - DNA, 42 bp, Homo sapiens
tgtgctgtgagtgtttctggttctgcaaggcaactgaccttt

SEQ ID NO: 75 - DNA, 15 bp, Homo sapiens
tcaggtcatactgcc

SEQ ID NO: 76 - DNA, 18 bp, Homo sapiens
ttccaaggcacgggtgcg

SEQ ID NO: 77 - DNA, 42 bp, Homo sapiens
tgtgccagcagccaccgtctagcgggagctgagcagttcttc

SEQ ID NO: 78 - protein, 6 aa, Homo sapiens
ATGYPS

SEQ ID NO: 79 - protein, 7 aa, Homo sapiens
ATKADDK

SEQ ID NO: 80 - protein, 14 aa, Homo sapiens
CALTLNSGYSTLTF

SEQ ID NO: 81 - protein, 6 aa, Homo sapiens
DFQATT

SEQ ID NO: 82 - protein, 7 aa, Homo sapiens
SNEGSKA

SEQ ID NO: 83 - protein, 14 aa, Homo sapiens
CSARDPGTLYEQYF

SEQ ID NO: 84 - DNA, 18 bp, Homo sapiens
gccacaggatacccttcc

SEQ ID NO: 85 - DNA, 21 bp, Homo sapiens
gccacgaaggctgatgacaag

SEQ ID NO: 86 - DNA, 42 bp, Homo sapiens
tgtgctctgaccctgaattcaggatacagcaccctcaccttt

SEQ ID NO: 87 - DNA, 18 bp, Homo sapiens
gactttcaggccacaact

SEQ ID NO: 88 - DNA, 21 bp, Homo sapiens
tccaatgagggctccaaggcc

SEQ ID NO: 89 - DNA, 42 bp, Homo sapiens
tgcagtgctagagacccaggaaccttatacgagcagtacttc

SEQ ID NO: 90 - protein, 6 aa, Homo sapiens
VSNAYN

SEQ ID NO: 91 - protein, 4 aa, Homo sapiens
GSKP

SEQ ID NO: 92 - protein, 13 aa, Homo sapiens
CAVERPGFQKLVF

SEQ ID NO: 93 - protein, 5 aa, Homo sapiens
LGHDT

SEQ ID NO: 94 - protein, 6 aa, Homo sapiens
YNNKEL

SEQ ID NO: 95 - protein, 14 aa, Homo sapiens
CASRTTGSGTEAFF

SEQ ID NO: 96 - DNA, 18 bp, Homo sapiens
gtgtccaatgcttacaac

SEQ ID NO: 97 - DNA, 12 bp, Homo sapiens
ggctcaaagcct

SEQ ID NO: 98 - DNA, 39 bp, Homo sapiens
tgtgctgtggagcggccaggctttcagaaacttgtattt

SEQ ID NO: 99 - DNA, 15 bp, Homo sapiens
ctgggccatgatact

SEQ ID NO: 100 - DNA, 18 bp, Homo sapiens
tacaataataaggagctc

SEQ ID NO: 101 - DNA, 42 bp, Homo sapiens
tgtgccagcaggacgacagggtcggggactgaagctttcttt

SEQ ID NO: 102 - protein, 5 aa, Homo sapiens
TSINN

SEQ ID NO: 103 - protein, 7 aa, Homo sapiens
IRSNERE

SEQ ID NO: 104 - protein, 13 aa, Homo sapiens
CATPRRGADGLTF

SEQ ID NO: 105 - protein, 5 aa, Homo sapiens
SVFSS

SEQ ID NO: 106 - protein, 7 aa, Homo sapiens
VVTGGEV

SEQ ID NO: 107 - protein, 9 aa, Homo sapiens
CAFCNQFYF

SEQ ID NO: 108 - protein, 5 aa, Homo sapiens
LGHNA

SEQ ID NO: 109 - protein, 6 aa, Homo sapiens
YNFKEQ

SEQ ID NO: 110 - protein, 17 aa, Homo sapiens
CASSPGGSGRLGDTQYF

SEQ ID NO: 111 - DNA, 15 bp, Homo sapiens
actagtataaacaat

SEQ ID NO: 112 - DNA, 21 bp, Homo sapiens
atacgttcaaatgaaagagag

SEQ ID NO: 113 - DNA, 39 bp, Homo sapiens
tgtgctacgccgaggcgaggtgctgacggactcaccttt

SEQ ID NO: 114 - DNA, 15 bp, Homo sapiens
agtgttttttccagc

SEQ ID NO: 115 - DNA, 21 bp, Homo sapiens
gtagttacgggtggagaagtg

SEQ ID NO: 116 - DNA, 27 bp, Homo sapiens
tgtgccttttgtaaccagttctatttt

SEQ ID NO: 117 - DNA, 15 bp, Homo sapiens
ctggggcataacgct

SEQ ID NO: 118 - DNA, 18 bp, Homo sapiens
tacaactttaaagaacag

SEQ ID NO: 119 - DNA, 51 bp, Homo sapiens
tgtgccagcagcccggggggtagcgggaggttgggagatacgcagtatttt

SEQ ID NO: 120 - protein, 6 aa, Homo sapiens
VSNAYN

SEQ ID NO: 121 - protein, 4 aa, Homo sapiens
GSKP

SEQ ID NO: 122 - protein, 14 aa, Homo sapiens
CAVEDSRGNNRLAF

SEQ ID NO: 123 - protein, 5 aa, Homo sapiens
MNHEY

SEQ ID NO: 124 - protein, 6 aa, Homo sapiens
SVGAGI

SEQ ID NO: 125 - protein, 14 aa, Homo sapiens
CASGGTGGNYGYTF

SEQ ID NO: 126 - DNA, 18 bp, Homo sapiens
gtgtccaatgcttacaac

SEQ ID NO: 127 - DNA, 12 bp, Homo sapiens
ggctcaaagcct

SEQ ID NO: 128 - DNA, 42 bp, Homo sapiens
tgtgctgtggaggattcgcgggggaacaacagactcgctttt

SEQ ID NO: 129 - DNA, 15 bp, Homo sapiens
atgaaccatgaatac

SEQ ID NO: 130 - DNA, 18 bp, Homo sapiens
tcagttggtgctggtatc

SEQ ID NO: 131 - DNA, 42 bp, Homo sapiens
tgtgccagcggagggacaggggggaactatggctacaccttc

SEQ ID NO: 132 - protein, 7 aa, Homo sapiens
TSENNYY

SEQ ID NO: 133 - protein, 8 aa, Homo sapiens
QEAYKQQN

SEQ ID NO: 134 - protein, 15 aa, Homo sapiens
CAFMKSLFGNEKLTF

SEQ ID NO: 135 - protein, 6 aa, Homo sapiens
DFQATT

SEQ ID NO: 136 - protein, 7 aa, Homo sapiens
SNEGSKA

SEQ ID NO: 137 - protein, 11 aa, Homo sapiens
CSARSGEKLFF

SEQ ID NO: 138 - DNA, 21 bp, Homo sapiens
accagtgagaataattattat

SEQ ID NO: 139 - DNA, 24 bp, Homo sapiens
caagaagcttataagcaacagaat

SEQ ID NO: 140 - DNA, 45 bp, Homo sapiens
tgtgctttcatgaagtcgctatttggaaatgagaaattaaccttt

SEQ ID NO: 141 - DNA, 18 bp, Homo sapiens
gactttcaggccacaact

SEQ ID NO: 142 - DNA, 21 bp, Homo sapiens
tccaatgagggctccaaggcc

SEQ ID NO: 143 - DNA, 33 bp, Homo sapiens
tgcagtgctaggtcgggcgaaaaactgtttttt

SEQ ID NO: 144 - protein, 7 aa, Homo sapiens
TSENNYY

SEQ ID NO: 145 - protein, 8 aa, Homo sapiens
QEAYKQQN

SEQ ID NO: 146 - protein, 15 aa, Homo sapiens
CAFMKSLFGNEKLTF

SEQ ID NO: 147 - protein, 6 aa, Homo sapiens
DFQATT

SEQ ID NO: 148 - protein, 7 aa, Homo sapiens
SNEGSKA

SEQ ID NO: 149 - protein, 12 aa, Homo sapiens
CSARWGDQPQHF

SEQ ID NO: 150 - DNA, 21 bp, Homo sapiens
accagtgagaataattattat

SEQ ID NO: 151 - DNA, 24 bp, Homo sapiens
caagaagcttataagcaacagaat

SEQ ID NO: 152 - DNA, 45 bp, Homo sapiens
tgtgctttcatgaagtctccctttggaaatgagaaattaaccttt

SEQ ID NO: 153 - DNA, 18 bp, Homo sapiens
gactttcaggccacaact

SEQ ID NO: 154 - DNA, 21 bp, Homo sapiens
tccaatgagggctccaaggcc

SEQ ID NO: 155 - DNA, 36 bp, Homo sapiens
tgcagtgctaggtggggtgatcagccccagcatttt

SEQ ID NO: 156 - protein, 7 aa, Homo sapiens
TSENNYY

SEQ ID NO: 157 - protein, 8 aa, Homo sapiens
QEAYKQQN

SEQ ID NO: 158 - protein, 15 aa, Homo sapiens
CAFMKTNFGNEKLTF

SEQ ID NO: 159 - protein, 6 aa, Homo sapiens
DFQATT

SEQ ID NO: 160 - protein, 7 aa, Homo sapiens
SNEGSKA

SEQ ID NO: 161 - protein, 11 aa, Homo sapiens
CSARSGTSKFF

SEQ ID NO: 162 - DNA, 21 bp, Homo sapiens
accagtgagaataattattat

SEQ ID NO: 163 - DNA, 24 bp, Homo sapiens
caagaagcttataagcaacagaat

SEQ ID NO: 164 - DNA, 45 bp, Homo sapiens
tgtgctttcatgaagactaactttggaaatgagaaattaaccttt

SEQ ID NO: 165 - DNA, 18 bp, Homo sapiens
gactttcaggccacaact

SEQ ID NO: 166 - DNA, 21 bp, Homo sapiens
tccaatgagggctccaaggcc

SEQ ID NO: 167 - DNA, 33 bp, Homo sapiens
tgcagtgctaggtccgggactagcaaattcttc

SEQ ID NO: 168 - protein, 7 aa, Homo sapiens
TSENNYY

SEQ ID NO: 169 - protein, 8 aa, Homo sapiens
QEAYKQQN

SEQ ID NO: 170 - protein, 15 aa, Homo sapiens
CAFTKSNFGNEKLTF

SEQ ID NO: 171 - protein, 6 aa, Homo sapiens
DFQATT

SEQ ID NO: 172 - protein, 7 aa, Homo sapiens
SNEGSKA

SEQ ID NO: 173 - protein, 11 aa, Homo sapiens
CSARTGDEQYF

SEQ ID NO: 174 - DNA, 21 bp, Homo sapiens
accagtgagaataattattat

SEQ ID NO: 175 - DNA, 24 bp, Homo sapiens
caagaagcttataagcaacagaat

SEQ ID NO: 176 - DNA, 45 bp, Homo sapiens
tgtgctttcacgaagtctaactttggaaatgagaaattaaccttt

SEQ ID NO: 177 - DNA, 18 bp, Homo sapiens
gactttcaggccacaact

SEQ ID NO: 178 - DNA, 21 bp, Homo sapiens
tccaatgagggctccaaggcc

SEQ ID NO: 179 - DNA, 33 bp, Homo sapiens
tgcagtgctcggacaggagacgagcagtacttc

SEQ ID NO: 180 - protein, 7 aa, Homo sapiens
TSENNYY

SEQ ID NO: 181 - protein, 8 aa, Homo sapiens
QEAYKQQN

SEQ ID NO: 182 - protein, 15 aa, Homo sapiens
CAFIKSNFGNEKLTF

SEQ ID NO: 183 - protein, 6 aa, Homo sapiens
DFQATT

SEQ ID NO: 184 - protein, 7 aa, Homo sapiens
SNEGSKA

SEQ ID NO: 185 - protein, 11 aa, Homo sapiens
CSARRGTEAFF

SEQ ID NO: 186 - DNA, 21 bp, Homo sapiens
accagtgagaataattattat

SEQ ID NO: 187 - DNA, 24 bp, Homo sapiens
caagaagcttataagcaacagaat

SEQ ID NO: 188 - DNA, 45 bp, Homo sapiens
tgtgctttcatcaaatctaactttggaaatgagaaattaaccttt

SEQ ID NO: 189 - DNA, 18 bp, Homo sapiens
gactttcaggccacaact

SEQ ID NO: 190 - DNA, 21 bp, Homo sapiens
tccaatgagggctccaaggcc

SEQ ID NO: 191 - DNA, 33 bp, Homo sapiens
tgcagtgcccggagggggactgaagctttcttt

SEQ ID NO: 192 - protein, 6 aa, Homo sapiens
TSGFNG

SEQ ID NO: 193 - protein, 6 aa, Homo sapiens
NVLDGL

SEQ ID NO: 194 - protein, 12 aa, Homo sapiens
CAVMDSSYKLIF

SEQ ID NO: 195 - protein, 7 aa, Homo sapiens
NIATNDY

SEQ ID NO: 196 - protein, 5 aa, Homo sapiens
GYKTK

SEQ ID NO: 197 - protein, 7 aa, Homo sapiens
CLVKIIF

SEQ ID NO: 198 - protein, 5 aa, Homo sapiens
MRHNA

SEQ ID NO: 199 - protein, 6 aa, Homo sapiens
SNTAGT

SEQ ID NO: 200 - protein, 13 aa, Homo sapiens
CASSEREHYGYTF

SEQ ID NO: 201 - DNA, 18 bp, Homo sapiens
acatctgggttcaacggg

SEQ ID NO: 202 - DNA, 18 bp, Homo sapiens
aatgttctggatggtttg

SEQ ID NO: 203 - DNA, 36 bp, Homo sapiens
tgtgctgtgatggatagcagctataaattgatcttc

SEQ ID NO: 204 - DNA, 21 bp, Homo sapiens
aacattgctacaaatgattat

SEQ ID NO: 205 - DNA, 15 bp, Homo sapiens
ggatacaagacaaaa

SEQ ID NO: 206 - DNA, 21 bp, Homo sapiens
tgcctcgtgaagataatcttt

SEQ ID NO: 207 - DNA, 15 bp, Homo sapiens
atgagacataatgcc

SEQ ID NO: 208 - DNA, 18 bp, Homo sapiens
tcaaatactgcaggtacc

SEQ ID NO: 209 - DNA, 39 bp, Homo sapiens
tgtgccagcagtgaaagggagcactatggctacaccttc

SEQ ID NO: 210 - protein, 5 aa, Homo sapiens
TSINN

SEQ ID NO: 211 - protein, 7 aa, Homo sapiens
IRSNERE

SEQ ID NO: 212 - protein, 13 aa, Homo sapiens
CATPYTGGFKTIF

SEQ ID NO: 213 - protein, 5 aa, Homo sapiens
MGHRA

SEQ ID NO: 214 - protein, 6 aa, Homo sapiens
YSYEKL

SEQ ID NO: 215 - protein, 14 aa, Homo sapiens
CASSQLGSGNTIYF

SEQ ID NO: 216 - DNA, 15 bp, Homo sapiens
actagtataaacaat

SEQ ID NO: 217 - DNA, 21 bp, Homo sapiens
atacgttcaaatgaaagagag

SEQ ID NO: 218 - DNA, 39 bp, Homo sapiens
tgtgctacgccatatactggaggcttcaaaactatcttt

SEQ ID NO: 219 - DNA, 15 bp, Homo sapiens
atggggcacagggct

SEQ ID NO: 220 - DNA, 18 bp, Homo sapiens
tacagctatgagaaactc

SEQ ID NO: 221 - DNA, 42 bp, Homo sapiens
tgcgccagcagccaacttggctctggaaacaccatatatttt

SEQ ID NO: 222 - protein, 6 aa, Homo sapiens
DSAIYN

SEQ ID NO: 223 - protein, 7 aa, Homo sapiens
IQSSQRE

SEQ ID NO: 224 - protein, 16 aa, Homo sapiens
CAVKAQGGAGSYQLTF

SEQ ID NO: 225 - protein, 5 aa, Homo sapiens
SGHNT

SEQ ID NO: 226 - protein, 6 aa, Homo sapiens
YYREEE

SEQ ID NO: 227 - protein, 13 aa, Homo sapiens
CGSGRERTDTQYF

SEQ ID NO: 228 - DNA, 18 bp, Homo sapiens
gatagcgctatttacaac

SEQ ID NO: 229 - DNA, 21 bp, Homo sapiens
attcagtcaagtcagagagag

SEQ ID NO: 230 - DNA, 48 bp, Homo sapiens
tgtgctgtgaaggcccagggtggggctgggagttaccaactcactttc

SEQ ID NO: 231 - DNA, 15 bp, Homo sapiens
tctgggcacaacact

SEQ ID NO: 232 - DNA, 18 bp, Homo sapiens
tattatagggaggaagag

SEQ ID NO: 233 - DNA, 39 bp, Homo sapiens
tgtgggtccgggcgggaacgcacagatacgcagtatttt

SEQ ID NO: 234 - protein, 5 aa, Homo sapiens
SVFSS

SEQ ID NO: 235 - protein, 7 aa, Homo sapiens
VVTGGEV

SEQ ID NO: 236 - protein, 12 aa, Homo sapiens
CAGANSGYALNF

SEQ ID NO: 237 - protein, 5 aa, Homo sapiens
MDHEN

SEQ ID NO: 238 - protein, 6 aa, Homo sapiens
SYDVKM

SEQ ID NO: 239 - protein, 15 aa, Homo sapiens
CASSSLPGQLYGYTF

SEQ ID NO: 240 - DNA, 15 bp, Homo sapiens
agtgttttttccagc

SEQ ID NO: 241 - DNA, 21 bp, Homo sapiens
gtagttacgggtggagaagtg

SEQ ID NO: 242 - DNA, 36 bp, Homo sapiens
tgtgcaggagctaattccgggtatgcactcaacttc

SEQ ID NO: 243 - DNA, 15 bp, Homo sapiens
atggaccatgaaaat

SEQ ID NO: 244 - DNA, 18 bp, Homo sapiens
tcatatgatgttaaaatg

SEQ ID NO: 245 - DNA, 45 bp, Homo sapiens
tgtgccagcagttccctaccgggacagctttatggctacaccttc

SEQ ID NO: 246 - protein, 6 aa, Homo sapiens
TSGFYG

SEQ ID NO: 247 - protein, 6 aa, Homo sapiens
NALDGL

SEQ ID NO: 248 - protein, 10 aa, Homo sapiens
CAEGGFKTIF

SEQ ID NO: 249 - protein, 5 aa, Homo sapiens
KGHDR

SEQ ID NO: 250 - protein, 6 aa, Homo sapiens
SFDVKD

SEQ ID NO: 251 - protein, 15 aa, Homo sapiens
CATSGPLTGPPEQFF

SEQ ID NO: 252 - DNA, 18 bp, Homo sapiens
acatctgggttttatggg

SEQ ID NO: 253 - DNA, 18 bp, Homo sapiens
aatgctctggatggtttg

SEQ ID NO: 254 - DNA, 30 bp, Homo sapiens
tgcgctgagggaggcttcaaaactatcttt

SEQ ID NO: 255 - DNA, 15 bp, Homo sapiens
aagggtcatgataga

SEQ ID NO: 256 - DNA, 18 bp, Homo sapiens
tcctttgatgtcaaagat

SEQ ID NO: 257 - DNA, 45 bp, Homo sapiens
tgtgccaccagtggccccctcacagggccccccgagcagttcttc

SEQ ID NO: 258 - protein, 15 aa, Homo sapiens
TLLVDVEPKVKSKKR

SEQ ID NO: 259 - protein, 15 aa, Homo sapiens
DYRMRCILQDGRIFI

SEQ ID NO: 260 - protein, 119 aa, Homo sapiens
MKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPRR

SEQ ID NO: 261 - protein, 6 aa, Homo sapiens
VSGLRG

SEQ ID NO: 262 - protein, 7 aa, Homo sapiens
LYSAGEE

SEQ ID NO: 263 - protein, 12 aa, Homo sapiens
CAAAIGSYIPTF

SEQ ID NO: 264 - protein, 5 aa, Homo sapiens
SGHRS

SEQ ID NO: 265 - protein, 6 aa, Homo sapiens
YFSETQ

SEQ ID NO: 266 - protein, 14 aa, Homo sapiens
CASSLGNSNTEAFF

SEQ ID NO: 267 - DNA, 18 bp, Homo sapiens
gtcagcggtttaagaggg

SEQ ID NO: 268 - DNA, 21 bp, Homo sapiens
ctgtattcagctggggaagaa

SEQ ID NO: 269 - DNA, 36 bp, Homo sapiens
tgtgctgcggccataggaagctacatacctacattt

SEQ ID NO: 270 - DNA, 15 bp, Homo sapiens
tctgggcataggagt

SEQ ID NO: 271 - DNA, 18 bp, Homo sapiens
tacttcagtgagacacag

SEQ ID NO: 272 - DNA, 42 bp, Homo sapiens
tgcgccagcagcttggggaattcgaacactgaagctttcttt

SEQ ID NO: 273 - protein, 6 aa, Homo sapiens
DRGSQS

SEQ ID NO: 274 - protein, 6 aa, Homo sapiens
IYSNGD

SEQ ID NO: 275 - protein, 15 aa, Homo sapiens
CAVRESGGGADGLTF

SEQ ID NO: 276 - protein, 5 aa, Homo sapiens
SQVTM

SEQ ID NO: 277 - protein, 7 aa, Homo sapiens
ANQGSEA

SEQ ID NO: 278 - protein, 14 aa, Homo sapiens
CSVGEQGMTQPQHF

SEQ ID NO: 279 - DNA, 18 bp, Homo sapiens
gaccgaggttcccagtcc

SEQ ID NO: 280 - DNA, 18 bp, Homo sapiens
atatactccaatggtgac

SEQ ID NO: 281 - DNA, 45 bp, Homo sapiens
tgtgccgtgagggagtcaggaggaggtgctgacggactcaccttt

SEQ ID NO: 282 - DNA, 15 bp, Homo sapiens
agccaagtcaccatg

SEQ ID NO: 283 - DNA, 21 bp, Homo sapiens
gcaaatcagggctctgaggcc

SEQ ID NO: 284 - DNA, 42 bp, Homo sapiens
tgcagcgttggggaacagggtatgacgcagccccagcatttt

SEQ ID NO: 285 - protein, 7 aa, Homo sapiens
TISGTDY

SEQ ID NO: 286 - protein, 5 aa, Homo sapiens
GLTSN

SEQ ID NO: 287 - protein, 13 aa, Homo sapiens
CILRYQGAQKLVF

SEQ ID NO: 288 - protein, 5 aa, Homo sapiens
MGHRA

SEQ ID NO: 289 - protein, 6 aa, Homo sapiens
YSYEKL

SEQ ID NO: 290 - protein, 17 aa, Homo sapiens
CASSQVRERKNTGELFF

SEQ ID NO: 291 - DNA, 21 bp, Homo sapiens
acaatcagtggaactgattac

SEQ ID NO: 292 - DNA, 15 bp, Homo sapiens
ggtcttacaagcaat

SEQ ID NO: 293 - DNA, 39 bp, Homo sapiens
tgcatcctgagatatcagggagcccagaagctggtattt

SEQ ID NO: 294 - DNA, 15 bp, Homo sapiens
atggggcacagggct

SEQ ID NO: 295 - DNA, 18 bp, Homo sapiens
tacagctatgagaaactc

SEQ ID NO: 296 - DNA, 51 bp, Homo sapiens
tgcgccagcagccaagtaagggagcgcaagaacaccggggagctgtttttt

SEQ ID NO: 297 - protein, 5 aa, Homo sapiens
KALYS

SEQ ID NO: 298 - protein, 7 aa, Homo sapiens
LLKGGEQ

SEQ ID NO: 299 - protein, 12 aa, Homo sapiens
CGTGAGSYQLTF

SEQ ID NO: 300 - protein, 5 aa, Homo sapiens
MGHRA

SEQ ID NO: 301 - protein, 6 aa, Homo sapiens
YSYEKL

SEQ ID NO: 302 - protein, 19 aa, Homo sapiens
CASSHPGLRAGASYNEQFF

SEQ ID NO: 303 - DNA, 15 bp, Homo sapiens
aaggctttatattct

SEQ ID NO: 304 - DNA, 21 bp, Homo sapiens
ttactgaagggtggagaacag

SEQ ID NO: 305 - DNA, 36 bp, Homo sapiens
tgcggcactggggctgggagttaccaactcactttc

SEQ ID NO: 306 - DNA, 15 bp, Homo sapiens
atggggcacagggct

SEQ ID NO: 307 - DNA, 18 bp, Homo sapiens
tacagctatgagaaactc

SEQ ID NO: 308 - DNA, 57 bp, Homo sapiens
tgcgccagcagccaccccggcttaagggcgggagcctcctacaatgagcagttcttc

SEQ ID NO: 309 - protein, 6 aa, Homo sapiens
VTNFRS

SEQ ID NO: 310 - protein, 7 aa, Homo sapiens
LTSSGIE

SEQ ID NO: 311 - protein, 12 aa, Homo sapiens
CAVLNTGNQFYF

SEQ ID NO: 312 - protein, 5 aa, Homo sapiens
MGHRA

SEQ ID NO: 313 - protein, 6 aa, Homo sapiens
YSYEKL

SEQ ID NO: 314 - protein, 16 aa, Homo sapiens
CASSRFPGGGYNEQFF

SEQ ID NO: 315 - DNA, 18 bp, Homo sapiens
gtgactaactttcgaagc

SEQ ID NO: 316 - DNA, 21 bp, Homo sapiens
ctaacttcaagtggaattgaa

SEQ ID NO: 317 - DNA, 36 bp, Homo sapiens
tgtgctgtcttgaacaccggtaaccagttctatttt

SEQ ID NO: 318 - DNA, 15 bp, Homo sapiens
atggggcacagggct

SEQ ID NO: 319 - DNA, 18 bp, Homo sapiens
tacagctatgagaaactc

SEQ ID NO: 320 - DNA, 48 bp, Homo sapiens
tgcgccagcagtcggtttccgggcgggggctacaatgagcagttcttc

SEQ ID NO: 321 - protein, 6 aa, Homo sapiens
NSASQS

SEQ ID NO: 322 - protein, 6 aa, Homo sapiens
VYSSGN

SEQ ID NO: 323 - protein, 13 aa, Homo sapiens
CVVNWSGNEKLTF

SEQ ID NO: 324 - protein, 5 aa, Homo sapiens
KGHDR

SEQ ID NO: 325 - protein, 6 aa, Homo sapiens
SFDVKD

SEQ ID NO: 326 - protein, 13 aa, Homo sapiens
CATRGQGVGELFF

SEQ ID NO: 327 - DNA, 18 bp, Homo sapiens
aacagtgcttctcagtct

SEQ ID NO: 328 - DNA, 18 bp, Homo sapiens
gtatactccagtggtaat

SEQ ID NO: 329 - DNA, 39 bp, Homo sapiens
tgtgtggtgaactggtccggaaatgagaaattaaccttt

SEQ ID NO: 330 - DNA, 15 bp, Homo sapiens
aagggtcatgataga

SEQ ID NO: 331 - DNA, 18 bp, Homo sapiens
tcctttgatgtcaaagat

SEQ ID NO: 332 - DNA, 39 bp, Homo sapiens
tgtgccaccaggggacagggggtcggggagctgtttttt

SEQ ID NO: 333 - protein, 6 aa, Homo sapiens
NSAFQY

SEQ ID NO: 334 - protein, 6 aa, Homo sapiens
TYSSGN

SEQ ID NO: 335 - protein, 14 aa, Homo sapiens
CAMSVQAAGNKLTF

SEQ ID NO: 336 - protein, 5 aa, Homo sapiens
SGHAT

SEQ ID NO: 337 - protein, 6 aa, Homo sapiens
FQNNGV

SEQ ID NO: 338 - protein, 13 aa, Homo sapiens
CASSLDRNYGYTF

SEQ ID NO: 339 - DNA, 18 bp, Homo sapiens
aacagtgcttttcaatac

SEQ ID NO: 340 - DNA, 18 bp, Homo sapiens
acatactccagtggtaac

SEQ ID NO: 341 - DNA, 42 bp, Homo sapiens
tgtgcaatgagcgtacaggctgcaggcaacaagctaactttt

SEQ ID NO: 342 - DNA, 15 bp, Homo sapiens
tctggccatgctacc

SEQ ID NO: 343 - DNA, 18 bp, Homo sapiens
tttcagaataacggtgta

SEQ ID NO: 344 - DNA, 39 bp, Homo sapiens
tgtgccagcagcttagacaggaactatggctacaccttc

SEQ ID NO: 345 - protein, 6 aa, Homo sapiens
ATGYPS

SEQ ID NO: 346 - protein, 7 aa, Homo sapiens
ATKADDK

SEQ ID NO: 347 - protein, 14 aa, Homo sapiens
CALSDNTNAGKSTF

SEQ ID NO: 348 - protein, 6 aa, Homo sapiens
DFQATT

SEQ ID NO: 349 - protein, 7 aa, Homo sapiens
SNEGSKA

SEQ ID NO: 350 - protein, 13 aa, Homo sapiens
CSARTSGQYEQYF

SEQ ID NO: 351 - DNA, 18 bp, Homo sapiens
gccacaggatacccttcc

SEQ ID NO: 352 - DNA, 21 bp, Homo sapiens
gccacgaaggctgatgacaag

SEQ ID NO: 353 - DNA, 42 bp, Homo sapiens
tgtgctctgagtgataacaccaatgcaggcaaatcaaccttt

SEQ ID NO: 354 - DNA, 18 bp, Homo sapiens
gactttcaggccacaact

SEQ ID NO: 355 - DNA, 21 bp, Homo sapiens
tccaatgagggctccaaggcc

SEQ ID NO: 356 - DNA, 39 bp, Homo sapiens
tgcagtgctaggactagcggtcaatacgagcagtacttc

SEQ ID NO: 357 - protein, 6 aa, Homo sapiens
ATGYPS

SEQ ID NO: 358 - protein, 7 aa, Homo sapiens
ATKADDK

SEQ ID NO: 359 - protein, 17 aa, Homo sapiens
CALSDLLDSGNTGKLIF

SEQ ID NO: 360 - protein, 5 aa, Homo sapiens
SGDLS

SEQ ID NO: 361 - protein, 6 aa, Homo sapiens
YYNGEE

SEQ ID NO: 362 - protein, 12 aa, Homo sapiens
CASRGSYNEQFF

SEQ ID NO: 363 - DNA, 18 bp, Homo sapiens
gccacaggatacccttcc

SEQ ID NO: 364 - DNA, 21 bp, Homo sapiens
gccacgaaggctgatgacaag

SEQ ID NO: 365 - DNA, 51 bp, Homo sapiens
tgtgctctgagtgatctgttggactctggcaacacaggcaaactaatcttt

SEQ ID NO: 366 - DNA, 15 bp, Homo sapiens
tctggagacctctct

SEQ ID NO: 367 - DNA, 18 bp, Homo sapiens
tattataatggagaagag

SEQ ID NO: 368 - DNA, 36 bp, Homo sapiens
tgtgccagtagaggatcctacaatgagcagttcttc

SEQ ID NO: 369 - protein, 6 aa, Homo sapiens
NSASQS

SEQ ID NO: 370 - protein, 6 aa, Homo sapiens
VYSSGN

SEQ ID NO: 371 - protein, 12 aa, Homo sapiens
CVVNTPSSKIIF

SEQ ID NO: 372 - protein, 6 aa, Homo sapiens
DFQATT

SEQ ID NO: 373 - protein, 7 aa, Homo sapiens
SNEGSKA

SEQ ID NO: 374 - protein, 11 aa, Homo sapiens
CSARSGQGQFF

SEQ ID NO: 375 - DNA, 18 bp, Homo sapiens
aacagtgcttctcagtct

SEQ ID NO: 376 - DNA, 18 bp, Homo sapiens
gtatactccagtggtaat

SEQ ID NO: 377 - DNA, 36 bp, Homo sapiens
tgtgtggtgaacactccctcttccaagataatcttt

SEQ ID NO: 378 - DNA, 18 bp, Homo sapiens
gactttcaggccacaact

SEQ ID NO: 379 - DNA, 21 bp, Homo sapiens
tccaatgagggctccaaggcc

SEQ ID NO: 380 - DNA, 33 bp, Homo sapiens
tgcagtgctagatcgggacaggggcagttcttc

SEQ ID NO: 381 - protein, 6 aa, Homo sapiens
NSASQS

SEQ ID NO: 382 - protein, 6 aa, Homo sapiens
VYSSGN

SEQ ID NO: 383 - protein, 13 aa, Homo sapiens
CVVPPGGGFKTIF

SEQ ID NO: 384 - protein, 5 aa, Homo sapiens
MNHEY

SEQ ID NO: 385 - protein, 6 aa, Homo sapiens
SMNVEV

SEQ ID NO: 386 - protein, 14 aa, Homo sapiens
CASRTRTASYGYTF

SEQ ID NO: 387 - DNA, 18 bp, Homo sapiens
aacagtgcttctcagtct

SEQ ID NO: 388 - DNA, 18 bp, Homo sapiens
gtatactccagtggtaat

SEQ ID NO: 389 - DNA, 39 bp, Homo sapiens
tgtgtggtgcctcctgggggaggcttcaaaactatcttt

SEQ ID NO: 390 - DNA, 15 bp, Homo sapiens
atgaaccatgagtat

SEQ ID NO: 391 - DNA, 18 bp, Homo sapiens
tcaatgaatgttgaggtg

SEQ ID NO: 392 - DNA, 42 bp, Homo sapiens
tgtgccagcaggaccaggacagccagttatggctacaccttc

SEQ ID NO: 393 - protein, 6 aa, Homo sapiens
TSGFNG

SEQ ID NO: 394 - protein, 6 aa, Homo sapiens
NVLDGL

SEQ ID NO: 395 - protein, 12 aa, Homo sapiens
CAVLDSNYQLIW

SEQ ID NO: 396 - protein, 5 aa, Homo sapiens
MNHNS

SEQ ID NO: 397 - protein, 6 aa, Homo sapiens
SASEGT

SEQ ID NO: 398 - protein, 15 aa, Homo sapiens
CASSEEASGSYEQYF

SEQ ID NO: 399 - DNA, 18 bp, Homo sapiens
acatctgggttcaacggg

SEQ ID NO: 400 - DNA, 18 bp, Homo sapiens
aatgttctggatggtttg

SEQ ID NO: 401 - DNA, 36 bp, Homo sapiens
tgtgctgtcctggatagcaactatcagttaatctgg

SEQ ID NO: 402 - DNA, 15 bp, Homo sapiens
atgaaccataactcc

SEQ ID NO: 403 - DNA, 18 bp, Homo sapiens
tcagcttctgagggtacc

SEQ ID NO: 404 - DNA, 45 bp, Homo sapiens
tgtgccagcagtgaagaggctagcgggtcctacgagcagtacttc

SEQ ID NO: 405 - protein, 6 aa, Homo sapiens
DRGSQS

SEQ ID NO: 406 - protein, 6 aa, Homo sapiens
IYSNGD

SEQ ID NO: 407 - protein, 10 aa, Homo sapiens
CAVSGNQFYF

SEQ ID NO: 408 - protein, 5 aa, Homo sapiens
SEHNR

SEQ ID NO: 409 - protein, 6 aa, Homo sapiens
FQNEAQ

SEQ ID NO: 410 - protein, 16 aa, Homo sapiens
CASSPLGSGRYNEQFF

SEQ ID NO: 411 - DNA, 18 bp, Homo sapiens
gaccgaggttcccagtcc

SEQ ID NO: 412 - DNA, 18 bp, Homo sapiens
atatactccaatggtgac

SEQ ID NO: 413 - DNA, 30 bp, Homo sapiens
tgtgccgtctcagggaaccagttctatttt

SEQ ID NO: 414 - DNA, 15 bp, Homo sapiens
tctgaacacaaccgc

SEQ ID NO: 415 - DNA, 18 bp, Homo sapiens
ttccagaatgaagctcaa

SEQ ID NO: 416 - DNA, 48 bp, Homo sapiens
tgtgccagcagccccctgggtagcgggaggtacaatgagcagttcttc

SEQ ID NO: 417 - protein, 6 aa, Homo sapiens
YGATPY

SEQ ID NO: 418 - protein, 8 aa, Homo sapiens
YFSGDTLV

SEQ ID NO: 419 - protein, 15 aa, Homo sapiens
CAVGPFSGGYQKVTF

SEQ ID NO: 420 - protein, 5 aa, Homo sapiens
SNHLY

SEQ ID NO: 421 - protein, 6 aa, Homo sapiens
FYNNEI

SEQ ID NO: 422 - protein, 15 aa, Homo sapiens
CASREVHSGANVLTF

SEQ ID NO: 423 - DNA, 18 bp, Homo sapiens
tatggggcaacaccttat

SEQ ID NO: 424 - DNA, 24 bp, Homo sapiens
tacttttcaggagacactctggtt

SEQ ID NO: 425 - DNA, 45 bp, Homo sapiens
tgtgctgtgggtcccttttctgggggttaccagaaagttaccttt

SEQ ID NO: 426 - DNA, 15 bp, Homo sapiens
tctaatcacttatac

SEQ ID NO: 427 - DNA, 18 bp, Homo sapiens
ttttataataatgaaatc

SEQ ID NO: 428 - DNA, 45 bp, Homo sapiens
tgtgccagcagagaggtacactctggggccaacgtcctgactttc

SEQ ID NO: 429 - protein, 6 aa, Homo sapiens
YGATPY

SEQ ID NO: 430 - protein, 8 aa, Homo sapiens
YFSGDTLV

SEQ ID NO: 431 - protein, 12 aa, Homo sapiens
CAFYNQGGKLIF

SEQ ID NO: 432 - protein, 5 aa, Homo sapiens
SNHLY

SEQ ID NO: 433 - protein, 6 aa, Homo sapiens
FYNNEI

SEQ ID NO: 434 - protein, 12 aa, Homo sapiens
CASRVMNTEAFF

SEQ ID NO: 435 - DNA, 18 bp, Homo sapiens
tatggggcaacaccttat

SEQ ID NO: 436 - DNA, 24 bp, Homo sapiens
tacttttcaggagacactctggtt

SEQ ID NO: 437 - DNA, 36 bp, Homo sapiens
tgtgctttttataaccagggaggaaagcttatcttc

SEQ ID NO: 438 - DNA, 15 bp, Homo sapiens
tctaatcacttatac

SEQ ID NO: 439 - DNA, 18 bp, Homo sapiens
ttttataataatgaaatc

SEQ ID NO: 440 - DNA, 36 bp, Homo sapiens
tgtgccagcagggtcatgaacactgaagctttcttt

SEQ ID NO: 441 - protein, 6 aa, Homo sapiens
YGATPY

SEQ ID NO: 442 - protein, 8 aa, Homo sapiens
YFSGDTLV

SEQ ID NO: 443 - protein, 14 aa, Homo sapiens
CAVGFLEYGNKLVF

SEQ ID NO: 444 - protein, 5 aa, Homo sapiens
SGHRS

SEQ ID NO: 445 - protein, 6 aa, Homo sapiens
YFSETQ

SEQ ID NO: 446 - protein, 14 aa, Homo sapiens
CASSSLAGANEQFF

SEQ ID NO: 447 - DNA, 18 bp, Homo sapiens
tatggggcaacaccttat

SEQ ID NO: 448 - DNA, 24 bp, Homo sapiens
tacttttcaggagacactctggtt

SEQ ID NO: 449 - DNA, 42 bp, Homo sapiens
tgtgctgtgggcttcttggaatatggaaacaaactggtcttt

SEQ ID NO: 450 - DNA, 15 bp, Homo sapiens
tctgggcataggagt

SEQ ID NO: 451 - DNA, 18 bp, Homo sapiens
tacttcagtgagacacag

SEQ ID NO: 452 - DNA, 42 bp, Homo sapiens
tgcgccagcagctccctagcgggagccaatgagcagttcttc

SEQ ID NO: 453 - protein, 6 aa, Homo sapiens
VSPFSN

SEQ ID NO: 454 - protein, 7 aa, Homo sapiens
MTFSENT

SEQ ID NO: 455 - protein, 16 aa, Homo sapiens
CVVSAGYGGSQGNLIF

SEQ ID NO: 456 - protein, 5 aa, Homo sapiens
SEHNR

SEQ ID NO: 457 - protein, 6 aa, Homo sapiens
FQNEAQ

SEQ ID NO: 458 - protein, 14 aa, Homo sapiens
CASTPGGSSREQYF

SEQ ID NO: 459 - DNA, 18 bp, Homo sapiens
gtgagccccttcagcaac

SEQ ID NO: 460 - DNA, 21 bp, Homo sapiens
atgactttcagtgagaacaca

SEQ ID NO: 461 - DNA, 48 bp, Homo sapiens
tgtgtggtgagcgcgggatatggaggaagccaaggaaatctcatcttt

SEQ ID NO: 462 - DNA, 15 bp, Homo sapiens
tctgaacacaaccgc

SEQ ID NO: 463 - DNA, 18 bp, Homo sapiens
ttccagaatgaagctcaa

SEQ ID NO: 464 - DNA, 42 bp, Homo sapiens
tgtgccagcacccctgggggctcctcccgcgagcagtacttc

SEQ ID NO: 465 - protein, 7 aa, Homo sapiens
TRDTTYY

SEQ ID NO: 466 - protein, 8 aa, Homo sapiens
RNSFDEQN

SEQ ID NO: 467 - protein, 12 aa, Homo sapiens
CALSTHGNKLVF

SEQ ID NO: 468 - protein, 5 aa, Homo sapiens
SEHNR

SEQ ID NO: 469 - protein, 6 aa, Homo sapiens
FQNEAQ

SEQ ID NO: 470 - protein, 13 aa, Homo sapiens
CASSLDRENEQFF

SEQ ID NO: 471 - DNA, 21 bp, Homo sapiens
acccgtgatactacttattac

SEQ ID NO: 472 - DNA, 24 bp, Homo sapiens
cggaactcttttgatgagcaaaat

SEQ ID NO: 473 - DNA, 36 bp, Homo sapiens
tgtgctctgagtacccatggaaacaaactggtcttt

SEQ ID NO: 474 - DNA, 15 bp, Homo sapiens
tctgaacacaaccgc

SEQ ID NO: 475 - DNA, 18 bp, Homo sapiens
ttccagaatgaagctcaa

SEQ ID NO: 476 - DNA, 39 bp, Homo sapiens
tgtgccagcagcttagacagggaaaatgagcagttcttc

SEQ ID NO: 477 - protein, 6 aa, Homo sapiens
VSNAYN

SEQ ID NO: 478 - protein, 4 aa, Homo sapiens
GSKP

SEQ ID NO: 479 - protein, 14 aa, Homo sapiens
CAVEDRDNNARLMF

SEQ ID NO: 480 - protein, 5 aa, Homo sapiens
ENHRY

SEQ ID NO: 481 - protein, 6 aa, Homo sapiens
SYGVKD

SEQ ID NO: 482 - protein, 16 aa, Homo sapiens
CAISERPVGEQETQYF

SEQ ID NO: 483 - DNA, 18 bp, Homo sapiens
gtgtccaatgcttacaac

SEQ ID NO: 484 - DNA, 12 bp, Homo sapiens
ggctcaaagcct

SEQ ID NO: 485 - DNA, 42 bp, Homo sapiens
tgtgctgtggaggatagggataacaatgccagactcatgttt

SEQ ID NO: 486 - DNA, 15 bp, Homo sapiens
gagaaccaccgctat

SEQ ID NO: 487 - DNA, 18 bp, Homo sapiens
tcatatggtgttaaagat

SEQ ID NO: 488 - DNA, 48 bp, Homo sapiens
tgtgccatcagtgaacggccggtcggggagcaagagacccagtacttc

SEQ ID NO: 489 - protein, 6 aa, Homo sapiens
VSGLRG

SEQ ID NO: 490 - protein, 7 aa, Homo sapiens
LYSAGEE

SEQ ID NO: 491 - protein, 14 aa, Homo sapiens
CAVQIGGYGNKLVF

SEQ ID NO: 492 - protein, 5 aa, Homo sapiens
SGHDY

SEQ ID NO: 493 - protein, 6 aa, Homo sapiens
FNNNVP

SEQ ID NO: 494 - protein, 13 aa, Homo sapiens
CASSPRPSEKLFF

SEQ ID NO: 495 - DNA, 18 bp, Homo sapiens
gtcagcggtttaagaggg

SEQ ID NO: 496 - DNA, 21 bp, Homo sapiens
ctgtattcagctggggaagaa

SEQ ID NO: 497 - DNA, 42 bp, Homo sapiens
tgtgctgtgcagatcggagggtatggaaacaaactggtcttt

SEQ ID NO: 498 - DNA, 15 bp, Homo sapiens
tcaggacacgactac

SEQ ID NO: 499 - DNA, 18 bp, Homo sapiens
tttaacaacaacgttccg

SEQ ID NO: 500 - DNA, 39 bp, Homo sapiens
tgtgccagcagcccccggccgagtgaaaaactgtttttt

SEQ ID NO: 501 - protein, 155 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSSYGNKLVFGAGTILRVKSYIQNPDPAVYQLRDSKSSDKSVCLF

SEQ ID NO: 502 - protein, 239 aa, Homo sapiens
MRGQGRPSSSAFAHSDPDWAKLPSFPDPAMGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSSLSGSSYEQYFGPGTRLTVTEDLKNVFPPEVAVFEPSEAEISHTQKATLVCLATGFYPDHVELSWWVNGKEVHSGVSTDPQPLKEQPALNDSRYCL

SEQ ID NO: 503 - protein, 178 aa, Homo sapiens
MHTSTFQNRPQLFLLIWKKLVPGNPFRRSWMKREREMLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGHAGAQKLVFGQGTRLTINPNIQNPDPAVYQLRD

SEQ ID NO: 504 - protein, 239 aa, Homo sapiens
MKSQNDPLESTVPLSPMHRPRRPLHPVAPAMSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEVWVGSTDTQYFGPGTRLTVLEDLKNVFPPEVAVFEPSEAEISHTQKATLVCLATGFYPDHVELSWWVNGKEVHSGVSTDPPLKEQPALNDSRYCL

SEQ ID NO: 505 - protein, 153 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASRRFGNEKLTFGTGTRLTIIPNIQNPDPAVYQLRD

SEQ ID NO: 506 - protein, 270 aa, Homo sapiens
MVHIRVAAILDLTHQYQVILRLSDVTVGTALWRQGRPSSSAPAHSDPDLVKLPSCPDPAMGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASRLWSRGRTTEAFFGQGTRLTVVEDLNKVFPPEVAVFEPSEAEISHTQKATLVCLATGFYPDHVELSWWVNGKEVHSGVSTDPQPLKEQPALNDSRYCL

SEQ ID NO: 507 - protein, 155 aa, Homo sapiens
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASKTGTASKLTFGTGTRLQVTLDIQNPDPAVYQLRD

SEQ ID NO: 508 - protein, 193 aa, Homo sapiens
MRVGRDNDITDHQPTAWSWEKTYSFFKAAMGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSVNEQFFGPGTRLTVLEDLKNVFPPEVAVFEPSEAEISHTQKATLVCLATGF

SEQ ID NO: 509 - protein, 141 aa, Homo sapiens
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVKSWNNRKLIWGLGTSLAVNPNIQNPDPAVYQLRD

SEQ ID NO: 510 - protein, 211 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASLKRTGGHNQPQHFGDGTRLSILEDLNKVFPPEVAVFEPSEAEISHTQKATLVCLATGFYPDHVELSWWVNGKEVHSGVSTDPQPLKEQPALNDSRYCL

SEQ ID NO: 511 - protein, 210 aa, Homo sapiens
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSVSGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAF

SEQ ID NO: 512 - protein, 209 aa, Homo sapiens
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSHRLAGAEQFFGPGTRLTVLEDLKNVFPPEVAVFEPSEAEISHTQKATLVCLATGFYPDHVELSWWVNGKEVHSGVSTDPQPLKEQPALNDSRYCL

SEQ ID NO: 513 - protein, 161 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALTLNSGYSTLTFGKGTMLLVSPDIQNPDPAVYQLRDSKSSDKSVCLFTDFD

SEQ ID NO: 514 - protein, 166 aa, Homo sapiens
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDPGTLYEQYFGPGTRLTVTEDLKNVFPPEVAVFEPS

SEQ ID NO: 515 - protein, 145 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVERPGFQKLVFGTGTRLLVSPNIQNPDPAVYQLRD

SEQ ID NO: 516 - protein, 208 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASRTTGSGTEAFFGQGTRLTVVEDLNKVFPPEVAVFEPSEAEISHTQKATLVCLATGFFPDHVELSWWVNGKEVHSGVSTDPQPLKEQPALNDSRYCL

SEQ ID NO: 517 - protein, 145 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATPRRGADGLTFGKGTHLIIQPYIQNPDPAVYQLRD

SEQ ID NO: 518 - protein, 139 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAFCNQFYFGTGTSLTVIPNIQNPDPAVYQLRD

SEQ ID NO: 519 - protein, 211 aa, Homo sapiens
MGCRLLCCAVLCLLGAVPMETGVTQTPRHLVMGMTNKKSLKCEQHLGHNAMYWYKQSAKKPLELMFVYNFKEQTENNSVPSRFSPECPNSSHLFLHLHTLQPEDSALYLCASSPGGSGRLGDTQYFGPGTRLTVLEDLKNVFPPEVAVFEPSEAEISHTQKATLVCLATGFFPDHVELSWWVNGKEVHSGVSTDPQPLKEQPALNDSRYCL

SEQ ID NO: 520 - protein, 154 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVEDSRGNNRLAFGKGNQVVVIPNIQNPDPAVYQLRDSKSSDKSV

SEQ ID NO: 521 - protein, 237 aa, Homo sapiens
MSQNDFLESPAPLSSMHRYRRPLRHAASAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASGGTGGNYGYTFGSGTRLTVVEDLNKVFPPEVAVFEPSEAEISHTQKATLVCLATGFFPDHVELSWWVNGKEVHSGVSTDPQPLKEQPALNDSRYCL

SEQ ID NO: 522 - protein, 167 aa, Homo sapiens
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMKSLFGNEKLTFGTGTRLTIIPNIQNPDPAVYQLRDSKSSDKSVCLFTDFDSQ

SEQ ID NO: 523 - protein, 222 aa, Homo sapiens
MEAVVTTLPREGGVRPSRKMLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARSGEKLFFGSGTQLSVLEDLNKVFPPEVAVFEPSEAEISHTQKATLVCLATGFFPDHVELSWWVNGKEVHSGVSTDPQPLKEQPALNDSRYCL

SEQ ID NO: 524 - protein, 213 aa, Homo sapiens
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMKSPFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAF

SEQ ID NO: 525 - protein, 223 aa, Homo sapiens
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARWGDQPQHFGDGTRLSILEDLNKVFPPEVAVFEPSEAEISHTQKATLVCLATGFYPDHVELSWWVNGKEVHSGVSTDPQPLKEQPALNDSRYCL

SEQ ID NO: 526 - protein, 150 aa, Homo sapiens
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMKTNFGNEKLTFGTGTRLTIIPNIQNPDPAVYQLRD

SEQ ID NO: 527 - protein, 204 aa, Homo sapiens
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARSGTSKFFGPGTRLTVLEDLKNVFPPEVAVFEPSEAEISHTQKATLVCLATGFFPDHVELSWWVNGKEVHSGVST

SEQ ID NO: 528 - protein, 150 aa, Homo sapiens
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFTKSNFGNEKLTFGTGTRLTIIPNIQNPDPAVYQLRD

SEQ ID NO: 529 - protein, 222 aa, Homo sapiens
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARTGDEQYFGPGTRLTVTEDLKNVFPPEVAVFEPSEAEISHTQKATLVCLATGFYPDHVELSWWVNGKEVHSGVSTDPQPLKEQPALNDSRYCL

SEQ ID NO: 530 - protein, 193 aa, Homo sapiens
MGIAFPALKENSHQAQRRTHQSRRLFTLQGSAVSMTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFIKSNFGNEKLTFGTGTRLTIIPNIQNPDPAVYQLRDSKSSDKSVC

SEQ ID NO: 531 - protein, 222 aa, Homo sapiens
MEAVVTTLPREGGVRPSRKMLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARRGTEAFFGQGTRLTVVEDLNKVFPPEVAVFEPSEAEISHTQKATLVCLATGFFPDHVELSWWVNGKEVHSGVSTDPQPLKEQPALNDSRYCL

SEQ ID NO: 532 - protein, 171 aa, Homo sapiens
MAQELGMQCQARGILQQMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVMDSSYKLIFGSGTRLLVRPDIQNPDPAVYQLRDSKSSDKSVCLFTDF

SEQ ID NO: 533 - protein, 149 aa, Homo sapiens
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVKIIFGSGTRLSIRPNIQNPDPAVYQLRDSKSSDKSVCLFTDF

SEQ ID NO: 534 - protein, 237 aa, Homo sapiens
MRSQDDFLDSPVRLSSTHRPRRPLCLVASAMRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSEREHYGYTFGSGTRLTVVEDLNKVFPPEVAVFEPSEAEISHTQKATLVCLATGFYPDHVELSWWVNGKEVHSGVSTDPQPLKEQPALNDSRYCL

SEQ ID NO: 535 - protein, 160 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATPYTGGFKTIFGAGTRLFVKANIQNPDPAVYQLRDSKSSDKSVCLFTDFD

SEQ ID NO: 536 - protein, 208 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQLGSGNTIYFGEGSWLTVVEDLNKVFPPEVAVFEPSEAEISHTQKATLVCLATGFYPDHVELSWWVNGKEVHSGVSTDPQPLKEQPALNDSRYCL

SEQ ID NO: 537 - protein, 161 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVKAQGGAGSYQLTFGKGTKLSVIPNIQNPDPAVYQLRDSKSSDKSVCLFTD

SEQ ID NO: 538 - protein, 172 aa, Homo sapiens
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCGSGRERTDTQYFGPGTRLTVLEDLKNVFPPEVAVFEPSEAEISHTQKATLVCLATGFYPDHV

SEQ ID NO: 539 - protein, 142 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGANSGYALNFGKGTSLLVTPHIQNPDPAVYQLRD

SEQ ID NO: 540 - protein, 238 aa, Homo sapiens
MRVGRDNDITDHQPTAWSWEKTYSFFKAAMGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSSLPGQLYGYTFGSGTRLTVVEDLNKVFPPEVAVFEPSEAEISHTQKATLVCLATGFYPDHVELSWWVNGKEVHSGVSTDPQPLKEQPALNDSRYCL

SEQ ID NO: 541 - protein, 149 aa, Homo sapiens
MQCQAHGILQQMWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAEGGFKTIFGAGTRLFVKANIQNPDPAVYQLRD

SEQ ID NO: 542 - protein, 196 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGPLTGPPEQFFGPGTRLTVLEDLKNVFPPEVAVFEPSEAEISHTQKATLVCLATGFYPDHVELSWWVNGKEVHSGVSTDPQPL

SEQ ID NO: 543 - DNA, 553 bp, Homo sapiens
actgcgcggtcacccttttcttatatggggactgtgatttcttcatgttaaggatcaagaccattatttgggtaacacactaaagatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgagtagctatggaaacaagctgsgtctttggcgcaggaaccattctgagagtcaagtcctatatccagaaccctgaccctgccgtgtaccagctgagagactctaaatccagtgacaagtctgtctgcctattcac

SEQ ID NO: 544 - DNA, 960 bp, Homo sapiens
gcactaactgatatctctgtttcgagttgccttcaactcgaaacatccagcagaggatgggcctagagatggagtaggagatccagtccccaagccctagagatgtgagtgaggacacttggctgaacttacacagtctgtgaatacagtaaagcaacttcgtgctctcttttagcttataataattggtttctaacaatgtaggcatttgtggaggcaatgatgtcactgtgggaactgccatgagaggacagggacgtccctcctcctctgcttttgctcacagtgaccctgattgggcaaagctcccatccttccctgaccctgccatgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagctcgcttagcgggagctcctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 545 - DNA, 633 bp, Homo sapiens
aaaacatagactctcctctgagagggtgggcaaaatagtgtgattggttttctcaacttgcagtcacctctttccctttctttccaaaatgcacacatctactttccaaaatagaccgcaactatttctattgatctggaagaagcttgtaccaggcaacccatttaggagaagttggatgaagagggagagggagatgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcagggcacgcaggagcccagaagctggtatttggccaaggaaccaggctgactatcaacccaaatatccagaaccctgaccctgccgtgtaccagctgagagactagatcggaag

SEQ ID NO: 546 - DNA, 831 bp, Homo sapiens
ggattcttcccattcaaacggttcaccggtgcatgaatcttgaatttgaccatctggggaaggggcgtggcctctcctgacaggaaggctctggggcccaggcagggagaatgaagtctcagaatgacccccttgagagtactgttcccctatcaccgatgcacagacccagaagacccctccatcctgtagcacctgccatgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgaggtatgggtcggtagcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 547 - DNA, 589 bp, Homo sapiens
ttatatgggggttcatatgtaaaatgaagggtctgtggaaggacatgaataaagcacaggaggttgaagtcagatttgcagctttctaggcaggagataagacaatctgcatcttcacaggagggatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcaggcggtttggaaatgagaaattaacctttgggactggaacaagactcaccatcatacccaatatccagaaccctgaccctgccgtgtaccagctgagagactagat

SEQ ID NO: 548 - DNA, 985 bp, Homo sapiens
ccacttcccaaacacctcccactcctacccagaccgtggatgggcaggaaatgcaggaacagagccagaaacaggagatctccaaggaaggttgacagtcagcactgggatcgtctgtgtaaagtgctgctgaagcagccaggtggcatgtccagccgacaatgcgaaaggaaaaatggtgcacatcagagttgctgccatcttagacttaactcatcagtatcaggtgatcctgaggctcagtgatgtcactgtgggaactgctctgtggcgacaaggacgtccctcatcctctgctcctgctcacagtgaccctgatctggtaaagctcccatcctgccctgaccctgccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcaggctctggagcagggggcggaccactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctg

SEQ ID NO: 549 - DNA, 649 bp, Homo sapiens
ttctaaatttggacaaagtggggaagtgcttcctttgacagagacagctttaagtgaaagcacttgtgaaagggcggggcctgctgaaagaattcagttgagggtgaatttacagagtttcagctggttgggaagactggaagaccacctgggctgtcattgagctctggtgccaggaggaatggacaagatcttaggagcatcatttttagttctgtggcttcaactatgctgggtgagtggccaacagaaggagaaaagtgaccagcagcaggtgaaacaaagtcctcaatctttgatagtccagaaaggagggatttcaattataaactgtgcttatgagaacactgcgtttgactactttccatggtaccaacaattccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgtgcagcaagcaagaccggcactgccagtaaactcacctttgggactggaacaagacttcaggtcacgctcgatatccagaaccctgaccctgccgtgtaccagctgagagactag

SEQ ID NO: 550 - DNA, 832 bp, Homo sapiens
gggagtctagacacactaagccatgcttaccagttactatagtgggaacactcccaaaattcaagttcgcagatgcaaatcaagagccaaccatgcaagcagtcccttctaaggccagcagtctcaggcccgctgtgtcaactcttttctatacgtgcacatacacacagtgacacttccatgagcacaagggatgtccttcactctctagcacctctagaccctttctagtctagacaatggctgagggattatgagagtgggccgggacaatgacatcacagaccatcaacccactgcctggtcctgggagaagacctattctttcttcaaagcagccatgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtgtgaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttc

SEQ ID NO: 551 - DNA, 876 bp, Homo sapiens
gacagagctcactataggcttctgggatttaatgacactttccacaagctccatggagcagaggaaactttatttggtacattgaggaagaaatgatggaagaaaaggtctggatagatacatccttatgatcagttttcctcattctgtgccagcctcttagactttaaacctcataaatgatttcagtagacaccccctgagttgaggatgtctggttgatatcctcaccgtcttctcaggaacatgactaaacctggggaggaagacatagaaaagcaactgctctatcattggctgacaagatcctgacaacaacacaatgagagctgcactccttgcaagtctttactagtagctgttgcttctgttctctggaaattctttaaacctagaatcagacacaaaaactgaactctgggtccacaatcctcatttgtccttgaagtatgaggctggtggcaagagtaactgtgtttctgacctttggaactataattgatgctaagaccacccagcccccctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcaaaagttggaacaaccgtaagctgatttggggattgggaacaagcctggcagtaaatccgaatatccagaaccctgaccctgccgtgtaccagctgagagactaga

SEQ ID NO: 552 - DNA, 790 bp, Homo sapiens
gccatcttagacttaactcatcagtatcaggtgatcctgaggctcagtgatgtcactgtgggaactgctctgtggcgacaaggacgtccctcatcctctgctcctgctcacagtgaccctgatctggtaaagctcccatcctgccctgaccctgccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagccttaagagaacagggggccacaatcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 553 - DNA, 1152 bp, Homo sapiens
gagtcagattcttgctatgctgcccgggctggtcttgaacccctggcttcaagcaatcttccagcttcagccacacaaagtgtggggattacagaaggtaacatcatcacagattcaggaggaaagaaacgctttctgttgaagttttgatatcatgtctgaaatgagaaggaaacagcttgctcccttggatccgtggttccgcctgttgcctctagagggcgatgttgcatcaagtaggcatctgttttcatcgcagaccatcctcatccattgagcctcctccctgtagctggctgatgtagctcgttgatatctgtgtggatagggagctgtgacgagggcaagaggtcagaacacatccaggctccttaagggaaagcctctttctgtttctgaaacttttcaaagccagggacttgtccaatccaacctcctcacagttcctagctcctgaggctcagcgcccttggcttctgtccgcccagctcaaggtcctgcagcattgccactgctcagccatgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgtgctgtgagtgtttctggttctgcaaggcaactgacctttggatctgggacacaattgactgttttacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttca

SEQ ID NO: 554 - DNA, 924 bp, Homo sapiens
gtggtgaggagataaactcagaaatgcagcatgaggcctgtagctccagacagctctagagcacaaagtgaagaccgatgcattgatgttgtttaaaaggagctaataaatatctaaagcagtcatccaagtgtgttctaatataaatcctgtgttcctgaggttgtggggattgagagaggaagtgatgtcactgtgggtactgttctgtgtcaggacaaggacgtccctcctcctctgctcctgctcacagtgaccctgatctggtaaagctcccatcctgccctgactctgtcatgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagccaccgtctagcgggagctgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 555 - DNA, 546 bp, Homo sapiens
tatggggactgtgatttcttcatgttaaggatcaagaccattatttgggtaacacactaaagatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgaccctgaattcaggatacagcaccctcacctttgggaaggggactatgcttctagtctctccagatatccagaaccctgaccctgccgtgtaccagctgagagactctaaatccagtgacaagtctgtctgcctattcaccgattttgatt

SEQ ID NO: 556 - DNA, 553 bp, Homo sapiens
gagactagggcatccttgggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagacccaggaaccttatacgagcagtacttcgggccgggcaccaggctcacggtcacagaggacctgaaaaacgtgttcccacccgaggtcgctgtgtttgagccatcaga

SEQ ID NO: 557 - DNA, 578 bp, Homo sapiens
gtcattactaagcagcctcacaactgtagcaacccttctaaaggttgtagattctggctgatgatgtcactgacacaaaggaaaaaatgcaaaacaggtagtcttaaataagcattctggtgagaccaactgcattttggccatggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggagcggccaggctttcagaaacttgtatttggaactggcacccgacttctggtcagtccaaatatccagaaccctgaccctgccgtgtaccagctgagagact

SEQ ID NO: 558 - DNA, 735 bp, Homo sapiens
ggctcactggcgcagcacctctcagcggcagtggaaaccacagcctagtcctctcaccactgcagaccagaatcctgccctgggccttgcctggtctgcctcactctgccatgggctgcaggctcctctgctgtgtggtcttctgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcaggacgacagggtcggggactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctga

SEQ ID NO: 559 - DNA, 716 bp, Homo sapiens
ggtaatttcttctgagtcagctctgaacacacgtcctgatggcagcaggatgatgttacgatcacaagagggcagtgcctccctctcctcagactaacttggctttaaatcagcctatctgcattgaaaggaaaggactgagcttgcctgtgactggctagggaggaacctgagactaggggacagaaagactagggattcacccagtaaagagagctcatctgtgactgaggagccttgctccatttcaggtcttctgtgatttcaataaggaagaagaatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacgccgaggcgaggtgctgacggactcacctttggcaaagggactcatctaatcatccagccctatatccagaaccctgaccctgccgtgtaccagctgagagact

SEQ ID NO: 560 - DNA, 554 bp, Homo sapiens
gcctcctgcctttctatggaactcaaagtgggtggcagcaagcactcttctagcccagagaagtctgttccaggacgggctctttcaggagcagctaaagtcaggggccatgtccaccatgtgatagaaagacaagatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgccttttgtaaccagttctattttgggacagggacaagtttgacggtcattccaaatatccagaaccctgaccctgccgtgtaccagctgagagact

SEQ ID NO: 561 - DNA, 851 bp, Homo sapiens
agcccattcatcccagcccagacaattcaaatctgccttctatcaggacctagaaaggatgtaaaacggccgggtataaatatcccctgggtctggggaaactgtcaggagcagtgacatcacaggaaaaaccaccaaccaaggccaaggagaccagagcccagcacctcacccagaggaccccagtcagaggccccatctcagacccgaggctagcatgggctgcaggctgctctgctgtgcggttctctgtctcctgggagcggtccccatggaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagactcggccctgtatctctgtgccagcagcccggggggtagcgggaggttgggagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctga

SEQ ID NO: 562 - DNA, 584 bp, Homo sapiens
caactgtagcaacccttctaaaggttgtagattctggctgatgatgtcactgacacaaaggaaaaaatgcaaaacaggtagtcttaaataagcattctggtgagaccaactgcattttggccatggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggaggattcgcgggggaacaacagactcgcttttgggaaggggaaccaagtggtggtcataccaaatatccagaaccctgaccctgccgtgtaccagctgagagactctaaatccagtgacaagtctgtc

SEQ ID NO: 563 - DNA, 762 bp, Homo sapiens
ggggtgtggcctctcctggcctctccctccctggggcccaggcagggagaatgtctcagaatgacttccttgagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcggagggacaggggggaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctga

SEQ ID NO: 564 - DNA, 611 bp, Homo sapiens
tggggacctatcatagcatttcctgccctgaaggagaattctcaccaagcacagaggagaacccatcagagcaggagacttttcactctgcaggggagcgctgtcagcatgacacgtgttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgaagtcgctatttggaaatgagaaattaacctttgggactggaacaagactcaccatcatacccaatatccagaaccctgaccctgccgtgtaccagctgagagactctaaatccagtgacaagtctgtctgcctattcaccgattttgattctcaaac

SEQ ID NO: 565 - DNA, 749 bp, Homo sapiens
agtcatgggcaaagattaccaccagggggcagactagggcatccttgggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaggtcgggcgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctga

SEQ ID NO: 566 - DNA, 742 bp, Homo sapiens
atatggggagcatttcctgccctgaaggagaattctcaccaagcacagaggagaacccatcagagcaggagacttttcactctgcaggggagcgctgtcagcatgacacgtgttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgaagtctccctttggaaatgagaaattaacctttgggactggaacaagactcaccatcatacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttca

SEQ ID NO: 567 - DNA, 711 bp, Homo sapiens
ggggggggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaggtggggtgatcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 568 - DNA, 553 bp, Homo sapiens
tggggcatttcctgccctgaaggagaattctcaccaagcacagaggagaacccatcagagcaggagacttttcactctgcaggggagcgctgtcagcatgacacgtgttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgaagactaactttggaaatgagaaattaacctttgggactggaacaagactcaccatcatacccaatatccagaaccctgaccctgccgtgtaccagctgagagactagatc

SEQ ID NO: 569 - DNA, 694 bp, Homo sapiens
agtcatgggcaaagattaccaccagggggcagactagggcatccttgggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaggtccgggactagcaaattcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacg

SEQ ID NO: 570 - DNA, 599 bp, Homo sapiens
gagaaatgtttccctccttgaaacctagtcacccacccacctatcatagcatttcctgccctgaaggagaattctcaccaagcacagaggagaacccatcagagcaggagacttttcactctgcaggggagcgctgtcagcatgacacgtgttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcacgaagtctaactttggaaatgagaaattaacctttgggactggaacaagactcaccatcatacccaatatccagaaccctgaccctgccgtgtaccagctgagagactagatcgg

SEQ ID NO: 571 - DNA, 793 bp, Homo sapiens
tggggctgtatggttttttgacaggcagagatggacgtaacatcagtcatgggcaaagattaccaccagggggcagactagggcatccttgggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctcggacaggagacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 572 - DNA, 583 bp, Homo sapiens
atatggggatagcatttcctgccctgaaggagaattctcaccaagcacagaggagaacccatcagagcaggagacttttcactctgcaggggagcgctgtcagcatgacacgtgttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatcaaatctaactttggaaatgagaaattaacctttgggactggaacaagactcaccatcatacccaatatccagaaccctgaccctgccgtgtaccagctgagagactctaaatccagtgacaagtctgtctgcct

SEQ ID NO: 573 - DNA, 713 bp, Homo sapiens
ggggatccttgggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgcccggagggggactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctga

SEQ ID NO: 574 - DNA, 1077 bp, Homo sapiens
cggagtctcactttgtcgcccaggctggagtgcagtggtgcgatctctgctcactgcaaactccgcctcccgggttcccgccattctcctgcctcagcctctcgagtagctgggactacaggcgcccgccacagcgcccagctaatttttttgtattttttggtagagacggggtttcaccgtgttagccaggatggtctcgatctcctgacctcgtgatccgcccacctcggcctcccaaagcgctgggattaaaggcgtgagccaccgcgcccggcctactttgtcttatgttattcccatttgccgtctctgttccttatacattatgctttttcaactttaccagaatcacttggattaaaacccgtggatttctcagtaggaaatgttcatgtgaagacacttctgtagtaacagaacctacagctgctcctgtagaaggaagttgaaagtcatcccttcaagaaaggggctcctccccttgtaattctactgggttttgcatccagactgagtttccttccctcacccacatgaagtgtctaccttctgcagactccaatggctcaggaactgggaatgcagtgccaggctcgtggtatcctgcagcagatgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgatggatagcagctataaattgatcttcgggagtgggaccagactgctggtcaggcctgatatccagaaccctgaccctgccgtgtaccagctgagagactctaaatccagtgacaagtctgtctgcctattcaccgatttt

SEQ ID NO: 575 - DNA, 672 bp, Homo sapiens
tatatggggagtaccagcagttttcactccctactcttcttggggctttagctgaactttgaggggagccctgctctgaggagctgctgccctgctattggctgctgtactggggcagcctgagtgacagctgctggtgtgggccctggcagttgctgctgggctcattgcagctcagacacagcaaaagagcctagaacctgggtcctagtttgcacctagaatatgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgaagataatctttggatcagggaccagactcagcatccggccaaatatccagaaccctgaccctgccgtgtaccagctgagagactctaaatccagtgacaagtctgtctgcctattcaccgatttt

SEQ ID NO: 576 - DNA, 978 bp, Homo sapiens
gtcggctgaagggaatgcttggctgcgtgctggcatcaggaggcggttggaattcagagtaggtcctgggactgcccagggcaaggaataaaatttatattgaattacaaactgtttgcacaaaagtcaagaaaaacttgaaagccttatatcaagaagcattcttcccatccaaacttcagtggtgcatttattttggatttgaccctctggggaaggggcatggcctctctcgacaagaaggttctggggaccaggcagagagaatgaggtctcaggatgacttccttgacagccctgttcgcctttcatcaacacacagacccagaagacctctctgtcttgtagcatctgccatgagaatcaggctcctgtgctgtgtggccttttctctcctgtgggcaggtccagtgattgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcagtgaaagggagcactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacggacccgcagcccctcaaggagcagcccgccctcaatgactccagatactgcctga

SEQ ID NO: 577 - DNA, 668 bp, Homo sapiens
gaacttggctttaaatcagcctatctgcattgaaaggaaaggactgagcttgcctgtgactggctagggaggaacctgagactaggggacagaaagactagggattcacccagtaaagagagctcatctgtgactgaggagccttgctccatttcaggtcttctgtgatttcaataaggaagaagaatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacgccatatactggaggcttcaaaactatctttggagcaggaacaagactatttgttaaagcaaatatccagaaccctgaccctgccgtgtaccagctgagagactctaaatccagtgacaagtctgtctgcctattcaccgattttgattc

SEQ ID NO: 578 - DNA, 798 bp, Homo sapiens
agaacttagaaaggatgtaaagcagtcaggaagaaacatcccctgggtctggggaaactatcaggagcagtgacatcacaagaaaaaccaccaaccagggccaaggagaccagagcccagcacctcgcccaaaggaccccagtcagaggccccatctcagacccgaggctagcatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaacttggctctggaaacaccatatattttggagagggaagttggctcactgttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 579 - DNA, 667 bp, Homo sapiens
agagagagagagagagagagagagagagagagacttgtctcagattggctgggccaactctgtgtttcctgtagaaatgtttgaacatatgagagagcaaagcagagtgtttatcttgtgagccattctccatatttcagatataagatttcagttctcagtgagtctaagtgacagaaggaatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaaggcccagggtggggctgggagttaccaactcactttcgggaaggggaccaaactctcggtcataccaaatatccagaaccctgaccctgccgtgtaccagctgagagactctaaatccagtgacaagtctgtctgcctattcaccgattt

SEQ ID NO: 580 - DNA, 807 bp, Homo sapiens
gagagggcaatggggcagcctgtgagctggggcaacgtaggcagagaaggaactgtgtcaccacagaaacttctgccttcacccatcccttcagctctgcaggacaggtagagactccaggatcatccactgagcactggacataaggaaggctgcatggggaggactcaggacagtgacatcacaggatacccctcctattaggaaaatcaaggcccagaattcactcggctcttccccaggaggaccaagccctgaatcaggtgcagtgctgcctgccccactgtgccatgggccctgggctcctctgctgggtgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgggtccgggcgggaacgcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtgg

SEQ ID NO: 581 - DNA, 562 bp, Homo sapiens
tggggggcctttctatggaactcaaagtgggtggcagcaagcactcttctagcccagagaagtctgttccaggacgggctctttcaggagcagctaaagtcaggggccatgtccaccatgtgatagaaagacaagatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagctaattccgggtatgcactcaacttcggcaaaggcacctcgctgttggtcacaccccatatccagaaccctgaccctgccgtgtaccagctgagagact

SEQ ID NO: 582 - DNA, 960 bp, Homo sapiens
agacacactaagccatgcttaccagttactatagtgggaacactcccaaaattcaagttcgcagatgcaaatcaagagccaaccatgcaagcagtcccttctaaggccagcagtctcaggcccgctgtgtcaactcttttctatacgtgcacatacacacagtgacacttccatgagcacaagggatgtccttcactctctagcacctctagaccctttctagtctagacaatggctgagggattatgagagtgggccgggacaatgacatcacagaccatcaacccactgcctggtcctgggagaagacctattctttcttcaaagcagccatgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagttccctaccgggacagctttatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 583 - DNA, 724 bp, Homo sapiens
ttctacctcagcgtcttgagtagctgggattacaggcataagccactgtgcccagcttaaaacctgtggatttatcagtagaaaatgttcatgtaaagatactcctgtaagagaaaccatagctgctccagtggaaggaagcttaaactcatcccttcaagaaagaagctcctccctttgtatttctactgggttttgcatccggactgatcttccttccctcacccacatgaagtgtctaccttctgcagactacagtggctcaggaaccggggatgcagtgccaggctcatggtatcctgcagcagatgtggggagctttccttctctatgtttccatgaagatgggaggcactgcaggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgagggaggcttcaaaactatctttggagcaggaacaagactatttgttaaagcaaatatccagaaccctgaccctgccgtgtaccagctgagagactt

SEQ ID NO: 584 - DNA, 827 bp, Homo sapiens
acaactctctggtggttgccagtaaagcgccagaaaaacagaagagaagcaagtagaagaatttcagcatctgctaagaacatataatacataagccaacatggcatctttctttaagacccagtaccttgggagggacaatgacatcacttcctgaatcctcccagttttctatttccatgccctgcttccctcaacatccagagctggaaacacctccatcctgcctcttcatgccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtggccccctcacagggccccccgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctca

SEQ ID NO: 585 - protein, 144 aa, Homo sapiens
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAAAIGSYIPTFGRGTSLIVHPYIQNPDPAVYQLRD

SEQ ID NO: 586 - protein, 208 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGNSNTEAFFGQGTRLTVVEDLNKVFPPEVAVFEPSEAEISHTQKATLVCLATGFYPDHVELSWWVNGKEVHSGVSTDPQPLKEQPALNDSRYCL

SEQ ID NO: 587 - protein, 154 aa, Homo sapiens
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVRESGGGADGLTFGKGTHLIIQPYIQNPDPAVYQLRDSKSSD

SEQ ID NO: 588 - protein, 166 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGEQGMTQPQHFGDGTRLSILEDLNKVFPPEVAVFEPSEAEISHTQKATLVCLATGF

SEQ ID NO: 589 - protein, 141 aa, Homo sapiens
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRYQGAQKLVFGQGTRLTINPNIQNPDPAVYQLRD

SEQ ID NO: 590 - protein, 211 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQVRERKNTGELFFGEGSRLTVLEDLKNVFPPEVAVFEPSEAEISHTQKATLVCLATGFYPDHVELSWWVNGKEVHSGVSTDPQPLKEQPALNDSRYCL

SEQ ID NO: 591 - protein, 160 aa, Homo sapiens
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTGAGSYQLTFGKGTKLSVIPNIQNPDPAVYQLRDSKSSDKSVCLFTDFDS

SEQ ID NO: 592 - protein, 194 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSHPGLRAGASYNEQFFGPGTRLTVLEDLKNVFPPEVAVFEPSEAEISHTQKATLVCLATGFYPDHVELSWWVNGKEVHSGVS

SEQ ID NO: 593 - protein, 151 aa, Homo sapiens
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCAVLNTGNQFYFGTGTSLTVIPNIQNPDPAVYQLRDSKSSDK

SEQ ID NO: 594 - protein, 182 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSRFPGGGYNEQFFGPGTRLTVLEDLKNVFPPEVAVFEPSEAEISHTQKATLVCLATGFYPDHVELSWWVN

SEQ ID NO: 595 - protein, 155 aa, Homo sapiens
MMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNWSGNEKLTFGTGTRLTIIPNIQNPDPAVYQLRDLKTTQTRVF

SEQ ID NO: 596 - protein, 164 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATRGQGVGELFFGEGSRLTVLEDLKNVFPPEVAVFEPSEAEISHTQKATLVCLA

SEQ ID NO: 597 - protein, 152 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSVQAAGNKLTFGGGTRVLVKPNIQNPDPAVYQLRDYDGP

SEQ ID NO: 598 - protein, 237 aa, Homo sapiens
MRGQGRPSSFAVAHSDPDWAKLPSFPDPAMGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDRNYGYTFGSGTRLTVVEDLNKVFPPEVAVFEPSEAEISHTQKATLVCLATGFYPDHVELSWWVNGKEVHSGVSTDPQPLKEQPALNDSRYCL

SEQ ID NO: 599 - protein, 224 aa, Homo sapiens
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARTSGQYEQYFGPGTRLTVTEDLKNVFPPEVAVFEPSEAEISHTQKATLVCLATGFYPDHVELSWWVNGKEVHSGVSTDPQPLKEQPALNDSRYCL

SEQ ID NO: 600 - protein, 224 aa, Homo sapiens; identical to SEQ ID NO: 599

SEQ ID NO: 601 - protein, 149 aa, Homo sapiens
MNYSPGLVSLILLLLGRTRGDSVTQMEGPVTLSEEACLTINCTYTATGYPSLFWYVQYPGEGLQLLLKATKADDKGSNKGFEATYRKETTSFHLEKGSVQVSDSAVYFCALSDLLDSGNTGKLIFGQGTTLQVKPDIQNPDPAVYQLRD

SEQ ID NO: 602 - protein, 180 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASRGSYNEQFFGPGTRLTVLEDLKNVFPPEVAVFEPSEAEISHTQKATLVCLATGFYPDHVELSWWVNGK

SEQ ID NO: 603 - protein, 145 aa, Homo sapiens
MMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNTPSSKIIFGSGTRLSIRPNIQNPDPAVYQLRD

SEQ ID NO: 604 - protein, 222 aa, Homo sapiens
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARSGQGQFFGPGTRLTVLEDLKNVFPPEVAVFEPSEAEISHTQKATLVCLATGFYPDHVELSWWVNGKEVHSGVSTDPQPLKEQPALNDSRYCL

SEQ ID NO: 605 - protein, 209 aa, Homo sapiens
MMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVPPGGGFKTIFGAGTRLFVKANIQNPDPAVYQLRDSKSSDKSVCLFTDFDSQTNVSQSKDSDVYITDKTVLDMRSMDFKSNSAVAWSNKSDFACANAF

SEQ ID NO: 606 - protein, 208 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRTRTASYGYTFGSGTRLTVVEDLNKVFPPEVAVFEPSEAEISHTQKATLVCLATGFFPDHVELSWWVNGKEVHSGVSTDPQPLKEQPALNDSRYCL

SEQ ID NO: 607 - protein, 157 aa, Homo sapiens
MAQELGMQCQARGILQQMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVLDSNYQLIWGAGTKLIIKPDIQNPDPAVYQLRD

SEQ ID NO: 608 - protein, 180 aa, Homo sapiens
MKSQNDPLESTVPLSPMHRPRRPLHPVAPAMSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEEASGSYEQYFGPGTRLTVTEDLKNVFPPEVAVFEPS

SEQ ID NO: 609 - protein, 144 aa, Homo sapiens
MMKSLRVLLVILWLQLSWVWSQQKEVEQNSGPLSVPEGAIASLNCTYSDRVSQSFFWYRQYSGKSPELIMSIYSNGDKEDGRFTAQLNKASQYVSLLIRDSQPSDSATYLCAVSGNQFYFGTGTSLTVIPNIQNPDPAVYQLRD

SEQ ID NO: 610 - protein, 211 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLGSGRYNEQFFGPGTRLTVLEDLKNVFPPEVAVFEPSEAEISHTQKATLVCLATGFYPDHVELSWWVNGKEVHSGVSTDPQPLKEQPALNDSRYCL

SEQ ID NO: 611 - protein, 148 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGPFSGGYQKVTFGTGTKLQVIPNIQNPDPAVYQLRD

SEQ ID NO: 612 - protein, 210 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASREVHSGANVLTFGAGSRLTVLEDLKNVFPPEVAVFEPSEAEISHTQKATLVCLATGFYPDHVELSWWVNGKEVHSGVSTDPQPLKEQPALNDSRYCL

SEQ ID NO: 613 - protein, 173 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAFYNQGGKLIFGQGTELSVKPNIQNPDPAVYQLRDSKSSDKSVCLFTDFDSQTNVSQSKDSDV

SEQ ID NO: 614 - protein, 207 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRVMNTEAFFGQGTRLTVVEDLNKVFPPEVAVFEPSEAEISHTQKATLVCLATGFFPDHVELSWWVNGKEVHSGVSTDPQPLKEQPALNDSRYCL

SEQ ID NO: 615 - protein, 202 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGFLEYGNKLVFGAGTILRVKSYIQNPDPAVYQLRDSKSSDKSVCLFTDFDSQTNVSQSKDSDVYITDKTVLDMRSMDFKSNSAVAWSNKS

SEQ ID NO: 616 - protein, 175 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSSLAGANEQFFGPGTRLTVLEDLKNVFPPEVAVFEPSEAEISHTQKATLVCLATGFYPDHVEL

SEQ ID NO: 617 - protein, 150 aa, Homo sapiens
MKKHLTTFLVILWLYFYRGNGKNQVEQSPQSLIILEGKNCTLQCNYTVSPFSNLRWYKQDTGRGPVSLTIMTFSENTKSNRRYTATLDADTKQSSLHITASQLSDSASYICVVSAGYGGSQGNLIFGKGTKLSVKPNIQNPDPAVYQLRD

SEQ ID NO: 618 - protein, 169 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASTPGGSSREQYFGPGTRLTVTEDLKNVFPPEVAVFEPSEAEISHTQKATLVCLATGF

SEQ ID NO: 619 - protein, 149 aa, Homo sapiens
MNMLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSTHGNKLVFGAGTILRVKSYIQNPDPAVYQLRD

SEQ ID NO: 620 - protein, 202 aa, Homo sapiens
MGDVTVGTALWRQGRPSSCAPAHSDPDLVKLPSCPDPAMGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLDRENEQFFGPGTRLTVLEDLKNVFPPEVAVFEPSEAEISHTQKATLVCL

SEQ ID NO: 621 - protein, 146 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVEDRDNNARLMFGDGTQLVVKPNIQNPDPAVYQLRD

SEQ ID NO: 622 - protein, 356 aa, Homo sapiens
MRSWPGPEMGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISERPVGEQETQYFGPGTRLLVLEDLKNVFPPEVAVFEPSEAEISHTQKATLVCLATGFMRSWPGPEMGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISERPVGEQETQYFGPGTRLLVLEDLKNVFPPEVAVFEPSEAEISHTQKATLVCLATGF

SEQ ID NO: 623 - protein, 146 aa, Homo sapiens
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQIGGYGNKLVFGAGTILRVKSYIQNPDPAVYQLRD

SEQ ID NO: 624 - protein, 208 aa, Homo sapiens
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSPRPSEKLFFGSGTQLSVLEDLNKVFPPEVAVFEPSEAEISHTQKATLVCLATGFYPDHVELSWWVNGKEVHSGVSTDPQPLKEQPALNDSRYCL

SEQ ID NO: 625 - DNA, 607 bp, Homo sapiens
atttcaaaagcatgaaaattactagaggacttgcatttgattggttgagcagccaacagaaaggctctttcctgcattacagaatatgaactaagatacagaagtggcgcctctgagaaaagaaggttggaattatcgtaatttgtttctaggctgagataccagcatggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgcggccataggaagctacatacctacatttggaagaggaaccagccttattgttcatccgtatatccagaaccctgaccctgccgtgtaccagctgagagactagatcggg

SEQ ID NO: 626 - DNA, 993 bp, Homo sapiens
agagtagtgatatcacagcctaacatccgatcagagatgcaatgcccaaaaccccagctctcagaggaccagtatccctcacagggtgacacctgaccagctctgtcccacctggccatgggctccaggtacctctgatgggaagacctttgtctcttgggaacaagtgaatccttggcacaggcccagtggattctgctgtgcagaacagagagcagtggacctcaggaggcctgcaaggggaggacataggacagtgacatcacagtatgcccctcccaccaggaaaagcaaggctgagaatttagctctttcccaggaggaccaagccctgagcacagacacagtgctgcctgcccctttgtgccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttggggaattcgaacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 627 - DNA, 656 bp, Homo sapiens
gagaggaggaggaggaggaggaggaaggatgaggggaggggcttagtggtgaattaggggtgttaaaaagagcatcatttttttgaactggtaaagcagattctttttatgatttttaaagtagaaatatccattccaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgagggagtcaggaggaggtgctgacggactcacctttggcaaagggactcatctaatcatccagccctatatccagaaccctgaccctgccgtgtaccagctgagagactctaaatccagtgac

SEQ ID NO: 628 - DNA, 612 bp, Homo sapiens
gacctgttagaggagggaaacagagtgggaaggaactacgctgtaggaaggaaagatgaacttgagtttcacttcttagtgccttttctcaggggagaggccatcacttgaagatgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttggggaacagggtatgacgcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttct

SEQ ID NO: 629 - DNA, 599 bp, Homo sapiens
gaggaagctgctctatctttagctgacgatttctggggagcactcaaattgaaacctgcctgatgtgggatgtgctgtggctgctgctttgttgcttgggacctcctctgacctaggatcagacacagagtctgagttctggggcctggaacctcaatgtgcacttgaacaatgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagatatcagggagcccagaagctggtatttggccaaggaaccaggctgactatcaacccaaatatccagaaccctgaccctgccgtgtaccagctgagagactagat

SEQ ID NO: 630 - DNA, 847 bp, Homo sapiens
gattcatcctagcccagcaaattcaaatctaccttctatcagaacttagaaaggatgtaaagcagtcaggaagaaacatcccctgggtctggggaaactatcaggagcagtgacatcacaagaaaaaccaccaaccagggccaaggagaccagagcccagcacctcgcccaaaggaccccagtcagaggccccatctcagacccgaggctagcatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaagtaagggagcgcaagaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 631 - DNA, 700 bp, Homo sapiens
cattcctcagtctggccacaggggagagctgtttaaaaagtattctaggtagatgcatttagttctctgcagttaaactgttgtgatgtgtctctgattggttgcccccaaagttgcaatatcctggaagcaattgcaacaggcctcattctgagttcaaagcaactcctgttaaggaagcccattcagaagctgactggatattctggcaggccaaggatggagactctcctgaaagtgctttcaggcaccttgttgtggcagttgacctgggtgagaagccaacaaccagtgcagagtcctcaagccgtgatcctccgagaaggggaagatgctgtcatcaactgcagttcctccaaggctttatattctgtacactggtacaggcagaagcatggtgaagcacccgtcttcctgatgatattactgaagggtggagaacagaagggtcatgaaaaaatatctgcttcatttaatgaaaaaaagcagcaaagctccctgtaccttacggcctcccagctcagttactcaggaacctacttctgcggcactggggctgggagttaccaactcactttcgggaaggggaccaaactctcggtcataccaaatatccagaaccctgaccctgccgtgtaccagctgagagactctaaatccagtgacaagtctgtctgcctattcaccgattttgattctc

SEQ ID NO: 632 - DNA, 759 bp, Homo sapiens
gcagaacttagaaaggatgtaaagcagtcaggaagaaacatcccctgggtctggggaaactatcaggagcagtgacatcacaagaaaaaccaccaaccagggccaaggagaccagagcccagcacctcgcccaaaggaccccagtcagaggccccatctcagacccgaggctagcatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaccccggcttaagggcgggagcctcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcac

SEQ ID NO: 633 - DNA, 617 bp, Homo sapiens
ctatttccctttttctgagtgtgtgtctgtgtgcatgtgtgtgtgcacgcacgcgcacacaggcagacgctaggggtagagtgtgatggttcaaaaagaaaaggaaacgcttaaaggtagtgaatcacgttttgcccaggaaaacacacttgataactgaaggatgatgaagtgtccacaggctttactagctatcttttggcttctactgagctgggtgagcagtgaagacaaggtggtacaaagccctctatctctggttgtccacgagggagacactgtaactctcaattgcagttatgaagtgactaactttcgaagcctactatggtacaagcaggaaaagaaagctcccacatttctatttatgctaacttcaagtggaattgaaaagaagtcaggaagactaagtagcatattagataagaaagaacttttcagcatcctgaacatcacagccacccagaccggagactcggccgtctacctctgtgctgtcttgaacaccggtaaccagttctattttgggacagggacaagtttgacggtcattccaaatatccagaaccctgaccctgccgtgtaccagctgagagactctaaatccagtgacaagt

SEQ ID NO: 634 - DNA, 713 bp, Homo sapiens
agaaaggatgtaaagcagtcaggaagaaacatcccctgggtctggggaaactatcaggagcagtgacatcacaagaaaaaccaccaaccagggccaaggagaccagagcccagcacctcgcccaaaggaccccagtcagaggccccatctcagacccgaggctagcatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagtcggtttccgggcgggggctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatg

SEQ ID NO: 635 - DNA, 753 bp, Homo sapiens
cgtgtgtgtgtgtgtctgtgtgtgtgtgtgtgtgcttgagagagagagaaggagagaaagagagagaatgggaagggcgataagaggaggggttagtgatataccaggggttgtgaaaataacctcttttttctaattggtaggacagattctttttacgattcctaaagtggaagaaataaagtatctctgctatgttcatttctttttggattgaaaattttaatcctcagtgaaccagggcagaaaagaatgatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaactggtccggaaatgagaaattaacctttgggactggaacaagactcaccatcatacccaatatccagaaccctgaccctgccgtgtaccagctgagagacttaaagacaacccaaaccagagtcttttgacaggatatattcagacagatctgcgtgggatga

SEQ ID NO: 636 - DNA, 627 bp, Homo sapiens
gcatctttctttaagacccagtaccttgggagggacaatgacatcacttcctgaatcctcccagttttctatttccatgccctgcttccctcaacatccagagctggaaacacctccatcctgcctcttcatgccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccaggggacagggggtcggggagctgttttttggagaaggctctaggctgaccgtactggaggacctgaaaaacgtgttcccacccgaggtcgctgtgtttgagccatcagaagcagagatctcccacacccaaaaggccacactggtgtgcctggcc

SEQ ID NO: 637 - DNA, 683 bp, Homo sapiens
atgtgcacttgcaagtgcacacacacacatgaataagagcaagacagagggagagatgagggaggagcttaatgatggagcagaggtgttaaaaagaacatcctttttctaattggtaggacagatttcttttatgattcctacagcagaaaaatgagaaacgtttgttattatttttttttcgtgtttaaggtttgaatcctcagtgaaccagggcagaaaagaatgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagcgtacaggctgcaggcaacaagctaacttttggaggaggaaccagggtgctagttaaaccaaatatccagaaccctgaccctgccgtgtaccagctgagagactatgatggaccatg

SEQ ID NO: 638 - DNA, 736 bp, Homo sapiens
aatgatgtcactgtgggaactgccatgagaggacagggacgtccctcctcctttgctgttgctcacagtgaccctgattgggcaaagctcccatccttccctgaccctgccatgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagcttagacaggaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 639 - DNA, 650 bp, Homo sapiens
ggggactgtgatttcttcatgttaaggatcaagaccattatttgggtaacacactaaagatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgagtgataacaccaatgcaggcaaatcaacctttggggatgggactacgctcactgtgaagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctgg

SEQ ID NO: 640 - DNA, 754 bp, Homo sapiens
gtcatgggcaaagattaccaccagggggcagactagggcatccttgggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaggactagcggtcaatacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 641 - DNA, 542 bp, Homo sapiens
aacacaaactcatttcctgctatcactgtaggtttcactgtgatttcttcatgttaaggatcaagaccattatttgggtaacacactaaagatgaactattctccaggcttagtatctctgatactcttactgcttggaagaacccgtggagattcagtgacccagatggaagggccagtgactctctcagaagaggcctgcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaggaaaccacttctttccacttggagaaaggctcagttcaagtgtcagactcagcggtgtacttctgtgctctgagtgatctgttggactctggcaacacaggcaaactaatctttgggcaagggacaactttacaagtaaaaccagatatccagaaccctgaccctgccgtgtaccagctgagagactaga

SEQ ID NO: 642 - DNA, 775 bp, Homo sapiens
gtactgtgttcagtcctcctcacctttagcttctggcctgtgcaagacaggaatagtacatggagtagcagaacctgaggcctgccaaagagtacagtgggctggagtggcctgcaggggaaagtatgtcagtgtggtgacatcacaggtcaatgctcctatcaggaagaagggagcttaggaacttcagaatgcttactacagagacaccagccccaagctaggagatcctgccatgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagtagaggatcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaag

SEQ ID NO: 643 - DNA, 694 bp, Homo sapiens
agtgctcgtgtgtgtgtgtgtctgtgtgtgtgtgtgtgtgcttgagagagagagaaggagagaaagagagagaatgggaagggcgataagaggaggggttagtgatataccaggggttgtgaaaataacctcttttttctaattggtaggacagattctttttacgattcctaaagtggaagaaataaagtatctctgctatgttcatttctttttggattgaaaattttaatcctcagtgaaccagggcagaaaagaatgatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaacactccctcttccaagataatctttggatcagggaccagactcagcatccggccaaatatccagaaccctgaccctgccgtgtaccagctgagagact

SEQ ID NO: 644 - DNA, 726 bp, Homo sapiens
gagggggcagactagggcatccttgggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagatcgggacaggggcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctg

SEQ ID NO: 645 - DNA, 890 bp, Homo sapiens
ggctcgtgtgtatgtgtgtgtgtgtgtgtgtgtgtgtgtgtgtgcttgagagagagagaaggagagaaagagagagaatgggaagggcgataagaggaggggttagtgatataccaggggttgtgaaaataaccttttttttctaattggtaggacagattctttttatgattcctaaagtggaagaaataaagtatctctgctatgttcatttctttttggattgaaaattttaatcctcagtgaaccagggcagaaaagaatgatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgcctcctgggggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttca

SEQ ID NO: 646 - DNA, 814 bp, Homo sapiens
ggacattgattacaaatgtcctcagcctgagttggctgtgttgcgtttgtacacttcagccctctgagctgaagtgggagtggtttctctcctgaaaatgtttctgaggcccaaatagctgaagaggtggagacgttacagaaaccacctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcaggaccaggacagccagttatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctga

SEQ ID NO: 647 - DNA, 978 bp, Homo sapiens
aactccgcctcccgggttcccgccattctcctgcctcagcctctcgagtagctgggactacaggcgcccgccacagcgcccagctaatttttttgtattttttggtagagacggggtttcaccgtgttagccaggatggtctcgatctcctgacctcgtgatccgcccacctcggcctcccaaagcgctgggattaaaggcgtgagccaccgcgcccggcctactttgtcttatgttattcccatttgccgtctctgttccttatacattatgctttttcaactttaccagaatcacttggattaaaacccgtggatttctcagtaggaaatgttcatgtgaagacacttctgtagtaacagaacctacagctgctcctgtagaaggaagttgaaagtcatcccttcaagaaaggggctcctccccttgtaattctactgggttttgcatccagactgagtttccttccctcacccacatgaagtgtctaccttctgcagactccaatggctcaggaactgggaatgcagtgccaggctcgtggtatcctgcagcagatgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtcctggatagcaactatcagttaatctggggcgctgggaccaagctaattataaagccagatatccagaaccctgaccctgccgtgtaccagctgagagact

SEQ ID NO: 648 - DNA, 599 bp, Homo sapiens
tgggggaaggggcgtggcctctcctgacaggaaggctctggggcccaggcagggagaatgaagtctcagaatgacccccttgagagtactgttcccctatcaccgatgcacagacccagaagacccctccatcctgtagcacctgccatgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgaagaggctagcgggtcctacgagcagtacttcgggccgggcaccaggctcacggtcacagaggacctgaaaaacgtgttcccacccgaggtcgctgtgtttgagccatcaga

SEQ ID NO: 649 - DNA, 671 bp, Homo sapiens
tgtgtgtgtgtgtgtgtgtgtgtgtgagagagagagagagagagagaggaggaggaggaggaggaggaaggatgaggggaggggcttagtggtgaattaggggtgttaaaaagagcatcatttttttgaactggtaaagcagattctttttatgatttttaaagtagaaatatccattccaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgagtttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtctcagggaaccagttctattttgggacagggacaagtttgacggtcattccaaatatccagaaccctgaccctgccgtgtaccagctgagagacta

SEQ ID NO: 650 - DNA, 740 bp, Homo sapiens
ggtcactgtgggaactgctctgtggcgacaaggacgtccctcatcctgtgctcctgctcacagtgaccctgatctggtaaagctcccatcctgccctgaccctgccatgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccctgggtagcgggaggtacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga

SEQ ID NO: 651 - DNA, 826 bp, Homo sapiens
atgttttgatacatctcagaaattaggagaaacacactaatgagctctctctgctaaactggttctgcacttggcctccagagggcgatgctgcacacactggagcttttgtttctgttgaagatcaatccactgctcagtttcttcttcctgcagctggttgagttctttccagacaaagacaagtgacaagaattagaggtttaaaaagcaaccagattcatctcagcagcttttgtagttttaaataagcaaggagtttctccagcgaaacttcctcacacctcttggtcttggtctcttcagacactttccttcctgttctctggagatcttgcagaaaagagcctgcagtgtttcccttgttcagccatgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtcccttttctgggggttaccagaaagttacctttggaactggaacaaagctccaagtcatcccaaatatccagaaccctgaccctgccgtgtaccagctgagagactagatcggaa

SEQ ID NO: 652 - DNA, 754 bp, Homo sapiens
tccgtccatttcttatatgggagaccttgcctgtggggccatgggagctcaaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagagaggtacactctggggccaacgtcctgactttcggggccggcagcaggctgaccgtgctggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctg

SEQ ID NO: 653 - DNA, 796 bp, Homo sapiens
gacactggagcttttgtttctgttgaagatcaatccactgctcagtttcttcttcctgcagctggttgagttctttccagacaaagacaagtgacaagaattagaggtttaaaaagcaaccagattcatctcagcagcttttgtagttttaaataagcaaggagtttctccagcgaaacttcctcacacctcttggtcttggtctcttcagacactttccttcctgttctctggagatcttgcagaaaagagcctgcagtgtttcccttgttcagccatgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctttttataaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtg

SEQ ID NO: 654 - DNA, 736 bp, Homo sapiens
taagggggggccagaccttgcctgtggggccatgggagctcaaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagggtcatgaacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctg

SEQ ID NO: 655 - DNA, 876 bp, Homo sapiens
ggcttttgtttctgttgaagatcaatccactgctcagtttcttcttcctgcagctggttgagttctttccagacaaagacaagtgacaagaattagaggtttaaaaagcaaccagattcatctcagcagcttttgtagttttaaataagcaaggagtttctccagcgaaacttcctcacacctcttggtcttggtctcttcagacactttccttcctgttctctggagatcttgcagaaaagagcctgcagtgtttcccttgttcagccatgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggcttcttggaatatggaaacaaactggtctttggcgcaggaaccattctgagagtcaagtcctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctg

SEQ ID NO: 656 - DNA, 868 bp, Homo sapiens
ggatcagagatgcaatgcccaaaaccccagctctcagaggaccagtatccctcacagggtgacacctgaccagctctgtcccacctggccatgggctccaggtacctctgatgggaagacctttgtctcttgggaacaagtgaatccttggcacaggcccagtggattctgctgtgcagaacagagagcagtggacctcaggaggcctgcaaggggaggacataggacagtgacatcacagtatgcccctcccaccaggaaaagcaaggctgagaatttagctctttcccaggaggaccaagccctgagcacagacacagtgctgcctgcccctttgtgccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagctccctagcgggagccaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgag

SEQ ID NO: 657 - DNA, 530 bp, Homo sapiens
atatggggagtgttaaaaaaaaagagaagatgttgaatacacaagtcaacttctgggagcagatctctgcagaataaaaatgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacagaagatatacagcaactctggatgcagacacaaagcaaagctctctgcacatcacagcctcccagctcagcgattcagcctcctacatctgtgtggtgagcgcgggatatggaggaagccaaggaaatctcatctttggaaaaggcactaaactctctgttaaaccaaatatccagaaccctgaccctgccgtgtaccagctgagagact

SEQ ID NO: 658 - DNA, 617 bp, Homo sapiens
tggggtcactgtgggaactgctctgtggcgacaaggacgtccctcatcctctgctcctgctcacagtgaccctgatctggtaaagctcccatcctgccctgaccctgccatgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcacccctgggggctcctcccgcgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttct

SEQ ID NO: 659 - DNA, 579 bp, Homo sapiens
gggggacttcctgtaaaaggcagaagcctcacacagcccagtaactttgctagtacctcttgagtgcaaggtggagaattaagatctggatttgagacggagcacggaacatttcactcaggggaagagctatgaacatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtacccatggaaacaaactggtctttggcgcaggaaccattctgagagtcaagtcctatatccagaaccctgaccctgccgtgtaccagctgagagact

SEQ ID NO: 660 - DNA, 607 bp, Homo sapiens
atgggggatgtcactgtgggaactgctctgtggcgacaaggacgtccctcatcctgtgctcctgctcacagtgaccctgatctggtaaagctcccatcctgccctgaccctgccatgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagacagggaaaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctgg

SEQ ID NO: 661 - DNA, 517 bp, Homo sapiens
gtcactgacacaaagaaaaaaatgcaaaacaggtagtcttaaataagcattctggtgagaccaactgcattttggccatggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggaggatagggataacaatgccagactcatgtttggagatggaactcagctggtggtgaagcccaatatccagaaccctgaccctgccgtgtaccagctgagagactc

SEQ ID NO: 662 - DNA, 585 bp, Homo sapiens
tatggggactgagaacctaagttctatttccccaggcagggctgggagagatgagatcctggcctggacctgaaatgggcacaaggttgttcttctatgtggccctttgtctcctgtggacaggacacatggatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgaacggccggtcggggagcaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttct

SEQ ID NO: 663 - DNA, 588 bp, Homo sapiens
ccttatttgggggcatttgattggttgagcagccaacagaaaggctctttcctgcattacagaatatgaactaagatacagaagtggcgcctctgagaaaagaaggttggaattatcgtaatttgtttctaggctgagataccagcatggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcagatcggagggtatggaaacaaactggtctttggcgcaggaaccattctgagagtcaagtcctatatccagaaccctgaccctgccgtgtaccagctgagagactaga

SEQ ID NO: 664 - DNA, 702 bp, Homo sapiens
ggtcacaaaattcatttctttgctcatgttcacagagggcctggtctggaatattccacatctgctctcactctgccatggactcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagcccccggccgagtgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctga